SEQUENCE LISTING (153 sequences)

SEQ ID NO: 1 - protein, 189 aa, Homo sapiens [sequence omitted]

SEQ ID NO: 2 - protein, 10 aa, Homo sapiens
  source 1..10: note = KRAS 7-16 G12V
VVVGAVGVGK

SEQ ID NO: 3 - protein, 9 aa, Homo sapiens
  source 1..9: note = KRAS 8-16 G12V
VVGAVGVGK

SEQ ID NO: 4 - protein, 9 aa, synthetic construct
  source 1..9: note = KRAS 8-16 G12V binding motif for TCR 11N4A
XVGAXGXXK

SEQ ID NO: 5 - DNA, 843 bp, synthetic construct
  source 1..843: mol_type = other DNA; note = TCR 11N4A alpha chain with signal peptide original (WT) nucleotide sequence
atggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaagtggggtttcaggaaacacacctcttgtctttggaaagggcacaagactttctgtgattgcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 6 - DNA, 930 bp, synthetic construct
  source 1..930: mol_type = other DNA; note = TCR 11N4A beta chain with signal peptide original (WT) nucleotide sequence
atgggctccaggctgctctgttgggtgctgctttgtctcctgggagcaggcccagtaaaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcagcgtcgggactgtggagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 7 - DNA, 1836 bp, synthetic construct
  source 1..1836: mol_type = other DNA; note = TCR 11N4A TCRbeta-P2A-TCRalpha polynucleotide - Codon-optimization A
atgggctctagactgttgtgttgggttctgctgtgtctgcttggagctggacctgtgaaagctggagttacccagacacccagatatctgatcaagaccagaggacagcaggtgacactgagctgtagccctatttctggccacaggagcgttagctggtatcagcaaacacccgggcagggactacaatttctattcgagtacttcagcgagacccagcggaataagggcaattttcctggcagatttagcggcaggcagttcagcaacagcagaagcgagatgaacgtgagcaccctggaattaggcgattctgctctgtacctgtgtgcctcttctgtgggaacagtggagcagtactttggccccggcacgagactgacagtgacagaggacctgaagaacgtgttccccccagaggtggccgtgttcgagcctagcgaggccgagatcagccacacccagaaagccaccctcgtgtgcctggccaccggcttttaccccgaccacgtggaactgtcttggtgggtcaacggcaaagaggtgcacagcggcgtctgcaccgacccccagcccctgaaagagcagcccgccctgaacgacagccggtactgtctgagcagcagactgagagtgtccgccaccttctggcagaacccccggaaccacttcagatgccaggtgcagttctacggcctgagcgagaacgacgagtggacccaggaccgggccaagcccgtgacccagatcgtgtctgctgaggcctggggcagagccgattgcggcttcaccagcgagagctaccagcagggcgtgctgagcgccaccatcctgtacgagatcctgctgggcaaggccaccctgtacgccgtgctggtgtccgccctggtgctgatggccatggtcaagcggaaggacagccggggcggttccggagccacgaacttctctctgttaaagcaagcaggagacgtggaagaaaaccccggtcccatggccatgttactaggagcgagcgtgctgattctgtggttacagcctgattgggtgaactctcagcagaagaacgacgatcagcaggtgaagcagaatagcccctctctgtctgtgcaggagggcagaatctctatcctgaattgcgactacaccaacagcatgttcgactattttctgtggtacaaaaaataccccgccgagggccctacattcctgatcagcatcagctctatcaaggacaagaacgaggatggcagatttaccgtgttcctgaacaagagcgccaagcacctgagcctgcacattgtgccttctcaacctggcgattctgctgtgtacttttgtgctgcctctggagtgagcggcaatacacctctagtgttcgggaagggcacaagactgtctgttattgcaaacattcaaaaccccgaccctgctgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcattatccccgaggacacattcttcccaagccccgagagcagctgcgacgtgaagctggtggaaaagagcttcgagacagacaccaacctgaacttccagaacctcagcgtgatcggcttccggatcctgctgctgaaggtggccggcttcaacctgctgatgaccctgcggctgtggtccagctga

SEQ ID NO: 8 - DNA, 1836 bp, synthetic construct
  source 1..1836: mol_type = other DNA; note = 11N4A TCRbeta-P2A-alpha polynucleotide Codon-optimization B
atgggatctagattgctttgttgggtgctgctgtgcctgctcggagccggacctgtgaaagctggcgttacccagacacctagatacctgatcaagaccagaggccagcaagtgaccctgagctgctctcctatcagcggccacagaagcgtgtcctggtatcagcagacacctggacagggcctgcagttcctgttcgagtacttcagcgagacacagcggaacaagggcaacttccccggcagattttccggcagacagttcagcaacagccgcagcgagatgaacgtgtccacactggaactgggcgacagcgccctgtatctgtgtgcctcttctgtgggcaccgtggaacagtactttggccctggcaccagactgaccgtgaccgaggatctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtctgtaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggctttaccagcgagagctaccagcagggcgtgctgtctgccacaatcctgtacgagatcctgctgggcaaagccactctgtacgccgtgctggtttctgccctggtgctgatggccatggtcaagcggaaggattctagaggcggatccggagccaccaacttcagcctgcttaaacaggccggcgacgtggaagagaaccctggacctatggctatgctgctgggagcctctgtgctgatcctgtggctgcaacccgattgggtcaacagccagcagaagaacgacgaccagcaagtcaagcagaacagccccagcctgagcgtgcaagagggcagaatcagcatcctgaactgcgactacaccaactctatgttcgactactttctgtggtacaagaagtaccccgccgagggacccaccttcctgatcagcatcagcagcatcaaggacaagaacgaggacggccggttcaccgtgtttctgaacaagagcgccaagcacctgagcctgcacatcgtgccttctcagcctggcgatagcgccgtgtacttttgtgctgccagcggcgtgtcaggcaacacccctctggtttttggcaagggcacacgcctgtccgtgatcgccaacattcagaaccctgatcctgccgtgtaccagctgagagacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagtccgatttcgcctgcgccaacgccttcaacaacagcattatccccgaggacacattcttcccaagtcctgagtccagctgcgacgtgaagctggtggaaaagagcttcgagacagacaccaacctgaacttccagaatctgagcgtgatcggcttcagaatcctgctgctgaaggtggccggattcaacctgctgatgaccctcagactgtggtccagctga

SEQ ID NO: 9 - DNA, 3300 bp, synthetic construct
  source 1..3300: mol_type = other DNA; note = CD8alpha-T2A-CD8beta-P2A-11N4A TCRbeta-P2A-alpha polynucleotide Codon-optimization A
atggctctgcctgtgacagctctgctgctgcctctggctctgcttctgcatgccgctagacccagccagttcagagtgtcccctctggacagaacctggaacctgggcgagacagtggaactgaagtgccaggtgctgctgagcaatcctaccagcggctgcagctggctgtttcagcctagaggtgctgccgcctctcctacctttctgctgtacctgagccagaacaagcccaaggccgccgaaggactggacacccagagattcagcggcaagagactgggcgacaccttcgtgctgaccctgagcgacttcagaagagagaacgagggctactacttctgcagcgccctgagcaacagcatcatgtacttcagccacttcgtgcccgtgtttctgcccgccaagcctacaacaacccctgctcctagacctcctacaccagctcctacaatcgccagccagcctctgtctctgaggccagaagcttgtagacctgctgctggcggagccgtgcatacaagaggactggatttcgcctgcgacatctacatctgggcccctctggctggaacatgtggcgtgctgctgctgtccctggtcatcaccctgtactgcaaccaccggaacaggcggagagtgtgcaagtgccctagacctgtggtcaagagcggcgacaagcctagcctgagcgccagatatgttggcagcggagaaggcagaggctccctgcttacatgcggcgacgtggaagagaaccccggacctatgaggcctagactgtggctgcttctggctgcccagctgacagtgctgcacggcaattctgtcctgcagcagacccctgcctacatcaaggtgcagaccaacaagatggtcatgctgagctgcgaggccaagatcagcctgtccaacatgcggatctactggctgcggcagagacaggcccctagctctgatagccaccacgagtttctggccctgtgggattctgccaagggcaccattcacggcgaggaagtggaacaagagaagatcgccgtgttccgggacgccagcagattcatcctgaacctgaccagcgtgaagcccgaggacagcggcatctatttctgcatgatcgtgggcagccccgagctgacatttggcaagggaacacagctgagcgtggtggacttcctgcctactacagcccagcctaccaagaagtctaccctgaagaaacgcgtgtgcagactgcccaggcctgagacacaaaagggccctctgtgcagccctatcacactgggattgctggtggctggcgttctggtcctgctggtgtctctgggagttgccatccacctgtgctgtagaagaaggcgggccagactgcggttcatgaagcagttctacaaaggcagcggcgccaccaacttcagcctgctgaaacaagccggcgacgtcgaggaaaatcctggacctatgggctctagactgttgtgttgggttctgctgtgtctgcttggagctggacctgtgaaagctggagttacccagacacccagatatctgatcaagaccagaggacagcaggtgacactgagctgtagccctatttctggccacaggagcgttagctggtatcagcaaacacccgggcagggactacaatttctattcgagtacttcagcgagacccagcggaataagggcaattttcctggcagatttagcggcaggcagttcagcaacagcagaagcgagatgaacgtgagcaccctggaattaggcgattctgctctgtacctgtgtgcctcttctgtgggaacagtggagcagtactttggccccggcacgagactgacagtgacagaggacctgaagaacgtgttccccccagaggtggccgtgttcgagcctagcgaggccgagatcagccacacccagaaagccaccctcgtgtgcctggccaccggcttttaccccgaccacgtggaactgtcttggtgggtcaacggcaaagaggtgcacagcggcgtctgcaccgacccccagcccctgaaagagcagcccgccctgaacgacagccggtactgtctgagcagcagactgagagtgtccgccaccttctggcagaacccccggaaccacttcagatgccaggtgcagttctacggcctgagcgagaacgacgagtggacccaggaccgggccaagcccgtgacccagatcgtgtctgctgaggcctggggcagagccgattgcggcttcaccagcgagagctaccagcagggcgtgctgagcgccaccatcctgtacgagatcctgctgggcaaggccaccctgtacgccgtgctggtgtccgccctggtgctgatggccatggtcaagcggaaggacagccggggcggttccggagccacgaacttctctctgttaaagcaagcaggagacgtggaagaaaaccccggtcccatggccatgttactaggagcgagcgtgctgattctgtggttacagcctgattgggtgaactctcagcagaagaacgacgatcagcaggtgaagcagaatagcccctctctgtctgtgcaggagggcagaatctctatcctgaattgcgactacaccaacagcatgttcgactattttctgtggtacaaaaaataccccgccgagggccctacattcctgatcagcatcagctctatcaaggacaagaacgaggatggcagatttaccgtgttcctgaacaagagcgccaagcacctgagcctgcacattgtgccttctcaacctggcgattctgctgtgtacttttgtgctgcctctggagtgagcggcaatacacctctagtgttcgggaagggcacaagactgtctgttattgcaaacattcaaaaccccgaccctgctgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcattatccccgaggacacattcttcccaagccccgagagcagctgcgacgtgaagctggtggaaaagagcttcgagacagacaccaacctgaacttccagaacctcagcgtgatcggcttccggatcctgctgctgaaggtggccggcttcaacctgctgatgaccctgcggctgtggtccagctga

SEQ ID NO: 10 - DNA, 3300 bp, synthetic construct
  source 1..3300: mol_type = other DNA; note = CD8alpha-T2A-CD8beta-P2A-11N4A TCRbeta-P2A-alpha polynucleotide Codon-optimization B
atggcattgcctgttacagctctgctgctgcccctggctctgcttctgcatgctgctagacccagccagttcagagtgtcccctctggacagaacctggaacctgggcgagacagtggaactgaagtgccaggtgctgctgagcaatcctaccagcggctgcagctggctgtttcagcctagaggtgctgccgcctctcctacctttctgctgtacctgagccagaacaagcccaaggccgccgaaggactggacacccagagattcagcggcaagagactgggcgacaccttcgtgctgaccctgagcgacttcagaagagagaacgagggctactacttctgcagcgccctgagcaacagcatcatgtacttcagccacttcgtgcccgtgtttctgcccgccaagcctacaacaacccctgctcctagacctcctacaccagctcctacaatcgccagccagcctctgtctctgaggccagaagcttgtagacctgctgctggcggagccgtgcatacaagaggactggatttcgcctgcgacatctacatctgggcccctctggctggaacatgtggcgtgctgctgctgtctctggtcatcaccctgtactgcaaccaccggaacaggcggagagtgtgcaagtgccctagacctgtggtcaagagcggcgacaagcctagcctgagcgccagatatgttggcagcggagaaggcagaggcagcctgcttacatgcggcgacgtggaagagaaccccggacctatgaggcctagactgtggctgcttctggctgcccagctgacagtgctgcacggcaattctgtcctgcagcagacccctgcctacatcaaggtgcagaccaacaagatggtcatgctgagctgcgaggccaagatcagcctgtccaacatgcggatctactggctgcggcagagacaggcccctagcagcgattctcaccacgagtttctggccctgtgggatagcgccaagggaaccattcacggcgaggaagtggaacaagagaagatcgccgtgttccgggacgccagcagattcatcctgaacctgaccagcgtgaagcccgaggacagcggcatctatttctgcatgatcgtgggcagccccgagctgacatttggcaagggaacacagctgagcgtggtggacttcctgcctactacagcccagcctaccaagaagtctaccctgaagaaacgcgtgtgcagactgcccaggcctgagacacaaaagggccctctgtgcagccctatcacactgggattgctggtggctggcgttctggtcctgctggtttctctgggagttgccatccacctgtgctgcagacgcagaagggccagactgcggttcatgaagcagttctacaaaggcagcggcgccaccaacttcagcctgctgaaacaagccggcgacgtcgaagaaaatcctggaccaatgggcagcagactgctgtgctgggttctgctgtgtctgcttggagccggacctgtgaaagctggcgtgacccagacacctagatacctgatcaagaccagaggccagcaagtgacactgagctgtagccccatcagcggccacagaagcgtgtcctggtatcagcagactcctggacagggcctgcagttcctgttcgagtacttctccgagacacagaggaacaagggcaacttccccggcagattctccggcagacagttcagcaactcccgcagcgagatgaacgtgtccacactggaactgggagatagcgccctgtacctgtgtgcctcttctgtgggaaccgtggaacagtacttcggccctggcacaagactgaccgtgaccgaggacctgaagaacgtgttcccacctgaggtggccgtgttcgagccttctgaggccgagatctctcacacccagaaagccacactcgtgtgtctggccaccggcttctatcccgatcacgtggaactgtcttggtgggtcaacggcaaagaggtgcacagcggcgtctgtaccgatcctcagccactgaaagagcagcccgctctgaacgacagcagatactgcctgtcctccagactgagagtgtccgccaccttctggcagaaccccagaaaccacttcaggtgtcaggtgcagttttacggcctgagcgagaacgacgagtggacccaggatagagccaagcctgtgacacagatcgtgtctgccgaagcctggggcagagccgattgtggctttaccagcgagagctaccagcagggcgttctgtctgccaccatcctgtacgagatcctgctgggcaaagccactctgtacgccgtgttggtgtctgccctggtgctgatggccatggtcaagcggaaggattctagaggcggatccggagccacaaatttctcactgctgaagcaggccggggatgttgaggaaaacccaggacctatggctatgctgctgggagcctctgtgctgatcctgtggctgcaacccgattgggtcaacagccagcagaagaacgacgaccagcaagtcaagcagaacagccccagcctgagcgtgcaagagggcagaatcagcatcctgaactgcgactacaccaactctatgttcgactactttctgtggtacaagaagtaccccgccgagggacccaccttcctgatcagcatcagcagcatcaaggacaagaacgaggacggccggttcaccgtgtttctgaacaagagcgccaagcacctgagcctgcacatcgtgccttctcagcctggcgatagcgccgtgtacttttgtgctgccagcggcgtgtcaggcaacacccctctggtttttggcaagggcacacgcctgtccgtgatcgccaacattcagaaccctgatcctgccgtgtaccagctgagagacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagtccgatttcgcctgcgccaacgccttcaacaacagcattatccccgaggacacattcttcccaagtcctgagtccagctgcgacgtgaagctggtggaaaagagcttcgagacagacaccaacctgaacttccagaatctgagcgtgatcggcttcagaatcctgctgctgaaggtggccggattcaacctgctgatgaccctcagactgtggtccagctga

SEQ ID NO: 11 - protein, 280 aa, synthetic construct
  source 1..280: note = 11N4A TCR alpha chain original protein, with signal peptide
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASGVSGNTPLVFGKGTRLSVIANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 12 - protein, 259 aa, synthetic construct
  source 1..259: note = 11N4A TCR alpha chain original protein, without signal peptide
QQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASGVSGNTPLVFGKGTRLSVIANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 13 - protein, 118 aa, synthetic construct
  source 1..118: note = 11N4A TCR alpha chain variable domain, without signal peptide
QQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASGVSGNTPLVFGKGTRLSVIA

SEQ ID NO: 14 - protein, 6 aa, synthetic construct
  source 1..6: note = 11N4A TCR alpha chain variable domain CDR1alpha
NSMFDY

SEQ ID NO: 15 - protein, 7 aa, synthetic construct
  source 1..7: note = 11N4A TCR alpha chain variable domain CDR2alpha
ISSIKDK

SEQ ID NO: 16 - protein, 14 aa, synthetic construct
  source 1..14: note = 11N4A TCR alpha chain variable domain CDR3alpha IMGT junction
CAASGVSGNTPLVF

SEQ ID NO: 17 - protein, 12 aa, synthetic construct
  source 1..12: note = 11N4A TCR alpha chain variable domain CDR3alpha IMGT
AASGVSGNTPLV

SEQ ID NO: 18 - protein, 141 aa, synthetic construct
  source 1..141: note = 11N4A TCR alpha chain constant domain (original protein)
NIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 19 - protein, 141 aa, synthetic construct
  source 1..141: note = 11N4A TCR alpha chain constant domain (cys-modified protein)
NIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 20 - protein, 259 aa, synthetic construct
  source 1..259: note = 11N4A TCR alpha chain, without signal peptide, cys-modified
QQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASGVSGNTPLVFGKGTRLSVIANIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 21 - protein, 309 aa, synthetic construct
  source 1..309: note = 11N4A TCR beta chain - original protein, with signal peptide
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSVGTVEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 22 - protein, 288 aa, synthetic construct
  source 1..288: note = 11N4A TCR beta chain original protein, without signal peptide
GVTQTPRYLIKTRGQQVTLSCSPISGHRSVSWYQQTPGQGLQFLFEYFSETQRNKGNFPGRFSGRQFSNSRSEMNVSTLELGDSALYLCASSVGTVEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 23 - protein, 109 aa, synthetic construct
  source 1..109: note = 11N4A TCR beta chain variable domain, without signal peptide
GVTQTPRYLIKTRGQQVTLSCSPISGHRSVSWYQQTPGQGLQFLFEYFSETQRNKGNFPGRFSGRQFSNSRSEMNVSTLELGDSALYLCASSVGTVEQYFGPGTRLTVT

SEQ ID NO: 24 - protein, 5 aa, synthetic construct
  source 1..5: note = 11N4A TCR beta chain variable domain CDR1beta
SGHRS

SEQ ID NO: 25 - protein, 6 aa, synthetic construct
  source 1..6: note = 11N4A TCR beta chain variable domain CDR2beta
YFSETQ

SEQ ID NO: 26 - protein, 12 aa, synthetic construct
  source 1..12: note = 11N4A TCR beta chain variable domain CDR3beta - IMGT junction
CASSVGTVEQYF

SEQ ID NO: 27 - protein, 10 aa, synthetic construct
  source 1..10: note = 11N4A TCR beta chain variable domain CDR3beta - IMGT
ASSVGTVEQY

SEQ ID NO: 28 - protein, 179 aa, synthetic construct
  source 1..179: note = 11N4A TCR beta chain constant domain (original protein)
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 29 - protein, 179 aa, synthetic construct
  source 1..179: note = 11N4A TCR beta chain constant domain (cys-modified protein)
EDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 30 - protein, 288 aa, synthetic construct
  source 1..288: note = 11N4A TCR beta chain, without signal peptide (cys-modified protein)
GVTQTPRYLIKTRGQQVTLSCSPISGHRSVSWYQQTPGQGLQFLFEYFSETQRNKGNFPGRFSGRQFSNSRSEMNVSTLELGDSALYLCASSVGTVEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 31 - protein, 611 aa, synthetic construct
  source 1..611: note = 11N4A TCRbeta-P2A-alpha Protein, with signal peptides
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSVGTVEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASGVSGNTPLVFGKGTRLSVIANIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 32 - protein, 1099 aa, synthetic construct
  source 1..1099: note = CD8alpha-T2A-CD8beta-P2A-11N4A TCRbeta-P2A-alpha Protein, with signal peptides
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVGSGEGRGSLLTCGDVEENPGPMRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYKGSGATNFSLLKQAGDVEENPGPMGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSVGTVEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASGVSGNTPLVFGKGTRLSVIANIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 33 - DNA, 825 bp, synthetic construct
  source 1..825: mol_type = other DNA; note = 11N6 TCR alpha, original nucleotide sequence
atggaaactctcctgggagtgtctttggtgattctatggcttcaactggctagggtgaacagtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctacggaccctatgaacaccaatgcaggcaaatcaacctttggggatgggactacgctcactgtgaagccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 34 - DNA, 945 bp, synthetic construct
  source 1..945: mol_type = other DNA; note = 11N6 TCR beta, original nucleotide sequence
atgggcaccaggctcctctgctgggcggccctctgtctcctgggagcagaactcacagaagctggagttgcccagtctcccagatataagattatagagaaaaggcagagtgtggctttttggtgcaatcctatatctggccatgctaccctttactggtaccagcagatcctgggacagggcccaaagcttctgattcagtttcagaataacggtgtagtggatgattcacagttgcctaaggatcgattttctgcagagaggctcaaaggagtagactccactctcaagatccaacctgcaaagcttgaggactcggccgtgtatctctgtgccagcagcccctacggggggagcgtctcctacgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 35 - DNA, 1833 bp, synthetic construct
  source 1..1833: mol_type = other DNA; note = 11N6 TCRbeta-P2A-alpha Codon-optimized
atgggcacaagacttctctgttgggctgcactgtgcttgcttggagctgagctgacagaagctggagttgcccaatctcctaggtacaagatcatcgagaagcggcagtctgtggccttttggtgcaatcccattagcggacatgccaccctgtactggtatcagcaaattctgggacagggccctaaactgctgatccagttccagaataacggcgtggtggacgattctcaactgcctaaggaccggttttctgccgagagactgaaaggcgttgatagcaccctgaagatccaacctgccaaactggaggattctgccgtgtacctgtgtgctagcagcccttatggaggatctgtgtcttatgagcagtacttcggacctggcaccagactgaccgtgactgaagacctgaagaacgtgttccccccagaggtggccgtgttcgagcctagcgaggccgagatcagccacacccagaaagccaccctcgtgtgcctggccaccggcttttaccccgaccacgtggaactgtcttggtgggtcaacggcaaagaggtgcacagcggcgtctgcaccgacccccagcccctgaaagagcagcccgccctgaacgacagccggtactgtctgagcagcagactgagagtgtccgccaccttctggcagaacccccggaaccacttcagatgccaggtgcagttctacggcctgagcgagaacgacgagtggacccaggaccgggccaagcccgtgacccagatcgtgtctgctgaggcctggggcagagccgattgcggcttcaccagcgagagctaccagcagggcgtgctgagcgccaccatcctgtacgagatcctgctgggcaaggccaccctgtacgccgtgctggtgtccgccctggtgctgatggccatggtcaagcggaaggacagccggggcggttccggagccacgaacttctctctgttaaagcaagcaggagacgtggaagaaaaccccggtcccatggagacactgcttggcgtatcactggtgattctgtggctgcaactggctagagtgaactctcagcagggagaagaggatcctcaagctctgagcattcaggaaggcgaaaacgcaaccatgaattgctcatacaagaccagcatcaacaacctgcagtggtaccggcagaatagcggaagaggactggttcacctgattttaatcaggtctaatgaaagggagaagcacagcggcagactgagagttaccctggacacatccaagaaatcttcttctctgctgattacagcctctagagccgccgatacagccagctacttttgtgccacagatcccatgaacaccaatgccggaaagagcacattcggcgatggcacaaccctgacagttaagcccaatatccagaatcccgatcctgccgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcattatccccgaggacacattcttcccaagccccgagagcagctgcgacgtgaagctggtggaaaagagcttcgagacagacaccaacctgaacttccagaacctcagcgtgatcggcttccggatcctgctgctgaaggtggccggcttcaacctgctgatgaccctgcggctgtggtccagctga

SEQ ID NO: 36 - DNA, 3297 bp, synthetic construct
  source 1..3297: mol_type = other DNA; note = CD8alpha-T2A-CD8beta-P2A-11N6 TCRbeta-P2A-alpha Codon-optimized
atggctctgcctgtgacagctctgctgctgcctctggctctgcttctgcatgccgctagacccagccagttcagagtgtcccctctggacagaacctggaacctgggcgagacagtggaactgaagtgccaggtgctgctgagcaatcctaccagcggctgcagctggctgtttcagcctagaggtgctgccgcctctcctacctttctgctgtacctgagccagaacaagcccaaggccgccgaaggactggacacccagagattcagcggcaagagactgggcgacaccttcgtgctgaccctgagcgacttcagaagagagaacgagggctactacttctgcagcgccctgagcaacagcatcatgtacttcagccacttcgtgcccgtgtttctgcccgccaagcctacaacaacccctgctcctagacctcctacaccagctcctacaatcgccagccagcctctgtctctgaggccagaagcttgtagacctgctgctggcggagccgtgcatacaagaggactggatttcgcctgcgacatctacatctgggcccctctggctggaacatgtggcgtgctgctgctgtccctggtcatcaccctgtactgcaaccaccggaacaggcggagagtgtgcaagtgccctagacctgtggtcaagagcggcgacaagcctagcctgagcgccagatatgttggcagcggagaaggcagaggctccctgcttacatgcggcgacgtggaagagaaccccggacctatgaggcctagactgtggctgcttctggctgcccagctgacagtgctgcacggcaattctgtcctgcagcagacccctgcctacatcaaggtgcagaccaacaagatggtcatgctgagctgcgaggccaagatcagcctgtccaacatgcggatctactggctgcggcagagacaggcccctagctctgatagccaccacgagtttctggccctgtgggattctgccaagggcaccattcacggcgaggaagtggaacaagagaagatcgccgtgttccgggacgccagcagattcatcctgaacctgaccagcgtgaagcccgaggacagcggcatctatttctgcatgatcgtgggcagccccgagctgacatttggcaagggaacacagctgagcgtggtggacttcctgcctactacagcccagcctaccaagaagtctaccctgaagaaacgcgtgtgcagactgcccaggcctgagacacaaaagggccctctgtgcagccctatcacactgggattgctggtggctggcgttctggtcctgctggtgtctctgggagttgccatccacctgtgctgtagaagaaggcgggccagactgcggttcatgaagcagttctacaaaggcagcggcgccaccaacttcagcctgctgaaacaagccggcgacgtcgaggaaaatcctggacctatgggcacaagacttctctgttgggctgcactgtgcttgcttggagctgagctgacagaagctggagttgcccaatctcctaggtacaagatcatcgagaagcggcagtctgtggccttttggtgcaatcccattagcggacatgccaccctgtactggtatcagcaaattctgggacagggccctaaactgctgatccagttccagaataacggcgtggtggacgattctcaactgcctaaggaccggttttctgccgagagactgaaaggcgttgatagcaccctgaagatccaacctgccaaactggaggattctgccgtgtacctgtgtgctagcagcccttatggaggatctgtgtcttatgagcagtacttcggacctggcaccagactgaccgtgactgaagacctgaagaacgtgttccccccagaggtggccgtgttcgagcctagcgaggccgagatcagccacacccagaaagccaccctcgtgtgcctggccaccggcttttaccccgaccacgtggaactgtcttggtgggtcaacggcaaagaggtgcacagcggcgtctgcaccgacccccagcccctgaaagagcagcccgccctgaacgacagccggtactgtctgagcagcagactgagagtgtccgccaccttctggcagaacccccggaaccacttcagatgccaggtgcagttctacggcctgagcgagaacgacgagtggacccaggaccgggccaagcccgtgacccagatcgtgtctgctgaggcctggggcagagccgattgcggcttcaccagcgagagctaccagcagggcgtgctgagcgccaccatcctgtacgagatcctgctgggcaaggccaccctgtacgccgtgctggtgtccgccctggtgctgatggccatggtcaagcggaaggacagccggggcggttccggagccaccaacttcagcctgcttaaacaggccggcgacgtggaagagaaccctggacctatggagacactgcttggcgtatcactggtgattctgtggctgcaactggctagagtgaactctcagcagggagaagaggatcctcaagctctgagcattcaggaaggcgaaaacgcaaccatgaattgctcatacaagaccagcatcaacaacctgcagtggtaccggcagaatagcggaagaggactggttcacctgattttaatcaggtctaatgaaagggagaagcacagcggcagactgagagttaccctggacacatccaagaaatcttcttctctgctgattacagcctctagagccgccgatacagccagctacttttgtgccacagatcccatgaacaccaatgccggaaagagcacattcggcgatggcacaaccctgacagttaagcccaatatccagaatcccgatcctgccgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcattatccccgaggacacattcttcccaagccccgagagcagctgcgacgtgaagctggtggaaaagagcttcgagacagacaccaacctgaacttccagaacctcagcgtgatcggcttccggatcctgctgctgaaggtggccggcttcaacctgctgatgaccctgcggctgtggtccagctga

SEQ ID NO: 37 - protein, 274 aa, synthetic construct
  source 1..274: note = 11N6 TCR alpha chain, original protein, with signal peptide
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDPMNTNAGKSTFGDGTTLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 38 - protein, 253 aa, synthetic construct
  source 1..253: note = 11N6 TCR alpha chain - original protein, without signal peptide
QQGEEDPQALSIQEGENATMNCSYKTSINNLQWYRQNSGRGLVHLILIRSNEREKHSGRLRVTLDTSKKSSSLLITASRAADTASYFCATDPMNTNAGKSTFGDGTTLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 39 - protein, 112 aa, synthetic construct
  source 1..112: note = 11N6 TCR alpha chain variable domain, without signal peptide
QQGEEDPQALSIQEGENATMNCSYKTSINNLQWYRQNSGRGLVHLILIRSNEREKHSGRLRVTLDTSKKSSSLLITASRAADTASYFCATDPMNTNAGKSTFGDGTTLTVKP

SEQ ID NO: 40 - protein, 5 aa, synthetic construct
  source 1..5: note = 11N6 TCR alpha chain variable domain CDR1alpha
TSINN

SEQ ID NO: 41 - protein, 7 aa, synthetic construct
  source 1..7: note = 11N6 TCR alpha chain variable domain CDR2alpha
IRSNERE

SEQ ID NO: 42 - protein, 15 aa, synthetic construct
  source 1..15: note = 11N6 TCR alpha chain variable domain CDR3alpha - IMGT junction
CATDPMNTNAGKSTF

SEQ ID NO: 43 - protein, 13 aa, synthetic construct
  source 1..13: note = 11N6 TCR alpha chain variable domain CDR3alpha - IMGT
ATDPMNTNAGKST

SEQ ID NO: 44 - protein, 141 aa, synthetic construct; identical to SEQ ID NO: 18
  source 1..141: note = 11N6 TCR alpha chain constant domain (original protein)

SEQ ID NO: 45 - protein, 141 aa, synthetic construct; identical to SEQ ID NO: 19
  source 1..141: note = 11N6 TCR alpha chain constant domain (cys-modified protein)

SEQ ID NO: 46 - protein, 253 aa, synthetic construct
  source 1..253: note = 11N6 TCR alpha chain, without signal peptide, cys-modified
QQGEEDPQALSIQEGENATMNCSYKTSINNLQWYRQNSGRGLVHLILIRSNEREKHSGRLRVTLDTSKKSSSLLITASRAADTASYFCATDPMNTNAGKSTFGDGTTLTVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 47 - protein, 314 aa, synthetic construct
  source 1..314: note = 11N6 TCR beta chain original protein, with signal peptide
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSPYGGSVSYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 48 - protein, 293 aa, synthetic construct
  source 1..293: note = 11N6 TCR beta chain original protein, without signal peptide
GVAQSPRYKIIEKRQSVAFWCNPISGHATLYWYQQILGQGPKLLIQFQNNGVVDDSQLPKDRFSAERLKGVDSTLKIQPAKLEDSAVYLCASSPYGGSVSYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 49 - protein, 114 aa, synthetic construct
  source 1..114: note = 11N6 TCR beta chain variable domain, without signal peptide
GVAQSPRYKIIEKRQSVAFWCNPISGHATLYWYQQILGQGPKLLIQFQNNGVVDDSQLPKDRFSAERLKGVDSTLKIQPAKLEDSAVYLCASSPYGGSVSYEQYFGPGTRLTVT

SEQ ID NO: 50 - protein, 5 aa, synthetic construct
  source 1..5: note = 11N6 TCR beta chain variable domain CDR1beta
SGHAT

SEQ ID NO: 51 - protein, 6 aa, synthetic construct
  source 1..6: note = 11N6 TCR beta chain variable domain CDR2beta
FQNNGV

SEQ ID NO: 52 - protein, 16 aa, synthetic construct
  source 1..16: note = 11N6 TCR beta chain variable domain CDR3beta-IMGT junction
CASSPYGGSVSYEQYF

SEQ ID NO: 53 - protein, 14 aa, synthetic construct
  source 1..14: note = 11N6 TCR beta chain variable domain CDR3beta-IMGT
ASSPYGGSVSYEQY

SEQ ID NO: 54 - protein, 179 aa, synthetic construct; identical to SEQ ID NO: 28
  source 1..179: note = 11N6 TCR beta chain constant domain (original protein)

SEQ ID NO: 55 - protein, 179 aa, synthetic construct; identical to SEQ ID NO: 29
  source 1..179: note = 11N6 TCR beta chain constant domain (cys-modified protein)

SEQ ID NO: 56 - protein, 293 aa, synthetic construct
  source 1..293: note = 11N6 TCR beta chain (cys-modified protein)
GVAQSPRYKIIEKRQSVAFWCNPISGHATLYWYQQILGQGPKLLIQFQNNGVVDDSQLPKDRFSAERLKGVDSTLKIQPAKLEDSAVYLCASSPYGGSVSYEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 57 - protein, 610 aa, synthetic construct
  source 1..610: note = 11N6 TCRbeta-P2A-alpha-Protein, with signal peptides
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSPYGGSVSYEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMETLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDPMNTNAGKSTFGDGTTLTVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 58 - protein, 1098 aa, synthetic construct
  source 1..1098: note = CD8alpha-T2A-CD8beta-P2A-11N6 TCRbeta-P2A-alpha-Protein, with signal peptides
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVGSGEGRGSLLTCGDVEENPGPMRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYKGSGATNFSLLKQAGDVEENPGPMGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSPYGGSVSYEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMETLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDPMNTNAGKSTFGDGTTLTVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 59 - protein, 132 aa, synthetic construct
  source 1..132: note = TCR BNT Vbeta, with signal peptide
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLADIYEQYFGPGTRLTVT

SEQ ID NO: 60 - protein, 132 aa, synthetic construct
  source 1..132: note = TCR BNT Valpha, with signal peptide
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDRQSSGDKLTFGTGTRLAVRP

SEQ ID NO: 61 - protein, 113 aa, synthetic construct
  source 1..113: note = TCR 220_21 Valpha
GEDVEQSLFLSVREGDSSVINCTYTDSSSTYLYWYKQEPGAGLQLLTYIFSNMDMKQDQRLTVLLNKKDKHLSLRIADTQTGDSAIYFCAEPIIGGNTPLVFGKGTRLSVIAN

SEQ ID NO: 62 - protein, 119 aa, synthetic construct
  source 1..119: note = TCR 220_21 Vbeta
GAGVSQSPRYKVAKRGQDVALRCDPISGHVSLFWYQQALGQGPEFLTYFQNEAQLDKSGLPSDRFFAERPEGSVSTLKIQRTQQEDSAVYLCASSSEGLAGGPTAGELFFGEGSRLTVL

SEQ ID NO: 63 - protein, 114 aa, synthetic construct
  source 1..114: note = TCR 129_5 Valpha
AQSVTQPDIHITVSEGASLELRCNYSYGATPYLFWYVQSPGQGLQLLLKYFSGDTLVQGIKGFEAEFKRSQSSFNLRKPSVHWSDAAEYFCAVGASGTYKYIFGTGTRLKVLAN

SEQ ID NO: 64 - protein, 113 aa, synthetic construct
  source 1..113: note = TCR 129_5 Vbeta
DAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSLALSYEQYFGPGTRLTVT

SEQ ID NO: 65-68 - skipped (4 entries)

SEQ ID NO: 69 - protein, 141 aa, synthetic construct
  source 1..141: note = TCR Calpha amino acid sequence engineered to include threonine-to-cysteine and LVL mutations
NIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 70 - protein, 177 aa, Homo sapiens
  source 1..177: note = TRBC1 amino acid sequence (UniProt KB P01850)
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 71 - protein, 177 aa, synthetic construct
  source 1..177: note = TRBC1 amino acid sequence (cys-modified)
EDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 72 - protein, 179 aa, Homo sapiens
  source 1..179: note = TRBC2 amino acid sequence (UniProt KB A0A5B9)
EDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 73 - protein, 179 aa, synthetic construct
  source 1..179: note = TRBC2 amino acid sequence (cys-modified)
EDLKNVFPPKVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 74 - protein, 22 aa, synthetic construct
  source 1..22: note = Porcine teschovirus-1 2A (P2A) self-cleaving peptide with N-terminal GSG linker
GSGATNFSLLKQAGDVEENPGP

SEQ ID NO: 75 - protein, 24 aa, synthetic construct
  source 1..24: note = Thoseaasigna virus 2A (T2A) self-cleaving peptide
LEGGGEGRGSLLTCGDVEENPGPR

SEQ ID NO: 76 - protein, 20 aa, synthetic construct
  source 1..20: note = Equine rhinitis A virus (ERAV) 2A (E2A) self-cleaving peptide
QCTNYALLKLAGDVESNPGP

SEQ ID NO: 77 - protein, 25 aa, synthetic construct
  source 1..25: note = Foot-and-Mouth disease virus 2A (F2A) self-cleaving peptide with N-terminal G-S-G linker
GSGVKQTLNFDLLKLAGDVESNPGP

SEQ ID NO: 78 - protein, 189 aa, Homo sapiens
  source 1..189: note = NRAS (Uniprot KB P01111)
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 79 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 80 - protein, 9 aa, synthetic construct
  source 1..9: note = KRAS 8-16 wild-type (G12)
VVGAGGVGK

SEQ ID NO: 81 - protein, 10 aa, synthetic construct
  source 1..10: note = KRAS 7-16 wild-type (G12)
VVVGAGGVGK

SEQ ID NO: 82 - protein, 21 aa, synthetic construct
  source 1..21: note = T2A self-cleaving peptide with N-terminal GSG linker
GSGEGRGSLLTCGDVEENPGP

SEQ ID NO: 83 - protein, 309 aa, synthetic construct
  source 1..309: note = 11N4A TCR beta chain, with signal peptide (cys-modified protein), with signal peptide
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSVGTVEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 84 - DNA, 928 bp, synthetic construct
  source 1..928: mol_type = other DNA; note = Polynucleotide encoding 11N4A TCR beta chain, with signal peptide (cys-modified protein)
atgggcagcagactgctgtgctgggttctgctgtgtctgcttggagccggacctgtgaaagctggcgtgacccagacacctagatacctgatcaagaccagaggccagcaagtgacactgagctgtagccccatcagcggccacagaagcgtgtcctggtatcagcagactcctggacagggcctgcagttcctgttcgagtacttctccgagacacagaggaacaagggcaacttccccggcagattctccggcagacagttcagcaactcccgcagcgagatgaacgtgtccacactggaactgggagatagcgccctgtacctgtgtgcctcttctgtgggaaccgtggaacagtacttcggccctggcacaagactgaccgtgaccgaggacctgaagaacgtgttcccacctgaggtggccgtgttcgagccttctgaggccgagatctctcacacccagaaagccacactcgtgtgtctggccaccggcttctatcccgatcacgtggaactgtcttggtgggtcaacggcaaagaggtgcacagcggcgtctgtaccgatcctcagccactgaaagagcagcccgctctgaacgacagcagatactgcctgtcctccagactgagagtgtccgccaccttctggcagaaccccagaaaccacttcaggtgtcaggtgcagttttacggcctgagcgagaacgacgagtggacccaggatagagccaagcctgtgacacagatcgtgtctgccgaagcctggggcagagccgattgtggctttaccagcgagagctaccagcagggcgttctgtctgccaccatcctgtacgagatcctgctgggcaaagccactctgtacgccgtgttggtgtctgccctggtgctgatggccatggtcaagcggaaggattctagaggcg

SEQ ID NO: 85 - protein, 280 aa, synthetic construct
  source 1..280: note = 11N4A TCR alpha chain (cys-modified protein), with signal peptide
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASGVSGNTPLVFGKGTRLSVIANIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 86 - DNA, 843 bp, synthetic construct
  source 1..843: mol_type = other DNA; note = Polynucleotide encoding 11N4A TCR alpha chain, with signal peptide (cys-modified)
atggctatgctgctgggagcctctgtgctgatcctgtggctgcaacccgattgggtcaacagccagcagaagaacgacgaccagcaagtcaagcagaacagccccagcctgagcgtgcaagagggcagaatcagcatcctgaactgcgactacaccaactctatgttcgactactttctgtggtacaagaagtaccccgccgagggacccaccttcctgatcagcatcagcagcatcaaggacaagaacgaggacggccggttcaccgtgtttctgaacaagagcgccaagcacctgagcctgcacatcgtgccttctcagcctggcgatagcgccgtgtacttttgtgctgccagcggcgtgtcaggcaacacccctctggtttttggcaagggcacacgcctgtccgtgatcgccaacattcagaaccctgatcctgccgtgtaccagctgagagacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagtccgatttcgcctgcgccaacgccttcaacaacagcattatccccgaggacacattcttcccaagtcctgagtccagctgcgacgtgaagctggtggaaaagagcttcgagacagacaccaacctgaacttccagaatctgagcgtgatcggcttcagaatcctgctgctgaaggtggccggattcaacctgctgatgaccctcagactgtggtccagctga

SEQ ID NO: 87 - protein, 235 aa, synthetic construct
  source 1..235: note = CD8 alpha with signal peptide
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 88 - DNA, 705 bp, synthetic construct
  source 1..705: mol_type = other DNA; note = Polynucleotide encoding CD8 alpha (with signal peptide)
atggcattgcctgttacagctctgctgctgcccctggctctgcttctgcatgctgctagacccagccagttcagagtgtcccctctggacagaacctggaacctgggcgagacagtggaactgaagtgccaggtgctgctgagcaatcctaccagcggctgcagctggctgtttcagcctagaggtgctgccgcctctcctacctttctgctgtacctgagccagaacaagcccaaggccgccgaaggactggacacccagagattcagcggcaagagactgggcgacaccttcgtgctgaccctgagcgacttcagaagagagaacgagggctactacttctgcagcgccctgagcaacagcatcatgtacttcagccacttcgtgcccgtgtttctgcccgccaagcctacaacaacccctgctcctagacctcctacaccagctcctacaatcgccagccagcctctgtctctgaggccagaagcttgtagacctgctgctggcggagccgtgcatacaagaggactggatttcgcctgcgacatctacatctgggcccctctggctggaacatgtggcgtgctgctgctgtctctggtcatcaccctgtactgcaaccaccggaacaggcggagagtgtgcaagtgccctagacctgtggtcaagagcggcgacaagcctagcctgagcgccagatatgtt

SEQ ID NO: 89 - protein, 210 aa, synthetic construct
  source 1..210: note = CD8 beta with signal peptide
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYK

SEQ ID NO: 90 - DNA, 630 bp, synthetic construct
  source 1..630: mol_type = other DNA; note = Polynucleotide encoding CD8 beta, with signal peptide
atgaggcctagactgtggctgcttctggctgcccagctgacagtgctgcacggcaattctgtcctgcagcagacccctgcctacatcaaggtgcagaccaacaagatggtcatgctgagctgcgaggccaagatcagcctgtccaacatgcggatctactggctgcggcagagacaggcccctagcagcgattctcaccacgagtttctggccctgtgggatagcgccaagggaaccattcacggcgaggaagtggaacaagagaagatcgccgtgttccgggacgccagcagattcatcctgaacctgaccagcgtgaagcccgaggacagcggcatctatttctgcatgatcgtgggcagccccgagctgacatttggcaagggaacacagctgagcgtggtggacttcctgcctactacagcccagcctaccaagaagtctaccctgaagaaacgcgtgtgcagactgcccaggcctgagacacaaaagggccctctgtgcagccctatcacactgggattgctggtggctggcgttctggtcctgctggtttctctgggagttgccatccacctgtgctgcagacgcagaagggccagactgcggttcatgaagcagttctacaaa

SEQ ID NO: 91 - protein, 26 aa, synthetic construct
  source 1..26: note = 11N4A alpha FR1 (IMGT)
DQQVKQNSPSLSVQEGRISILNCDYT

SEQ ID NO: 92 - protein, 17 aa, synthetic construct
  source 1..17: note = 11N4A alpha FR2 (IMGT)
FLWYKKYPAEGPTFLIS

SEQ ID NO: 93 - protein, 34 aa, synthetic construct
  source 1..34: note = 11N4A alpha FR3 (IMGT)
NEDGRFTVFLNKSAKHLSLHIVPSQPGDSAVYFC

SEQ ID NO: 94 - protein, 11 aa, synthetic construct
  source 1..11: note = 11N4A alpha FR4 (IMGT)
FGKGTRLSVIA

SEQ ID NO: 95 - protein, 33 aa, synthetic construct
  source 1..33: note = 11N4A alpha FR3 (IMGT junction)
NEDGRFTVFLNKSAKHLSLHIVPSQPGDSAVYF

SEQ ID NO: 96 - protein, 10 aa, synthetic construct
  source 1..10: note = 11N4A alpha FR4 (IMGT junction)
GKGTRLSVIA

SEQ ID NO: 97 - protein, 24 aa, synthetic construct
  source 1..24: note = sequence from 11N4A beta FR1 (IMGT)
GVTQTPRYLIKTRGQQVTLSCSPI

SEQ ID NO: 98 - protein, 17 aa, synthetic construct
  source 1..17: note = 11N4A beta FR2 (IMGT)
VSWYQQTPGQGLQFLFE

SEQ ID NO: 99 - protein, 37 aa, synthetic construct
  source 1..37: note = 11N4A beta FR3 (IMGT)
RNKGNFPGRFSGRQFSNSRSEMNVSTLELGDSALYLC

SEQ ID NO: 100 - protein, 9 aa, synthetic construct
  source 1..9: note = sequence from 11N4A beta FR4 (IMGT)
FGPGTRLTV

SEQ ID NO: 101 - protein, 36 aa, synthetic construct
  source 1..36: note = 11N4A beta FR3 (IMGT junction)
RNKGNFPGRFSGRQFSNSRSEMNVSTLELGDSALYL

SEQ ID NO: 102 - protein, 8 aa, synthetic construct
  source 1..8: note = sequence from 11N4A beta FR4 (IMGT junction)
GPGTRLTV

SEQ ID NO: 103 - protein, 25 aa, synthetic construct
  source 1..25: note = 11N6 alpha FR1 (IMGT)
QQGEEDPQALSIQEGENATMNCSYK

SEQ ID NO: 104 - protein, 17 aa, synthetic construct
  source 1..17: note = 11N6 alpha FR2 (IMGT)
LQWYRQNSGRGLVHLIL

SEQ ID NO: 105 - protein, 34 aa, synthetic construct
  source 1..34: note = 11N6 alpha FR3 (IMGT)
KHSGRLRVTLDTSKKSSSLLITASRAADTASYFC

SEQ ID NO: 106 - protein, 11 aa, synthetic construct
  source 1..11: note = 11N6 alpha FR4 (IMGT)
FGDGTTLTVKP

SEQ ID NO: 107 - protein, 33 aa, synthetic construct
  source 1..33: note = 11N6 alpha FR3 (IMGT junction)
KHSGRLRVTLDTSKKSSSLLITASRAADTASYF

SEQ ID NO: 108 - protein, 10 aa, synthetic construct
  source 1..10: note = 11N6 alpha FR4 (IMGT junction)
GDGTTLTVKP

SEQ ID NO: 109 - protein, 24 aa, synthetic construct
  source 1..24: note = 11N6 beta FR1 (IMGT)
GVAQSPRYKIIEKRQSVAFWCNPI

SEQ ID NO: 110 - protein, 17 aa, synthetic construct
  source 1..17: note = 11N6 beta FR2 (IMGT)
LYWYQQILGQGPKLLIQ

SEQ ID NO: 111 - protein, 38 aa, synthetic construct
  source 1..38: note = 11N6 beta FR3 (IMGT)
VDDSQLPKDRFSAERLKGVDSTLKIQPAKLEDSAVYLC

SEQ ID NO: 112 - protein, 10 aa, synthetic construct
  source 1..10: note = 11N6 beta FR4 (IMGT)
FGPGTRLTVT

SEQ ID NO: 113 - protein, 37 aa, synthetic construct
  source 1..37: note = 11N6 beta FR3 (IMGT junction)
VDDSQLPKDRFSAERLKGVDSTLKIQPAKLEDSAVYL

SEQ ID NO: 114 - protein, 9 aa, synthetic construct
  source 1..9: note = 11N6 beta FR4 (IMGT junction)
GPGTRLTVT

SEQ ID NO: 115 - protein, 31 aa, synthetic construct
  source 1..31: note = 11N4A alpha FR1 (IMGT)
QQKNDDQQVKQNSPSLSVQEGRISILNCDYT

SEQ ID NO: 116 - protein, 10 aa, synthetic construct
  source 1..10: note = 11N4A beta FR4 (IMGT)
FGPGTRLTVT

SEQ ID NO: 117 - protein, 9 aa, synthetic construct
  source 1..9: note = 11N4A beta FR4 (IMGT junction)
GPGTRLTVT

SEQ ID NO: 118 - protein, 9 aa, synthetic construct
  source 1..9: note = CRNS1 peptide
VVGAGGVSK

SEQ ID NO: 119 - protein, 9 aa, synthetic construct
  source 1..9: note = RASE peptide
VVGASGVGK

SEQ ID NO: 120 - protein, 9 aa, synthetic construct
  source 1..9: note = RAB7B peptide
IVGAIGVGK

SEQ ID NO: 121 - skipped

SEQ ID NO: 122 - protein, 9 aa, synthetic construct
  source 1..9: note = ITA8 peptide
IVGAFGTGK

SEQ ID NO: 123 - protein, 9 aa, synthetic construct
  source 1..9: note = MIRO2 peptide
VVGARGVGK

SEQ ID NO: 124 - protein, 9 aa, synthetic construct
  source 1..9: note = ARRD4 peptide
AVGAEGRVK

SEQ ID NO: 125 - protein, 9 aa, synthetic construct
  source 1..9: note = SMC5 peptide
IVGANGTGK

SEQ ID NO: 126 - protein, 9 aa, synthetic construct
  source 1..9: note = MOGS peptide
EVGAKGQLK

SEQ ID NO: 127 - protein, 9 aa, synthetic construct
  source 1..9: note = GKN2 peptide
NVGAGGCAK

SEQ ID NO: 128 - protein, 9 aa, synthetic construct
  source 1..9: note = AFDDT peptide
QMGAAGSGR

SEQ ID NO: 129 - protein, 9 aa, synthetic construct
  source 1..9: note = ANKR9 peptide
PVGAAGSAR

SEQ ID NO: 130 - protein, 9 aa, synthetic construct
  source 1..9: note = CFTR peptide
VAGSTGAGK

SEQ ID NO: 131 - protein, 9 aa, synthetic construct
  source 1..9: note = DYH2 peptide
IVGCTGSGK

SEQ ID NO: 132 - protein, 9 aa, synthetic construct
  source 1..9: note = EP300 peptide
MNGSIGAGR

SEQ ID NO: 133 - protein, 9 aa, synthetic construct
  source 1..9: note = FOXA2 peptide
AAGAAGSGK

SEQ ID NO: 134 - protein, 9 aa, synthetic construct
  source 1..9: note = HTR5B peptide
ASGAVGSAK

SEQ ID NO: 135 - protein, 9 aa, synthetic construct
  source 1..9: note = LARP1 peptide
AAGAAGAGR

SEQ ID NO: 136 - protein, 9 aa, synthetic construct
  source 1..9: note = MED1 peptide
NVGSTGVAK

SEQ ID NO: 137 - protein, 9 aa, synthetic construct
  source 1..9: note = MOD5 peptide
ILGATGTGK

SEQ ID NO: 138 - protein, 9 aa, synthetic construct
  source 1..9: note = MRP5 peptide
ICGSVGSGK

SEQ ID NO: 139 - protein, 9 aa, synthetic construct
  source 1..9: note = NAL12 peptide
MQGAAGIGK

SEQ ID NO: 140 - protein, 9 aa, synthetic construct
  source 1..9: note = PCGF6 peptide
TAGSVGAAK

SEQ ID NO: 141 - protein, 9 aa, synthetic construct
  source 1..9: note = RAB4B peptide
VIGSAGTGK

SEQ ID NO: 142 - protein, 9 aa, synthetic construct
  source 1..9: note = SCGR4 peptide
TCGSCGCGY

SEQ ID NO: 143 - protein, 9 aa, synthetic construct
  source 1..9: note = 3HIDH peptide
VSGGVGAAR

SEQ ID NO: 144 - protein, 9 aa, synthetic construct
  source 1..9: note = AFDDT-2 peptide
PMGGTGSGR

SEQ ID NO: 145 - protein, 9 aa, synthetic construct
  source 1..9: note = BOLA3 peptide
ISGGCGAMY

SEQ ID NO: 146 - protein, 9 aa, synthetic construct
  source 1..9: note = DYH9 peptide
VVGGAGTGK

SEQ ID NO: 147 - protein, 9 aa, synthetic construct
  source 1..9: note = KRA53 peptide
SCGGCGSGY

SEQ ID NO: 148 - protein, 9 aa, synthetic construct
  source 1..9: note = SHRM1 peptide
VNGSVGISR

SEQ ID NO: 149 - protein, 9 aa, synthetic construct
  source 1..9: note = Alanine scan peptide search string, 9-mer
VXGAXGXXK

SEQ ID NO: 150 - protein, 10 aa, synthetic construct
  source 1..10: note = Alanine scan peptide search string, 10-mer
XXVGAVGXXK

SEQ ID NO: 151 - protein, 9 aa, synthetic construct
  source 1..9: note = Alanine scan peptide search string, 9-mer
XXGAXGXXK

SEQ ID NO: 152 - protein, 46 aa, synthetic construct
  source 1..46: note = Xscan peptide search string
ANCQIMPSTVANCQILMSTVGCSAACQHIMTVGACISTVAEMGRYK

SEQ ID NO: 153 - protein, 26 aa, synthetic construct
  source 1..26: note = 11N4A beta FR1 (IMGT)
KAGVTQTPRYLIKTRGQQVTLSCSPI

(19) United States
(12) Patent Application Publication
(10) Pub. No.: US 2025/0177531 A1
(43) Pub. Date: Jun. 5, 2025

(54) BINDING PROTEINS SPECIFIC FOR RAS NEOANTIGENS AND USES THEREOF

(71) Applicant: FRED HUTCHINSON CANCER CENTER, Seattle, WA (US)

(72) Inventors: Thomas M. SCHMITT, Seattle, WA (US); Philip D. GREENBERG, Mercer Island, WA (US); Tijana MARTINOV, Seattle, WA (US); Rachel PERRET, Wellington (NZ)

(21) Appl. No.: 18/945,416

(22) Filed: Nov. 12, 2024

Related U.S. Application Data
(63) Continuation of application No. PCT/US2023/066937, filed on May 12, 2023.
(60) Provisional application No. 63/342,025, filed on May 13, 2022.
(60) Provisional application No. 63/380,551, filed on Oct. 21, 2022.
(60) Provisional application No. 63/488,758, filed on Mar. 6, 2023.

(51) Int. Cl. A61K 40/42; A61K 40/11; A61K 40/32; A61P 35/00; C07K 14/725; C07K 16/32; C07K 16/40

(52) U.S. Cl. CPC A61K 40/4253; A61K 40/11; A61K 40/32; A61P 35/00; C07K 14/7051; C07K 16/32; C07K 16/40

(57) ABSTRACT

The present disclosure provides compositions and methods for targeting a Ras antigen to, for example, treat or prevent cancer. Disclosed embodiments include binding proteins, such as T cell receptors bind to a Ras antigen:HLA complex.

Disclosed binding proteins are highly sensitive to antigen, capable of inducing activation of host T cells at low concentrations of peptide antigen. In certain embodiments, binding proteins of the present disclosure are non-alloreactive against, are substantially non-alloreactive against, and/or have a low risk of alloreactivity against (i) amino acid sequences from the human proteome and/or (ii) against human HLA alleles. Polynucleotides encoding such binding protein can introduced into a host cell, such as a T cell, and the cell can be used in immunotherapy for treating various cancers.

### CROSS-REFERENCE TO RELATED APPLICATIONS

This application is a Continuation application of International Application No. PCT/US2023/066937, filed May 12, 2023, which claims the benefit of U.S. Provisional Patent Application No. 63/342,025, filed May 13, 2022, U.S. Provisional Patent Application No. 63/380,551, filed Oct. 21, 2022, and U.S. Provisional Patent Application No. 63/488,758, filed Mar. 6, 2023, the entire disclosures of which are herein incorporated by reference in their entirety.

### REFERENCE TO AN ELECTRONIC SEQUENCE LISTING

The contents of the electronic sequence listing (502C1_SeqListing.xml; Size: 196,159 bytes; and Date of Creation: Feb. 21, 2025) is herein incorporated by reference in its entirety.

### BACKGROUND

Ras family proteins are small GTPases that are involved with transmitting signals within cells, including, for example, transduction of cell proliferation. Exemplary RAS proteins include KRAS (also called C-K-RAS, CFC2, K-RAS2A, K-RAS2B, K-RAS4A, K-RAS4B, KI-RAS, KRAS1, KRAS2, NS, NS3, RALD, RASK2, K-ras, KRAS proto-oncogene, GTPase, and c-Ki-ras2), HRAS, and NRAS. Mutations in RAS proteins that disrupt negative growth signaling can lead to continuous proliferation of the cell. KRAS is one of the most frequently mutated proto-oncogenes in a variety of human cancers, including melanomas, endometrial, thyroid, pancreatic, colorectal, breast, ovarian, and lung cancers, as well as some instances of myeloid leukemias such as AML. Pharmacological inhibitors have been developed that target KRAS G12C, but primary and adaptive resistance of cancers to these inhibitors has been reported (e.g., Awad et al. NEJM 384:2382-2393 (2021)). New therapies targeting mutant RAS proteins are required.

### BRIEF DESCRIPTION OF THE FIGURES

FIGS. 1A-1E relate to identification of KRAS G12V-specific T cell receptors (TCRs) from the T cell repertoire of healthy human donors. (FIG. 1A) (left) Schematic showing a process for identifying HLA-A11-restricted mutant KRAS (mKRAS)-specific T cell lines from donor samples and (right) TNFα production by CD8+ T cells expressing a mKRAS-specific TCR in the absence (left) or presence (right) of mKRAS G12V peptide. (FIG. 1B) Schematic diagrams of processes for (top) sorting and sequencing mKRAS-reactive CD8+ T cells and (bottom) engineering CD8+ T cells to heterologously express a mKRAS-specific TCR. Fifty-six mKRAS-specific TCRs (G12V-specific or G12D-specific) were isolated, and sensitivity and cytotoxicity assays were performed. (FIG. 1C) Fold-enrichment of T cell clones in vitro with and without KRAS G12V mutant peptide. (FIG. 1D) Activation of TCR-transduced T cells in vitro as assessed by the percentage of T cells expressing GFP under the control of Nur77 locus, in the presence of varying concentrations of KRAS G12V mutant peptide. T cells were transduced to express a TCR as shown in the figure key. (FIG. 1E) Log EC50 KRAS G12V 9-mer peptide values (representing the concentration of KRAS G12V peptide required for TCR-transduced T cells to produce their half-maximal response of Nur77 expression).

FIGS. 2A-2C show functional avidity of TCR 11NA4 (see Table 1) compared with that of TCR 220_21 (V-domain amino acid sequences shown in SEQ ID NOs.: 61 (Vα) and 62 (Vβ)) and TCR “BNT” (Vα domain amino acid sequence (with signal peptide) shown in SEQ ID NO.: 60; Vβ domain amino acid sequence (with signal peptide) shown in SEQ ID NO.: 59). (FIG. 2A) Percent of TCR-transduced primary CD8+ T cells expressing CD137 at the indicated concentrations of KRAS G12V peptide; (FIG. 2B) log EC50 of the TCRs for KRAS G12V peptide; (FIG. 2C) Percent of TCR-transduced primary CD8+ T cells expressing IFN-γ at the indicated concentrations of KRAS G12V peptide.

FIGS. 2D-2F show functional avidity (assessed as CD137 expression by host T cells) of TCR 11N4A. FIG. 2D demonstrates that T cells transduced with TCR 11N4A recognize both KRAS G12V 9-mer and 10-mer peptides; FIG. 2E shows log EC50 of the TCRs for KRAS G12V 9-mer (left) and 10-mer (right) peptides; values are with reference to the curves in FIG. 2D, and show potent functional avidity (low picomolar EC50) for TCR 11N4A; and FIG. 2F shows no recognition of KRAS G12G wild-type peptide by TCR 11N4A. In FIG. 2E, y-axis values on the graph (bottom-to-top) are: −11, −10, −9, −8, −7, −6, −5. In FIG. 2F, y-axis values on the graph (bottom-to-top) are: 0, 20, 40, 60, 80, 100; the x-axis text (from left to right) is: No peptide; G12V₇₋₁₈ 10mer; G12V₈₋₁₈ 9mer; G12G₇₋₁₈ WT; G12G₈₋₁₈ WT.

FIG. 2G shows that transduced T cells expressing TCR 11N4A are activated specifically in response to mutant KRAS G12V peptide but not to wildtype KRAS G12G 9mer or 10mer peptides. Sort-purified populations of donor T cells expressing TCR 11N4A were exposed to mutant KRAS G12V 9mer, wildtype KRAS G12G 9mer or 10mer at 1 μg/mL, or no peptide control for 16 hours and T cell activation was assessed by CD137 (4-1BB) expression.

FIGS. 3A-3G show recognition by TCR-transduced T cells (assessed by percentage of TCR-transduced T cells expressing CD137) of HLA-A11+KRAS G12V-expressing tumor cell lines. “UT”=Untransduced, negative control. FIG. 3C shows activation of untransduced T cells or 11N4A-TCR and CD8αβ co-receptor engineered T cells by endogenous KRAS G12V presentation across diverse tumor cell lines.

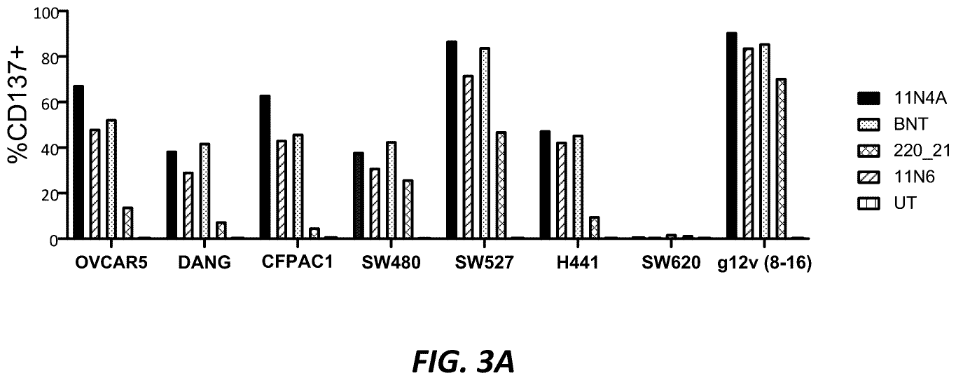

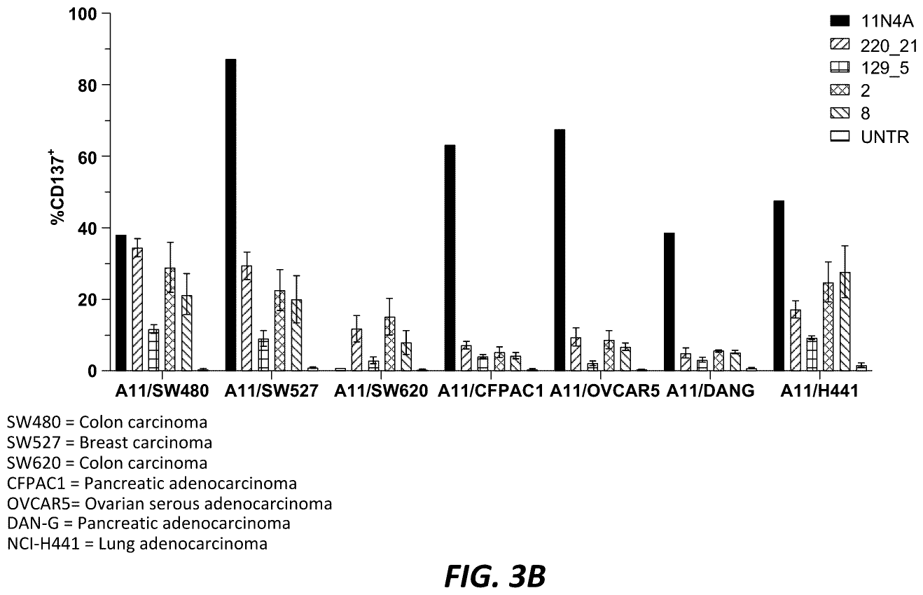

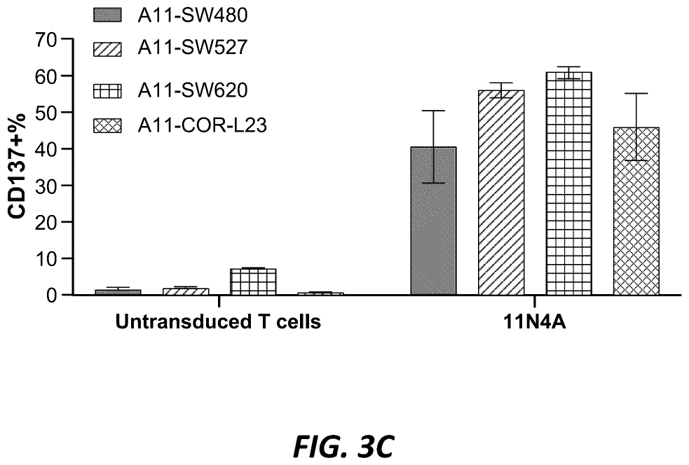

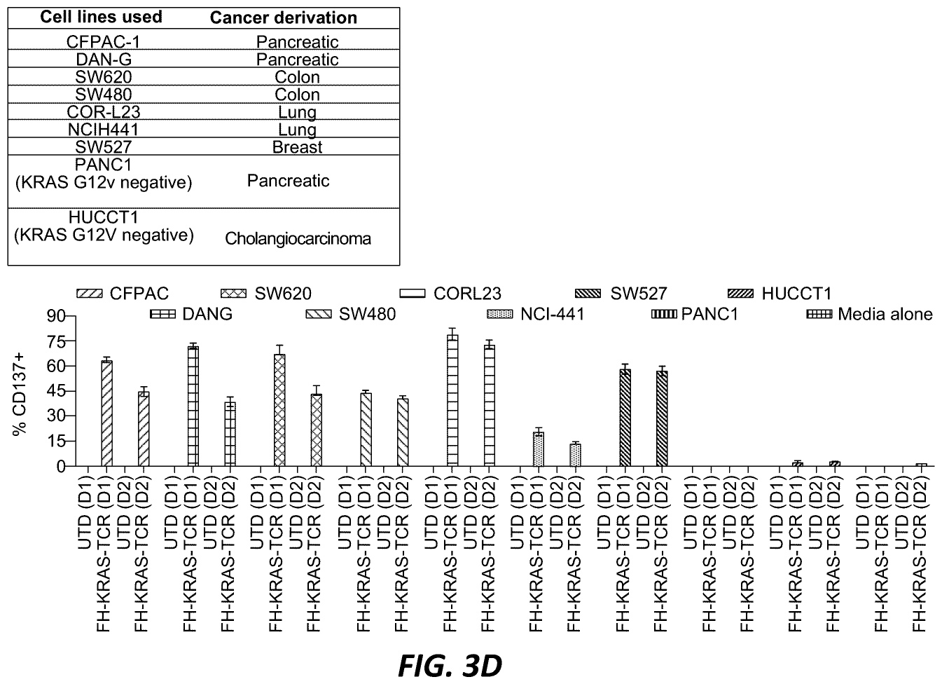

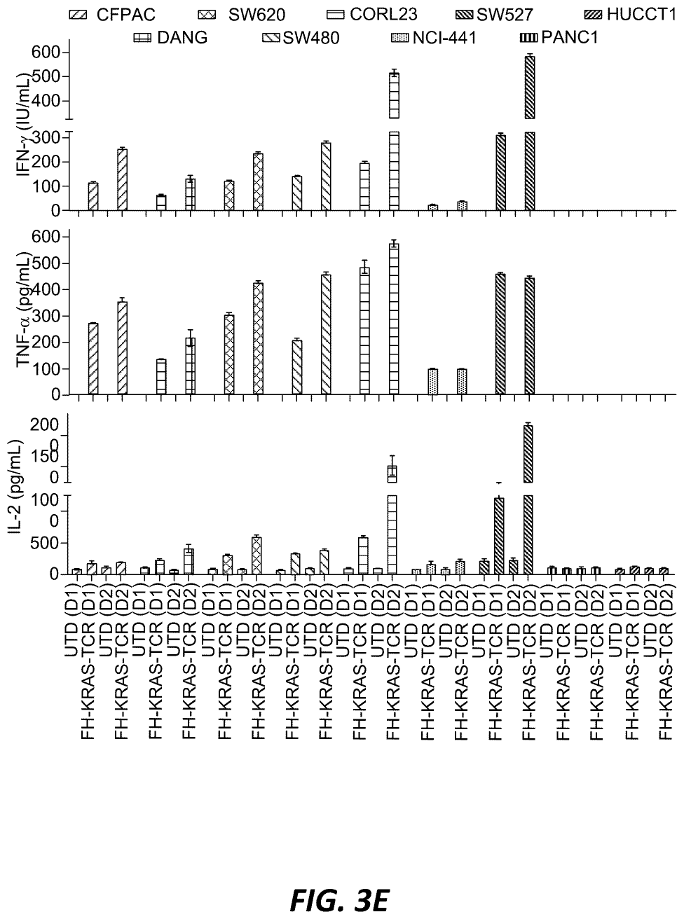

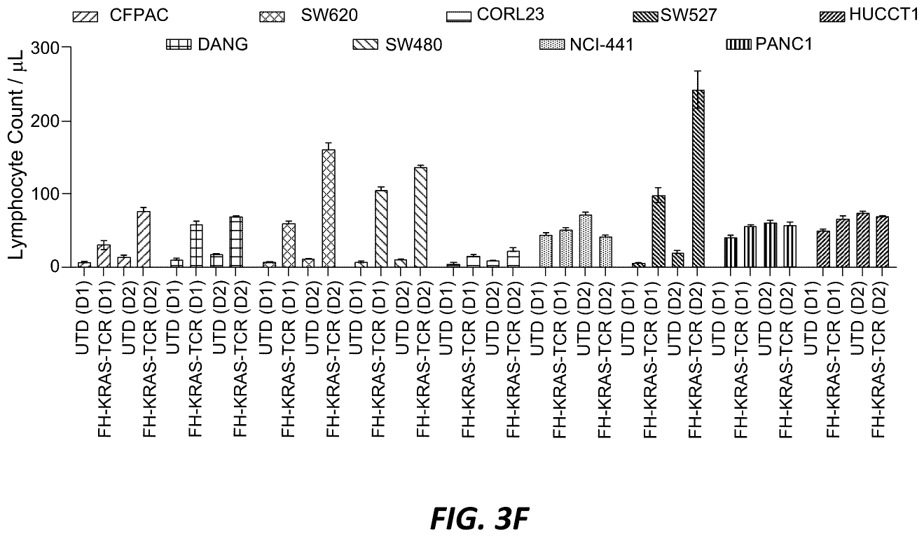

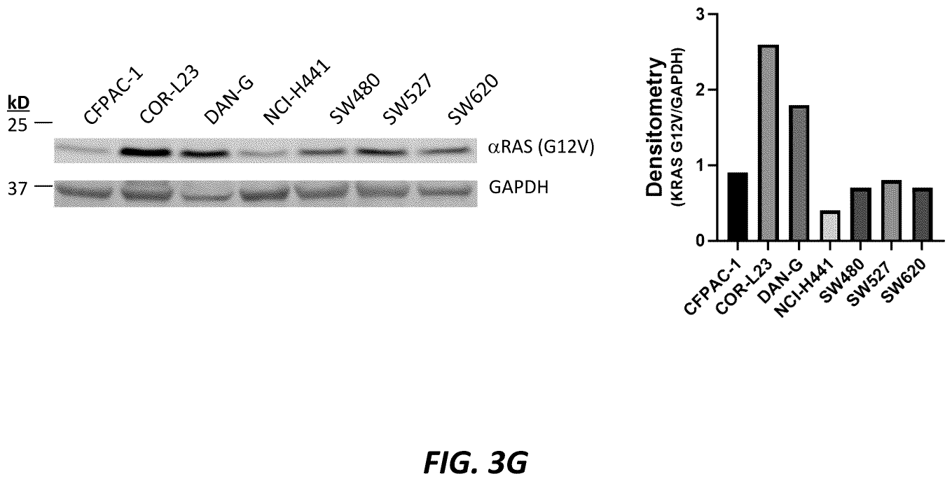

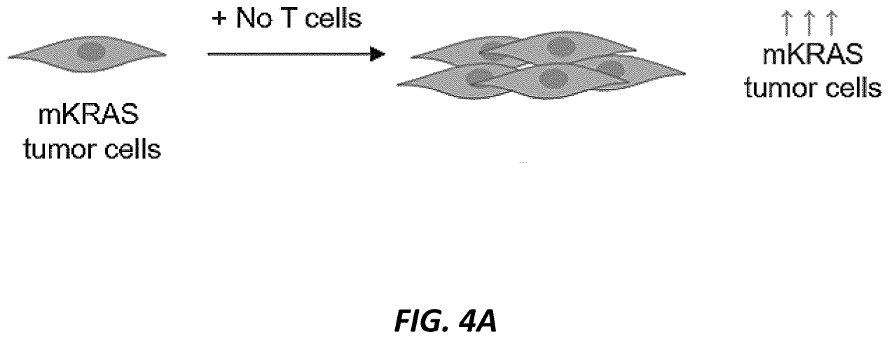

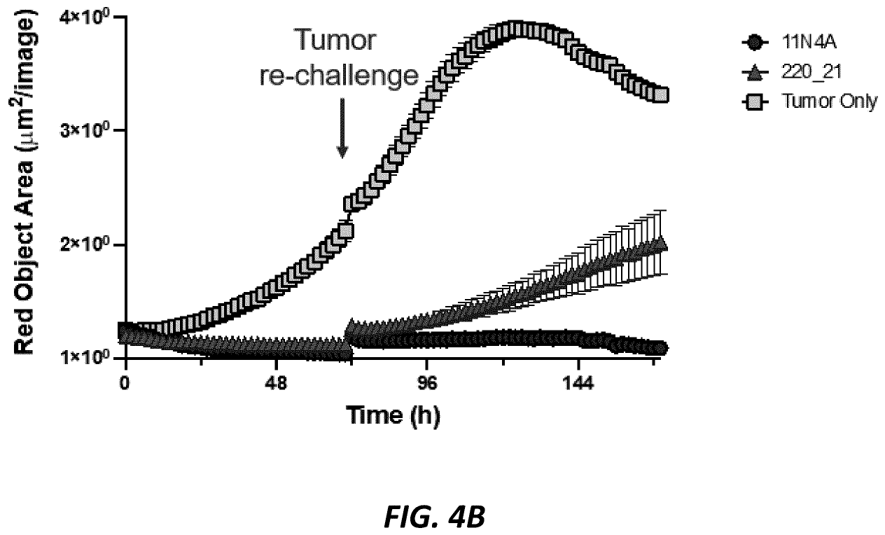

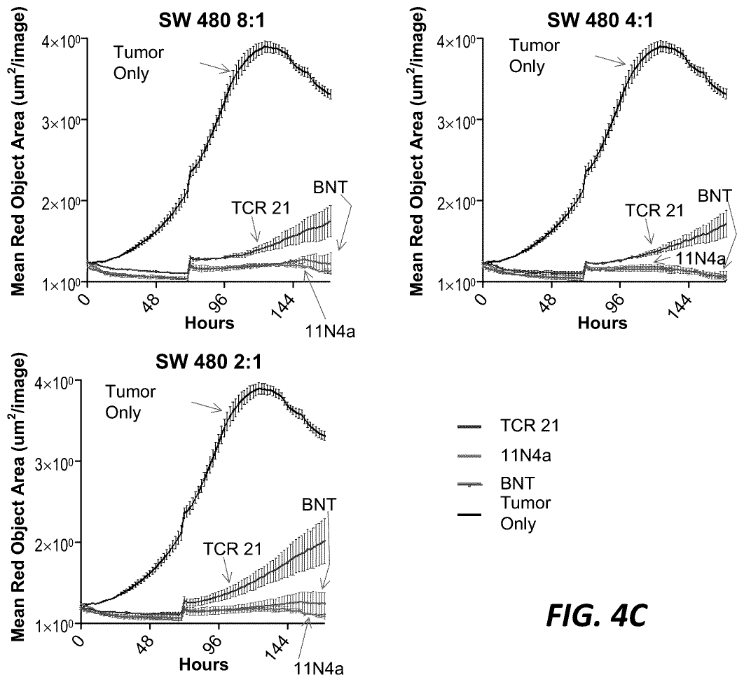

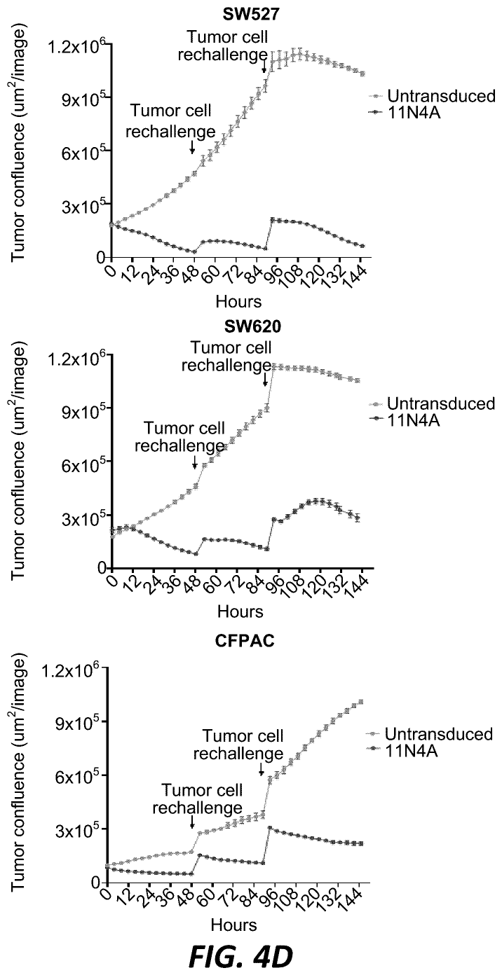

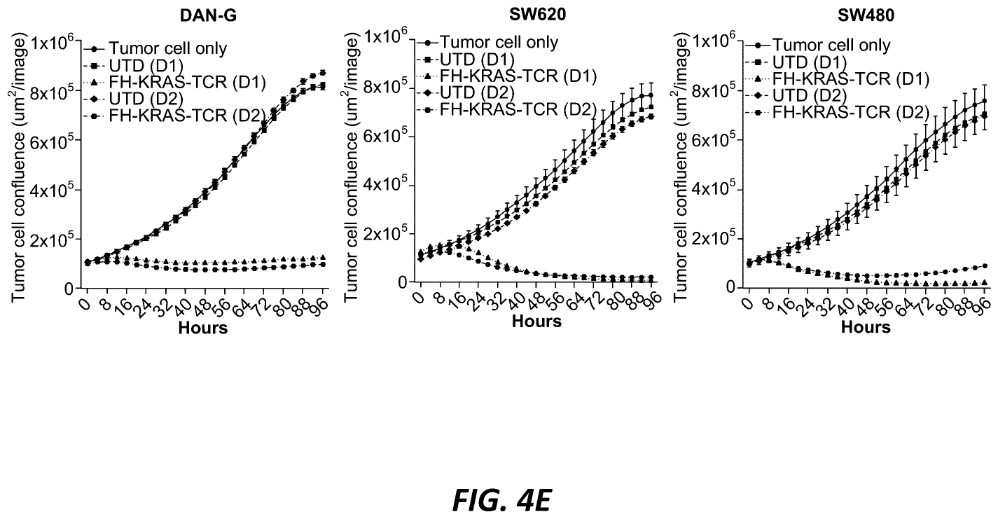

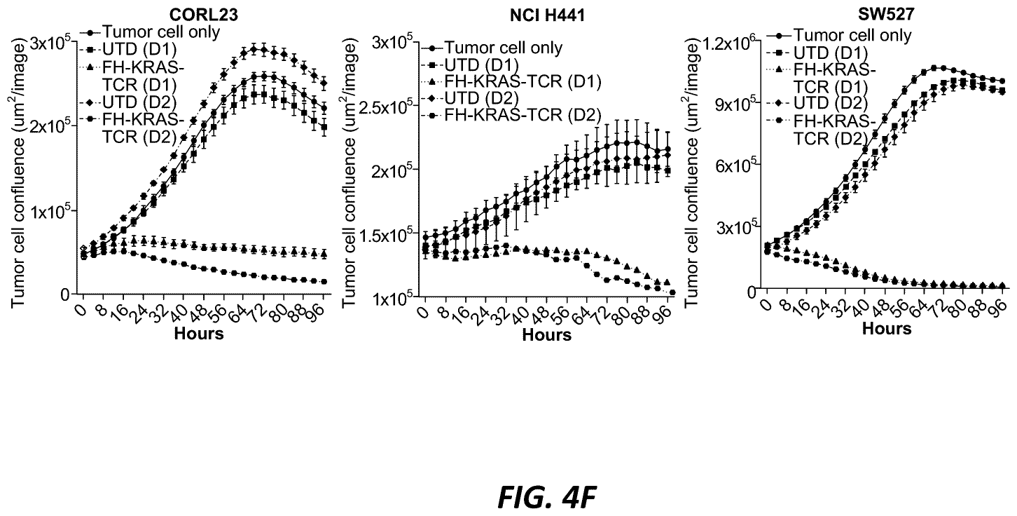

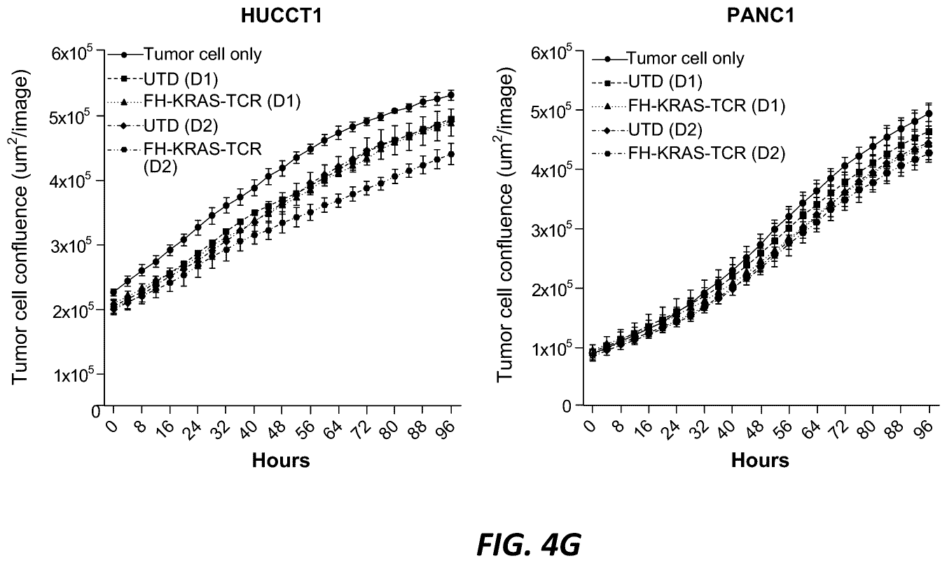

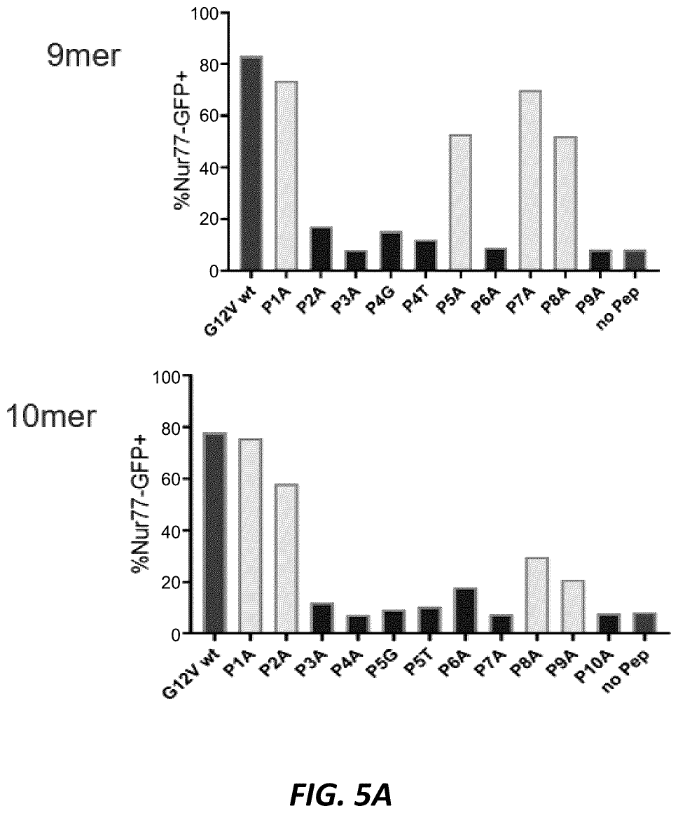

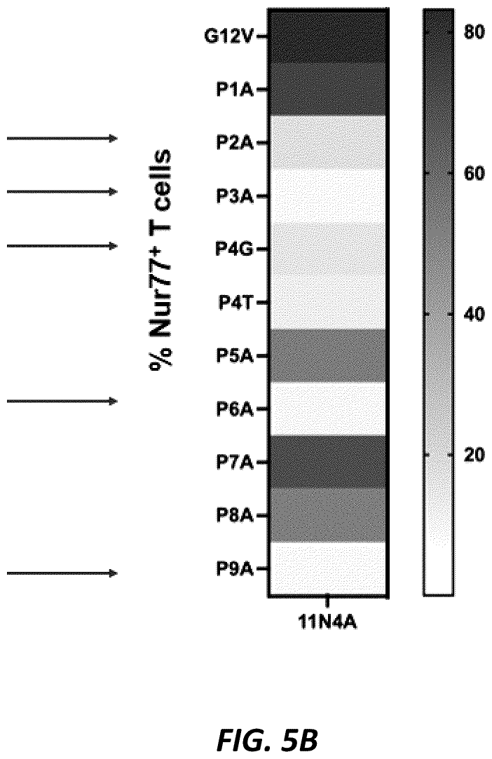

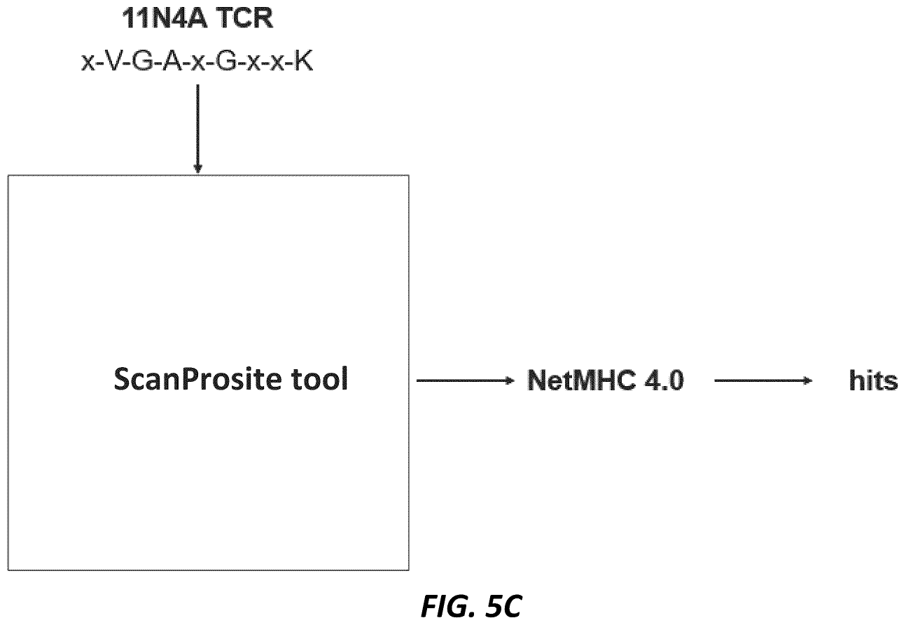

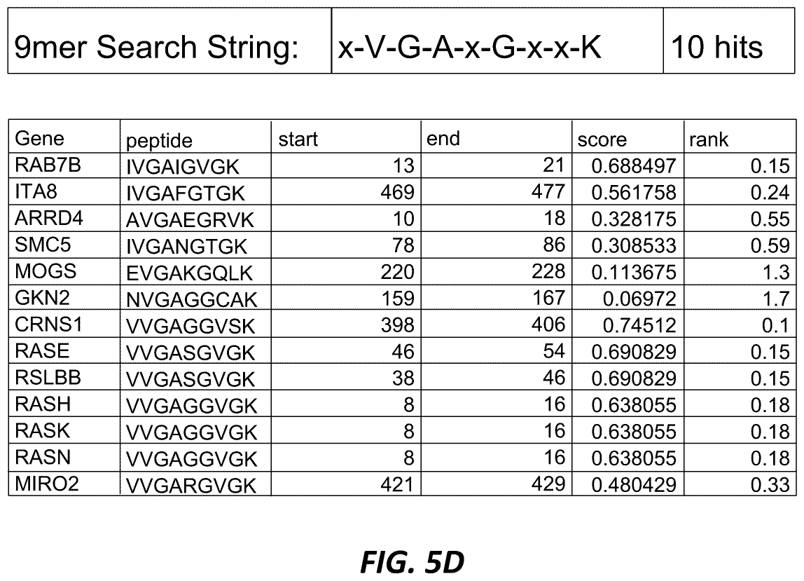

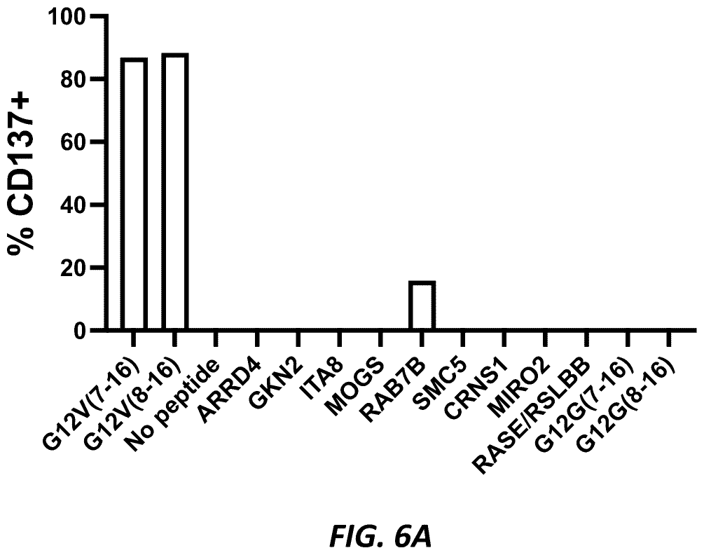

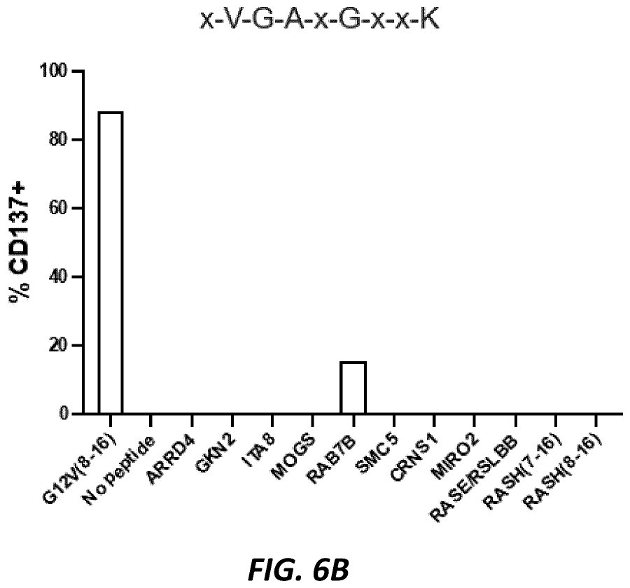

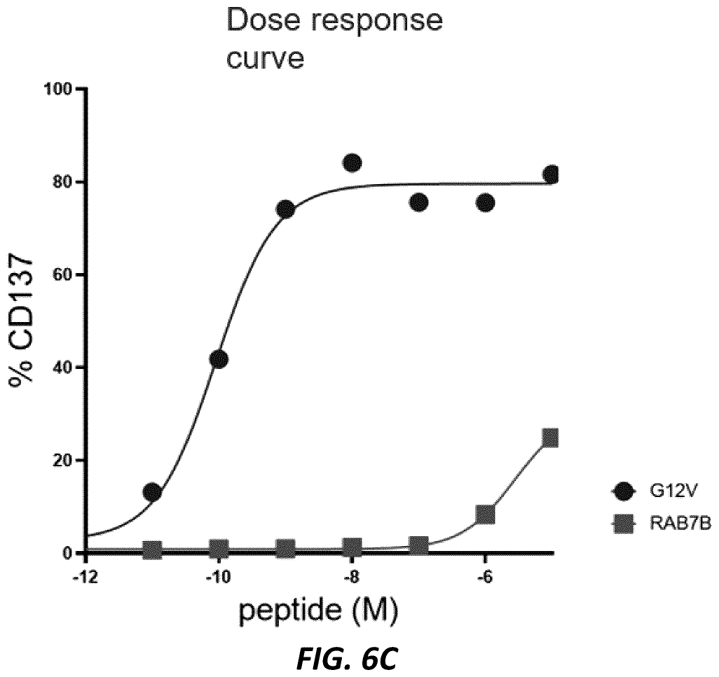

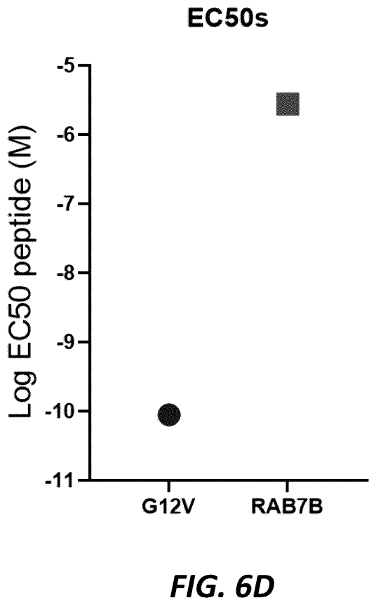

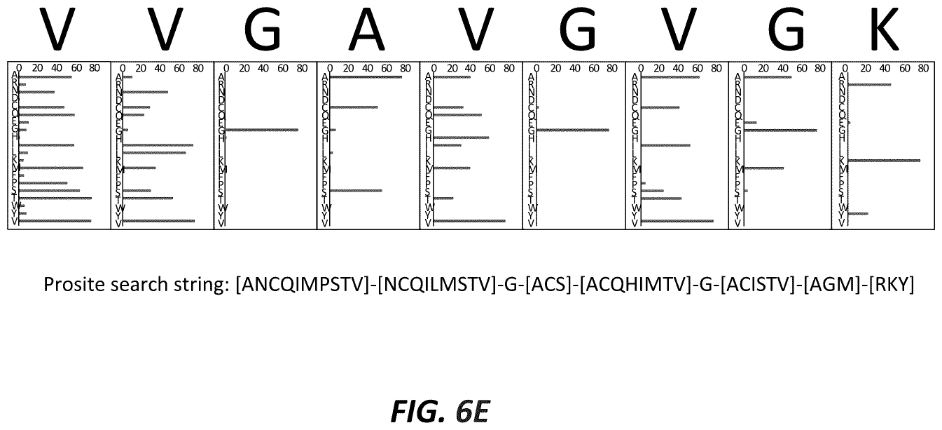

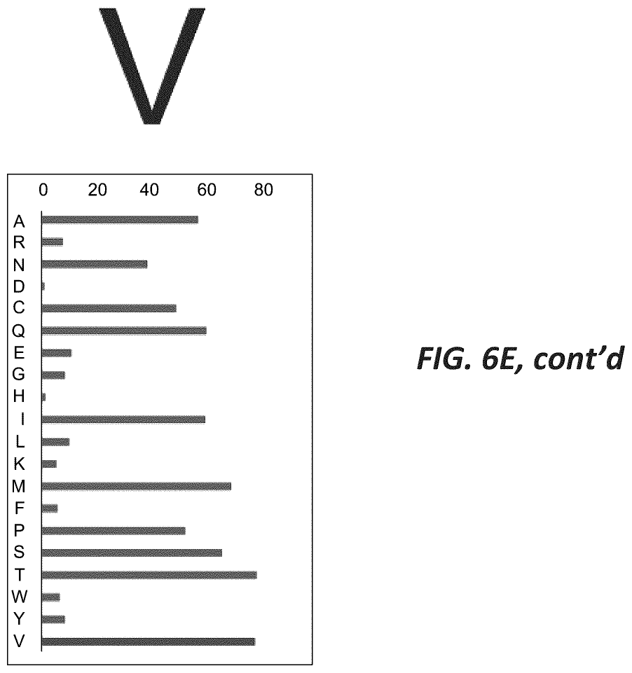

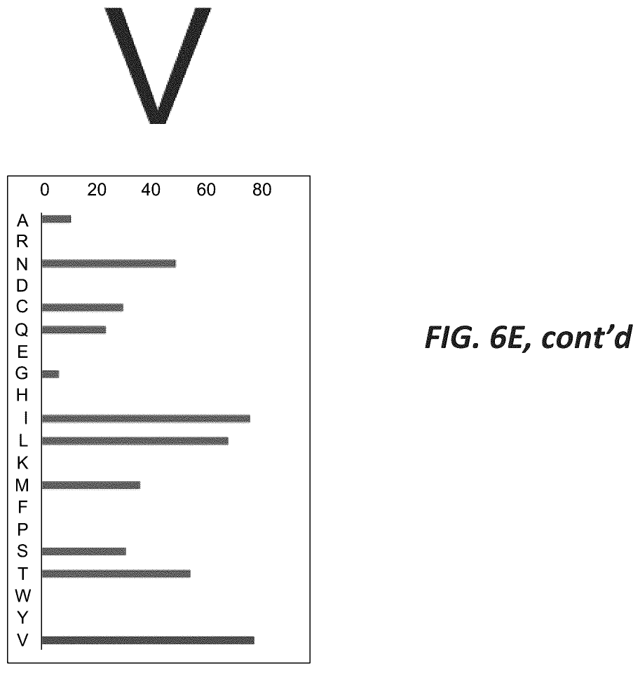

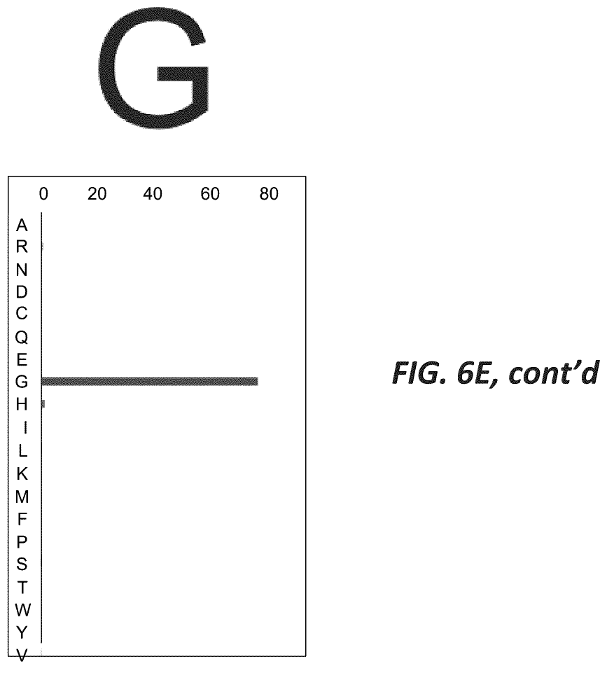

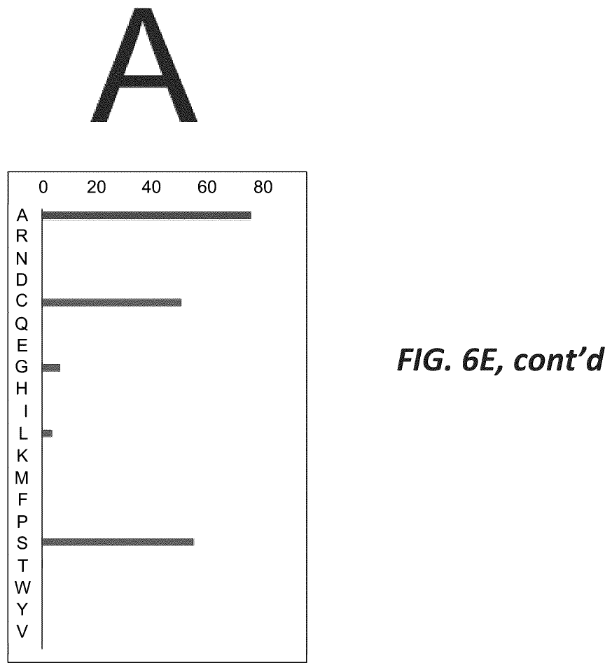

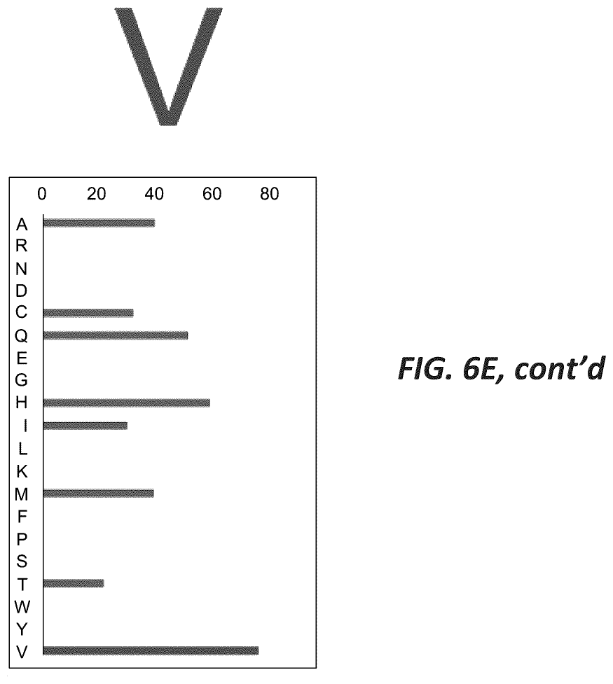

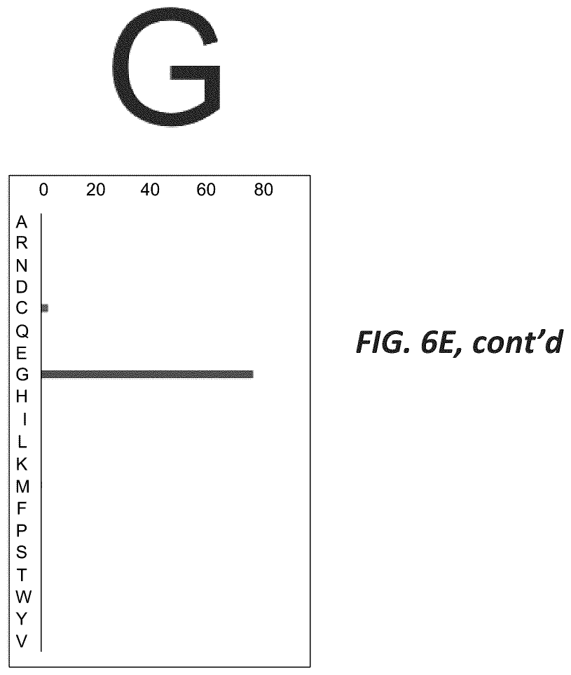

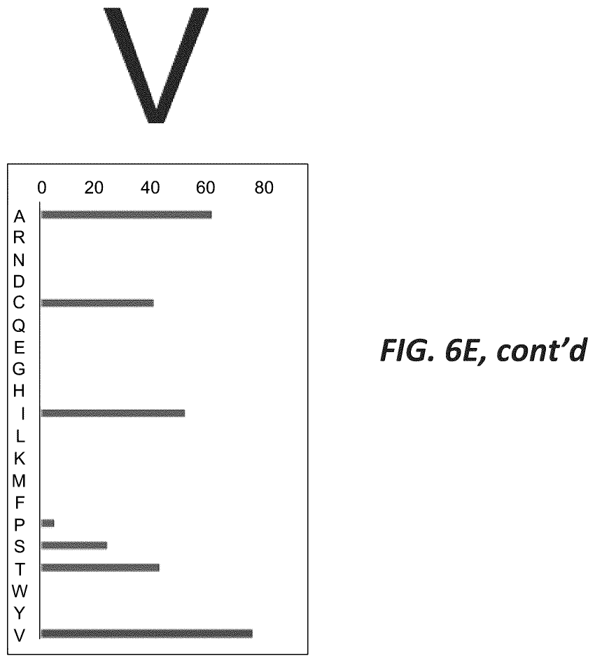

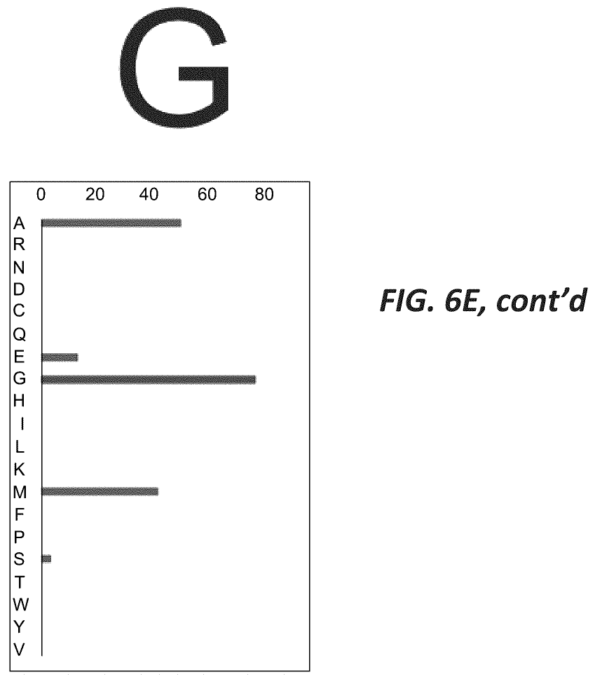

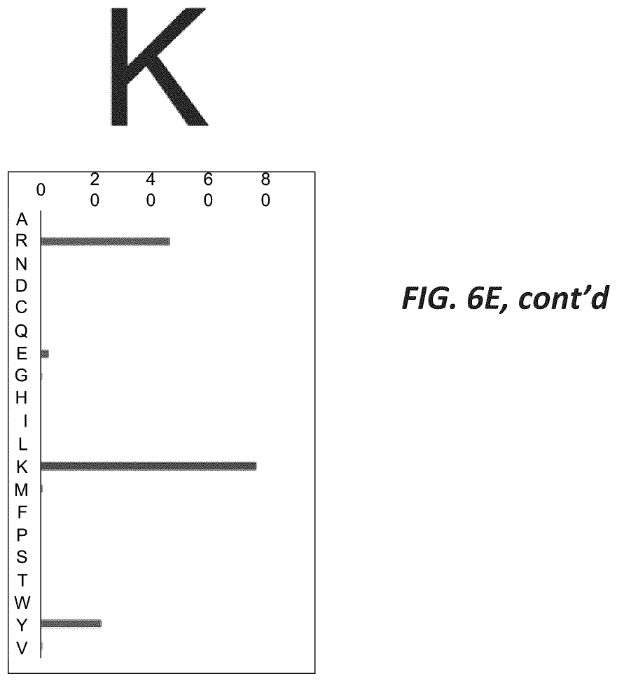

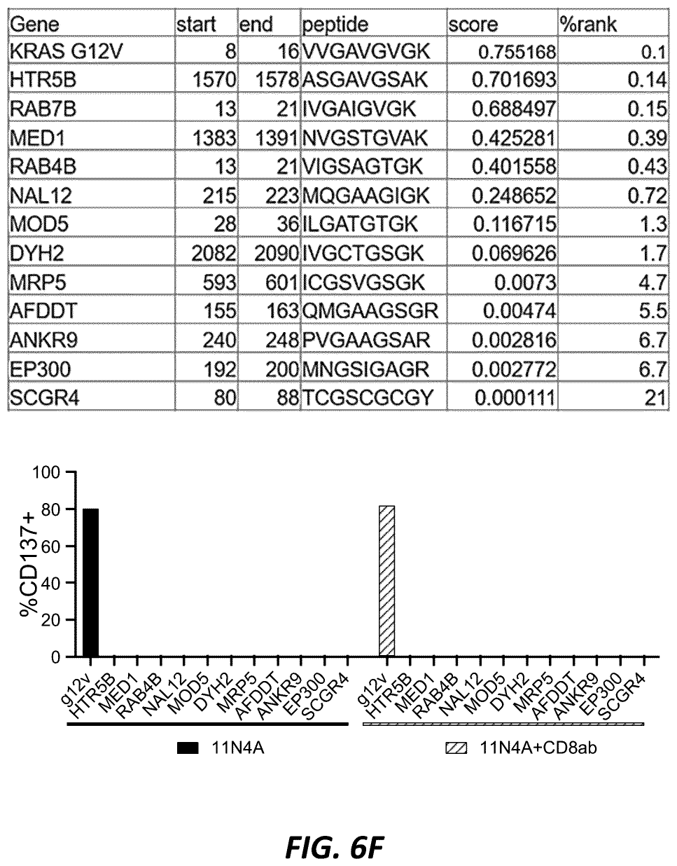

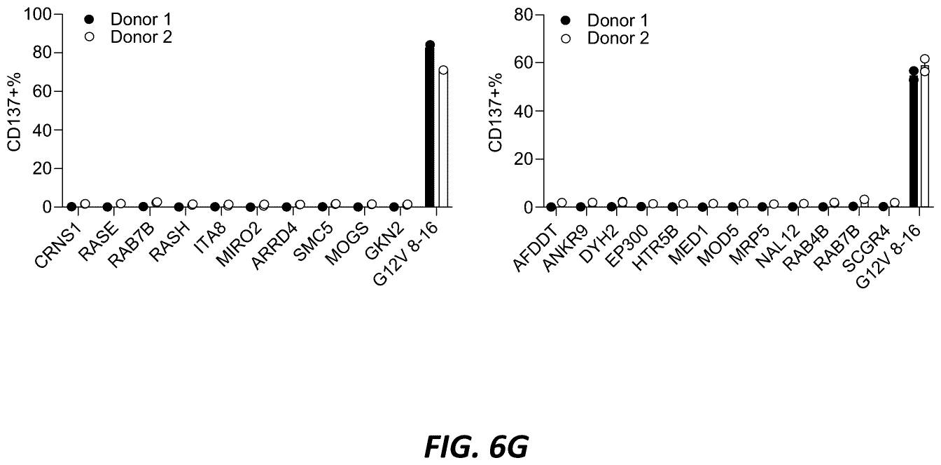

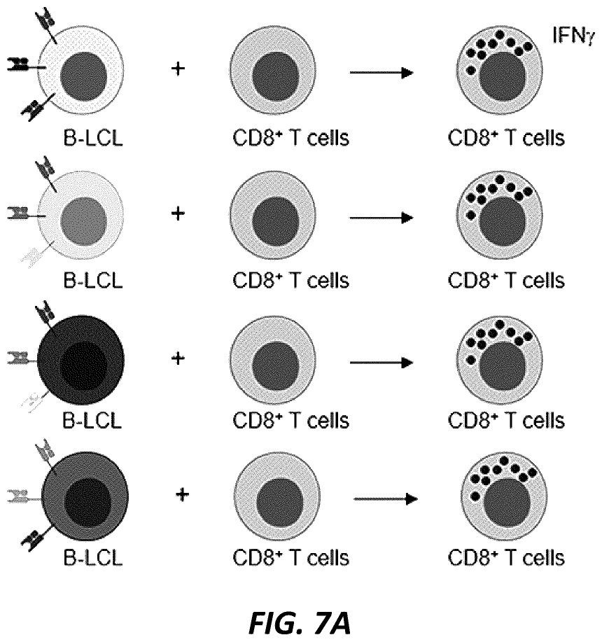

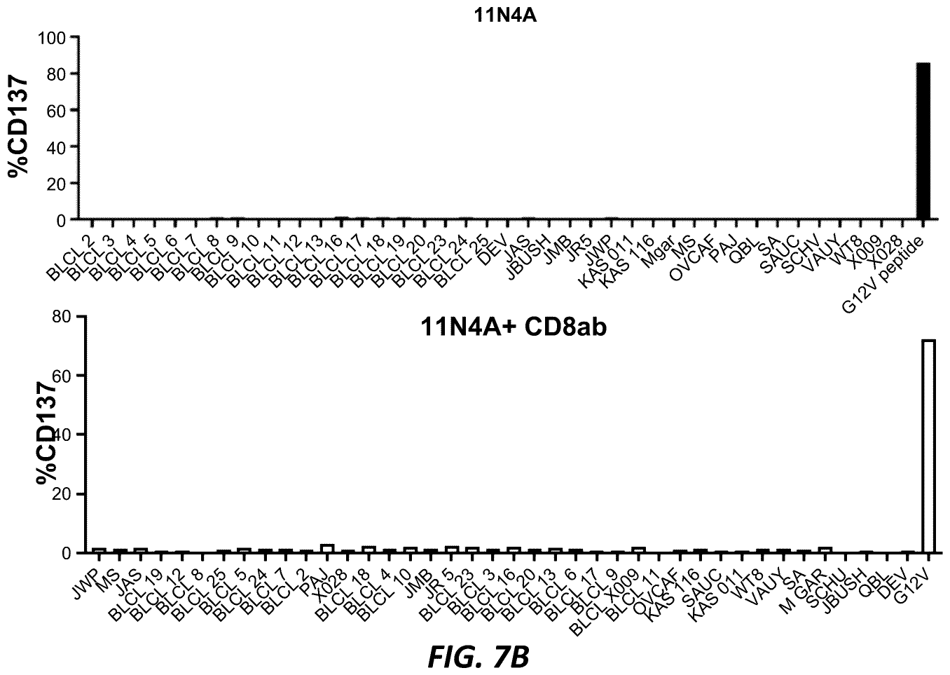

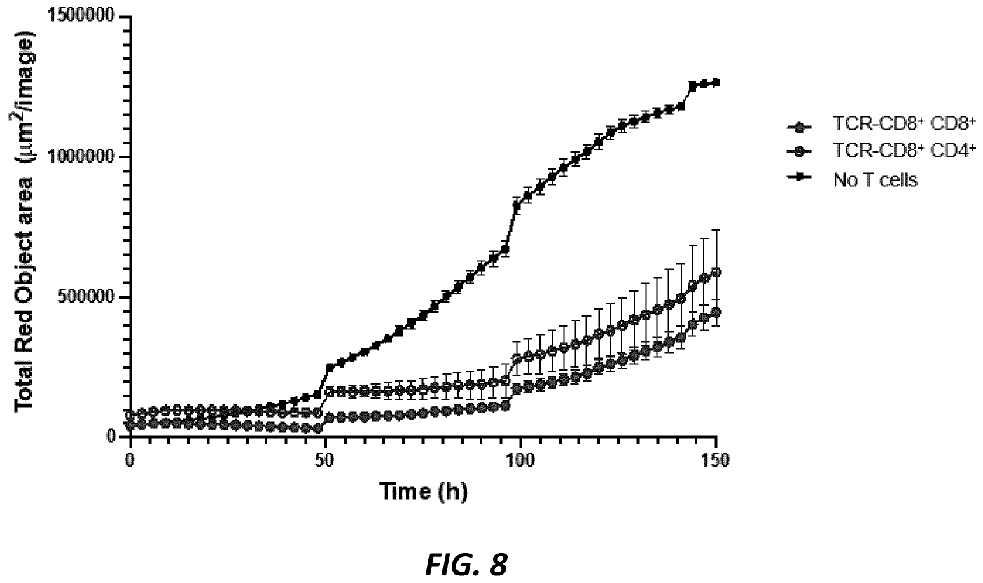

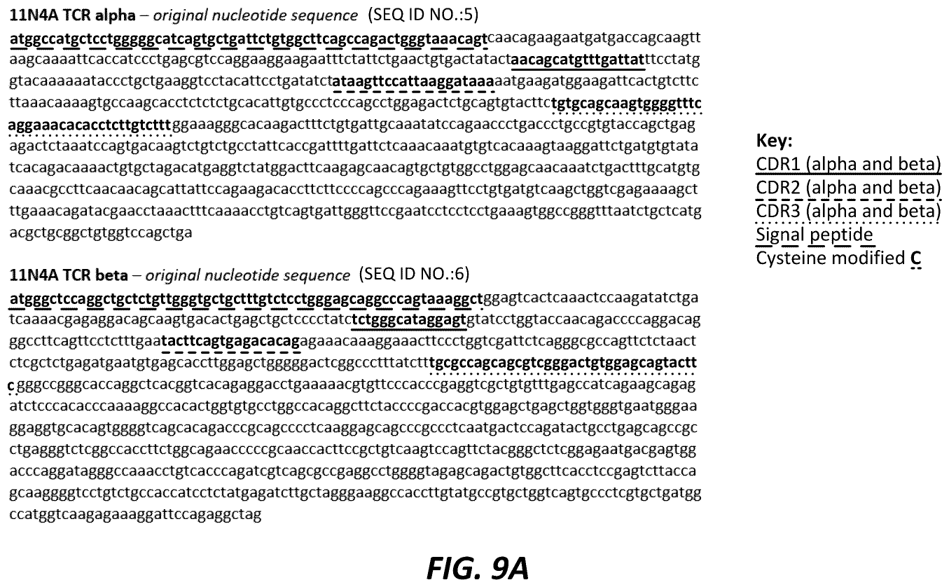

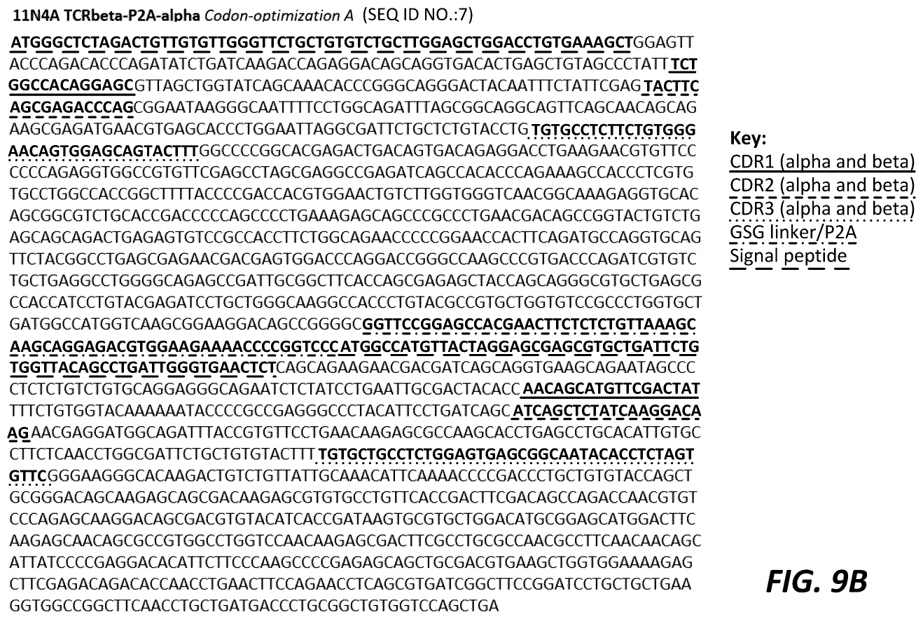

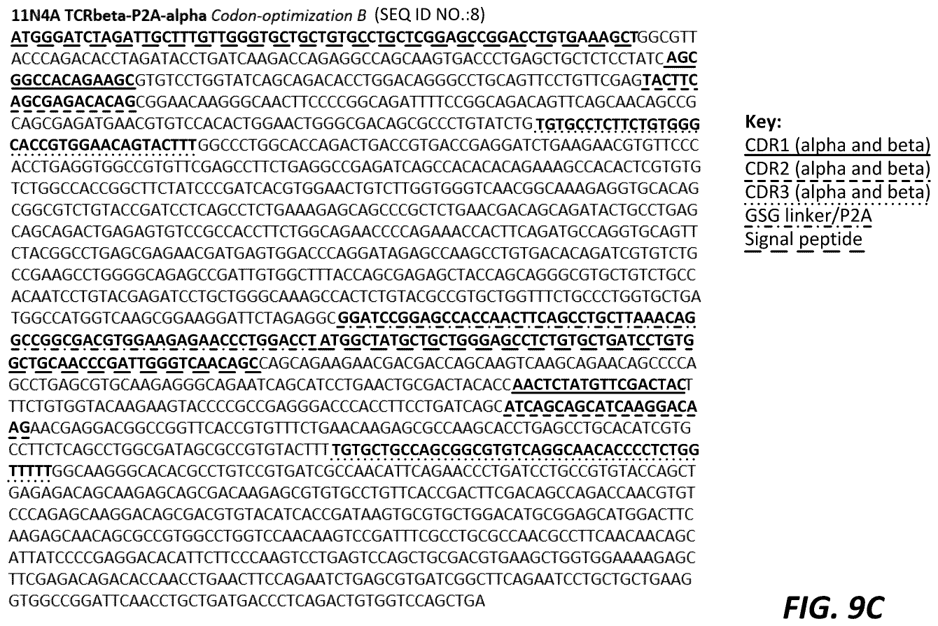

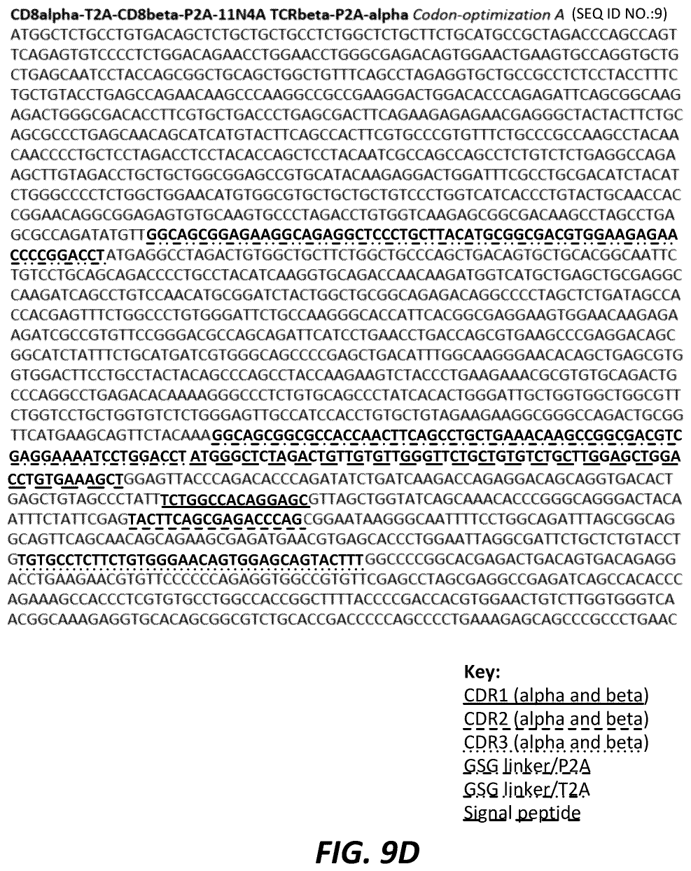

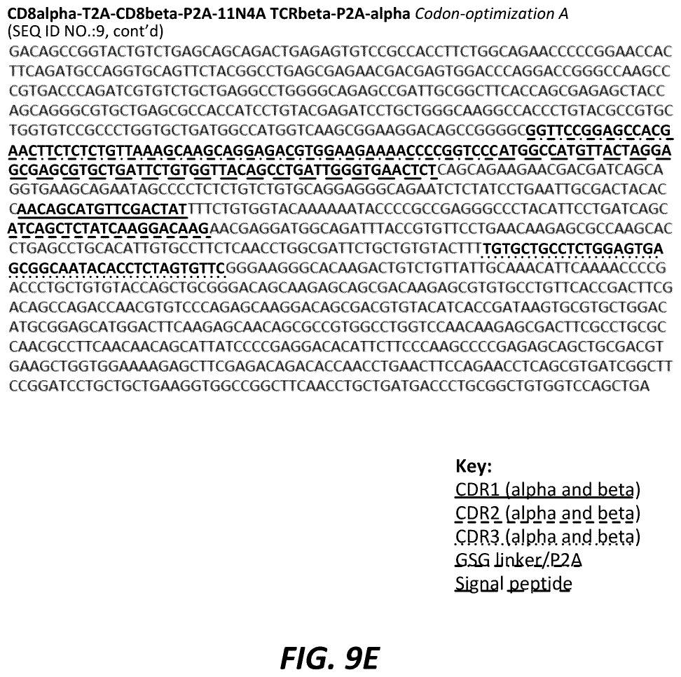

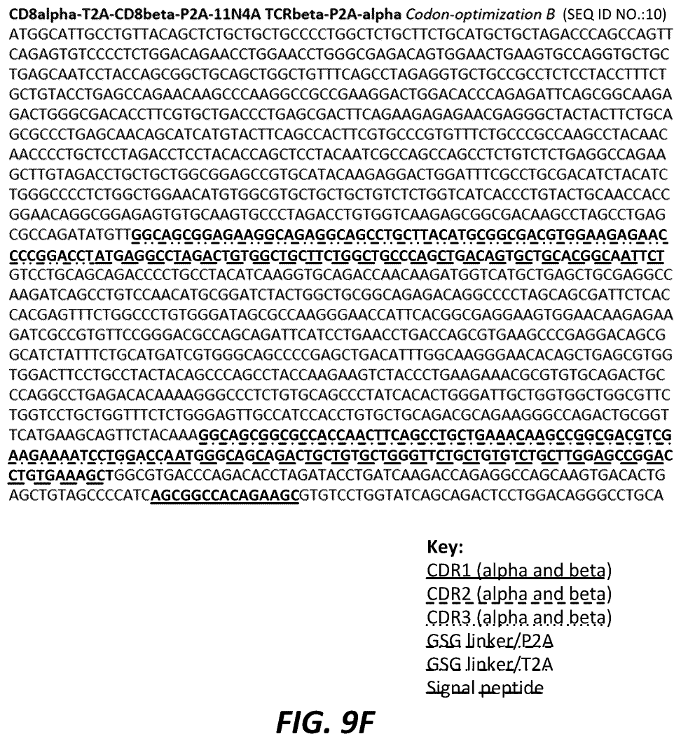

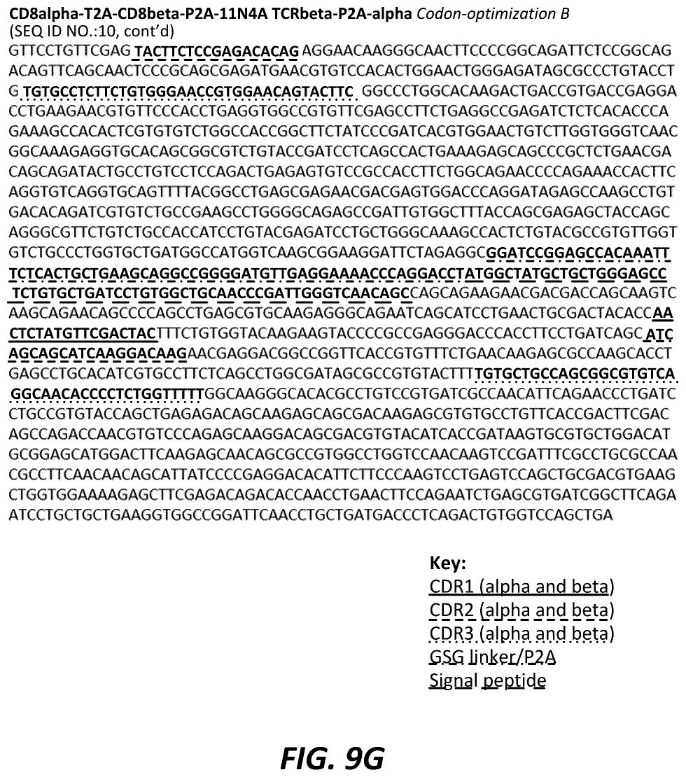

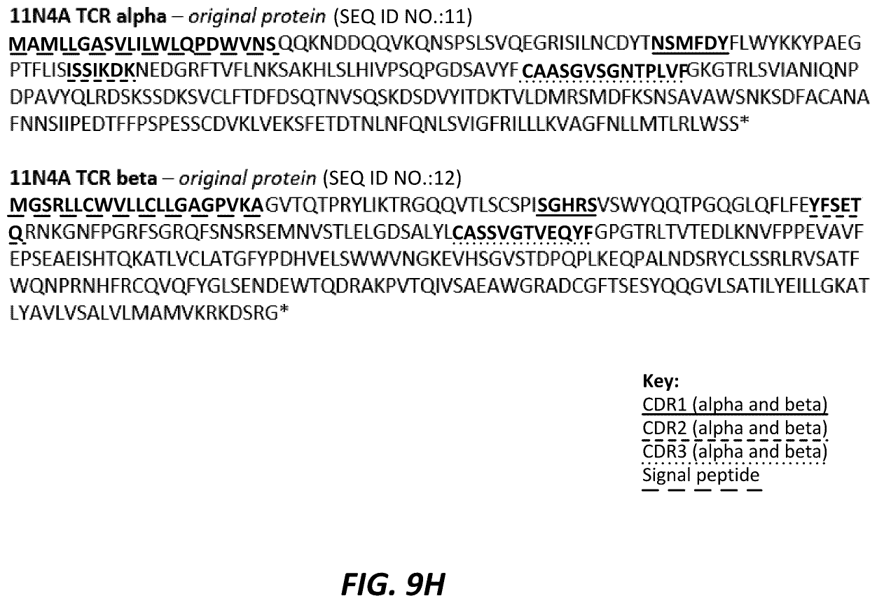

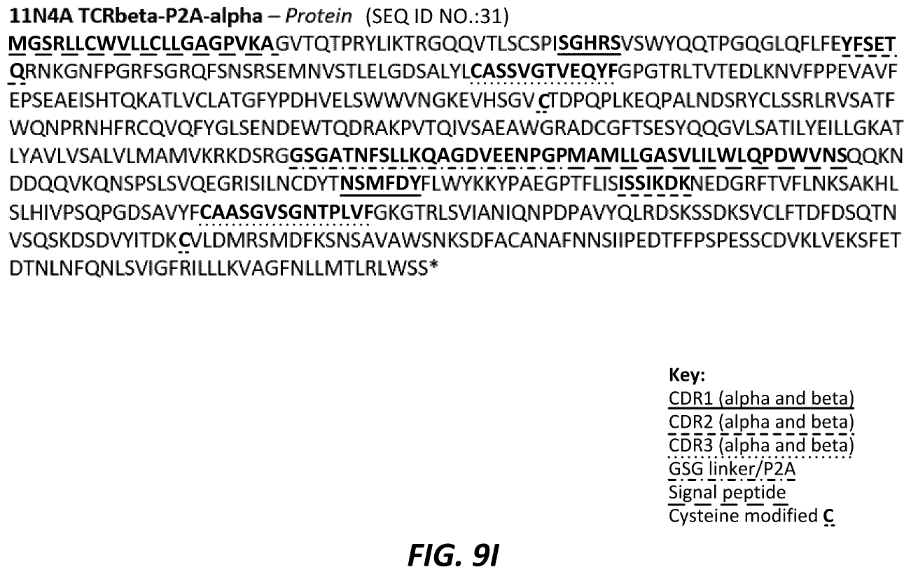

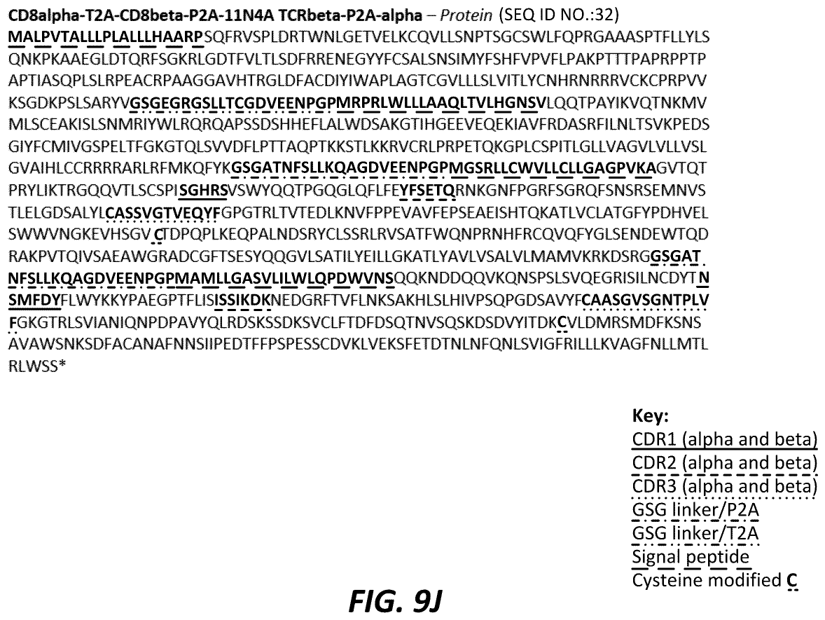

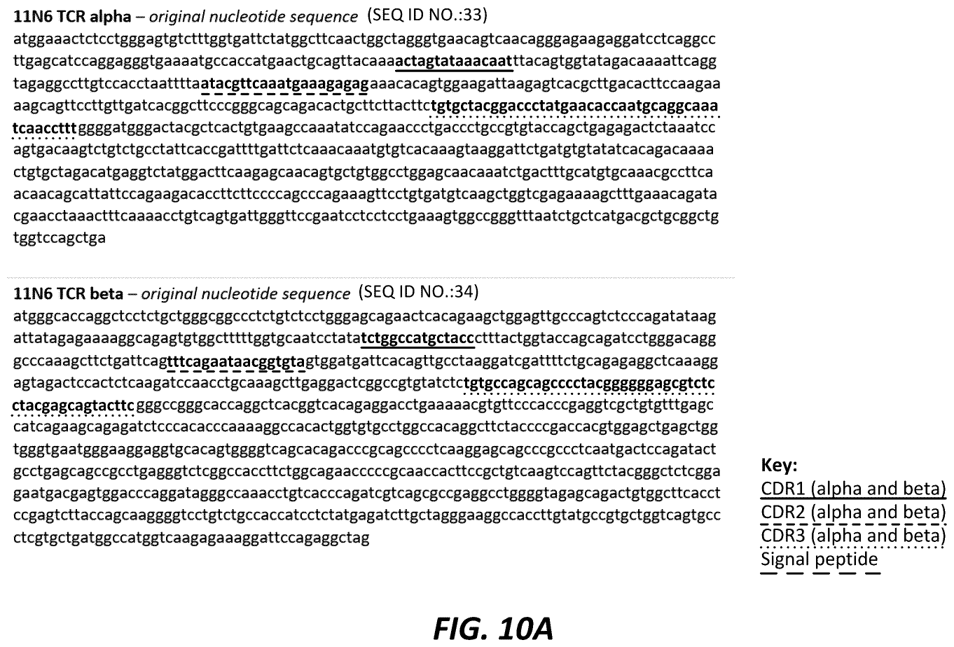

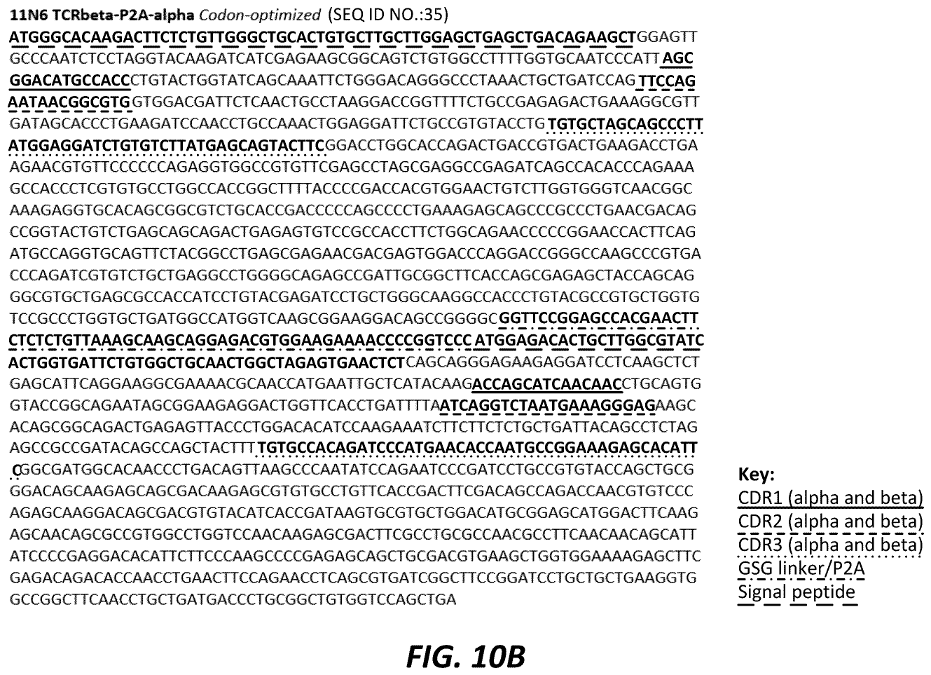

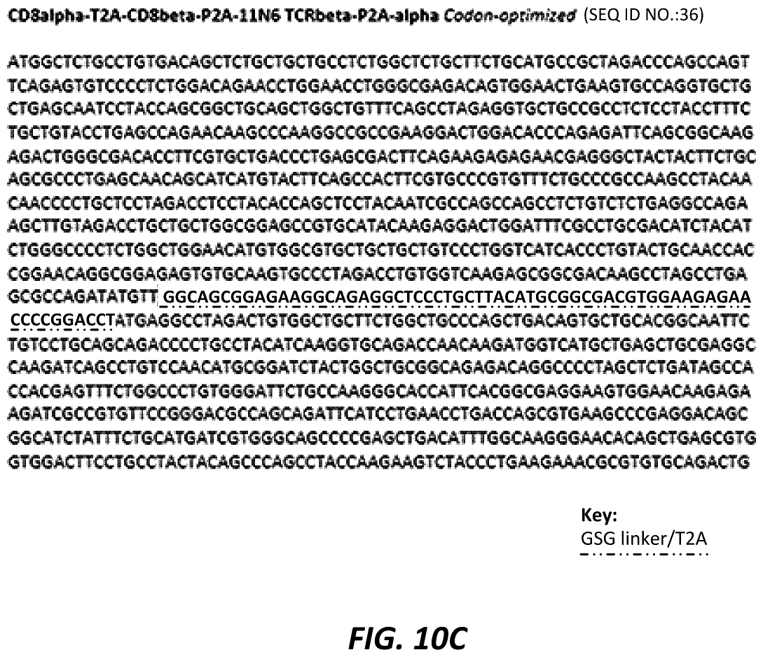

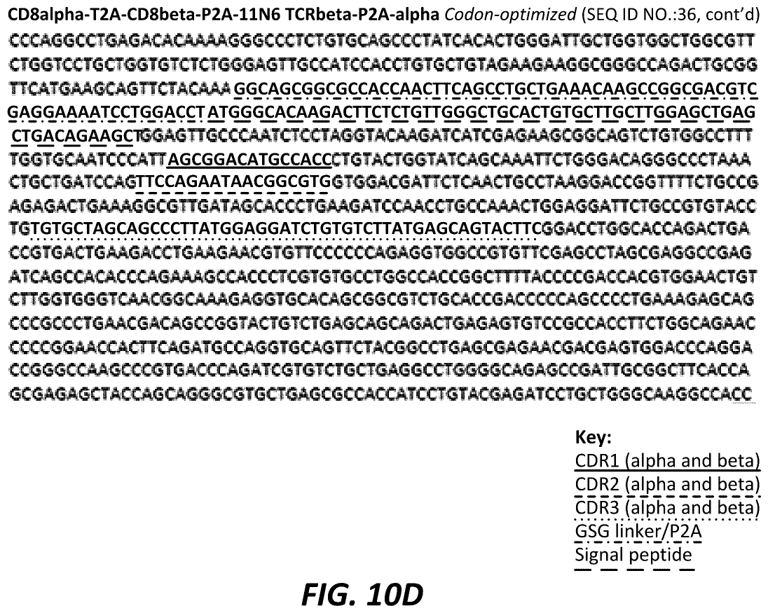

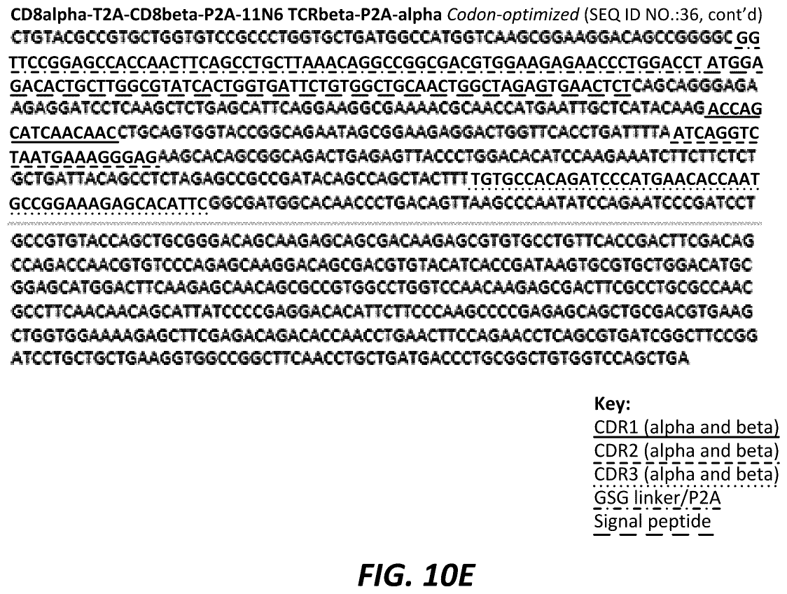

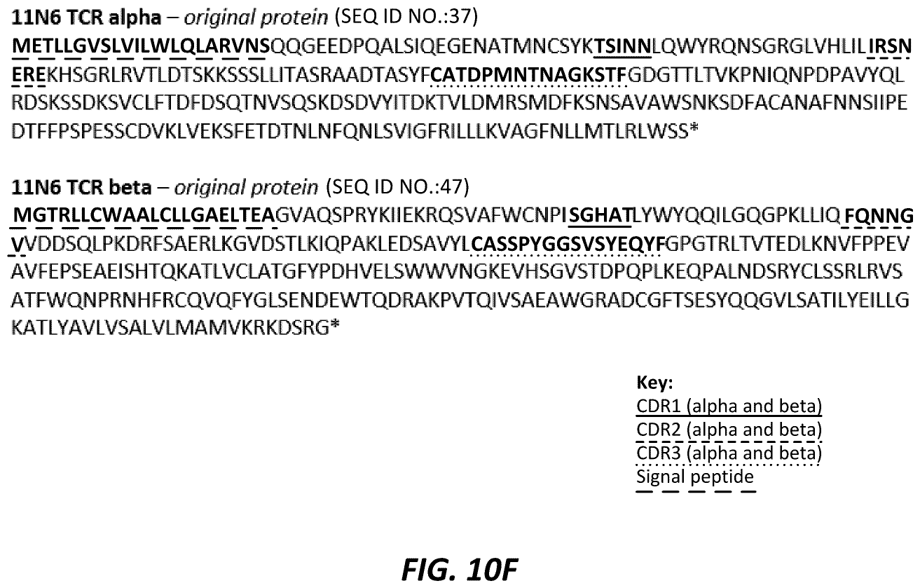

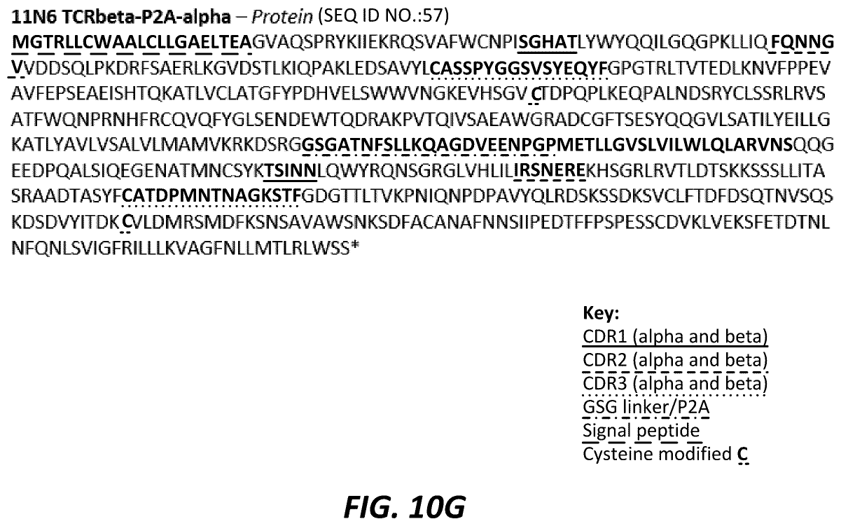

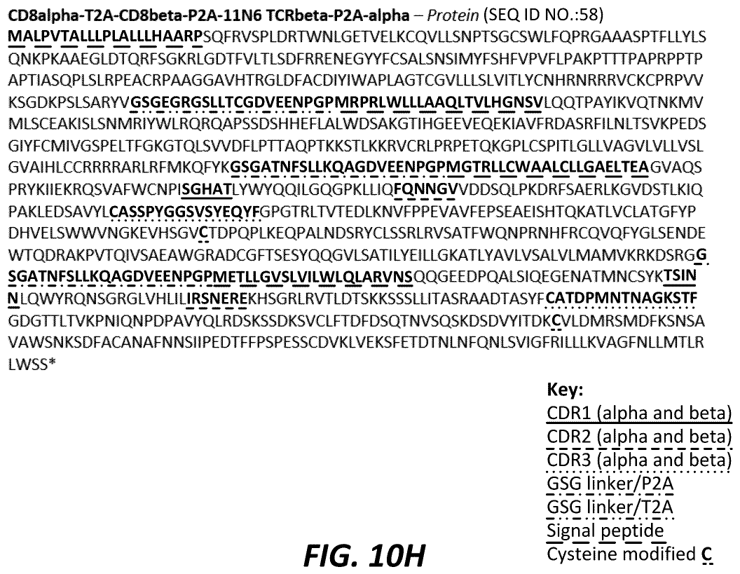

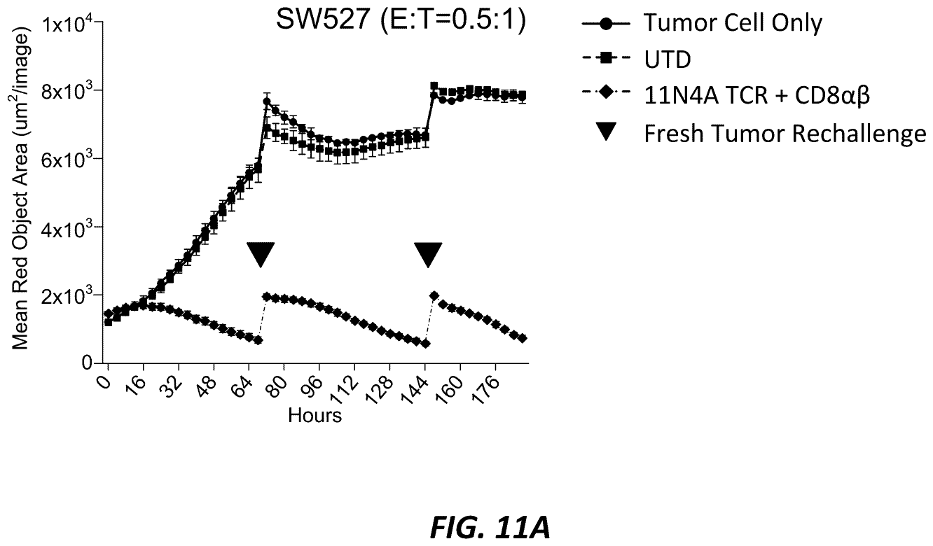

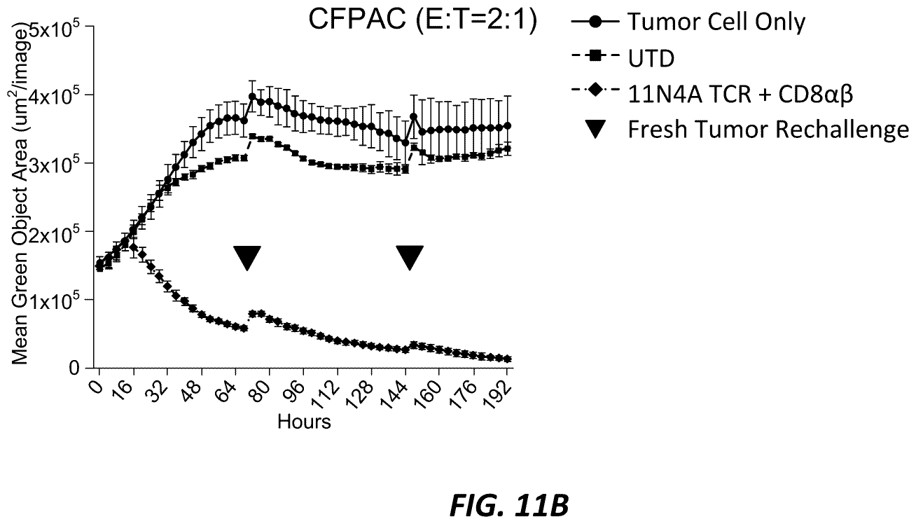

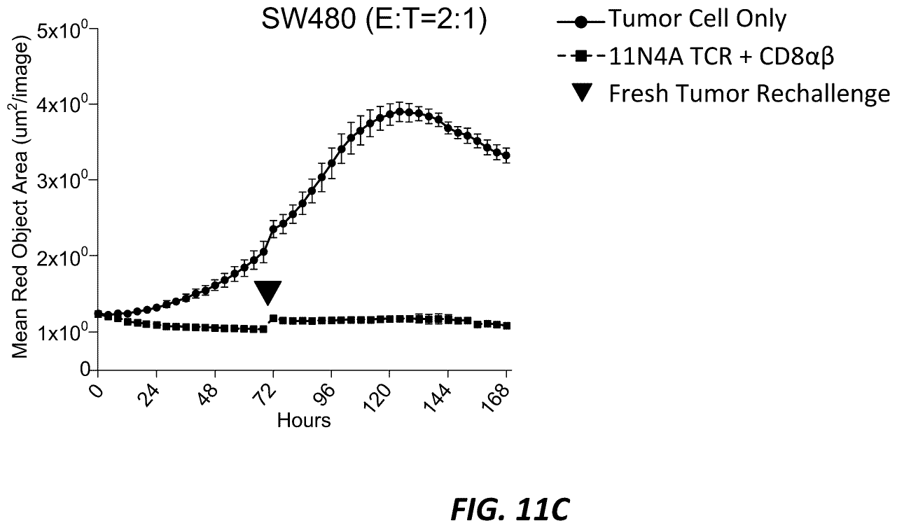

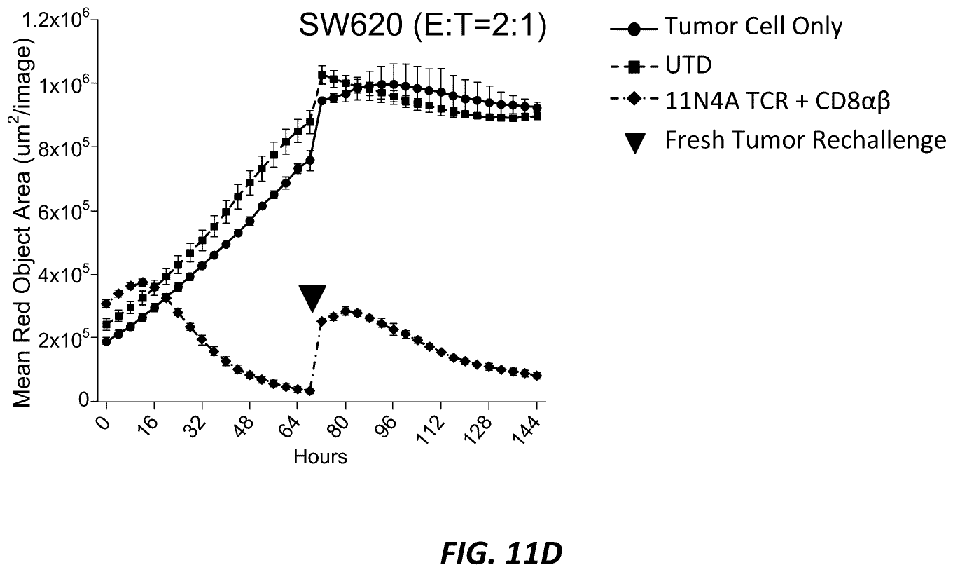

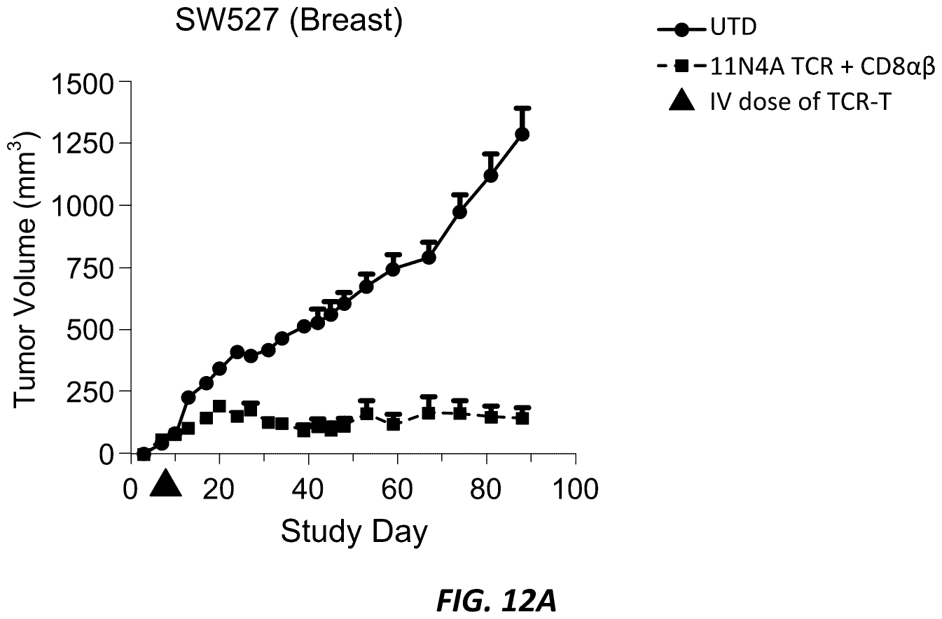

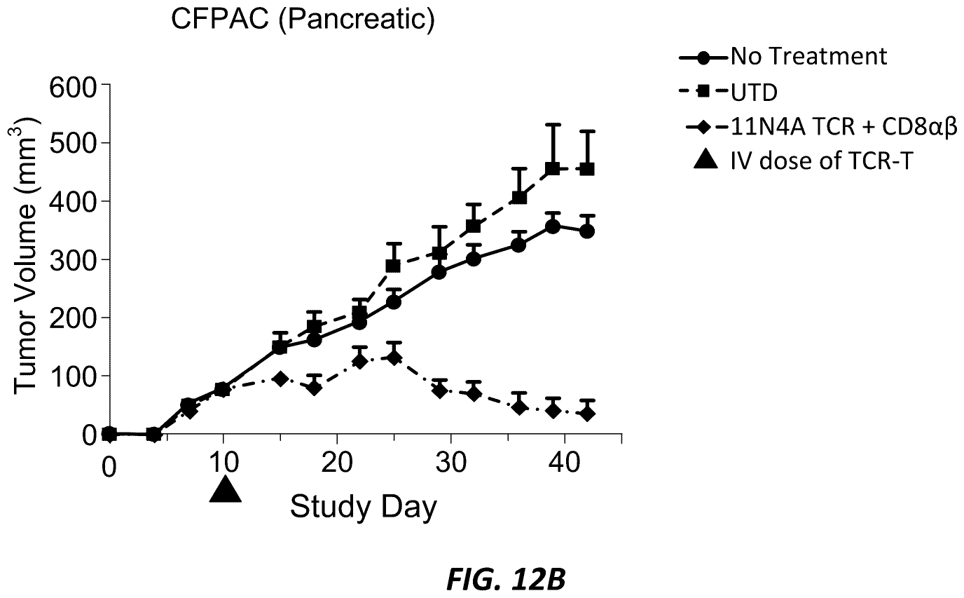

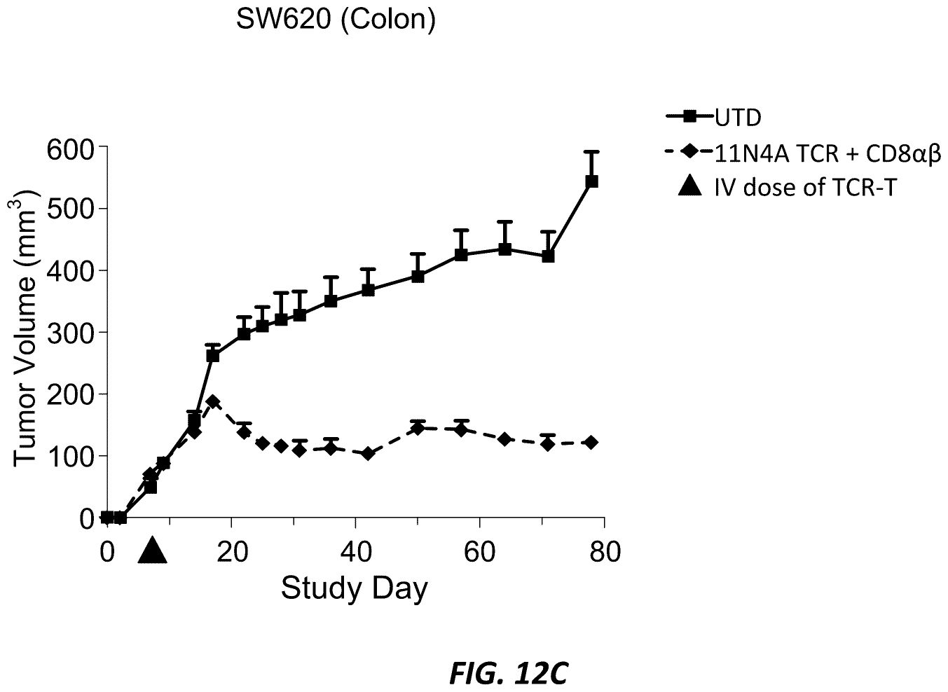

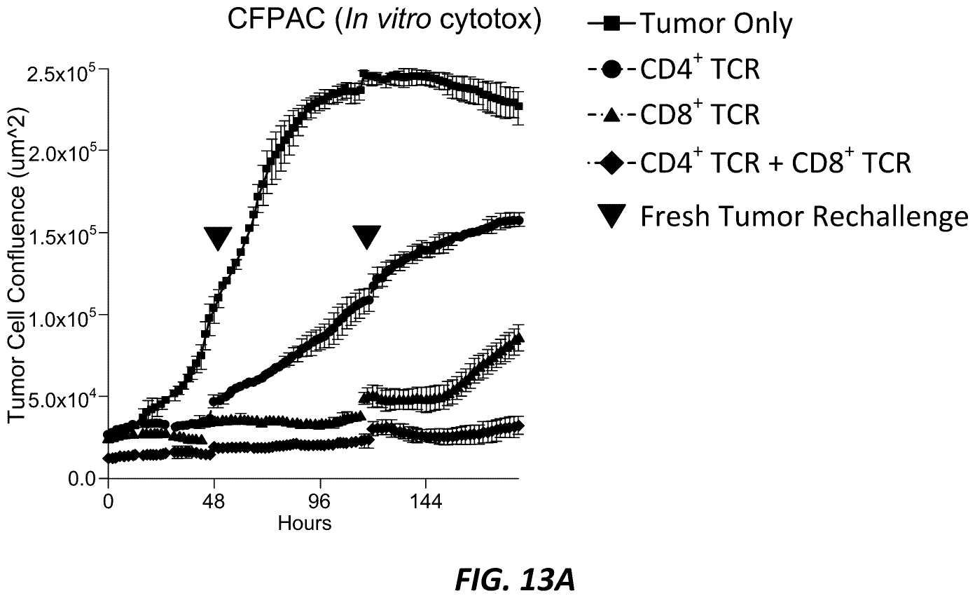

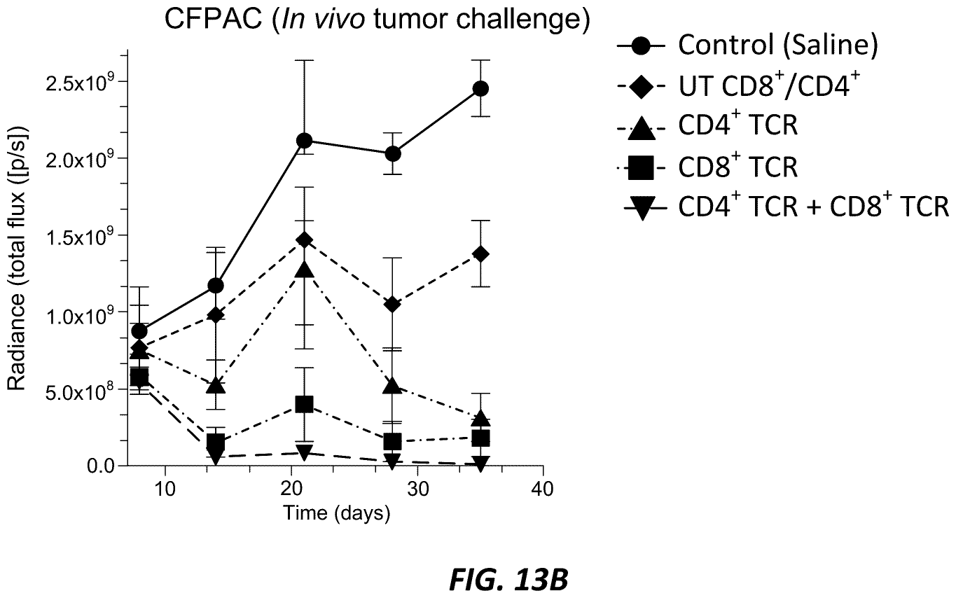

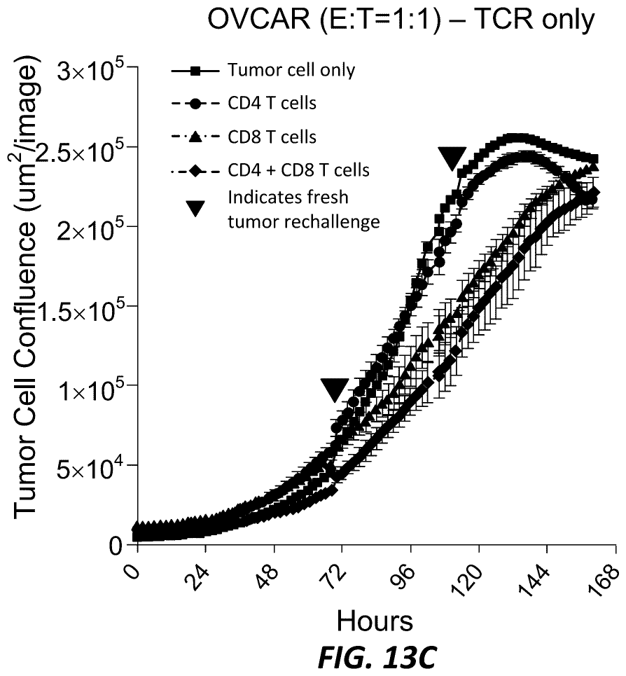

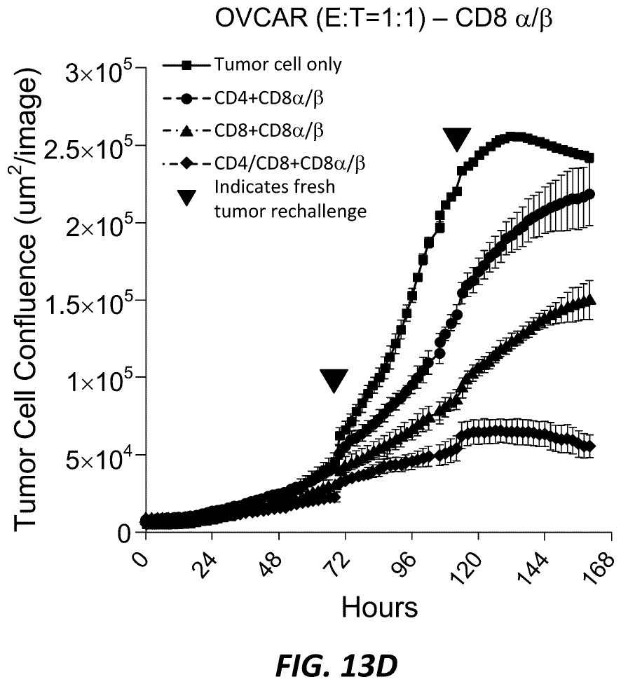

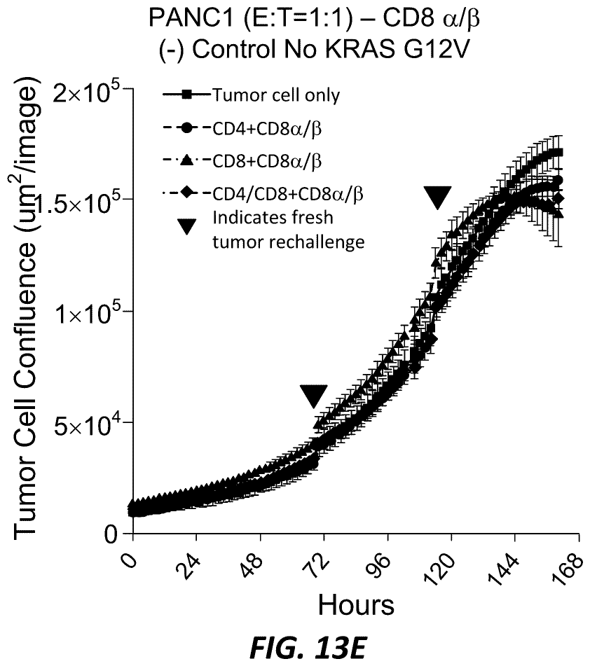

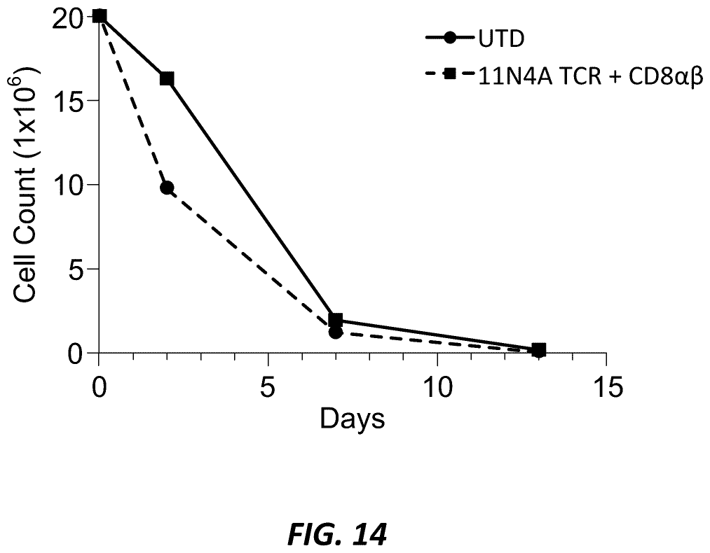

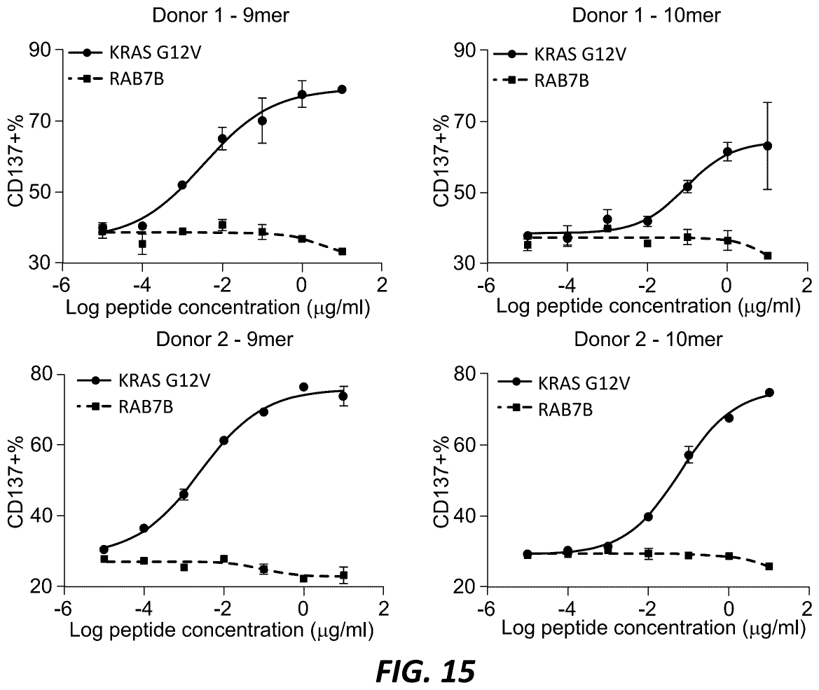

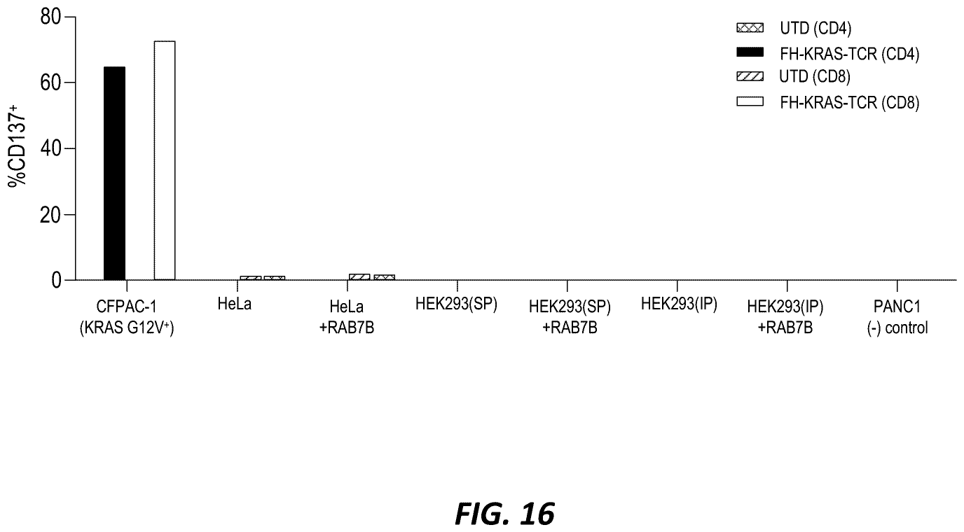

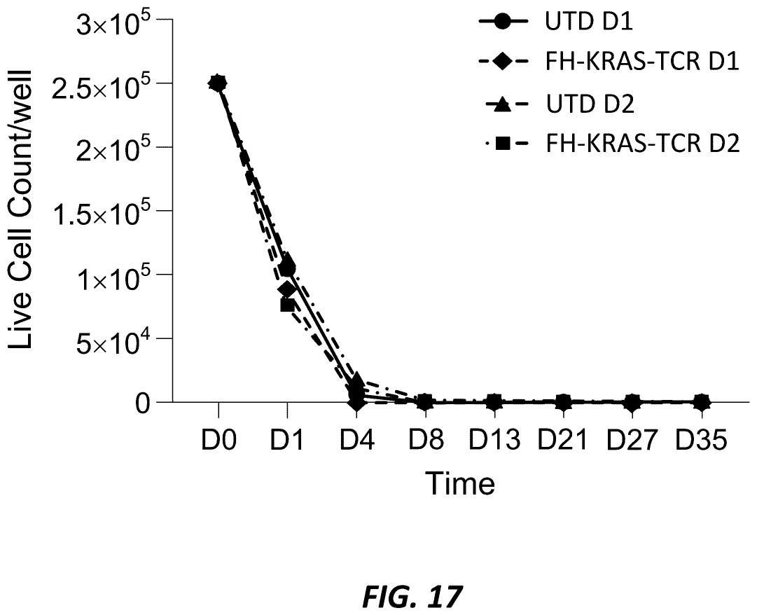

In FIGS. 3D-3F, 11N4A-TCR T cells are shown as “FH-KRAS-TCR”. FIG. 3D shows that 11N4A-TCR cells are activated by endogenously processed and presented KRAS G12V antigen across a diverse panel of tumor cell lines. (Left) Description of tumor cell lines used in this study. (Right) Indicated tumor cell lines expressing HLA-A*11:01 and KRAS G12V were cultured with untransduced or 11N4A-TCR T cells from two healthy donors (D1 and D2) for 20 hours at a 1:1 effector to target ratio. T cell activation was measured by flow cytometric CD137 surface staining. FIG. 3E shows that 11N4A-TCR cells secrete effector cytokines in response to endogenously processed and presented KRAS G12V antigen across a diverse panel of tumor cell lines. Indicated tumor cell lines expressing HLA-A*11:01 and KRAS G12V were cultured with untransduced or 11N4A-TCR T cells from two healthy donors (D1 and D2) for 20 hr. Supernatant from the co-culture activation assay shown in FIG. 3D were collected and IFNγ, TNFα and IL-2 cytokine secretion was analyzed by ELISA. IU=international units calculated based on standard curve of IFNγ recombinant protein. FIG. 3F shows that 11N4A-TCR T cells proliferate in response to endogenously processed and presented KRAS G12V antigen across a diverse panel of tumor cell lines. Indicated tumor cell lines expressing HLA-A*11:01 and KRAS G12V were cultured with untransduced or 11N4A-TCR T cells from two healthy donors (D1 and D2) for 6 days at a 1:1 effector to target ratio. T cell proliferation was measured by flow cytometric lymphocyte counts. T cell counts are plotted as Lymphocyte counts/μL. FIG. 3G shows that the diverse panel of tumor cell lines tested exhibit a range of KRAS G12V antigen expression. (Left) Western blot analysis of the indicated tumor cell lines. Cell lysates were prepared from tumor cells that were normalized by cell number. KRAS^{G12V}-specific antibody was used (MA5-42375, Invitrogen), with GAPDH-specific antibody used as a loading control (AB9483, Abcam). (Right) Densitometry analysis of the Western blot data at left, quantifying the ratio of KRAS G12V expression to GAPDH expression for all tumor cell lines tested.

FIGS. 4A-4G relate to specific killing of HLA-A11+ KRAS G12V-expressing tumor cell lines by CD8+ T cells expressing a KRAS G12V-specific TCR in an Incuyte killing assay. In this assay, the Red Object Area indicates the presence of tumor cells. (FIG. 4A) Schematic illustration of mKRAS tumor cell growth in the absence of mKRAS-specific T cells. (FIG. 4B) mKRAS+/HLA-A11+ tumor cell growth curves in an IncuCyte® killing assay. Tested conditions were tumor cells only, tumor cells+T cells transduced to express TCR 11N4A, and tumor cells transduced to express comparator TCR 220_21. The red object area on the y-axis shows tumor cell growth. Additional tumor cells were added at 72 h. (FIG. 4C) Data from another killing assay experiment. T cells and SW480 tumor cell line were present at the indicated effector:target ratios. (FIG. 4D) 11N4A-transduced primary CD4+ and CD8+ T cells are cytotoxic to the SW527, SW620, and CFPAC-1 tumor cell lines across multiple tumor cell challenges. Growth kinetics of indicated HLA-A*11:01+, KRAS G12V-expressing tumor cell lines in a live tumor-visualization assay in the presence of 11N4A-transduced (lower curve in each graph) or untransduced (upper curve in each graph) primary T cells. Tumor cells expressing a red fluorescent protein (SW527 and SW620 tumor cells) or a green fluorescent protein (CFPAC-1 tumor cells) were cultured with TCR-transduced or untransduced T cells for approximately 145 hours at a 10:1 effector to target ratio, tumor confluence is reported as a metric of tumor cell growth/viability throughout the study as indicated. Additional tumor cells were added at approximately 50 and 90 hours. (FIG. 4E)-(FIG. 4G): 11N4A-TCR cells are cytotoxic to a diverse panel of tumor cell lines. Growth kinetics of various indicated HLA-A*11:01+, KRAS G12V-expressing tumor cell lines cultured with untransduced or 11N4A-TCR T cells from two healthy donors (D1 and D2) in a live tumor-visualization assay. Tumor cells expressing a red fluorescent protein were cultured alone or with 11N4A-TCR T cells at a 10:1 effector:target ratio for 96 hours. Tumor cell confluence as measured by total red object area (labelled as tumor cell confluence) was reported as metric of tumor cell growth/viability throughout the study as indicated. In (E)-(G), 11N4A-TCR T cells are shown as “FH-KRAS-TCR”.

FIGS. 5A-5D relate to mutagenesis scanning experiments using KRAS G12 9-mer and 10-mer peptides to characterize the peptide binding motif of TCR 11N4A. (FIG. 5A) Percent of TCR-transduced T cells expressing Nur77-GFP when in the presence of G12V peptide (shown as “G12V WT”) or a variant of the G12V peptide with the amino acid at the indicated position replaced with alanine, glycine, or threonine, as indicated. Top: results from mutational scanning of KRAS G12 9-mer peptide. Bottom: results from mutational scanning of KRAS G12 10-mer peptide. (FIG. 5B) Percentage of 11N4A-transduced CD8+ T cells expressing the activation marker Nur77 (linked to a reporter gene) when in the presence of the indicated 9-mer peptide. (FIG. 5C) Schematic of workflow for identifying sequences from the human proteome that contain a sequence similar to the TCR 11N4A binding motif, and results from the workflow. (FIG. 5D) Results from searching the human proteome using the workflow shown in FIG. 5C. Peptides from the human proteome were scored for predicted binding to HLA-A11.

FIGS. 6A-6G show that TCR 11N4A has a low risk of autoreactivity in humans. (FIG. 6A, FIG. 6B) Reactivity of 11N4A-transduced T cells to a panel of potentially cross-reactive peptides (see FIG. 5B). (FIG. 6C) Peptide dose-response curve and (FIG. 6D) calculated negative log EC50 of 11N4A-transduced T cells against RAB7B peptide versus cognate KRAS G12V peptide. (FIG. 6E) Percentage of 11N4A-transduced CD8+ T cells expressing CD137 in response to overnight culture with a comprehensive panel of positional scanning peptides containing a substitution of every possible amino acid at each position of the cognate KRAS G12V peptide (172 peptides). Peptides that elicited a response of greater than 15% were considered positive in this assay. (FIG. 6F) (left) Potentially cross-reactive peptides identified from searching ScanProsite for the potentially cross-reactive motif identified from the data of (FIG. 6E). (Right) CD137 expression (determined by flow cytometry) by sort-purified primary CD8+ T cells transduced to express TCR 11N4A or TCR 11N4A+CD8αβ and cultured overnight with 100 ng/ml potentially cross-reactive peptide. (FIG. 6G) TCR 11N4A does not lead to cross-reactive peptide responses in vitro. T cell activation assay using 11N4A-TCR peptide-stimulated T cells generated from the PBMCs of two healthy donors. TCR 11N4A-T cells were incubated with 1 μg/ml of each indicated peptide for 18 hours followed by flow cytometric analysis of CD137 expression as a measure of T cell activation. (Left) Human self-peptides identified from alanine scan motif and (Right) Human self-peptides identified from XScan scan motif (see Table 2).

FIGS. 7A and 7B relate to screening to assess potential alloreactivity of TCR 11N4A. (FIG. 7A) B lymphoblastoid cell line (B-LCL) expressing different HLA alleles were incubated with 11N4A-transduced CD8+ T cells and the T cells were assessed for reactivity, as determined by expression of IFN-γ or CD137. (FIG. 7B) Results from the alloreactivity screen: percent of CD137+11N4A-transduced T cells with (top) or without (bottom) CD8αβ) against B-LCLs expressing common HLA alleles.

FIG. 8 shows killing activity of CD8+ and CD4+ T cells engineered to express TCR 11N4A and a CD8αβ co-receptor against mKRAS:HLA-A11+ tumor cells.

FIGS. 9A-9J show nucleotide (FIGS. 9A-9G) and amino acid (FIGS. 9H-9J) sequences relating to TCR 11N4A and expression constructs encoding or comprising the same.

FIGS. 10A-10H show nucleotide (FIGS. 10A-10E) and amino acid (FIGS. 10F-10H) sequences relating to TCR 11N6 and expression constructs encoding or comprising the same.

It will be understood that not all of the sequences as shown in FIGS. 9A-10H contains or annotates every sequence feature indicated in the key. The CDR3 sequences are shown in accordance with the IMGT junction definition.

FIGS. 11A-11D show cytotoxicity of primary 11N4A TCR and CD8αβ co-receptor engineered T cells against various tumor cell lines including SW527 (FIG. 11A), CFPAC (FIG. 11B), SW480 (FIG. 11C) and SW620 (FIG. 11D) in repeat tumor challenge assay. T cells (“E”) and target cells (“T”) were at the indicated E:T ratios.

FIGS. 12A-12C show robust in vivo anti-tumor activity of primary CD4+ and CD8+ T cells engineered to express 11N4A TCR and CD8αβ co-receptor in SW527 (FIG. 12A), CFPAC (FIG. 12B) and SW620 (FIG. 12C) tumor challenge models.

FIGS. 13A-13E illustrate improved in vitro and in vivo anti-tumor efficacy by a combination treatment of 11N4A TCR and CD8αβ co-receptor engineered CD4+ T cells and 11N4A TCR and CD8αβ coreceptor engineered CD8+ T cells compared to the single treatment of 11N4A TCR and CD8αβ co-receptor engineered CD4+ T cells or 11N4A TCR and CD8αβ co-receptor engineered CD8+ T cells. (FIG. 13A) Growth kinetics of CFPAC-1 tumor cell lines expressing HLA-A11+ KRAS G12V were measured in the presence of 11N4A TCR-CD8αβ co-receptor engineered CD4+ T cells, 11N4A TCR-CD8αβ co-receptor engineered CD8+ T cells, or 11N4A TCR-CD8αβ co-receptor engineered CD4+ and CD8+ T; (FIG. 13B) Tumor kinetics of CFPAC-Luc tumor cell inoculated in NSG immunocompromised mice were measured after intraperitoneal treatment of untransduced CD4+ and CD8+ T cells, 11N4A TCR-CD8αβ co-receptor engineered CD4+ T cells, 11N4A TCR-CD8αβ co-receptor engineered CD8+ T cells, or 11N4A TCR-CD8αβ co-receptor engineered CD4+ and CD8+ T cells respectively. (FIG. 13C)-(FIG. 13E): Combination of CD8αβ co-receptor (which can also be written as “CD8α/β” co-receptor) with a Class I TCR improves anti-tumor responses of TCR engineered T cells. Growth kinetics of HLA-A*11:01, KRAS G12V-expressing tumor cell lines (OVCAR-5) in the presence of primary CD4⁺ T cells only, CD8⁺ T cells only, or combined CD4⁺/CD8⁺ T cells (1:1 ratio) transduced with TCR 11N4A (FIG. 13C) or TCR 11N4A+CD8αβ coreceptor (FIG. 13D). Negative control tumor cell line (PANC1) that does not express KRAS G12V was used in (FIG. 13E). Tumor cells expressing a red fluorescent protein were cultured alone or with TCR-transduced T cells for 172 hours at a 1:1 effector to target ratio, and tumor cell confluence as measured by NucLight Red total red object area was reported as metric of tumor cell growth/viability throughout the study as indicated. Additional tumor cells were added at 72 and 108 hours.

FIG. 14 shows that T cells transduced with TCR 11N4A and CD8αβ co-receptor did not show cytokine-independent growth in vitro.

FIG. 15 shows no response of 11N4A-TCR simulated T cell product to RAB7B peptide. Peptide doses of KRAS G12V index peptide (positive control) and RAB7B spanning 10-0.00001 mg/mL were tested to assess reactivity of 11N4A-TCR simulated T cell product generated from two donors. 9-mer and 10-mer KRAS G12V and RAB7B peptides were exogenously added at titrating doses to 11N4A-TCR T cells for 16 hours followed by flow cytometric analysis of CD137 expression by T cells.

FIG. 16 shows no response of 11N4A-TCR simulated T cell product to over-expressed, endogenously processed and presented RAB7B. HEK293 cells expressing standard (SP) or immunoproteasomal (IP) subunits or HeLa cells were engineered to express HLA-A11 and RAB7B full-length protein. CFPAC-1 (KRAS G12V) and PANC1 (KRAS G12V) were used as positive and negative control, respectively. Sorted CD4⁺ or CD8⁺ 11N4A-TCR or untransduced (UTD) T cell product were cocultured with each cell line for 16 hours followed by flow cytometric analysis of CD137 expression by T cells. In this figure, 11N4A-TCR T cells are shown as “FH-KRAS-TCR”

FIG. 17 shows that 11N4A-TCR simulated products do not exhibit cell growth in the absence of cytokine. 11N4A-TCR T cell product was enriched for KRAS-G12V A11-Tetramer positive T cells and expanded with anti-CD3 and anti-CD28 beads in X VIVO 15+5% serum replacement media+100 U/mL IL-2 for 10 days in two donors. Untransduced primary T cells (UTD) from the same donors were similarly expanded side-by-side for 10 days. On day 10, cells were washed and resuspended in X VIVO 15+5% serum media lacking cytokines and cell growth kinetics were measured over 35 days. In FIG. 17, 11N4A-TCR T cells are shown as “FH-KRAS-TCR”.

### DETAILED DESCRIPTION

The present disclosure generally relates to binding proteins specific for Ras neoantigens, modified host (e.g., immune) cells expressing the same, polynucleotides that encode the binding proteins, and related uses. Mutated Ras proteins (e.g., KRAS, NRAS, HRAS) can produce neoantigens, including a G→V mutation at position 12 of the full-length KRAS protein (SEQ ID NO.: 1; UniProt KB P01116) or at position 12 of the full-length NRAS protein (SEQ ID NO.: 78; Uniprot KB P01111) or at position 12 of the full-length HRAS protein (SEQ ID NO.: 79; Uniprot KB P01112).

In the present disclosure, binding proteins that are capable of binding to Ras neoantigens are provided. In certain aspects, binding proteins (and host cells, such as immune cells, that comprise a heterologous polynucleotide that encodes a Ras-specific binding protein of the present disclosure) are provided that comprise a TCR Vα domain and a TCR Vβ domain, wherein the binding proteins are capable of binding to a Ras peptide antigen:HLA complex, wherein the Ras peptide antigen comprises, consists essentially of, or consists of the amino acid sequence set forth in any one of SEQ ID NOs: 2 or 3. In certain embodiments, the HLA comprises HLA-A*11, such as HLA-A*11:01.

Disclosed binding proteins are highly sensitive to antigen, capable in certain embodiments of inducing activation of host T cells at low concentrations of peptide antigen. In certain embodiments, of a population or sample of (e.g., CD8+ or CD4+) T cells expressing a binding protein, the T cells have half-maximal expression of the activation marker Nur77 when in the presence of [Log EC50 less than −9M (e.g., between −9M and −10M)] peptide. In certain embodiments, of a population or sample of (e.g., CD8+ or CD4+) T cells expressing a binding protein, the T cells have half-maximal expression of CD137 when in the presence of [Log EC50 less than −10M (e.g., between −10M and −11M)]. In certain embodiments, of a population or sample of (e.g., CD8+ or CD4+) T cells expressing a binding protein, the T cells have half-maximal expression of IFN-γ when in the presence of [Log EC50 less than −10M (e.g., between −10M and −11M)] peptide.

Host (e.g., T) cells expressing a binding protein according to the present disclosure are activated (e.g., as determined by expression of CD137) in the presence of mutant KRAS-expressing cancer cell lines, including OVCAR5 (ovarian serous adenocarcinoma), DAN-G (pancreatic adenocarcinoma), CFPAC1 (pancreatic adenocarcinoma), SW480 (colon carcinoma), SW527 (breast carcinoma), and NCI-H441 (lung adenocarcinoma) cell lines.

In some embodiments, host cells (e.g., T cells, such as CD4+ T cells or CD8+ T cells) expressing a binding protein according to the present disclosure are capable of specifically killing mutant KRAS-expressing cells (e.g., SW480 cells, such as at an 8:1 effector:target ratio, a 4:1 effector:target ratio, or a 2:1 effector:target ratio) for over 144 hours in vitro, including when additional tumor cells (i.e., additional of the mutant KRAS-expressing cells) are added at 72 hours in a re-challenge setting.

In certain embodiments, binding proteins of the present disclosure are non-alloreactive against, are substantially non-alloreactive against, and/or have a low risk of alloreactivity against (i) amino acid sequences from the human proteome and/or (ii) against human HLA alleles.

In any of the herein disclosed embodiments, a binding protein can be human, humanized, or chimeric. Also provided are polynucleotides that encode a binding protein, vectors that comprise a polynucleotide, and host cells that comprise a polynucleotide and/or vector and/or that express a binding protein. Presently disclosed binding proteins and host cells (e.g., T cells, NK cells, NK-T cells) are useful for treating a disease or disorder associated with a KRAS neoantigen, such as, for example, a cancer. Presently disclosed binding proteins can also bind to G12V antigens arising in human NRAS or human HRAS, which proteins comprise an identical sequence to KRAS in the region near residue G12. Accordingly, the disclosed compositions are useful in treating disease or disorders associated with a KRAS neoantigen comprising a G12V mutation, with an NRAS neoantigen comprising a G12V mutation, or with an HRAS neoantigen comprising a G12V mutation, or any combination thereof.

Also provided are methods and uses of the presently disclosed binding proteins, polynucleotides, vectors, host cells, and related compositions, for the treatment of a disease or disorder associated with a KRAS, NRAS, and/or HRAS mutation as provided herein.

Also provided are methods that comprise introducing a polynucleotide encoding a presently disclosed binding protein (or introducing a vector comprising the polynucleotide) into a host cell or into a plurality or population or sample of host cells. In some embodiments, the polynucleotide or vector further encodes: a polypeptide comprising an extracellular portion of a CD8 co-receptor α chain; a polypeptide comprising an extracellular portion of a CD8 co-receptor β chain, or both. In certain embodiments, the host cell or cells comprises a T cell, such as a CD4+ T cell or a CD8+ T cell. In certain embodiments, the host cell or cells comprises a primary T cell. In certain embodiments, the host cell or cells comprises a peripheral blood mononuclear cell (PBMC). In some embodiments, a method further comprises culturing the host cell or cells. In some embodiments, the host cell or cells is from a subject having a disease or disorder associated with a KRAS G12V or NRAS G12V or HRAS G12V mutation. In some embodiments, the disease or disorder comprises a cancer. In some embodiments, the subject is positive for expression of an HLA-A11, such as HLA-A*11:01. In certain embodiments, the host cell or cells is from a healthy subject. In some embodiments, the method is performed in vitro. In other embodiments, the method is performed ex vivo. Also provided is a host cell, host cell population, or host cell sample made by the method. In some embodiments, a host cell population comprises CD8+ T cells, CD4+ T cells, or both. In some embodiments, a method further comprises selecting for and combining CD8+ T cells with CD4+ T cells to provide a composition that comprises the CD8+ T cells and CD4+ T cells in about a 1:1 ratio.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include”, “have”, and “comprise” are used synonymously, which terms and variants thereof are intended to be construed as non-limiting.

In addition, it should be understood that the individual compounds, or groups of compounds, derived from the various combinations of the structures and substituents described herein, are disclosed by the present application to the same extent as if each compound or group of compounds was set forth individually. Thus, selection of particular structures or particular substituents is within the scope of the present disclosure.

The term “consisting essentially of” is not equivalent to “comprising” and refers to the specified materials or steps of a claim, or to those that do not materially affect the basic characteristics of a claimed subject matter. For example, a protein domain, region, or module (e.g., a binding domain, hinge region, linker module) or a protein (which may have one or more domains, regions, or modules) “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity or avidity of a binding protein).

As used herein, “protein” or “polypeptide” refers to a polymer of amino acid residues. Proteins apply to naturally occurring amino acid polymers, as well as to amino acid polymers in which one or more amino acid residue is an artificial chemical mimetic of a corresponding naturally occurring amino acid and non-naturally occurring amino acid polymers. In some embodiments, a “peptide” (e.g., a peptide antigen) refers to a polymer of about 8-10 amino acid residues in length.

As used herein, a “hematopoietic progenitor cell” is a cell that can be derived from hematopoietic stem cells or fetal tissue and is capable of further differentiation into mature cell types (e.g., immune system cells). Exemplary hematopoietic progenitor cells include those with a CD241^{Lo} Lin⁻ CD117⁺ phenotype or those found in the thymus (referred to as progenitor thymocytes).

As used herein, an “immune system cell” means any cell of the immune system that originates from a hematopoietic stem cell in the bone marrow, which gives rise to two major lineages, a myeloid progenitor cell (which give rise to myeloid cells such as monocytes, macrophages, dendritic cells, megakaryocytes and granulocytes) and a lymphoid progenitor cell (which give rise to lymphoid cells such as T cells, B cells and natural killer (NK) cells). Exemplary immune system cells include a CD4⁺ T cell, a CD8⁺ T cell, a CD4⁻ CD8⁻ double negative T cell, a γδ T cell, a regulatory T cell, a natural killer cell, a natural killer T cell, and a dendritic cell. Macrophages and dendritic cells can be referred to as “antigen presenting cells” or “APCs,” which are specialized cells that can activate T cells when a major histocompatibility complex (MHC) receptor on the surface of the APC complexed with a peptide interacts with a TCR on the surface of a T cell.

A “T cell” or “T lymphocyte” is an immune system cell that matures in the thymus and produces a T cell receptor (TCR). T cells can be naïve (“T_{N}”; not exposed to antigen; increased expression of CD62L, CCR7, CD28, CD3, CD127, and CD45RA, and decreased or no expression of CD45RO as compared to TCM (described herein)), memory T cells (T_{M}) (antigen experienced and long-lived), including stem cell memory T cells, and effector cells (antigen-experienced, cytotoxic). T_{M} can be further divided into subsets of central memory T cells (T_{CM}, expresses CD62L, CCR7, CD28, CD95, CD45RO, and CD127) and effector memory T cells (T_{EM}, express CD45RO, decreased expression of CD62L, CCR7, CD28, and CD45RA). Effector T cells (T_{E}) refers to antigen-experienced CD8⁺ cytotoxic T lymphocytes that express CD45RA, have decreased expression of CD62L, CCR7, and CD28 as compared to T_{CM}, and are positive for granzyme and perforin (e.g., upon stimulation). Helper T cells (T_{H}) are CD4⁺ cells that influence the activity of other immune cells by releasing cytokines. CD4⁺ T cells can activate and suppress an adaptive immune response, and which of those two functions is induced will depend on presence of, e.g., transcription factors, and other cells and signals. T cells can be collected using known techniques, and the various subpopulations or combinations thereof can be enriched or depleted by known techniques, such as by affinity binding to antibodies, flow cytometry, or immunomagnetic selection. Other exemplary T cells include regulatory T cells, such as CD4⁺ CD25⁺ (Foxp3) regulatory T cells and Treg17 cells, as well as Tr1, Th3, CD8⁺CD28⁻, and Qa-1 restricted T cells.

“T cell receptor” (TCR) refers to an immunoglobulin superfamily member having a variable binding domain, a constant domain, a transmembrane region, and a short cytoplasmic tail; see, e.g., Janeway et al., Immunobiology: The Immune System in Health and Disease, 3rd Ed., Current Biology Publications, p. 433, 1997) capable of specifically binding to an antigen peptide bound to a MHC receptor. A TCR can be found on the surface of a cell or in soluble form and generally comprises a heterodimer having α and β chains (also known as TCR α and TCRβ, respectively), or γ and δ chains (also known as TCRγ and TCRδ, respectively). In certain embodiments, a polynucleotide encoding a binding protein of this disclosure, e.g., a TCR, can be codon optimized to enhance expression in a particular host cell, such, for example, as a cell of the immune system, a hematopoietic stem cell, a T cell, a primary T cell, a T cell line, a NK cell, or a natural killer T cell (Scholten et al., Clin. Immunol. 119:135, 2006). Exemplary T cells that can express binding proteins and TCRs of this disclosure include CD4⁺ T cells, CD8⁺ T cells, and related subpopulations thereof (e.g., naïve, central memory, stem cell memory, effector memory).

Like immunoglobulins (e.g., antibodies), the extracellular portion of TCR chains (e.g., α-chain, β-chain) can contain two immunoglobulin domains, a variable domain (e.g., α-chain variable domain or V_{α}, β-chain variable domain or V_{β}; typically amino acids 1 to 116 based on Kabat numbering (Kabat et al., “Sequences of Proteins of Immunological Interest, US Dept. Health and Human Services, Public Health Service National Institutes of Health, 1991, 5^{th} ed.)) at the N-terminus, and one constant domain (e.g., α-chain constant domain or C_{α}, typically 5 amino acids 117 to 259 based on Kabat, β-chain constant domain or C_{β}, typically amino acids 117 to 295 based on Kabat) adjacent the cell membrane. Also, like immunoglobulins, the variable domains contain complementary determining regions (CDRs) separated by framework regions (FRs) (see, e.g., Jores et al., Proc. Nat'l Acad. Sci. USA 87:9138, 1990; Chothia et al., EMBO J. 7:3745, 1988; see also Lefranc et al., Dev. Comp. Immunol. 27:55, 2003). The source of a TCR as used in the present disclosure may be from various animal species, such as a human, mouse, rat, rabbit, or other mammal.

The term “variable region” or “variable domain” refers to the domain of an immunoglobulin superfamily binding protein (e.g., a TCR α-chain or β-chain (or γ chain and δ chain for γδ TCRs)) that is involved in binding of the immunoglobulin superfamily binding protein (e.g., TCR) to antigen. The variable domains of the α-chain and β-chain (Vα and Vβ, respectively) of a native TCR generally have similar structures, with each domain comprising four generally conserved framework regions (FRs) and three CDRs. The Vα domain is encoded by two separate DNA segments, the variable gene segment, and the joining gene segment (V-J); the Vβ domain is encoded by three separate DNA segments, the variable gene segment, the diversity gene segment, and the joining gene segment (V-D-J). A single Vα or Vβ domain may be sufficient to confer antigen-binding specificity. Furthermore, TCRs that bind a particular antigen may be isolated using a Vα or Vβ domain from a TCR that binds the antigen to screen a library of complementary Vα or Vβ domains, respectively.

The terms “complementarity determining region,” and “CDR,” are synonymous with “hypervariable region” or “HVR,” and are known in the art to refer to sequences of amino acids within immunoglobulin (e.g., TCR) variable regions. CDRs confer antigen specificity and binding affinity and are separated from one another in primary amino acid sequence by framework regions. In general, there are three CDRs in each TCR α-chain variable region (αCDR1, αCDR2, αCDR3 (also identified as CDR1α, CDR2α, and CDR3α, respectively)) and three CDRs in each TCR β-chain variable region (βCDR1, βCDR2, βCDR3 (also identified as CDR1β, CDR2β, and CDR3β, respectively)). In TCRs, CDR3 is thought to be the main CDR responsible for recognizing processed antigen. In general, CDR1 and CDR2 interact mainly or exclusively with the MHC.

CDR1 and CDR2 are encoded within the variable gene segment of a TCR variable region-coding sequence, whereas CDR3 is encoded by the region spanning the variable and joining segments for Vα, or the region spanning variable, diversity, and joining segments for Vβ. Thus, if the identity of the variable gene segment of a Vα or Vβ is known, the sequences of their corresponding CDR1 and CDR2 can be deduced; e.g., according to a numbering scheme as described herein. Compared with CDR1 and CDR2, CDR3, and in particular CDR3β, is typically significantly more diverse due to the addition and loss of nucleotides during the recombination process.

TCR variable domain sequences can be aligned to a numbering scheme (e.g., Kabat, Chothia, EU, IMGT, Enhanced Chothia, and Aho), allowing equivalent residue positions to be annotated and for different molecules to be compared using, for example, ANARCI software tool (2016, Bioinformatics 15:298-300). A numbering scheme provides a standardized delineation of framework regions and CDRs in the TCR variable domains. In certain embodiments, a CDR of the present disclosure is identified or defined according to the IMGT numbering scheme (Lefranc et al., Dev. Comp. Immunol. 27:55, 2003; imgt.org/IMGTindex/V-QUEST.php). In some embodiments, a CDR (e.g., CDR3) is identified or defined in accordance with the IMGT junction definition. In some embodiments, a CDR (e.g., CDR3, or all six CDRs of a binding protein) is identified or defined in accordance with the IMGT definition (or scheme or method). Examples of CDRs identified or defined in accordance with IMGT are provided in SEQ ID NOs.: 14-17 (with reference to Vα or TCRα of 11N4A), 24-27 (with reference to Vβ or TCRβ of TCR 11N4A), 40-43 (with reference to Vα or TCRα of TCR 11N6), and 50-53 (with reference to Vβ or TCRβ of TCR 11N6). In some embodiments, a CDR (or all six CDRs of a binding protein) of the present disclosure is identified or defined according to the Kabat numbering scheme or method. In some embodiments, a CDR (or all six CDRs of a binding protein) of the present disclosure is identified or defined according to the Chothia numbering scheme or method. In some embodiments, a CDR (or all six CDRs of a binding protein) of the present disclosure is identified or defined according to the EU numbering scheme or method. In some embodiments, a CDR (or all six CDRs of a binding protein) of the present disclosure is identified or defined according to the enhanced Chothia numbering scheme or method. In some embodiments, a CDR (or all six CDRs of a binding protein) of the present disclosure is identified or defined of the present disclosure is identified according to the Aho numbering scheme or method.

The source of a TCR as used in the present disclosure may be from any of a variety of animal species, such as a human, mouse, rat, rabbit, or other mammal. TCR constant domain sequences may be from, for example, human, mouse, marsupial (e.g., opossum, bandicoot, wallaby), shark, or non-human primate. In certain preferred embodiments, TCR constant domain sequences are human or comprise engineered variants of human sequences. TCR constant domains may be engineered to, for example, improve pairing, expression, stability, or any combination of these. See, e.g., Cohen et al., Cancer Res, 2007; Kuball et al., Blood 2007; and Haga-Friedman et al., Journal of Immunology 2009. Examples of engineering in TCR Cα and Cβ include mutation of a native amino acid to a cysteine so that a disulfide bond forms between the introduced cysteine of one TCR constant domain and a native cysteine of the other TCR constant domain. Such mutations can include, e.g., T48C in Cα, T57C or S57C in Cβ, or both. Also provided are embodiments wherein cognate TCR constant domains comprise mutations so that, for example, one TCR constant domain (e.g., one of Cα and Cβ) comprises an introduced so-called “cavity” (e.g., obtainable by replacing one or more native amino acid with one or more amino acids having smaller side chains) and the other (e.g., the other of Cα and Cβ) comprises a compensatory so-called “protuberance” (e.g., obtainable by replacing one or more native amino acid with one or more amino acids having larger side chains), similar to a “knob-into-hole” configuration used to promote preferential pairing of antibody heavy chains. Also provided are embodiments wherein TCR constant domain amino acids are mutated to introduce or modify charge properties so as to favor pairing of the mutated constant domains. Examples of mutations that may be made in Cα and Cβ to promote specific pairing by a knobs-into-holes-type mechanism or by a charge-pairing mechanism are provided in Voss et al., J. Immunol 180(1): 391-401 (2008) doi.org/10.4049/jimmunol.180.1.391; see also U.S. Pat. No. 9,062,127. The TCR constant domain mutations, mutated TCR constant domains, and methods used to identify sites for mutation, described in these documents, are incorporated herein by reference.

Mutations to improve stability can include a mutation in the Cα transmembrane domain from the sequence LSVIGF to the sequence LLVIVL (“L-V-L” mutation; see Haga-Friedman et al., J Immunol 188:5538-5546 (2012), the TCR mutations and mutant TCR constant domain sequences of which are incorporated herein by reference).

As used herein, the term “CD8 co-receptor” or “CD8” means the cell surface glycoprotein CD8, either as an alpha-alpha homodimer or an alpha-beta heterodimer. The CD8 co-receptor assists in the function of cytotoxic T cells (CD8⁺) and functions through signaling via its cytoplasmic tyrosine phosphorylation pathway (Gao and Jakobsen, Immunol. Today 21:630-636, 2000; Cole and Gao, Cell. Mol. Immunol. 1:81-88, 2004). There are five (5) human CD8 beta chain isoforms (see UniProtKB identifier P10966) and a single human CD8 alpha chain isoform (see UniProtKB identifier P01732).

“CD4” is an immunoglobulin co-receptor glycoprotein that assists the TCR of CD4+ cells in communicating with antigen-presenting cells (see, Campbell & Reece, Biology 909 (Benjamin Cummings, Sixth Ed., 2002)). CD4 is found on the surface of immune cells such as T helper cells, monocytes, macrophages, and dendritic cells, and includes four immunoglobulin domains (D1 to D4) that are expressed at the cell surface. During antigen presentation, CD4 is recruited, along with the TCR complex, to bind to different regions of the MHCII molecule (CD4 binds MHCII β2, while the TCR complex binds MHCII α1/β1). Without wishing to be bound by theory, it is believed that close proximity to the TCR complex allows CD4-associated kinase molecules to phosphorylate the immunoreceptor tyrosine activation motifs (ITAMs) present on the cytoplasmic domains of CD3. This activity is thought to amplify the signal generated by the activated TCR in order to produce or recruit various types of immune system cells, including T helper cells, and immune responses.

In certain embodiments, a TCR is found on the surface of T cells (or T lymphocytes) and associates with a CD3 complex. “CD3” is a multi-protein complex of six chains (see, Abbas and Lichtman, 2003; Janeway et al., p. 172 and 178, 1999) that is associated with antigen signaling in T cells. In mammals, the complex comprises a CD3γ chain, a CD3δ chain, two CD3ε chains, and a homodimer of CD3ζ chains. The CD3γ, CD3β, and CD3ε chains are related cell surface proteins of the immunoglobulin superfamily containing a single immunoglobulin domain. The transmembrane regions of the CD3γ, CD3β, and CD3ε chains are negatively charged, which is believed to allow these chains to associate with positively charged regions of T cell receptor chains. The intracellular tails of the CD3γ, CD3β, and CD3ε chains each contain a single conserved motif known as an immunoreceptor tyrosine based activation motif or ITAM, whereas each CD3ζ chain has three. Without wishing to be bound by theory, it is believed that the ITAMs are important for the signaling capacity of a TCR complex. CD3 as used in the present disclosure may be from various animal species, including human, mouse, rat, or other mammals.

As used herein, “TCR complex” refers to a complex formed by the association of CD3 with TCR. For example, a TCR complex can be composed of a CD3γ chain, a CD3β chain, two CD3ε chains, a homodimer of CD3ζ chains, a TCRα chain, and a TCRβ chain. Alternatively, a TCR complex can be composed of a CD3γ chain, a CD3β chain, two CD3ε chains, a homodimer of CD3ζ chains, a TCRγ chain, and a TCRβ chain.

A “component of a TCR complex”, as used herein, refers to a TCR chain (i.e., TCRα, TCRβ, TCRγ or TCRδ), a CD3 chain (i.e., CD3γ, CD3δ, CD3ε or CD3ζ), or a complex formed by two or more TCR chains or CD3 chains (e.g., a complex of TCRα and TCRβ, a complex of TCRγ and TCRδ, a complex of CD3ε and CD3δ, a complex of CD3γ and CD3ε, or a sub-TCR complex of TCRα, TCRβ, CD3γ, CD3δ, and two CD3ε chains).

“Chimeric antigen receptor” (CAR) refers to a fusion protein that is engineered to contain two or more naturally occurring amino acid sequences, domains, or motifs, linked together in a way that does not occur naturally or does not occur naturally in a host cell, which fusion protein can function as a receptor when present on a surface of a cell. CARs can include an extracellular portion comprising an antigen-binding domain (e.g., obtained or derived from an immunoglobulin or immunoglobulin-like molecule, such as a TCR binding domain derived or obtained from a TCR specific for a cancer antigen, a scFv derived or obtained from an antibody, or an antigen-binding domain derived or obtained from a killer immunoreceptor from an NK cell) linked to a transmembrane domain and one or more intracellular signaling domains (optionally containing co-stimulatory domain(s)) (see, e.g., Sadelain et al., Cancer Discov., 3(4): 388 (2013); see also Harris and Kranz, Trends Pharmacol. Sci., 37(3): 220 (2016), Stone et al., Cancer Immunol. Immunother., 63(11): 1163 (2014), and Walseng et al., Scientific Reports 7:10713 (2017), which CAR constructs and methods of making the same are incorporated by reference herein). CARs of the present disclosure that specifically bind to a Ras antigen (e.g., in the context of a peptide:HLA complex) comprise a TCR Vα domain and a Vβ domain.

Any polypeptide of this disclosure can, as encoded by a polynucleotide sequence, comprise a “signal peptide” (also known as a leader sequence, leader peptide, or transit peptide). Signal peptides target newly synthesized polypeptides to their appropriate location inside or outside the cell (e.g., to be inserted into or localize to a cell membrane, or to be secreted by the cell, or to be contained within the cell). In some contexts, signal peptides are from about 15 to about 22 amino acids in length. A signal peptide may be removed from the polypeptide during or once localization (e.g., membrane insertion) or secretion is completed. In some embodiments, a signal peptide is completely removed from the polypeptide. In some embodiments, less than all, but typically no more than one, two, three, four, five, or six amino acids of the signal peptide remain with the polypeptide and the rest of the signal peptide is removed. Polypeptides that have a signal peptide are referred to herein as a “pre-protein” and polypeptides having their signal peptide removed are referred to herein as “mature” proteins or polypeptides. In any of the herein disclosed embodiments, a binding protein or fusion protein comprises, or is, a mature protein, or is or comprises a pre-protein.

A “linker” refers to an amino acid sequence that connects two proteins, polypeptides, peptides, domains, regions, or motifs and may provide a spacer function compatible with interaction of the two sub-binding domains so that the resulting polypeptide retains a specific binding affinity (e.g., scTCR) to a target molecule or retains signaling activity (e.g., TCR complex). In certain embodiments, a linker is comprised of about two to about 35 amino acids, for instance, or about four to about 20 amino acids or about eight to about 15 amino acids or about 15 to about 25 amino acids. Exemplary linkers include glycine-serine linkers.

“Antigen” or “Ag” as used herein refers to an immunogenic molecule that provokes an immune response. This immune response may involve antibody production, activation of specific immunologically competent cells (e.g., T cells), or both. An antigen (immunogenic molecule) may be, for example, a peptide, glycopeptide, polypeptide, glycopolypeptide, polynucleotide, polysaccharide, lipid, or the like. It is readily apparent that an antigen can be synthesized, produced recombinantly, or derived from a biological sample. Exemplary biological samples that can contain one or more antigens include tissue samples, tumor samples, cells, biological fluids, or combinations thereof. Antigens can be produced by cells that have been modified or genetically engineered to express an antigen, or that endogenously (e.g., without modification or genetic engineering by human intervention) express a mutation or polymorphism that is immunogenic.

A “neoantigen,” as used herein, refers to a host cellular product containing a structural change, alteration, or mutation that creates a new antigen or antigenic epitope that has not previously been observed in the subject's genome (i.e., in a sample of healthy tissue from the subject) or been “seen” or recognized by the host's immune system, which: (a) is processed by the cell's antigen-processing and transport mechanisms and presented on the cell surface in association with an MHC (e.g., HLA) molecule; and (b) elicits an immune response (e.g., a cellular (T cell) response). Neoantigens may originate, for example, from coding polynucleotides having alterations (substitution, addition, deletion) that result in an altered or mutated product, or from the insertion of an exogenous nucleic acid molecule or protein into a cell, or from exposure to environmental factors (e.g., chemical, radiological) resulting in a genetic change. Neoantigens may arise separately from a tumor antigen, or may arise from or be associated with a tumor antigen. “Tumor neoantigen” (or “tumor-specific neoantigen”) refers to a protein comprising a neoantigenic determinant associated with, arising from, or arising within a tumor cell or plurality of cells within a tumor. Tumor neoantigenic determinants are found on, for example, antigenic tumor proteins or peptides that contain one or more somatic mutations or chromosomal rearrangements encoded by the DNA of tumor cells (e.g., pancreas cancer, lung cancer, colorectal cancers), as well as proteins or peptides from viral open reading frames associated with virus-associated tumors (e.g., cervical cancers, some head and neck cancers). The terms “antigen” and “neoantigen” are used interchangeably herein when referring to a Ras antigen comprising a mutation as disclosed herein.

The term “epitope” or “antigenic epitope” includes any molecule, structure, amino acid sequence or protein determinant that is recognized and specifically bound by a cognate binding molecule, such as an immunoglobulin, T cell receptor (TCR), chimeric antigen receptor, or other binding molecule, domain, or protein. Epitopic determinants generally contain chemically active surface groupings of molecules, such as amino acids or sugar side chains, and can have specific three-dimensional structural characteristics, as well as specific charge characteristics.

As used herein, the term “KRAS (or NRAS or HRAS) antigen (or neoantigen)” or “KRAS (or NRAS or HRAS) peptide antigen (or neoantigen)” or “KRAS (NRAS or HRAS) peptide” refers to a naturally or synthetically produced peptide portion of a KRAS or NRAS or HRAS protein ranging in length from about 7 amino acids, about 8 amino acids, about 9 amino acids, or about 10 amino acids, up to about 20 amino acids, and comprising at least one amino acid alteration caused by a G12 (e.g., G12V) mutation (wherein position 12 is in reference to the full-length KRAS protein sequence set forth in SEQ ID NO:1; and is also in reference to the full-length NRAS and HRAS protein sequence set forth in SEQ ID NOs.: 78 and 79, respectively), which peptide can form a complex with a MHC (e.g., HLA) molecule, and a binding protein of this disclosure specific for a KRAS or NRAS or HRAS peptide:MHC (e.g., HLA) complex can specifically bind to such as complex. An exemplary KRAS (or NRAS or HRAS) antigen comprises, consists essentially of, or consists of a peptide having the amino acid sequence of SEQ ID NO.: 2 or 3.

“Major histocompatibility complex” (MHC) refers to glycoproteins that deliver peptide antigens to a cell surface of all nucleated cells. MHC class I molecules are heterodimers having a membrane spanning a chain (with three α domains) and a non-covalently associated β₂ microglobulin. MHC class II molecules are composed of two transmembrane glycoproteins, a and B, both of which span the membrane. Each chain comprises two domains. MHC class I molecules deliver peptides originating in the cytosol to the cell surface, where a peptide:MHC complex is recognized by CD8⁺ T cells. MHC class II molecules deliver peptides originating in the vesicular system to the cell surface, where they are recognized by CD4⁺ T cells. Human MHC is referred to as human leukocyte antigen (HLA). HLAs corresponding to “class I” MHC present peptides from inside the cell and include, for example, HLA-A, HLA-B, and HLA-C. Alleles include, for example, HLA A*11, such as HLA-A*11:01. HLAs corresponding to “class II” MHC present peptides from outside the cell and include, for example, HLA-DP, HLA-DM, HLA-DOA, HLA-DOB, HLA-DQ, and HLA-DR.

Principles of antigen processing by antigen presenting cells (APC) (such as dendritic cells, macrophages, lymphocytes or other cell types), and of antigen presentation by APC to T cells, including major histocompatibility complex (MHC)-restricted presentation between immunocompatible (e.g., sharing at least one allelic form of an MHC gene that is relevant for antigen presentation) APC and T cells, are well-established (see, e.g., Murphy, Janeway's Immunobiology (8^{th} Ed.) 2011 Garland Science, NY; chapters 6, 9 and 16). For example, processed antigen peptides originating in the cytosol (e.g., tumor antigen, intracellular pathogen) are generally from about 7 amino acids to about 11 amino acids in length and will associate with class I MHC (HLA) molecules, whereas peptides processed in the vesicular system (e.g., bacterial, viral) will generally vary in length from about 10 amino acids to about 25 amino acids and associate with class II MHC (HLA) molecules.

The term “KRAS-specific binding protein,” as used herein, refers to a protein or polypeptide, such as, for example, a TCR, a scTv, a scTCR, or CAR, that binds to a KRAS peptide antigen or a NRAS peptide antigen or a HRAS peptide antigen (or to a KRAS or NRAS or HRAS peptide antigen:HLA complex, e.g., on a cell surface), and does not bind a peptide that does not contain the KRAS or NRAS or HRAS peptide antigen and does not bind to an HLA complex containing such a peptide.

Binding proteins of this disclosure, such as TCRs, scTvs, scTCRs, and CARs, contain a binding domain specific for a target. A “binding domain” (also referred to as a “binding region” or “binding moiety”), as used herein, refers to a molecule or portion thereof (e.g., peptide, oligopeptide, polypeptide, protein) that possesses the ability to specifically and non-covalently associate, unite, or combine with a target (e.g., KRAS or NRAS or HRAS peptide or KRAS or NRAS or HRAS peptide:MHC complex). A binding domain includes any naturally occurring, synthetic, semi-synthetic, or recombinantly produced binding partner for a biological molecule, a molecular complex (i.e. complex comprising two or more biological molecules), or other target of interest. Exemplary binding domains include immunoglobulin variable regions or single chain constructs comprising the same (e.g., single chain TCR (scTCR) or scTv).

In certain embodiments, a Ras-specific binding protein binds to a KRAS (or NRAS or HRAS) peptide (or a KRAS (or NRAS or HRAS): HLA complex) with a K_{d} of less than about 10⁻⁸ M, less than about 10⁻⁹ M, less than about 10⁻¹⁰ M, less than about 10⁻¹¹ M, less than about 10⁻¹² M, or less than about 10⁻¹³ M, or with an affinity that is about the same as, at least about the same as, or is greater than at or about the affinity exhibited by an exemplary Ras-specific binding protein provided herein, such as any of the Ras-specific TCRs provided herein, for example, as measured by the same assay. In certain embodiments, a Ras-specific binding protein comprises a Ras-specific immunoglobulin superfamily binding protein or binding portion thereof.

As used herein “specifically binds” or “specific for” refers to an association or union of a binding protein (e.g., TCR receptor) or a binding domain (or fusion protein thereof) to a target molecule with an affinity or Kₐ (i.e., an equilibrium association constant of a particular binding interaction with units of 1/M) equal to or greater than 10⁵ M⁻¹ (which equals the ratio of the on-rate [kₒₙ] to the off-rate [K^{off}] for this association reaction), while not significantly associating or uniting with any other molecules or components in a sample. Binding proteins or binding domains (or fusion proteins thereof) may be classified as “high affinity” binding proteins or binding domains (or fusion proteins thereof) or as “low affinity” binding proteins or binding domains (or fusion proteins thereof). “High affinity” binding proteins or binding domains refer to those binding proteins or binding domains having a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. “Low affinity” binding proteins or binding domains refer to those binding proteins or binding domains having a Kₐ of up to 10⁷ M⁻¹, up to 10⁶ M⁻¹, up to 10⁵ M⁻¹. Alternatively, affinity can be defined as an equilibrium dissociation constant (K_{d}) of a particular binding interaction with units of M (e.g., 10⁻⁵ M to 10⁻¹³ M).

In certain embodiments, a receptor or binding domain may have “enhanced affinity,” which refers to a selected or engineered receptors or binding domain with stronger binding to a target antigen than a wild type (or parent) binding domain. For example, enhanced affinity may be due to a Kₐ (equilibrium association constant) for the target antigen that is higher than the wild type binding domain, due to a K_{d} (dissociation constant) for the target antigen that is less than that of the wild type binding domain, due to an off-rate (koff) for the target antigen that is less than that of the wild type binding domain, or a combination thereof.

A variety of assays are known for identifying binding domains of the present disclosure that specifically bind a particular target, as well as determining binding domain or fusion protein affinities, such as Western blot, ELISA, analytical ultracentrifugation, spectroscopy and surface plasmon resonance (Biacore®) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent). Binding and binding affinity can also be assessed using, for example, fluorescence intensity observed when a binding protein binds to a labelled HLA-peptide complex or labelled HLA-peptide complex multimer (e.g., tetramer).

In certain embodiments, a KRAS (or NRAS, or HRAS)-specific binding domain alone (i.e., without any other portion of a KRAS (or NRAS, or HRAS)-specific binding protein) can be soluble and can bind to KRAS (or NRAS, or HRAS) (or a KRAS (or NRAS, or HRAS) peptide, or a KRAS (or NRAS, or HRAS) peptide: HLA complex) with a K_{d} of less than about 10⁻⁸ M, less than about 10⁻⁹ M, less than about 10⁻¹⁰ M, less than about 10⁻¹¹ M, less than about 10⁻¹² M, or less than about 10-13 M. In particular embodiments, a KRAS (or NRAS, or HRAS)-specific binding domain includes a KRAS (or NRAS, or HRAS)-specific scTCR (e.g., single chain αβTCR proteins such as comprising Vα-L-Vβ, Vβ-L-Vα, Vα-Cα-L-Vα, or Vα-L-Vβ-Cβ, wherein Vα and Vβ are TCRα and β variable domains respectively, Cα and Cβ are TCRα and β constant domains, respectively, and L is a linker, such as a linker described herein). In some embodiments, a KRAS (or NRAS, or HRAS)-specific binding domain includes a KRAS (or NRAS, or HRAS)-specific scTv (e.g., single chain TCR variable domains proteins such as Vα-L-Vβ or Vβ-L-Vα wherein Vα and Vβ are TCRα and β variable domains respectively, and L is a linker, such as a linker described herein).

The term “functional avidity”, as used herein, refers to a biological measure or activation threshold of an in vitro immune cell (e.g., T cell, NK cell, NK-T cell) response to a given concentration of a ligand, wherein the biological measures can include cytokine production (e.g., IFN-γ production, IL-2 production, etc.), cytotoxic activity, activation markers (e.g., CD137, Nur77) and proliferation. For example, T cells that biologically (immunologically) respond in vitro to a low antigen dose by, for example, producing cytokines, exhibiting cytotoxic activity, or proliferating are considered to have high functional avidity, while T cells having lower functional avidity require higher amounts of antigen before an immune response, similar to the high-avidity T cells, is elicited. It will be understood that functional avidity is different from affinity and avidity. Affinity refers to the strength of any given bond between a binding protein and its antigen/ligand. Some binding proteins are multivalent and bind to multiple antigens—in this case, the strength of the overall connection is the avidity.

Numerous correlations exist between the functional avidity and the effectiveness of an immune response. Some ex vivo studies have shown that distinct T cell functions (e.g., proliferation, cytokines production, etc.) can be triggered at different thresholds (see, e.g., Betts et al., J. Immunol. 172:6407, 2004; Langenkamp et al., Eur. J. Immunol. 32:2046, 2002). Factors that affect functional avidity can include (a) the affinity of a TCR for the pMHC-complex, that is, the strength of the interaction between the TCR and pMHC (Cawthon et al., J. Immunol. 167:2577, 2001), (b) expression levels of the TCR, and, in some embodiments, CD4 or CD8 co receptors, on the host cell and (c) the distribution and composition of signaling molecules (Viola and Lanzavecchia, Science 273:104, 1996), as well as expression levels of molecules that attenuate T cell function and TCR signaling.

The concentration of antigen needed to induce a half-maximum response (e.g., production of a cytokine or activation marker by a host cell; fluorescence intensity when binding to a labeled peptide: HLA multimer) between the baseline and maximum response after a specified exposure time is referred to as the “half maximal effective concentration” or “EC50”. The EC50 value is generally presented as a molar (moles/liter) amount, but it is often converted into a logarithmic value as follows—log₁₀(EC50). For example, if the EC50 equals 1 μM (10⁻⁶ M), the log₁₀(EC50) value is −6. Another value used is pEC50, which is defined as the negative logarithm of the EC50 (−log₁₀(EC50)). In the above example, the EC50 equaling 1 μM has a pEC50 value of 6. In certain embodiments, the functional avidity of a binding protein of this disclosure will comprise a measure of an ability of the binding protein to promote activation and/or IFNγ production by T cells, which can be measured using assays known in the art and described herein. In certain embodiments, functional avidity will comprise a measure of the ability of the binding protein, upon binding to antigen, to activate a host cell, such as a T cell.

Binding proteins disclosed herein can comprise high functional avidity that can, for example, facilitate elicitation of immune cell effector functions (e.g., activation, proliferation, cytokine production, and/or cytotoxicity) against even lower levels of a (n e.g. HLA-A*11:01-) presented KRAS G12 mutant peptide, such as the KRAS G12V mutant peptide of SEQ ID NO: 2 or SEQ ID NO: 3.

In some embodiments, the binding protein has a log 10EC50 for the KRAS G12 (e.g., G12V) mutant peptide of about −6.0 or less, about −6.1 or less, about −6.2 or less, about −6.3 or less, about −6.4 or less, about −6.5 or less, about −6.6 or less, about −6.7 or less, about −6.8 or less, about −6.9 or less, about −7.0 or less, about −7.1 or less, about −7.2 or less, about −7.3 or less, about −7.4 or less, about −7.5 or less, about −7.6 or less, about −7.7 or less, about −7.8 or less, about −7.9 or less, about −8.0 or less, about −8.1 or less, about −8.2 or less, about −8.3 or less, about −8.4 or less, about −8.5 or less, about −8.6 or less, about −8.7 or less, about −8.8 or less, about −8.9 or less, about −9 or less, about −9.1 or less, about −9.2 or less, about −9.3 or less, about −9.4 or less, about −9.5 or less, about −9.6 or less, about −9.7 or less, about −9.8 or less, about −9.9 or less, or about −10 or less.

In some embodiments, a host cell disclosed herein comprises a binding protein (e.g., TCR) that binds a target antigen of the binding protein (for example, a KRAS G12 mutant peptide, such as KRAS G12V mutant peptide, e.g., present in a peptide: HLA (e.g. HLA-A*11:01) complex) with an EC50 (e.g., peptide dose at which a half-maximal activation of a population of T cells expressing the binding protein is reached) of less than about 100 mM, less than about 10 mM, less than about 1 mM, less than about 500 μM, less than about 100 μM, less than about 50 μM, less than about 10 μM, less than about 5 μM, less than about 4 μM, less than about 3 μM, less than about 2 μM, less than about 1 μM, less than about 900 nM, less than about 800 nM, less than about 700 nM, less than about 600 nM, less than about 500 nM, less than about 400 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 1 nM, less than about 900 pM, less than about 800 pM, less than about 700 pM, less than about 600 pM, less than about 500 pM, less than about 400 pM, less than about 300 pM, less than about 200 pM, less than about 100 pM, less than about 90 pM, less than about 80 pM, less than about 70 pM, less than about 60 pM, less than about 50 pM, less than about 40 pM, less than about 30 pM, less than about 20 pM, less than about 10 pM, less than about 5 pM, or less than about 1 pM. The EC50 can be determined by an assay to identify a peptide dose at which a half-maximal activation of a T cell population is reached, e.g., as reflected by expression an activation marker (e.g., CD137, CD69, Granzyme B, CD107a, IFN-gamma, TNF-a, IL-12, Nur77, a cytokine, an interleukin, an interferon) upon exposure to target cells in the presence of various concentrations of the mutant peptide.

In some embodiments, a host cell disclosed herein comprises a binding protein (e.g., TCR) that binds a target antigen of the binding protein (for example, a KRAS G12 mutant peptide, such as KRAS G12V mutant peptide, e.g., present in a peptide: HLA (e.g. HLA-A*11:01) complex) with an EC50 (e.g., peptide dose at which a half-maximal activation of a population of T cells expressing the binding protein is reached) of at least about 100 mM, at least about 10 mM, at least about 1 mM, at least about 500 μM, at least about 100 μM, at least about 50 μM, at least about 10 μM, at least about 5 μM, at least about 4 μM, at least about 3 μM, at least about 2 μM, at least about 1 μM, at least about 900 nM, at least about 800 nM, at least about 700 nM, at least about 600 nM, at least about 500 nM, at least about 400 nM, at least about 300 nM, at least about 200 nM, at least about 100 nM, at least about 90 nM, at least about 80 nM, at least about 70 nM, at least about 60 nM, at least about 50 nM, at least about 40 nM, at least about 30 nM, at least about 20 nM, at least about 10 nM, at least about 5 nM, at least about 1 nM, at least about 900 pM, at least about 800 pM, at least about 700 pM, at least about 600 pM, at least about 500 pM, at least about 400 pM, at least about 300 pM, at least about 200 pM, at least about 100 pM, at least about 90 pM, at least about 80 pM, at least about 70 pM, at least about 60 pM, at least about 50 pM, at least about 40 pM, at least about 30 pM, at least about 20 pM, at least about 10 pM, at least about 5 pM, or at least about 1 pM.

In some embodiments, a binding protein (e.g., TCR) binds a target (for example, a KRAS G12 mutant peptide, such as KRAS G12V mutant peptide, e.g., present in a peptide: HLA (e.g. HLA-A*11:01) complex) with a KD of less than about 100 mM, less than about 10 mM, less than about 1 mM, less than about 500 μM, less than about 100 μM, less than about 50 μM, less than about 10 μM, less than about 5 μM, less than about 4 μM, less than about 3 μM, less than about 2 μM, less than about 1 μM, less than about 900 nM, less than about 800 nM, less than about 700 nM, less than about 600 nM, less than about 500 nM, less than about 400 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 1 nM, less than about 900 pM, less than about 800 pM, less than about 700 pM, less than about 600 pM, less than about 500 pM, less than about 400 pM, less than about 300 pM, less than about 200 pM, less than about 100 pM, less than about 90 pM, less than about 80 pM, less than about 70 pM, less than about 60 pM, less than about 50 pM, less than about 40 pM, less than about 30 pM, less than about 20 pM, less than about 10 pM, less than about 5 pM, or less than about 1 pM.

Also contemplated are fusion proteins comprising a scTCR or scTv of the present disclosure linked to a constant domain (e.g. heavy chain constant domain or combination thereof, such as a Fc, a CH2, a CH3, a CH4, and/or a CH1) of an antibody (e.g., IgG (1, 2, 3, 4), IgE, IgD, IgA, IgM, and variants thereof) or a fragment thereof (e.g., a fragment that, in some embodiments, retains binding to one or more Fc receptors, to C1q, to Protein A, to Protein G, or any combination thereof), and including immunoglobulin heavy chain monomers and multimers, such as Fc dimers; see, e.g., Wong et al., J. Immunol. 198:1 Supp. (2017). Variant Fc polypeptides comprising mutations that enhance, reduce, or abrogate binding to or by, e.g., FcRn or other Fc receptors, are known and are contemplated within this disclosure.

In certain embodiments, a binding protein or fusion protein (e.g., TCR, scTCR, CAR) of the present disclosure is expressed by a host cell (e.g., by a T cell, NK cell, or NK-T cell heterologously expressing the binding protein or fusion protein). Avidity of such a host cell for a KRAS (or NRAS, or HRAS) peptide antigen or KRAS (or NRAS, or HRAS) peptide antigen:HLA complex can be determined by, for example, exposing the host cell to the peptide, or to a peptide: HLA complex (e.g., organized as a tetramer), or to an antigen-presenting cell (APC) that presents the peptide to the host cell, optionally in a peptide: HLA complex, and then measuring an activity of the host cell, such as, for example, production or secretion of cytokines (e.g., IFN-γ; TNFα); increased expression of host cell signaling or activation components (e.g., CD137 (4-1BB)); proliferation of the host cell; or killing of the APC (e.g., using a labeled-chromium release assay).

As used herein, “nucleic acid” or “nucleic acid molecule” or “polynucleotide” refers to any of deoxyribonucleic acid (DNA), ribonucleic acid (RNA), oligonucleotides, polynucleotides, fragments thereof generated, for example, by the polymerase chain reaction (PCR) or by in vitro translation, and also to fragments generated by any of ligation, scission, endonuclease action, or exonuclease action. In certain embodiments, the nucleic acids of the present disclosure are produced by PCR. Nucleic acids can be composed of monomers that are naturally occurring nucleotides (such as deoxyribonucleotides and ribonucleotides), analogs of naturally occurring nucleotides (e.g., α-enantiomeric forms of naturally occurring nucleotides), or a combination of both. Modified nucleotides can have modifications in or replacement of sugar moieties, or pyrimidine or purine base moieties. Nucleic acid monomers can be linked by phosphodiester bonds or analogs of such linkages. Analogs of phosphodiester linkages include phosphonothioate, phosphonodithioate, phosphonoselenoate, phosphorodiselenoate, phosphoroanilothioate, phosphoranilidate, phosphoramidate, and the like. Nucleic acid monomers may comprise phosphorothioate linkages, phosphorodithioate linkages, or phosphoroselenoate linkages, or any combination thereof. Nucleic acid molecules can be either single-stranded or double-stranded.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such a nucleic acid could be part of a vector and/or such nucleic acid or polypeptide could be part of a composition (e.g., a cell lysate), and still be isolated in that such vector or composition is not part of the natural environment for the nucleic acid or polypeptide. In some embodiments, an isolated binding protein, polynucleotide, vector, or host cell is provided. The term “gene” means the segment of DNA involved in producing a polypeptide chain; it includes regions preceding and following the coding region (“leader and trailer”) as well as intervening sequences (introns) between individual coding segments (exons).

As used herein, the terms “recombinant”, “engineered”, and “modified” refer to a cell, microorganism, nucleic acid molecule, polypeptide, protein, plasmid, or vector that has been modified by introduction of an exogenous nucleic acid molecule, or refers to a cell or microorganism that has been genetically engineered by human intervention—that is, modified by introduction of a heterologous nucleic acid molecule, or refers to a cell or microorganism that has been altered such that expression of an endogenous nucleic acid molecule or gene is controlled, deregulated or constitutive, where such alterations or modifications can be introduced by genetic engineering. Human-generated genetic alterations can include, for example, modifications introducing nucleic acid molecules (which may include an expression control element, such as a promoter) encoding one or more proteins or enzymes, or other nucleic acid molecule additions, deletions, substitutions, or other functional disruption of or addition to a cell's genetic material. Exemplary modifications include those in coding regions or functional fragments thereof of heterologous or homologous polypeptides from a reference or parent molecule.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s). In certain embodiments, a mutation is a substitution of one or three codons or amino acids, a deletion of one to about 5 codons or amino acids, or a combination thereof.

A “conservative substitution” is recognized in the art as a substitution of one amino acid for another amino acid that has similar properties. Exemplary conservative substitutions are well known in the art (see, e.g., WO 97/09433 at page 10; Lehninger, Biochemistry, 2^{nd} Edition; Worth Publishers, Inc. NY, NY, pp. 71-77, 1975; Lewin, Genes IV, Oxford University Press, NY and Cell Press, Cambridge, MA, p. 8, 1990).

In certain embodiments, proteins (e.g., binding protein, immunogenic peptide) according to the present disclosure comprise a variant sequence as compared to a reference sequence (e.g., a variant TCR CDR (e.g., CDR3β) as compared to a reference TCR CDR (e.g., CDR3β) disclosed herein). As used herein, a “variant” amino acid sequence, peptide, or polypeptide, can refer to an amino acid sequence (or peptide or polypeptide) having one, two, or three amino acid substitutions, deletions, and/or insertions as compared to a reference amino acid sequence. In certain embodiments, a variant amino acid sequence, peptide, or polypeptide, retains substantially a same functionality (e.g., binding specificity and affinity for a peptide: HLA complex) as the reference molecule; for example, a variant TCR fragment as disclosed herein retains about 50%, about 60%, about 70%, about 80%, about 90%, about 95%, about 99%, at least about 50%, at least about 60%, at least about 70%, at least about 80%, at least about 90%, at least about 95%, at least about 99%, or 100% of the antigen-binding specificity and affinity as compared to a reference TCR binding fragment.

An “altered domain” or “altered protein” refers to a motif, region, domain, peptide, polypeptide, or protein with a non-identical sequence identity to a wild type motif, region, domain, peptide, polypeptide, or protein (e.g., a wild type TCRα chain, TCRβ chain, TCRα constant domain, TCRβ constant domain) of at least 85% (e.g., at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9%).

Altered domains or altered proteins or derivatives can include those based on all possible codon choices for the same amino acid and codon choices based on conservative amino acid substitutions. For example, the following six groups each contain amino acids that are conservative substitutions for one another: 1) alanine (ala; A), serine (ser; S), threonine (thr; T); 2) aspartic acid (asp; D), glutamic acid (glu; E); 3) asparagine (asn; N), glutamine (gln; Q); 4) arginine (arg; R), lysine (lys; K); 5) Isoleucine (ile; I), leucine (L), methionine (met; M), valine (val; V); and 6) phenylalanine (phe; F), tyrosine (tyr; Y), tryptophan (trp; W). (See also WO97/09433 at page 10, Lehninger, Biochemistry, 2nd Edition, Worth Publishers, Inc., NY, NY, pp. 71-77, 1975; Lewin Genes IV, Oxford University Press, NY and Cell Press, Cambridge, MA, p. 8, 1990; Creighton, Proteins, W.H. Freeman and Company 1984). In addition, individual substitutions, deletions, or additions that alter, add or delete, a single amino acid or a small percentage of amino acids in an encoded sequence are also “conservative substitutions.”

The term “construct” refers to any polynucleotide that contains a recombinant nucleic acid molecule. A “transgene” or “transgene construct” refers to a construct that contains two or more genes operably linked in an arrangement that is not found in nature. The term “operably-linked” (or “operably linked” herein) refers to the association of two or more nucleic acid molecules on a single nucleic acid fragment so that the function of one is affected by the other. For example, a promoter is operably-linked with a coding sequence when it can affect the expression of that coding sequence (i.e., the coding sequence is under the transcriptional control of the promoter). “Unlinked” means that the associated genetic elements are not closely associated with one another and the function of one does not affect the other. In some embodiments, the genes present in a transgene are operably linked to an expression control sequence (e.g., a promoter).

A construct (e.g., a transgene) can be present in a vector (e.g., a bacterial vector, a viral vector) or can be integrated into a genome. A “vector” is a nucleic acid molecule that is capable of transporting another nucleic acid molecule. Vectors can be, for example, plasmids, cosmids, viruses, a RNA vector or a linear or circular DNA or RNA molecule that can include chromosomal, non-chromosomal, semi-synthetic or synthetic nucleic acid molecules. Exemplary vectors are those capable of autonomous replication (episomal vector) or expression of nucleic acid molecules to which they are linked (expression vectors). Vectors useful in the compositions and methods of this disclosure are described further herein.

The term “expression”, as used herein, refers to the process by which a polypeptide is produced based on the encoding sequence of a nucleic acid molecule, such as a gene. The process can include transcription, post-transcriptional control, post-transcriptional modification, translation, post-translational control, post translational modification, or any combination thereof.

The term “introduced” in the context of inserting a nucleic acid molecule into a cell, means “transfection”, or “transformation”, or “transduction” and includes reference to the incorporation of a nucleic acid molecule into a eukaryotic or prokaryotic cell wherein the nucleic acid molecule can be incorporated into the genome of a cell (e.g., a chromosome, a plasmid, a plastid, or a mitochondrial DNA), converted into an autonomous replicon, or transiently expressed (e.g., transfected mRNA).

As used herein, “heterologous” or “exogenous” nucleic acid molecule, construct or sequence refers to a nucleic acid molecule or portion of a nucleic acid molecule that is not native to a host cell, but can be homologous to a nucleic acid molecule or portion of a nucleic acid molecule from the host cell. The source of the heterologous or exogenous nucleic acid molecule, construct or sequence can be from a different genus or species. In certain embodiments, a heterologous or exogenous nucleic acid molecule is added (i.e., not endogenous, or native) to a host cell or host genome by, for example, conjugation, transformation, transfection, transduction, electroporation, or the like, wherein the added molecule can integrate into the host genome or exist as extra-chromosomal genetic material (e.g., as a plasmid or other form of self-replicating vector), and can be present in multiple copies. In addition, “heterologous” refers to a non-native enzyme, protein or other activity encoded by an exogenous nucleic acid molecule introduced into the host cell, even if the host cell encodes a homologous protein or activity. Moreover, a cell comprising a “modification” or a “heterologous” polynucleotide or binding protein includes progeny of that cell, regardless of whether the progeny were themselves transduced, transfected, or otherwise manipulated or changed.

As described herein, more than one heterologous or exogenous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof. For example, as disclosed herein, a host cell can be modified to express one or more heterologous or exogenous nucleic acid molecule encoding desired TCR specific for a Ras antigen peptide (e.g., TCRα and TCRβ) and optionally, as disclosed herein, also encoding a CD8 co-receptor polypeptide comprising a α chain, a β chain, or a portion thereof, such as an extracellular portion capable of binding to MHC. When two or more exogenous nucleic acid molecules are introduced into a host cell, it is understood that the two or more exogenous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, and not necessarily to the number of separate nucleic acid molecules introduced into a host cell.

As used herein, the term “endogenous” or “native” refers to a gene, protein, or activity that is normally present in a host cell. Moreover, a gene, protein or activity that is mutated, overexpressed, shuffled, duplicated, or otherwise altered as compared to a parent gene, protein or activity is still considered to be endogenous or native to that particular host cell. For example, an endogenous control sequence from a first gene (e.g., a promoter, translational attenuation sequences) can be used to alter or regulate expression of a second native gene or nucleic acid molecule, wherein the expression or regulation of the second native gene or nucleic acid molecule differs from normal expression or regulation in a parent cell.

The term “homologous” or “homolog” refers to a molecule or activity found in or derived from a host cell, species, or strain. For example, a heterologous or exogenous nucleic acid molecule can be homologous to a native host cell gene, and can optionally have an altered expression level, a different sequence, an altered activity, or any combination thereof.

“Sequence identity,” as used herein, refers to the percentage of amino acid residues or nucleobases in one sequence that are identical with the amino acid residues or nucleobases (respectively) in a reference sequence after aligning the sequences and introducing gaps, if necessary, to achieve the maximum percent sequence identity, and not considering any conservative substitutions as part of the sequence identity. The percentage sequence identity values can be generated using the NCBI BLAST 2.0 software as defined by Altschul et al. (1997), Nucl. Acids Res. 25:3389-3402, with the parameters set to default values. Additionally or alternatively, the degree of sequence identity between two sequences can be determined, for example, by comparing the two sequences using computer programs designed for this purpose, such as global or local alignment algorithms. Non-limiting examples include BLASTp, BLASTn, Clustal W, MAFFT, Clustal Omega, AlignMe, Praline, GAP, BESTFIT, Needle (EMBOSS), Stretcher (EMBOSS), GGEARCH2SEQ, Water (EMBOSS), Matcher (EMBOSS), LALIGN, SSEARCH2SEQ, or another suitable method or algorithm. A global alignment algorithm, such as a Needleman and Wunsch algorithm, can be used to align two sequences over their entire length, maximizing the number of matches and minimizes the number of gaps. Default settings can be used.

To generate similarity scores for two amino acid sequences, scoring matrices can be used that assign positive scores for some non-identical amino acids (e.g., conservative amino acid substitutions, amino acids with similar physio-chemical properties, and/or amino acids that exhibit frequent substitutions in orthologs, homologs, or paralogs), Non-limiting examples of scoring matrices include PAM30, PAM70, PAM250, BLOSUM45, BLOSUM50, BLOUM62, BLOSUM80, and BLOSUM90.

Variants of nucleic acid molecules of this disclosure are also contemplated. Variant nucleic acid molecules are at least 70%, at least 75%, at least 80%, at least 85%, at least 90%, and are preferably at least 95%, at least 96%, at least 97%, at least 98%, at least 99%, or at least 99.9% identical a nucleic acid molecule of a defined or reference polynucleotide as described herein, or that hybridize to a polynucleotide under stringent hybridization conditions of 0.015M sodium chloride, 0.0015M sodium citrate at about 65-68° C. or 0.015M sodium chloride, 0.0015M sodium citrate, and 50% formamide at about 42° C. Nucleic acid molecule variants retain the capacity to encode a binding protein or a binding domain thereof having a functionality described herein, such as binding a target molecule.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide. The term “gene” means the segment of DNA involved in producing a polypeptide chain; it includes regions preceding and following the coding region (“leader and trailer”) as well as intervening sequences (introns) between individual coding segments (exons).

In some contexts, the term “variant” as used herein, refers to at least one fragment of the full-length sequence referred to, more specifically one or more amino acid or nucleic acid sequence which is, relative to the full-length sequence, truncated at one or both termini by one or more amino acids. Such a fragment includes or encodes a peptide having at least 6, 7, 8, 10, 12, 15, 20, 25, 50, 75, 100, 150, or 200 successive amino acids of the original sequence or a variant thereof. The total length of the variant may be at least 6, 7, 8, 9, 10, 11, 12, 20, 25, 30, 40, 50, 60, 70, 80, 90, 100, or more amino acids.

In some embodiments, the term “variant” relates not only to at least one fragment, but also to a polypeptide or a fragment thereof including amino acid sequences that are at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5% identical to the reference amino acid sequence referred to or the fragment thereof, wherein amino acids other than those essential for the biological activity or the fold or structure of the polypeptide are deleted or substituted, one or more such essential amino acids are replaced in a conservative manner, and/or amino acids are added such that the biological activity of the polypeptide is preserved. The state of the art includes various methods that may be used to align two given nucleic acid or amino acid sequences and to calculate the degree of identity (see, e.g., Arthur Lesk (2008), Introduction to bioinformatics, Oxford University Press, 2008, 3rd edition). In some embodiments, the Clustal W software can be used using default settings (Larkin, M. A., et al. (2007). Clustal W and Clustal X version 2.0. Bioinformatics, 23, 2947-2948).

In certain embodiments, variants may, in addition, include chemical modifications, for example, isotopic labels or covalent modifications such as glycosylation, phosphorylation, acetylation, decarboxylation, citrullination, hydroxylation and the like. Methods for modifying polypeptides are known and in general will be employed so as not to abolish or substantially diminish a desired activity of the polypeptide.

In an embodiment, the term “variant” of a nucleic acid molecule includes nucleic acids the complementary strand of which hybridizes, for example, under stringent conditions, to the reference or wild type nucleic acid. Stringency of hybridization reactions is readily determinable by one of ordinary skill in the art, and in general is an empirical calculation dependent on probe length, washing temperature, and salt concentration. In general, longer probes require higher temperatures for proper annealing, while shorter probes less so. Hybridization generally depends on the ability of denatured DNA to reanneal to complementary strands present in an environment below their melting temperature: the higher the degree of desired homology between the probe and hybridizable sequence, the higher the relative temperature which may be used. As a result, higher relative temperatures would tend to make the reaction conditions more stringent, while lower temperature less so. For additional details and explanation of stringency of hybridization reactions, see Ausubel, F. M. (1995), Current Protocols in Molecular Biology. John Wiley & Sons, Inc. Moreover, the person skilled in the art may follow the instructions given in the manual Boehringer Mannheim GmbH (1993) The DIG System Users Guide for Filter Hybridization, Boehringer Mannheim GmbH, Mannheim, Germany and in Liebl, W., Ehrmann, M., Ludwig, W., and Schleifer, K. H. (1991) International Journal of Systematic Bacteriology 41:255-260 on how to identify DNA sequences by means of hybridization. In an embodiment, stringent conditions are applied for any hybridization, i.e., hybridization occurs only if the probe is 70% or more identical to the target sequence. Probes having a lower degree of identity with respect to the target sequence may hybridize, but such hybrids are unstable and will be removed in a washing step under stringent conditions, for example, lowering the concentration of salt to 2× SSC or, optionally and subsequently, to 0.5×SSC, while the temperature is, for example, about 50° C.-68° C., about 52° C.-68° C., about 54° C.-68° C., about 56° C.-68° C., about 58° C.-68° C., about 60° C.-68° C., about 62° C.-68° C., about 64° C.-68° C., or about 66° C.-68° C. In an embodiment, the temperature is about 64° C.-68° C. or about 66° C.-68° C. It is possible to adjust the concentration of salt to 0.2×SSC or even 0.1×SSC. Nucleic acid sequences having a degree of identity with respect to the reference or wild type sequence of at least 70%, at least 80%, at least 85%, at least 90%, at least 91%, at least 92%, at least 93%, at least 94%, at least 95%, at least 96%, at least 97%, at least 98%, at least 99%, or at least 99.5% may be isolated. In an embodiment, the term variant of a nucleic acid sequence, as used herein, refers to any nucleic acid sequence that encodes the same amino acid sequence and variants thereof as the reference nucleic acid sequence, in line with the degeneracy of the genetic code.

A “functional variant” refers to a polypeptide or polynucleotide that is structurally similar or substantially structurally similar to a parent or reference compound of this disclosure, but differs, in some contexts slightly, in composition (e.g., one base, atom or functional group is different, added, or removed; or one or more amino acids are mutated, inserted, or deleted), such that the polypeptide or encoded polypeptide is capable of performing at least one function of the encoded parent polypeptide with at least 50% efficiency, preferably at least 55%, at least 60%, at least 70%, at least 75%, at least 80%, at least 85%, at least 90%, at least 95%, at least 96%, at least 97%, at least 98%, at least 99%, at least 99.9%, or at least 100% level of activity of the parent polypeptide. In other words, a functional variant of a polypeptide or encoded polypeptide of this disclosure has “similar binding,” “similar affinity” or “similar activity” when the functional variant displays no more than a 50% reduction in performance in a selected assay as compared to the parent or reference polypeptide, such as an assay for measuring binding affinity (e.g., Biacore® or tetramer staining measuring an association (Ka) or a dissociation (KD) constant), avidity, or activation of a host cell. As used herein, a “functional portion” or “functional fragment” refers to a polypeptide or polynucleotide that comprises only a domain, motif, portion or fragment of a parent or reference compound, and the polypeptide or encoded polypeptide retains at least 50% activity associated with the domain, portion or fragment of the parent or reference compound, preferably at least 55%, at least 60%, at least 70%, at least 75%, at least 80%, at least 85%, at least 90%, at least 95%, at least 96%, at least 97%, at least 98%, at least 99%, at least 99.9%, or at least 100% level of activity of the parent polypeptide, or provides a biological benefit (e.g., effector function).

A “functional portion” or “functional fragment” of a polypeptide or encoded polypeptide of this disclosure has “similar binding” or “similar activity” when the functional portion or fragment displays no more than a 50% reduction in performance in a selected assay as compared to the parent or reference polypeptide (preferably no more than 20% or 10%, or no more than a log difference as compared to the parent or reference with regard to affinity), such as an assay for measuring binding affinity or measuring effector function (e.g., cytokine release). Functional variants of specifically disclosed binding proteins and polynucleotides are contemplated.

An “altered domain” or “altered protein” refers to a motif, region, domain, peptide, polypeptide, or protein with a non-identical sequence identity to a wild type motif, region, domain, peptide, polypeptide, or protein (e.g., a wild type TCRα chain, TCRβ chain, TCRα constant domain, or TCRβ constant domain) of at least 85% (e.g., at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9%).

### Binding Proteins

In one aspect, the present disclosure provides a binding protein, comprising a T cell receptor (TCR) α chain variable (Vα) domain and a TCR β chain variable (Vβ) domain, wherein the binding protein is capable of binding to a peptide: HLA complex, wherein the peptide comprises, consists essentially of, or consists of the amino acid sequence set forth in SEQ ID NO.: 2 or SEQ ID NO.: 3. In certain embodiments, the HLA comprises an HLA-A*11, optionally HLA-A*11:01. In any of the presently disclosed embodiments, the binding protein can be heterologously expressed by a human immune system cell, such as, for example, a T cell.

In certain embodiments, the Vα domain and/or the Vβ domain are each independently human, humanized, or chimeric, and are preferably each human. In some embodiments, the Vα domain is human and the Vβ domain is human. Binding proteins, compositions, and methods disclosed herein can utilize a Vα domain, Vβ domain, or CDRs therefrom derived from a human subject, for example, from sequencing of an isolated T cell or population thereof from a human subject. TCR Vα domains, Vβ domains, and CDRs therefrom isolated from a human subject can have advantageous properties over variable domains and CDRs from other sources, such as mice transgenic for a single human HLA allele. For example, Vα domains, Vβ domains, and CDRs derived from a human subject can have undergone negative thymic selection against substantially the whole human peptidome presented by a full set of human HLA molecules in vivo, which can reduce the likelihood that the binding protein is cross-reactive to other human self-antigens.

In some embodiments, a binding protein disclosed herein is substantially non-reactive to a human proteome presented by one or more HLA alleles disclosed herein, for example, one or any combination of HLA alleles from Table 3. The reactivity can be determined by any suitable method, such as those disclosed in Examples 5-7 and 13 of the instant application. In some embodiments, no significant response by binding protein-transduced T cells to the human proteome presented by the one or more HLA allele(s) is observed or predicted with peptide concentrations of 500 nM or lower, 400 nM or lower, 300 nM or lower, 200 nM or lower, 100 nM or lower, 50 nM or lower, 10 nM or lower, 5 nM or lower, or 1 nM or lower. In some embodiments, a binding protein disclosed herein is substantially non-reactive to a peptide: HLA complex, wherein the peptide comprises or consists of the amino acid sequence set forth in any one of SEQ ID NOs.: 118-148. In some embodiments, the HLA comprises an HLA-A*11. In further embodiments, the HLA comprises HLA-A*11:01.

In some embodiments, a binding protein comprises one or more variable domains or one or more CDRs derived from (e.g., identified in) a T cell of a subject (e.g., a human subject) having a disease, such as a cancer. In some embodiments, a binding protein comprises one or more variable domains or one or more CDRs derived from a T cell of a human subject having a cancer disclosed herein. In some embodiments, a binding protein comprises one or more variable domains or one or more CDRs derived from a T cell of a subject (e.g., a human subject) having a disease associated with a KRAS G12 mutation, such as a KRAS G12V or G12D mutation. In some embodiments, a binding protein comprises one or more variable domains or one or more CDRs derived from a T cell of a subject (e.g., a human subject) with a cell that comprises a KRAS G12 mutation, such as a KRAS G12V or G12D mutation.

In some embodiments, a binding protein comprises one or more variable domains or one or more CDRs derived from a T cell of a healthy subject (e.g., a healthy human subject). In some embodiments, a healthy subject lacks a specific pathological diagnosis (e.g., disease diagnosis, such as a cancer diagnosis). In some embodiments, a healthy subject lacks a specific pathological diagnosis, but comprises a different pathological diagnosis, for example, lacks a cancer diagnosis but comprises a diagnosis of hypertension or type II diabetes.

Presently disclosed binding proteins are capable of being heterologously expressed by host cells, such as, for example, human immune cells, such as T cells. Furthermore, expression of a presently disclosed binding protein can confer advantageous properties upon a host cell; e.g., having binding specificity for a Ras antigen:HLA complex of the present disclosure, improved activation, proliferation, or killing activity in the presence of a Ras antigen:HLA presenting tumor cell, or the like.

For example, in certain embodiments, when the binding protein is expressed by an immune cell (e.g., a human T cell, optionally a CD8+ and/or CD4+ T cell, a NK cell, or a NK-T cell), the immune cell is capable of specifically killing a HLA-A*11:01 tumor cell that expresses a peptide comprising or consisting of the amino acid sequence set forth in SEQ ID NO.: 2 or 3. Killing of a target cell can be determined, for example, the Incucyte® bioimaging platform (Essen Bioscience). In certain embodiments, this platform uses activated caspase and labelled (e.g., RapidRed or NucRed) tumor cell signals, wherein overlap is measured and increased overlap area equals tumor cell death by apoptosis. Killing can also be determined using a 4-hour assay in which target cells are loaded with labeled chromium (⁵¹Cr), and ⁵¹Cr in the supernatant is measured, e.g., following 4-hour co-incubation with an immune cell expressing a binding protein of the present disclosure. In certain embodiments, a killing assay can be performed using an effector: target cell ratio of 0.5:1, 1:1, 2:1, 3:1, 4:1, 5:1, 6:1, 7:1, 8:1, 9:1, 10:1, 20:1, 25:1, 50:1, or 100:1, or the like.

In any of the presently disclosed embodiments, when the binding protein is expressed by an immune cell (e.g., a human T cell, optionally a CD8+ and/or CD4+ T cell, a NK cell, or a NK-T cell), the immune cell has elevated expression of Nur77 when in the presence of a HLA-A11:01⁺ tumor cell that expresses a peptide comprising or consisting of the amino acid sequence set forth in SEQ ID NO.: 2 or 3, optionally in the further presence of exogenous IFN-γ, wherein the Nur77 expression is elevated as compared to: (i) Nur77 expression by a reference immune cell (i.e., of the same cell type as, and otherwise phenotypically and/or genotypically at least substantially identical or functionally equivalent to, the immune cell expressing the binding protein) not expressing the binding protein, when the reference immune cell is in the presence of the tumor cell; and/or (ii) Nur77 expression by the immune cell expressing the binding protein when not in the presence of the tumor cell and/or when not in the presence of an antigen-presenting cell expressing a peptide: HLA complex, wherein the peptide comprises, consists essentially of, or consists of the amino acid sequence set forth in SEQ ID NO.: 2 or 3, and wherein the HLA is optionally HLA-A*11:01. Expression of Nur77 can be determined, for example, using a transgenic expression construct comprising a Nur77 locus operably linked to a sequence encoding a reporter construct; e.g., dTomato (see Ahsouri and Weiss, J Immunol 198(2): 657-668 (2017)).

In any of the presently disclosed embodiments, when the binding protein is expressed by an immune cell (e.g., a human T cell, optionally a CD8+ and/or CD4+ T cell, a NK cell, or a NK-T cell), the immune cell has elevated expression of CD137 (also known as 4-1BB) when in the presence of a HLA-A*02 tumor cell that expresses a peptide comprising or consisting of the amino acid sequence set forth in SEQ ID NO.: 2 or 3, optionally in the further presence of exogenous IFN-γ, wherein the CD137 expression is elevated as compared to: (i) CD137 expression by a reference immune cell not expressing the binding protein, when the reference immune cell is in the presence of the tumor cell; and/or (ii) CD137 expression by the immune cell expressing the binding protein when not in the presence of the tumor cell and/or when not in the presence of an antigen-presenting cell expressing a peptide: HLA complex, wherein the peptide comprises, consists essentially of, or consists of the amino acid sequence set forth in SEQ ID NO.: 2 or 3, and wherein the HLA is optionally HLA-A*11:01. CD137 expression can be determined using, for example, flow cytometry using a labeled anti-CD137 antibody. In certain embodiments, CD137 is measured following a 16-hour assay in which the immune cell is co-incubated with or stimulated with peptide or a target cell expressing the peptide.

In any of the presently disclosed embodiments: (i) the binding protein is encoded by a polynucleotide that is heterologous to the immune cell; (ii) the immune cell comprises a human CD8⁺ T cell, a human CD4+ T cell, or both; (iii) the tumor cell expressing a peptide comprising or consisting of the amino acid sequence set forth in SEQ ID NO.: 2 or 3 is HLA-A*11:01; and/or (iv) the tumor cell comprises a OVCAR5 (ovarian serous adenocarcinoma), DAN-G (pancreatic adenocarcinoma), CFPAC1 (pancreatic adenocarcinoma), SW480 (colon carcinoma), SW527 (breast carcinoma), or NCI-H441 (lung adenocarcinoma) cell.

In certain embodiments, the binding protein is capable of binding to the peptide: HLA complex independent of, or in the absence of, CD8. CD8-independent binding can be determined by expressing the binding protein in a CD8-negative cell (e.g., a CD4⁺ T cell, a Jurkat cell, or the like) and identifying binding of the cell to a target. In some embodiments, a binding protein is provided that comprises: (a) a T cell receptor (TCR) α chain variable (Vα) domain comprising the complementarity determining region 3 (CDR3α) amino acid sequence set forth in any one of SEQ ID NOs.: 16, 17, 42, and 43, or a variant thereof having one, two, or three, optionally conservative, amino acid substitutions; and/or (b) a TCR β chain variable (Vβ) domain comprising the CDR3β amino acid sequence set forth in any one of SEQ ID NOs.: 26, 27, 52, and 53, or a variant thereof having one, two, or three, optionally conservative, amino acid substitutions, wherein the binding protein is capable of binding to a peptide: HLA complex, wherein the peptide comprises, consists essentially of, or consists of the amino acid sequence VVVGAVGVGK (SEQ ID NO.: 2) or VVGAVGVGK (SEQ ID NO.: 3) and wherein the HLA comprises an HLA-A*11. In certain embodiments, the HLA comprises HLA-A*11:01. The binding protein can comprise the Vα domain and the Vβ domain.

The Vα domain and/or the Vβ domain can be human, humanized, or chimeric, and is preferably human.

In certain embodiments, the binding protein comprises the CDR3α and CDR3β amino acid sequences set forth in SEQ ID NOs.: (i) 17 and 27, respectively, or variants thereof having one, two, or three, optionally conservative, amino acid substitutions; (ii) 16 and 26, respectively, or variants thereof having one, two, or three, optionally conservative, amino acid substitutions; (iii) 53 and 43, respectively, or variants thereof having one, two, or three, optionally conservative, amino acid substitutions; or (iv) 52 and 42, respectively, or variants thereof having one, two, or three, optionally conservative, amino acid substitutions. In certain embodiments, the binding protein comprises the CDR3α and CDR3β amino acid sequences set forth in SEQ ID NOs.: (i) 17 and 27, respectively; (ii) 16 and 26, respectively; (iii) 53 and 43, respectively; or (iv) 52 and 42, respectively.

In some embodiments, the binding protein further comprises: (i) in the Vα domain, the CDR1α amino acid sequence set forth in SEQ ID NO.: 14 or 40, or a variant thereof having one or two, optionally conservative, amino acid substitutions; (ii) in the Vα domain, the CDR2α amino acid sequence set forth in SEQ ID NO.: 15 or 41, or a variant thereof having one or two, optionally conservative, amino acid substitutions; (iii) in the Vβ domain, the CDR1β acid sequence set forth in SEQ ID NO.: 24 or 50, or a variant thereof having one or two, optionally conservative, amino acid substitutions; (iv) in the Vβ domain, the CDR2β acid sequence set forth in SEQ ID NO.: 25 or 51, or a variant thereof having one or two, optionally conservative, amino acid substitutions; or (v) any combination of (i)-(iv).

In some embodiments, the binding protein further comprises: (i) in the Vα domain, the CDR1α amino acid sequence set forth in SEQ ID NO.: 14 or 40; (ii) in the Vα domain, the CDR2α amino acid sequence set forth in SEQ ID NO.: 15 or 41; (iii) in the Vβ domain, the CDR1β acid sequence set forth in SEQ ID NO.: 24 or 50; (iv) in the Vβ domain, the CDR2β acid sequence set forth in SEQ ID NO.: 25 or 51; or (v) any combination of (i)-(iv).

In some embodiments, the binding protein further comprises: (i) in the Vα domain, the CDR1α amino acid sequence set forth in SEQ ID NO.: 14 or 40; (ii) in the Vα domain, the CDR2α amino acid sequence set forth in SEQ ID NO.: 15 or 41; (iii) in the Vβ domain, the CDR1β acid sequence set forth in SEQ ID NO.: 24 or 50; and (iv) in the Vβ domain, the CDR2β acid sequence set forth in SEQ ID NO.: 25 or 51.

In certain embodiments, the binding protein comprises the CDR1α, CDR2α, CDR3α, CDR1β, CDR2β, and CDR3β amino acid sequences set forth in SEQ ID NOs.: 14, 15, 16 or 17, 24, 25, and 26 or 27, respectively.

In other embodiments, the binding protein comprises the CDR1α, CDR2α, CDR3α, CDR1β, CDR2β, and CDR3β amino acid sequences set forth in SEQ ID NOs.: 40, 41, 42 or 43, 50, 51, and 52 or 52, respectively.

In some embodiments, a binding protein disclosed herein comprises a CDR1α, CDR2α, CDR3α, CDR1β, CDR2β, and/or CDR3β as identified by the Kabat method or numbering scheme from the variable domain sequence of SEQ ID NO: 13, SEQ ID NO: 23, SEQ ID NO: 39, SEQ ID NO: 49, or a combination thereof. In some embodiments, a binding protein disclosed herein comprises a CDR1α, CDR2α, and/or CDR3α as identified by the Kabat method from the variable domain sequence of SEQ ID NO: 13 or SEQ ID NO: 39. In some embodiments, a binding protein disclosed herein comprises a CDR1β, CDR2β, and/or CDR3β as identified by the Kabat method from the variable domain sequence of SEQ ID NO: 23 or SEQ ID NO: 49. In some embodiments, a binding protein disclosed herein comprises a CDR1α, CDR2α, and/or CDR3α as identified by the Kabat method from the variable domain sequence of SEQ ID NO: 13, and a CDR1β, CDR2β, and/or CDR3β as identified by the Kabat method from the variable domain sequence of SEQ ID NO: 23. In some embodiments, a binding protein disclosed herein comprises a CDR1α, CDR2α, and CDR3α as identified by the Kabat method from the variable domain sequence of SEQ ID NO: 13, and a CDR1β, CDR2β, and CDR3β as identified by the Kabat method from the variable domain sequence of SEQ ID NO: 23. In some embodiments, a binding protein disclosed herein comprises a CDR1α, CDR2α, and/or CDR3α as identified by the Kabat method from the variable domain sequence of SEQ ID NO: 39, and a CDR1β, CDR2β, and/or CDR3β as identified by the Kabat method from the variable domain sequence of SEQ ID NO: 49. In some embodiments, a binding protein disclosed herein comprises a CDR1α, CDR2α, and CDR3α as identified by the Kabat method from the variable domain sequence of SEQ ID NO: 39, and a CDR1β, CDR2β, and CDR3β as identified by the Kabat method from the variable domain sequence of SEQ ID NO: 49.

In some embodiments, a binding protein disclosed herein comprises a CDR1α, CDR2α, CDR3α, CDR1β, CDR2β, and/or CDR3β as identified by the Chothia method or numbering scheme from the variable domain sequence of SEQ ID NO: 13, SEQ ID NO: 23, SEQ ID NO: 39, SEQ ID NO: 49, or a combination thereof. In some embodiments, a binding protein disclosed herein comprises a CDR1α, CDR2α, and/or CDR3α as identified by the Chothia method from the variable domain sequence of SEQ ID NO: 13 or SEQ ID NO: 39. In some embodiments, a binding protein disclosed herein comprises a CDR1β, CDR2β, and/or CDR3β as identified by the Chothia method from the variable domain sequence of SEQ ID NO: 23 or SEQ ID NO: 49. In some embodiments, a binding protein disclosed herein comprises a CDR1α, CDR2α, and/or CDR3α as identified by the Chothia method from the variable domain sequence of SEQ ID NO: 13, and a CDR1β, CDR2β, and/or CDR3β as identified by the Chothia method from the variable domain sequence of SEQ ID NO: 23. In some embodiments, a binding protein disclosed herein comprises a CDR1α, CDR2α, and CDR3α as identified by the Chothia method from the variable domain sequence of SEQ ID NO: 13, and a CDR1β, CDR2β, and CDR3β as identified by the Chothia method from the variable domain sequence of SEQ ID NO: 23. In some embodiments, a binding protein disclosed herein comprises a CDR1α, CDR2α, and/or CDR3α as identified by the Chothia method from the variable domain sequence of SEQ ID NO: 39, and a CDR1β, CDR2β, and/or CDR3β as identified by the Chothia method from the variable domain sequence of SEQ ID NO: 49. In some embodiments, a binding protein disclosed herein comprises a CDR1α, CDR2α, and CDR3α as identified by the Chothia method from the variable domain sequence of SEQ ID NO: 39, and a CDR1β, CDR2β, and CDR3β as identified by the Chothia method from the variable domain sequence of SEQ ID NO: 49.

In some embodiments, a binding protein disclosed herein comprises a CDR1α, CDR2α, CDR3α, CDR1β, CDR2β, and/or CDR3β as identified by the EU method or numbering scheme from the variable domain sequence of SEQ ID NO: 13, SEQ ID NO: 23, SEQ ID NO: 39, SEQ ID NO: 49, or a combination thereof. In some embodiments, a binding protein disclosed herein comprises a CDR1α, CDR2α, and/or CDR3α as identified by the EU method from the variable domain sequence of SEQ ID NO: 13 or SEQ ID NO: 39. In some embodiments, a binding protein disclosed herein comprises a CDR1β, CDR2β, and/or CDR3β as identified by the EU method from the variable domain sequence of SEQ ID NO: 23 or SEQ ID NO: 49. In some embodiments, a binding protein disclosed herein comprises a CDR1α, CDR2α, and/or CDR3α as identified by the EU method from the variable domain sequence of SEQ ID NO: 13, and a CDR1β, CDR2β, and/or CDR3β as identified by the EU method from the variable domain sequence of SEQ ID NO: 23. In some embodiments, a binding protein disclosed herein comprises a CDR1α, CDR2α, and CDR3α as identified by the EU method from the variable domain sequence of SEQ ID NO: 13, and a CDR1β, CDR2β, and CDR3β as identified by the EU method from the variable domain sequence of SEQ ID NO: 23. In some embodiments, a binding protein disclosed herein comprises a CDR1α, CDR2α, and/or CDR3α as identified by the EU method from the variable domain sequence of SEQ ID NO: 39, and a CDR1β, CDR2β, and/or CDR3β as identified by the EU method from the variable domain sequence of SEQ ID NO: 49. In some embodiments, a binding protein disclosed herein comprises a CDR1α, CDR2α, and CDR3α as identified by the EU method from the variable domain sequence of SEQ ID NO: 39, and a CDR1β, CDR2β, and CDR3β as identified by the EU method from the variable domain sequence of SEQ ID NO: 49.

In some embodiments, a binding protein disclosed herein comprises a CDR1α, CDR2α, CDR3α, CDR1β, CDR2β, and/or CDR3β as identified by the IMGT method or numbering scheme (including IMGT and/or IMGT-junction for CDR3) from the variable domain sequence of SEQ ID NO: 13, SEQ ID NO: 23, SEQ ID NO: 39, SEQ ID NO: 49, or a combination thereof. In some embodiments, a binding protein disclosed herein comprises a CDR1α, CDR2α, and/or CDR3α as identified by the IMGT method from the variable domain sequence of SEQ ID NO: 13 or SEQ ID NO: 39. In some embodiments, a binding protein disclosed herein comprises a CDR1β, CDR2β, and/or CDR3β as identified by the IMGT method from the variable domain sequence of SEQ ID NO: 23 or SEQ ID NO: 49. In some embodiments, a binding protein disclosed herein comprises a CDR1α, CDR2α, and/or CDR3α as identified by the IMGT method from the variable domain sequence of SEQ ID NO: 13, and a CDR1β, CDR2β, and/or CDR3β as identified by the IMGT method from the variable domain sequence of SEQ ID NO: 23. In some embodiments, a binding protein disclosed herein comprises a CDR1α, CDR2α, and CDR3α as identified by the IMGT method from the variable domain sequence of SEQ ID NO: 13, and a CDR1β, CDR2β, and CDR3β as identified by the IMGT method from the variable domain sequence of SEQ ID NO: 23. In some embodiments, a binding protein disclosed herein comprises a CDR1α, CDR2α, and/or CDR3α as identified by the IMGT method from the variable domain sequence of SEQ ID NO: 39, and a CDR1β, CDR2β, and/or CDR3β as identified by the IMGT method from the variable domain sequence of SEQ ID NO: 49. In some embodiments, a binding protein disclosed herein comprises a CDR1α, CDR2α, and CDR3α as identified by the IMGT method from the variable domain sequence of SEQ ID NO: 39, and a CDR1β, CDR2β, and CDR3β as identified by the IMGT method from the variable domain sequence of SEQ ID NO: 49.

In some embodiments, a binding protein disclosed herein comprises a CDR1α, CDR2α, CDR3α, CDR1β, CDR2β, and/or CDR3β as identified by the IMGT method or numbering scheme (including IMGT and/or IMGT-junction for CDR3) from the amino acid sequence set forth in SEQ ID NO.: 20, the amino acid sequence set forth in SEQ ID NO.: 30, the amino acid sequence set forth in SEQ ID NO.: 155, or a combination thereof. In some embodiments, a binding protein disclosed herein comprises a CDR1α, CDR2α, and/or CDR3α as identified by the IMGT method from the amino acid sequence set forth in SEQ ID NO.: 20. In some embodiments, a binding protein disclosed herein comprises a CDR1β, CDR2β, and/or CDR3β as identified by the IMGT method from the amino acid sequence set forth in SEQ ID NO.: 30 or SEQ ID NO.: 155. In some embodiments, a binding protein disclosed herein comprises a CDR1α, CDR2α, and CDR3α as identified by the IMGT method from the amino acid sequence set forth in SEQ ID NO.: 20, and a CDR1β, CDR2β, and CDR3β as identified by the IMGT method from the amino acid sequence set forth in SEQ ID NO.: 30 or SEQ ID NO.: 155.

In some embodiments, a binding protein disclosed herein comprises a CDR1α, CDR2α, CDR3α, CDR1β, CDR2β, and/or CDR3β as identified by the Enhanced Chothia method or numbering scheme from the variable domain sequence of SEQ ID NO: 13, SEQ ID NO: 23, SEQ ID NO: 39, SEQ ID NO: 49, or a combination thereof. In some embodiments, a binding protein disclosed herein comprises a CDR1α, CDR2α, and/or CDR3α as identified by the Enhanced Chothia method from the variable domain sequence of SEQ ID NO: 13 or SEQ ID NO: 39. In some embodiments, a binding protein disclosed herein comprises a CDR1β, CDR2β, and/or CDR3β as identified by the Enhanced Chothia method from the variable domain sequence of SEQ ID NO: 23 or SEQ ID NO: 49. In some embodiments, a binding protein disclosed herein comprises a CDR1α, CDR2α, and/or CDR3α as identified by the Enhanced Chothia method from the variable domain sequence of SEQ ID NO: 13, and a CDR1β, CDR2β, and/or CDR3β as identified by the Enhanced Chothia method from the variable domain sequence of SEQ ID NO: 23. In some embodiments, a binding protein disclosed herein comprises a CDR1α, CDR2α, and CDR3α as identified by the Enhanced Chothia method from the variable domain sequence of SEQ ID NO: 13, and a CDR1β, CDR2β, and CDR3β as identified by the Enhanced Chothia method from the variable domain sequence of SEQ ID NO: 23. In some embodiments, a binding protein disclosed herein comprises a CDR1α, CDR2α, and/or CDR3α as identified by the Enhanced Chothia method from the variable domain sequence of SEQ ID NO: 39, and a CDR1β, CDR2β, and/or CDR3β as identified by the Enhanced Chothia method from the variable domain sequence of SEQ ID NO: 49. In some embodiments, a binding protein disclosed herein comprises a CDR1α, CDR2α, and CDR3α as identified by the Enhanced Chothia method from the variable domain sequence of SEQ ID NO: 39, and a CDR1β, CDR2β, and CDR3β as identified by the Enhanced Chothia method from the variable domain sequence of SEQ ID NO: 49.

In some embodiments, a binding protein disclosed herein comprises a CDR1α, CDR2α, CDR3α, CDR1β, CDR2β, and/or CDR3β as identified by the Aho method or numbering scheme from the variable domain sequence of SEQ ID NO: 13, SEQ ID NO: 23, SEQ ID NO: 39, SEQ ID NO: 49, or a combination thereof. In some embodiments, a binding protein disclosed herein comprises a CDR1α, CDR2α, and/or CDR3α as identified by the Aho method from the variable domain sequence of SEQ ID NO: 13 or SEQ ID NO: 39. In some embodiments, a binding protein disclosed herein comprises a CDR1β, CDR2β, and/or CDR3β as identified by the Aho method from the variable domain sequence of SEQ ID NO: 23 or SEQ ID NO: 49. In some embodiments, a binding protein disclosed herein comprises a CDR1α, CDR2α, and/or CDR3α as identified by the Aho method from the variable domain sequence of SEQ ID NO: 13, and a CDR1β, CDR2β, and/or CDR3β as identified by the Aho method from the variable domain sequence of SEQ ID NO: 23. In some embodiments, a binding protein disclosed herein comprises a CDR1α, CDR2α, and CDR3α as identified by the Aho method from the variable domain sequence of SEQ ID NO: 13, and a CDR1β, CDR2β, and CDR3β as identified by the Aho method from the variable domain sequence of SEQ ID NO: 23. In some embodiments, a binding protein disclosed herein comprises a CDR1α, CDR2α, and/or CDR3α as identified by the Aho method from the variable domain sequence of SEQ ID NO: 39, and a CDR1β, CDR2β, and/or CDR3β as identified by the Aho method from the variable domain sequence of SEQ ID NO: 49. In some embodiments, a binding protein disclosed herein comprises a CDR1α, CDR2α, and CDR3α as identified by the Aho method from the variable domain sequence of SEQ ID NO: 39, and a CDR1β, CDR2β, and CDR3β as identified by the Aho method from the variable domain sequence of SEQ ID NO: 49.

In some embodiments, the binding protein comprises a CDR1α that comprises, consists essentially of, or consists of the amino acid sequence of SEQ ID NO: 14 or 40, or a CDR1α sequence as identified by the Kabat, Chothia, EU, IMGT, Enhanced Chothia, or Aho method from the variable domain of SEQ ID NO: 13 or 39.

In some embodiments, the binding protein comprises a CDR2α that comprises, consists essentially of, or consists of the amino acid sequence of SEQ ID NO: 15 or 41, or a CDR2a sequence as identified by the Kabat, Chothia, EU, IMGT, Enhanced Chothia, or Aho method from the variable domain of SEQ ID NO: 13 or 39.

In some embodiments, the binding protein comprises a CDR3α that comprises, consists essentially of, or consists of the amino acid sequence of SEQ ID NO: 16, 17, 42, or 43, or a CDR3α sequence as identified by the Kabat, Chothia, EU, IMGT, Enhanced Chothia, or Aho method from the variable domain of SEQ ID NO: 13 or 39.

In some embodiments, the binding protein comprises a CDR1β that comprises, consists essentially of, or consists of the amino acid sequence of SEQ ID NO: 24 or 50, or a CDR1β sequence as identified by the Kabat, Chothia, EU, IMGT, Enhanced Chothia, or Aho method from the variable domain of SEQ ID NO: 23 or 49.

In some embodiments, the binding protein comprises a CDR2β that comprises, consists essentially of, or consists of the amino acid sequence of SEQ ID NO: 25 or 51, or a CDR2β sequence as identified by the Kabat, Chothia, EU, IMGT, Enhanced Chothia, or Aho method from the variable domain of SEQ ID NO: 23 or 49.

In some embodiments, the binding protein comprises a CDR3β that comprises, consists essentially of, or consists of the amino acid sequence of SEQ ID NO: 26, 27, 52, or 53, or a CDR3β sequence as identified by the Kabat, Chothia, EU, IMGT, Enhanced Chothia, or Aho method from the variable domain of SEQ ID NO: 23 or 49.

In some embodiments, the binding protein comprises a CDR1α that comprises at most one, at most two, at most three, at most four, at most five, or at most six amino acid substitutions relative to the amino acid sequence of SEQ ID NO: 14 or 40, or a CDR1α sequence as identified by the Kabat, Chothia, EU, IMGT, Enhanced Chothia, or Aho method from the variable domain of SEQ ID NO: 13 or 39. The substitution(s) can be at the N-terminus of the CDR, the C-terminus of the CDR, within the amino acid sequence of the CDR, or a combination thereof. The substitutions can be conservative, non-conservative, or a combination thereof. In some embodiments, the substitutions are conservative.

In some embodiments, the binding protein comprises a CDR2α that comprises at most one, at most two, at most three, at most four, at most five, or at most six amino acid substitutions relative to the amino acid sequence of SEQ ID NO: 15 or 41, or a CDR2α sequence as identified by the Kabat, Chothia, EU, IMGT, Enhanced Chothia, or Aho method from the variable domain of SEQ ID NO: 13 or 39. The substitution(s) can be at the N-terminus of the CDR, the C-terminus of the CDR, within the amino acid sequence of the CDR, or a combination thereof. The substitutions can be conservative, non-conservative, or a combination thereof. In some embodiments, the substitutions are conservative.

In some embodiments, the binding protein comprises a CDR3α that comprises at most one, at most two, at most three, at most four, at most five, or at most six amino acid substitutions relative to the amino acid sequence of SEQ ID NO: 16, 17, 42, or 43, or a CDR3α sequence as identified by the Kabat, Chothia, EU, IMGT, Enhanced Chothia, or Aho method from the variable domain of SEQ ID NO: 13 or 39. The substitution(s) can be at the N-terminus of the CDR, the C-terminus of the CDR, within the amino acid sequence of the CDR, or a combination thereof. The substitutions can be conservative, non-conservative, or a combination thereof. In some embodiments, the substitutions are conservative.

In some embodiments, the binding protein comprises a CDR1β that comprises at most one, at most two, at most three, at most four, at most five, or at most six amino acid substitutions relative to the amino acid sequence of SEQ ID NO: 24 or 50, or a CDR1β sequence as identified by the Kabat, Chothia, EU, IMGT, Enhanced Chothia, or Aho method from the variable domain of SEQ ID NO: 23 or 49. The substitution(s) can be at the N-terminus of the CDR, the C-terminus of the CDR, within the amino acid sequence of the CDR, or a combination thereof. The substitutions can be conservative, non-conservative, or a combination thereof. In some embodiments, the substitutions are conservative.

In some embodiments, the binding protein comprises a CDR2β that comprises at most one, at most two, at most three, at most four, at most five, or at most six amino acid substitutions relative to the amino acid sequence of SEQ ID NO: 25 or 51, or a CDR2β sequence as identified by the Kabat, Chothia, EU, IMGT, Enhanced Chothia, or Aho method from the variable domain of SEQ ID NO: 23 or 49. The substitution(s) can be at the N-terminus of the CDR, the C-terminus of the CDR, within the amino acid sequence of the CDR, or a combination thereof. The substitutions can be conservative, non-conservative, or a combination thereof. In some embodiments, the substitutions are conservative.

In some embodiments, the binding protein comprises a CDR3β that comprises at most one, at most two, at most three, at most four, at most five, or at most six amino acid substitutions relative to the amino acid sequence of SEQ ID NO: 26, 27, 52, or 53, or a CDR3β sequence as identified by the Kabat, Chothia, EU, IMGT, Enhanced Chothia, or Aho method from the variable domain of SEQ ID NO: 23 or 49. The substitution(s) can be at the N-terminus of the CDR, the C-terminus of the CDR, within the amino acid sequence of the CDR, or a combination thereof. The substitutions can be conservative, non-conservative, or a combination thereof. In some embodiments, the substitutions are conservative.

In some embodiments, the binding protein comprises a CDR1α that comprises at most one, at most two, at most three, at most four, at most five, or at most six amino acid insertions and/or deletions relative to the amino acid sequence of SEQ ID NO: 14 or 40, or a CDR1α sequence as identified by the Kabat, Chothia, EU, IMGT, Enhanced Chothia, or Aho method from the variable domain of SEQ ID NO: 13 or 39. The insertion(s) and/or deletion(s) can be at the N-terminus of the CDR, the C-terminus of the CDR, within the amino acid sequence of the CDR, or a combination thereof.

In some embodiments, the binding protein comprises a CDR2α that comprises at most one, at most two, at most three, at most four, at most five, or at most six amino acid insertions and/or deletions relative to the amino acid sequence of SEQ ID NO: 15 or 41, or a CDR2a sequence as identified by the Kabat, Chothia, EU, IMGT, Enhanced Chothia, or Aho method from the variable domain of SEQ ID NO: 13 or 39. The insertion(s) and/or deletion(s) can be at the N-terminus of the CDR, the C-terminus of the CDR, within the amino acid sequence of the CDR, or a combination thereof.

In some embodiments, the binding protein comprises a CDR3α that comprises at most one, at most two, at most three, at most four, at most five, or at most six amino acid insertions and/or deletions relative to the amino acid sequence of SEQ ID NO: 16, 17, 42, or 43, or a CDR3α sequence as identified by the Kabat, Chothia, EU, IMGT, Enhanced Chothia, or Aho method from the variable domain of SEQ ID NO: 13 or 39. The insertion(s) and/or deletion(s) can be at the N-terminus of the CDR, the C-terminus of the CDR, within the amino acid sequence of the CDR, or a combination thereof.

In some embodiments, the binding protein comprises a CDR1β that comprises at most one, at most two, at most three, at most four, at most five, or at most six amino acid insertions and/or deletions relative to the amino acid sequence of SEQ ID NO: 24 or 50, or a CDR1β sequence as identified by the Kabat, Chothia, EU, IMGT, Enhanced Chothia, or Aho method from the variable domain of SEQ ID NO: 23 or 49. The insertion(s) and/or deletion(s) can be at the N-terminus of the CDR, the C-terminus of the CDR, within the amino acid sequence of the CDR, or a combination thereof.

In some embodiments, the binding protein comprises a CDR2β that comprises at most one, at most two, at most three, at most four, at most five, or at most six amino acid insertions and/or deletions relative to the amino acid sequence of SEQ ID NO: 25 or 51, or a CDR2β sequence as identified by the Kabat, Chothia, EU, IMGT, Enhanced Chothia, or Aho method from the variable domain of SEQ ID NO: 23 or 49. The insertion(s) and/or deletion(s) can be at the N-terminus of the CDR, the C-terminus of the CDR, within the amino acid sequence of the CDR, or a combination thereof.

In some embodiments, the binding protein comprises a CDR3β that comprises at most one, at most two, at most three, at most four, at most five, or at most six amino acid insertions and/or deletions relative to the amino acid sequence of SEQ ID NO: 26, 27, 52, or 53, or a CDR3β sequence as identified by the Kabat, Chothia, EU, IMGT, Enhanced Chothia, or Aho method from the variable domain of SEQ ID NO: 23 or 49. The insertion(s) and/or deletion(s) can be at the N-terminus of the CDR, the C-terminus of the CDR, within the amino acid sequence of the CDR, or a combination thereof.

A binding protein disclosed herein can comprise one or more framework regions (FRs). For example, a binding protein can comprise a variable domain comprising three CDRs and four FRs, or two variable domains each comprising three CDRs and four FRs. Illustrative FR amino acid sequences are provided by SEQ ID NOs: 91-117 and 153. A framework region used in a binding protein can be a mammalian framework region. A framework region used in a binding protein can be a human framework region. A framework region used in a binding protein can be an engineered framework region.

A binding protein can comprise an FR1, an FR2, and FR3, and/or an FR4 disclosed herein. In some embodiments, a binding protein comprises a Vα comprising an FR1 comprising, consisting essentially of, or consisting of the amino acid sequence set forth in any one of SEQ ID NOs: 91, 103, and 115, or a variant thereof, an FR2 comprising, consisting essentially of, or consisting of the amino acid sequence set forth in any one of SEQ ID NOs: 92, 104, or a variant thereof, an FR3 comprising, consisting essentially of, or consisting of the amino acid sequence set forth in any one of SEQ ID NOs: 93, 95, 105, 107 or a variant thereof, and an FR4 comprising, consisting essentially of, or consisting of the amino acid sequence set forth in any one of SEQ ID NOs: 94, 96, 106, and 108, or a variant thereof. In some embodiments, the binding protein comprises a Vα comprising the amino acid sequence set forth in SEQ ID NO.: 115. In some embodiments, the binding protein comprises a Vα comprising the amino acid sequence set forth in SEQ ID NO.: 91.

In some embodiments, a binding protein comprises a Vβ comprising an FR1 comprising, consisting essentially of, or consisting of the amino acid sequence set forth in any one of SEQ ID NOs: 97, 109, and 153, or a variant thereof, an FR2 comprising, consisting essentially of, or consisting of the amino acid sequence set forth in any one of SEQ ID NOs: 98, 110, or a variant thereof, an FR3 comprising, consisting essentially of, or consisting of the amino acid sequence set forth in any one of SEQ ID NOs: 99, 101, 111, 113, or a variant thereof, and an FR4 comprising, consisting essentially of, or consisting of the amino acid sequence set forth in any one of SEQ ID NOs: 100, 102, 112, 114, 116, and 117, or a variant thereof.

In some embodiments, the binding protein comprises a Vβ comprising the amino acid sequence set forth in SEQ ID NO.: 153. In some embodiments, the binding protein comprises a Vβ comprising the amino acid sequence set forth in SEQ ID NO.: 97.

In some embodiments, a binding protein comprises a Vα domain comprising an FR1, FR2, FR3, and FR4 as identified by the Kabat method or numbering scheme from the variable domain sequence of SEQ ID NO: 13 or SEQ ID NO: 39. In some embodiments, a binding protein comprises a Vβ domain comprising an FR1, FR2, FR3, and FR4 as identified by the Kabat method from the variable domain sequence of SEQ ID NO: 23 or SEQ ID NO: 49.

In some embodiments, a binding protein comprises a Vα domain comprising an FR1, FR2, FR3, and FR4 as identified by the Chothia method or numbering scheme from the variable domain sequence of SEQ ID NO: 13 or SEQ ID NO: 39. In some embodiments, a binding protein comprises a Vβ domain comprising an FR1, FR2, FR3, and FR4 as identified by the Chothia method from the variable domain sequence of SEQ ID NO: 23 or SEQ ID NO: 49.

In some embodiments, a binding protein comprises a Vα domain comprising an FR1, FR2, FR3, and FR4 as identified by the EU method or numbering scheme from the variable domain sequence of SEQ ID NO: 13 or SEQ ID NO: 39. In some embodiments, a binding protein comprises a Vβ domain comprising an FR1, FR2, FR3, and FR4 as identified by the EU method from the variable domain sequence of SEQ ID NO: 23 or SEQ ID NO: 49.

In some embodiments, a binding protein comprises a Vα domain comprising an FR1, FR2, FR3, and FR4 as identified by the IMGT method or numbering scheme from the variable domain sequence of SEQ ID NO: 13 or SEQ ID NO: 39. In some embodiments, a binding protein comprises a Vβ domain comprising an FR1, FR2, FR3, and FR4 as identified by the IMGT method from the variable domain sequence of SEQ ID NO: 23 or SEQ ID NO: 49.

In some embodiments, a binding protein comprises a Vα domain comprising an FR1, FR2, FR3, and FR4 as identified by the Enhanced Chothia method or numbering scheme from the variable domain sequence of SEQ ID NO: 13 or SEQ ID NO: 39. In some embodiments, a binding protein comprises a Vβ domain comprising an FR1, FR2, FR3, and FR4 as identified by the Enhanced Chothia method from the variable domain sequence of SEQ ID NO: 23 or SEQ ID NO: 49.

In some embodiments, a binding protein comprises a Vα domain comprising an FR1, FR2, FR3, and FR4 as identified by the Aho method or numbering scheme from the variable domain sequence of SEQ ID NO: 13 or SEQ ID NO: 39. In some embodiments, a binding protein comprises a Vβ domain comprising an FR1, FR2, FR3, and FR4 as identified by the Aho method from the variable domain sequence of SEQ ID NO: 23 or SEQ ID NO: 49.

In some embodiments, the binding protein comprises a Vα domain comprising an FR1 that comprises, consists essentially of, or consists of the amino acid sequence of SEQ ID NO: 91, 103, or 115, or an FR1 sequence as identified by the Kabat, Chothia, EU, IMGT, Enhanced Chothia, or Aho method from the variable domain of SEQ ID NO: 13 or 39.

In some embodiments, the binding protein comprises a Vα domain comprising an FR2 that comprises, consists essentially of, or consists of the amino acid sequence of SEQ ID NO: 92 or 104, or an FR2 sequence as identified by the Kabat, Chothia, EU, IMGT, Enhanced Chothia, or Aho method from the variable domain of SEQ ID NO: 13 or 39.

In some embodiments, the binding protein comprises a Vα domain comprising an FR3 that comprises, consists essentially of, or consists of the amino acid sequence of SEQ ID NO: 93, 95, 105, or 107, or an FR3 sequence as identified by the Kabat, Chothia, EU, IMGT, Enhanced Chothia, or Aho method from the variable domain of SEQ ID NO: 13 or 39.

In some embodiments, the binding protein comprises a Vα domain comprising an FR4 that comprises, consists essentially of, or consists of the amino acid sequence of SEQ ID NO: 94, 96, 106, or 108, or an FR4 sequence as identified by the Kabat, Chothia, EU, IMGT, Enhanced Chothia, or Aho method from the variable domain of SEQ ID NO: 13 or 39.

In some embodiments, the binding protein comprises a Vβ domain comprising an FR1 that comprises, consists essentially of, or consists of the amino acid sequence of SEQ ID NO: 97 or 109, or an FR1 sequence as identified by the Kabat, Chothia, EU, IMGT, Enhanced Chothia, or Aho method from the variable domain of SEQ ID NO: 23 or 49.

In some embodiments, the binding protein comprises a Vβ domain comprising an FR2 that comprises, consists essentially of, or consists of the amino acid sequence of SEQ ID NO: 98 or 110, or an FR2 sequence as identified by the Kabat, Chothia, EU, IMGT, Enhanced Chothia, or Aho method from the variable domain of SEQ ID NO: 23 or 49.

In some embodiments, the binding protein comprises a Vβ domain comprising an FR3 that comprises, consists essentially of, or consists of the amino acid sequence of SEQ ID NO: 99, 101, 111, or 113, or an FR3 sequence as identified by the Kabat, Chothia, EU, IMGT, Enhanced Chothia, or Aho method from the variable domain of SEQ ID NO: 23 or 49.

In some embodiments, the binding protein comprises a Vβ domain comprising an FR4 that comprises, consists essentially of, or consists of the amino acid sequence of SEQ ID NO: 100, 102, 112, 114, 116, or 117, or an FR4 sequence as identified by the Kabat, Chothia, EU, IMGT, Enhanced Chothia, or Aho method from the variable domain of SEQ ID NO: 23 or 49.

In some embodiments, the binding protein comprises a Vα domain comprising an FR1 that comprises at most one, at most two, at most three, at most four, at most five, or at most six amino acid substitutions relative to the amino acid sequence of SEQ ID NO: 91, 103, or 115, or an FR1 sequence as identified by the Kabat, Chothia, EU, IMGT, Enhanced Chothia, or Aho method from the variable domain of SEQ ID NO: 13 or 39. The substitution(s) can be at the N-terminus, the C-terminus, within the amino acid sequence, or a combination thereof. The substitutions can be conservative, non-conservative, or a combination thereof. In some embodiments, the substitutions are conservative.

In some embodiments, the binding protein comprises a Vα domain comprising an FR2 that comprises at most one, at most two, at most three, at most four, at most five, or at most six amino acid substitutions relative to the amino acid sequence of SEQ ID NO: 92 or 104, or an FR2 sequence as identified by the Kabat, Chothia, EU, IMGT, Enhanced Chothia, or Aho method from the variable domain of SEQ ID NO: 13 or 39. The substitution(s) can be at the N-terminus, the C-terminus, within the amino acid sequence, or a combination thereof. The substitutions can be conservative, non-conservative, or a combination thereof. In some embodiments, the substitutions are conservative.

In some embodiments, the binding protein comprises a Vα domain comprising an FR3 that comprises at most one, at most two, at most three, at most four, at most five, or at most six amino acid substitutions relative to the amino acid sequence of SEQ ID NO: 93, 95, 105, or 107, or an FR3 sequence as identified by the Kabat, Chothia, EU, IMGT, Enhanced Chothia, or Aho method from the variable domain of SEQ ID NO: 13 or 39. The substitution(s) can be at the N-terminus, the C-terminus, within the amino acid sequence, or a combination thereof. The substitutions can be conservative, non-conservative, or a combination thereof. In some embodiments, the substitutions are conservative.

In some embodiments, the binding protein comprises a Vα domain comprising an FR4 that comprises at most one, at most two, at most three, at most four, at most five, or at most six amino acid substitutions relative to the amino acid sequence of SEQ ID NO: 94, 96, 106, or 108, or an FR4 sequence as identified by the Kabat, Chothia, EU, IMGT, Enhanced Chothia, or Aho method from the variable domain of SEQ ID NO: 13 or 39. The substitution(s) can be at the N-terminus, the C-terminus, within the amino acid sequence, or a combination thereof. The substitutions can be conservative, non-conservative, or a combination thereof. In some embodiments, the substitutions are conservative.

In some embodiments, the binding protein comprises a Vβ domain comprising an FR1 that comprises at most one, at most two, at most three, at most four, at most five, or at most six amino acid substitutions relative to the amino acid sequence of SEQ ID NO: 97 or 109 or 153, or an FR1 sequence as identified by the Kabat, Chothia, EU, IMGT, Enhanced Chothia, or Aho method from the variable domain of SEQ ID NO: 23 or 49. The substitution(s) can be at the N-terminus, the C-terminus, within the amino acid sequence, or a combination thereof. The substitutions can be conservative, non-conservative, or a combination thereof. In some embodiments, the substitutions are conservative.

In some embodiments, the binding protein comprises a Vβ domain comprising an FR2 that comprises at most one, at most two, at most three, at most four, at most five, or at most six amino acid substitutions relative to the amino acid sequence of SEQ ID NO: 98 or 110, or an FR2 sequence as identified by the Kabat, Chothia, EU, IMGT, Enhanced Chothia, or Aho method from the variable domain of SEQ ID NO: 23 or 49. The substitution(s) can be at the N-terminus, the C-terminus, within the amino acid sequence, or a combination thereof. The substitutions can be conservative, non-conservative, or a combination thereof. In some embodiments, the substitutions are conservative.

In some embodiments, the binding protein comprises a Vβ domain comprising an FR3 that comprises at most one, at most two, at most three, at most four, at most five, or at most six amino acid substitutions relative to the amino acid sequence of SEQ ID NO: 99, 101, 111, or 113, or an FR3 sequence as identified by the Kabat, Chothia, EU, IMGT, Enhanced Chothia, or Aho method from the variable domain of SEQ ID NO: 23 or 49. The substitution(s) can be at the N-terminus, the C-terminus, within the amino acid sequence, or a combination thereof. The substitutions can be conservative, non-conservative, or a combination thereof. In some embodiments, the substitutions are conservative.

In some embodiments, the binding protein comprises a Vβ domain comprising an FR4 that comprises at most one, at most two, at most three, at most four, at most five, or at most six amino acid substitutions relative to the amino acid sequence of SEQ ID NO: 100, 102, 112, 114, 116, or 117, or an FR4 sequence as identified by the Kabat, Chothia, EU, IMGT, Enhanced Chothia, or Aho method from the variable domain of SEQ ID NO: 23 or 49. The substitution(s) can be at the N-terminus, the C-terminus, within the amino acid sequence, or a combination thereof. The substitutions can be conservative, non-conservative, or a combination thereof. In some embodiments, the substitutions are conservative.

In some embodiments, the binding protein comprises a Vα domain comprising an FR1 that comprises at most one, at most two, at most three, at most four, at most five, or at most six amino acid insertions and/or deletions relative to the amino acid sequence of SEQ ID NO: 91, 103, or 115, or an FR1 sequence as identified by the Kabat, Chothia, EU, IMGT, Enhanced Chothia, or Aho method from the variable domain of SEQ ID NO: 13 or 39. The insertion(s) and/or deletion(s) can be at the N-terminus, the C-terminus, within the amino acid sequence, or a combination thereof.

In some embodiments, the binding protein comprises a Vα domain comprising an FR2 that comprises at most one, at most two, at most three, at most four, at most five, or at most six amino acid insertions and/or deletions relative to the amino acid sequence of SEQ ID NO: 92 or 104, or an FR2 sequence as identified by the Kabat, Chothia, EU, IMGT, Enhanced Chothia, or Aho method from the variable domain of SEQ ID NO: 13 or 39. The insertion(s) and/or deletion(s) can be at the N-terminus, the C-terminus, within the amino acid sequence, or a combination thereof.

In some embodiments, the binding protein comprises a Vα domain comprising an FR3 that comprises at most one, at most two, at most three, at most four, at most five, or at most six amino acid insertions and/or deletions relative to the amino acid sequence of SEQ ID NO: 93, 95, 105, or 107, or an FR3 sequence as identified by the Kabat, Chothia, EU, IMGT, Enhanced Chothia, or Aho method from the variable domain of SEQ ID NO: 13 or 39. The insertion(s) and/or deletion(s) can be at the N-terminus, the C-terminus, within the amino acid sequence, or a combination thereof.

In some embodiments, the binding protein comprises a Vα domain comprising an FR4 that comprises at most one, at most two, at most three, at most four, at most five, or at most six amino acid insertions and/or deletions relative to the amino acid sequence of SEQ ID NO: 94, 96, 106, or 108, or an FR4 sequence as identified by the Kabat, Chothia, EU, IMGT, Enhanced Chothia, or Aho method from the variable domain of SEQ ID NO: 13 or 39. The insertion(s) and/or deletion(s) can be at the N-terminus, the C-terminus, within the amino acid sequence, or a combination thereof.

In some embodiments, the binding protein comprises a Vβ domain comprising an FR1 that comprises at most one, at most two, at most three, at most four, at most five, or at most six amino acid insertions and/or deletions relative to the amino acid sequence of SEQ ID NO: 97 or 109 or 153, or an FR1 sequence as identified by the Kabat, Chothia, EU, IMGT, Enhanced Chothia, or Aho method from the variable domain of SEQ ID NO: 23 or 49. The insertion(s) and/or deletion(s) can be at the N-terminus, the C-terminus, within the amino acid sequence, or a combination thereof.

In some embodiments, the binding protein comprises a Vβ domain comprising an FR2 that comprises at most one, at most two, at most three, at most four, at most five, or at most six amino acid insertions and/or deletions relative to the amino acid sequence of SEQ ID NO: 98 or 110, or an FR2 sequence as identified by the Kabat, Chothia, EU, IMGT, Enhanced Chothia, or Aho method from the variable domain of SEQ ID NO: 23 or 49. The insertion(s) and/or deletion(s) can be at the N-terminus, the C-terminus, within the amino acid sequence, or a combination thereof.

In some embodiments, the binding protein comprises a Vβ domain comprising an FR3 that comprises at most one, at most two, at most three, at most four, at most five, or at most six amino acid insertions and/or deletions relative to the amino acid sequence of SEQ ID NO: 99, 101, 111, or 113, or an FR3 sequence as identified by the Kabat, Chothia, EU, IMGT, Enhanced Chothia, or Aho method from the variable domain of SEQ ID NO: 23 or 49. The insertion(s) and/or deletion(s) can be at the N-terminus, the C-terminus, within the amino acid sequence, or a combination thereof.

In some embodiments, the binding protein comprises a Vβ domain comprising an FR4 that comprises at most one, at most two, at most three, at most four, at most five, or at most six amino acid insertions and/or deletions relative to the amino acid sequence of SEQ ID NO: 100, 102, 112, 114, 116, or 117, or an FR4 sequence as identified by the Kabat, Chothia, EU, IMGT, Enhanced Chothia, or Aho method from the variable domain of SEQ ID NO: 23 or 49. The insertion(s) and/or deletion(s) can be at the N-terminus, the C-terminus, within the amino acid sequence, or a combination thereof.

The binding protein can comprise a TCRα FR1, CDR1, FR2, CDR2, FR3, CDR3, or FR4 region; a TCRβ FR1, CDR1, FR2, CDR2, FR3, CDR3, or FR4 region, or a combination thereof.

In some embodiments: (i) the Vα domain comprises, consists essentially of, or consists of an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity or sequence similarity to the amino acid sequence set forth in SEQ ID NO.: 13 or 39; and/or (ii) the Vβ domain comprises, consists essentially of, or consists of an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity or sequence similarity to the amino acid sequence set forth in SEQ ID NO.: 23 or 154 or 49.

In some embodiments, the Vα domain comprises, consists essentially of, or consists of an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity or sequence similarity to the amino acid sequence set forth in SEQ ID NO.: 13, and the Vβ domain comprises, consists essentially of, or consists of an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity or sequence similarity to the amino acid sequence set forth in SEQ ID NO.: 23 or 154.

In some embodiments, the Vα domain comprises, consists essentially of, or consists of an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity or sequence similarity to the amino acid sequence set forth in SEQ ID NO.: 39, and wherein the Vβ domain comprises, consists essentially of, or consists of an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity or sequence similarity to the amino acid sequence set forth in SEQ ID NO.: 49.

In certain embodiments, the Vα domain comprises, consists essentially of, or consists of the amino acid sequence set forth in SEQ ID NO.: 13 and the Vβ domain comprises, consists essentially of, or consists of the amino acid sequence set forth in SEQ ID NO.: 23. In certain embodiments, the a binding protein comprises a TCRβ chain and the TCRβ chain comprises the amino acids K-A immediately N-terminal to the amino acid sequence set forth in SEQ ID NO.: 23. In certain embodiments, the Vα domain comprises the amino acid sequence set forth in SEQ ID NO.: 13 and the Vβ domain comprises the amino acid sequence set forth in SEQ ID NO.: 23. In certain embodiments, the Vα domain consists of the amino acid sequence set forth in SEQ ID NO.: 13 and the Vβ domain consists of the amino acid sequence set forth in SEQ ID NO.: 23. In certain embodiments, the Vα domain consists essentially of the amino acid sequence set forth in SEQ ID NO.: 13 and the Vβ domain consists essentially of the amino acid sequence set forth in SEQ ID NO.: 23.

In some embodiments a binding protein is provided that comprises a TCR α-chain and a TCR β-chain, wherein the TCR α-chain comprises the amino acid sequence set forth in SEQ ID NO.: 13 and the TCR β-chain comprises the amino acid sequence set forth in SEQ ID NO.: 23 or or 154.

In some embodiments a binding protein is provided that is capable of binding to a peptide: HLA complex, wherein the peptide comprises, consists essentially of, or consists of SEQ ID NO.: 2 or SEQ ID NO.: 3 and wherein the HLA is optionally an HLA-A*11, further optionally HLA-A*11:01. In certain embodiments, the binding protein comprises a first polypeptide and a second polypeptide, wherein the first polypeptide comprises the amino acid sequence set forth in SEQ ID NO.: 13 and the second polypeptide comprises the amino acid sequence set forth in SEQ ID NO.: 23 or or 154. The first polypeptide can be or comprise a TCRα chain and/or the second polypeptide can be or comprise a TCRβ chain. In some embodiments, the first polypeptide is or comprises a TCRα chain and/or the second polypeptide is or comprises a TCRβ chain.

In certain embodiments, the Vα domain comprises, consists essentially of, or consists of the amino acid sequence set forth in SEQ ID NO.: 39 and the and the Vβ domain comprises, consists essentially of, or consists of amino acid sequence set forth in SEQ ID NO.: 49. In certain embodiments, the Vα domain comprises the amino acid sequence set forth in SEQ ID NO.: 39 and the Vβ domain comprises the amino acid sequence set forth in SEQ ID NO.: 49. In certain embodiments, the Vα domain consists of the amino acid sequence set forth in SEQ ID NO.: 39 and the Vβ domain consists of the amino acid sequence set forth in SEQ ID NO.: 49. In certain embodiments, the Vα domain consists essentially of the amino acid sequence set forth in SEQ ID NO.: 39 and the Vβ domain consists essentially of the amino acid sequence set forth in SEQ ID NO.: 49.

In some embodiments a binding protein is provided that comprises a TCR α-chain and a TCR β-chain, wherein the TCR α-chain comprises the amino acid sequence set forth in SEQ ID NO.: 13 and the TCR β-chain comprises the amino acid sequence set forth in SEQ ID NO.: 23 or 154.

In some embodiments a binding protein is provided that comprises a TCR α-chain and a TCR β-chain, wherein the TCR α-chain comprises the amino acid sequence set forth in SEQ ID NO.: 20 and the TCR β-chain comprises the amino acid sequence set forth in SEQ ID NO.: 30.

In some embodiments a binding protein is provided that comprises a TCR α-chain and a TCR β-chain, wherein the TCR α-chain comprises the amino acid sequence set forth in SEQ ID NO.: 20 and the TCR β-chain comprises the amino acid sequence set forth in SEQ ID NO.: 155.

In some embodiments a binding protein is provided that is capable of binding to a peptide: HLA complex, wherein the peptide comprises, consists essentially of, or consists of SEQ ID NO.: 2 or SEQ ID NO.: 3 and wherein the HLA is optionally an HLA-A*11, further optionally HLA-A*11:01. In certain embodiments, the binding protein comprises a first polypeptide and a second polypeptide, wherein the first polypeptide comprises the amino acid sequence set forth in SEQ ID NO.: 20 and the second polypeptide comprises the amino acid sequence set forth in SEQ ID NO.: 155. The first polypeptide can be or comprise a TCRα chain and/or the second polypeptide can be or comprise a TCRβ chain. In some embodiments, the first polypeptide is or comprises a TCRα chain and/or the second polypeptide is or comprises a TCRβ chain.

In some embodiments, a variable domain comprises an amino acid sequence with one or more insertions, deletions, and/or substitutions relative to any one of SEQ ID NOs: 13, 23, 39, and 49.

For example, the variable domain can comprise an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid insertions relative to any one of SEQ ID NOs: 13, 23, 39, and 49.

In some embodiments, the variable domain comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid insertions relative to any one of SEQ ID NOs: 13, 23, 39, and 49.

In some embodiments, the variable domain comprises 1, 2, 3, 4, 5, 6, 7, 8, 9, 10, 11, 12, 13, 14, 15, 16, 17, 18, 19, 20, 25, 30, 35, 40, 45, or 50 amino acid insertions relative to any one of SEQ ID NOs: 13, 23, 39, and 49.

The one or more insertions can be at the N-terminus, the C-terminus, within the amino acid sequence, or a combination thereof. The one or more insertions can be contiguous, non-contiguous, or a combination thereof.

In some embodiments, a variable domain comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid deletions relative to any one of SEQ ID NOs: 13, 23, 39, and 49.

In some embodiments, the variable domain comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid deletions relative to any one of SEQ ID NOs: 13, 23, 39, and 49.

In some embodiments, the variable domain comprises 1, 2, 3, 4, 5, 6, 7, 8, 9, 10, 11, 12, 13, 14, 15, 16, 17, 18, 19, 20, 25, 30, 35, 40, 45, or 50 amino acid deletions relative to any one of SEQ ID NOs: 13, 23, 39, and 49.

The one or more deletions can be at the N-terminus, the C-terminus, within the amino acid sequence, or a combination thereof. The one or more deletions can be contiguous, non-contiguous, or a combination thereof.

In some embodiments, a variable domain comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid substitutions relative to any one of SEQ ID NOs: 13, 23, 39, and 49.

In some embodiments, the variable domain comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid substitutions relative to any one of SEQ ID NOs: 13, 23, 39, and 49.

In some embodiments, the variable domain comprises 1, 2, 3, 4, 5, 6, 7, 8, 9, 10, 11, 12, 13, 14, 15, 16, 17, 18, 19, 20, 25, 30, 35, 40, 45, or 50 amino acid substitutions relative to any one of SEQ ID NOs: 13, 23, 39, and 49.

The one or more substitutions can be at the N-terminus, the C-terminus, within the amino acid sequence, or a combination thereof. The one or more substitutions can be contiguous, non-contiguous, or a combination thereof.

The binding protein can further comprise a TCR α chain constant domain (Cα) and/or a TCR β chain constant domain (Cβ). The TCR α chain constant domain (Cα) and/or a TCR β chain constant domain (Cβ) can be human. The TCR α chain constant domain (Cα) and/or a TCR β chain constant domain (Cβ) can be mammalian. The TCR α chain constant domain (Cα) and/or a TCR β chain constant domain (Cβ) can be an engineered variant of a mammalian (e.g. human) constant domain. In some embodiments, the Cα is an engineered variant of a human Cα and/or the Cβ is an engineered variant of a human Cβ. In some embodiments, the Cα is an engineered variant of a human Cα and the Cβ is an engineered variant of a human Cβ.

In some embodiments, the Cα comprises, consists essentially of, or consists of an amino acid sequence having at least 70%, at least 75%, at least 80%, at least 85%, at least 90%, at least 91%, at least 92%, at least 93%, at least 94%, at least 95%, at least 96%, at least 97%, at least 98%, or at least 99% identity to, or comprising or consisting of, the amino acid sequence set forth in any one of SEQ ID NOs.: 18, 19, 44, 45, and 69.

In some embodiments, the Cβ comprises, consists essentially of, or consists of an amino acid sequence having at least 70%, at least 75%, at least 80%, at least 85%, at least 90%, at least 91%, at least 92%, at least 93%, at least 94%, at least 95%, at least 96%, at least 97%, at least 98%, or at least 99% identity to, or comprising or consisting of, the amino acid sequence set forth in any one of SEQ ID NOs.: 28, 29, 54, 55, and 70-73.

In certain embodiments, the Cα and the Cβ comprise or consist of amino acid sequences having at least 70%, at least 75%, at least 80%, at least 85%, at least 90%, at least 91%, at least 92%, at least 93%, at least 94%, at least 95%, at least 96%, at least 97%, at least 98%, or at least 99% identity to, or comprising or consisting of, the amino acid sequences set forth in SEQ ID NOs.: (i) 18 and 28, respectively; (ii) 19 and 29, respectively; (iii) 44 and 54, respectively; or (iv) 45 and 55, respectively.

The binding protein can comprise (i) an extracellular domain of a TCR alpha chain, TCR beta chain, TCR gamma chain, or TCR delta chain; (ii) a transmembrane domain of a TCR alpha chain, TCR beta chain, TCR gamma chain, or TCR delta chain; and/or (iii) a cytoplasmic domain of a TCR alpha chain, TCR beta chain, TCR gamma chain, or TCR delta chain. The binding protein can comprise a full length or substantially full length TCR alpha chain, TCR beta chain, TCR gamma chain, and/or TCR delta chain.

In some embodiments, the binding protein comprises a TCR α chain and a TCR β chain, wherein the TCR α chain and the TCR β chain comprise or consist of amino acid sequences having at least 70%, at least 75%, at least 80%, at least 85%, at least 90%, at least 91%, at least 92%, at least 93%, at least 94%, at least 95%, at least 96%, at least 97%, at least 98%, or at least 99% identity to, or comprising or consisting of, the amino acid sequences set forth in: (i) SEQ ID NOs.: 12 and 22, respectively; (ii) SEQ ID NOs.: 20 and 30, respectively; (iii) SEQ ID NOS.: 12 and 30, respectively; (iv) SEQ ID NOs.: 20 and 22, respectively; (v) SEQ ID NOs.: 38 and 48, respectively; (vi) SEQ ID NOs.: 46 and 56, respectively; (vii) SEQ ID NOs.: 38 and 56, respectively; or (viii) SEQ ID NOs.: 46 and 48, respectively.

In some embodiments, a first polypeptide and a second polypeptide are provided, wherein (i) the first polypeptide comprises, consists essentially of, or consists of the amino acid sequence SEQ ID NO.: 83 and (ii) the second polypeptide comprises, consists essentially of, or consists of the amino acid sequence of SEQ ID NO.: 85, wherein the first polypeptide and the second polypeptide can associate to form a polypeptide dimer.

In some embodiments, the binding protein comprises an amino acid sequence with one or more insertions, deletions, and/or substitutions relative to any one of SEQ ID NOs: 12, 18-22, 28-30, 38, 44-46, 48, 54-56, and 69.

For example, the binding protein can comprise an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid insertions relative to any one of SEQ ID NOs: 12, 18-22, 28-30, 38, 44-46, 48, 54-56, and 69.

In some embodiments, the binding protein comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid insertions relative to any one of SEQ ID NOs: 12, 18-22, 28-30, 38, 44-46, 48, 54-56, and 69.

In some embodiments, the binding protein comprises 1, 2, 3, 4, 5, 6, 7, 8, 9, 10, 11, 12, 13, 14, 15, 16, 17, 18, 19, 20, 25, 30, 35, 40, 45, or 50 amino acid insertions relative to any one of SEQ ID NOs: 12, 18-22, 28-30, 38, 44-46, 48, 54-56, and 69.

The one or more insertions can be at the N-terminus, the C-terminus, within the amino acid sequence, or a combination thereof. The one or more insertions can be contiguous, non-contiguous, or a combination thereof.

In some embodiments, the binding protein comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid deletions relative to any one of SEQ ID NOs: 12, 18-22, 28-30, 38, 44-46, 48, 54-56, and 69.

In some embodiments, the binding protein comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid deletions relative to any one of SEQ ID NOs: 12, 18-22, 28-30, 38, 44-46, 48, 54-56, and 69.

In some embodiments, the binding protein comprises 1, 2, 3, 4, 5, 6, 7, 8, 9, 10, 11, 12, 13, 14, 15, 16, 17, 18, 19, 20, 25, 30, 35, 40, 45, or 50 amino acid deletions relative to any one of SEQ ID NOs: 12, 18-22, 28-30, 38, 44-46, 48, 54-56, and 69.

The one or more deletions can be at the N-terminus, the C-terminus, within the amino acid sequence, or a combination thereof. The one or more deletions can be contiguous, non-contiguous, or a combination thereof.

In some embodiments, the binding protein comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid substitutions relative to any one of SEQ ID NOs: 12, 18-22, 28-30, 38, 44-46, 48, 54-56, and 69.

In some embodiments, the binding protein comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid substitutions relative to any one of SEQ ID NOs: 12, 18-22, 28-30, 38, 44-46, 48, 54-56, and 69.

In some embodiments, the binding protein comprises 1, 2, 3, 4, 5, 6, 7, 8, 9, 10, 11, 12, 13, 14, 15, 16, 17, 18, 19, 20, 25, 30, 35, 40, 45, or 50 amino acid substitutions relative to any one of SEQ ID NOs: 12, 18-22, 28-30, 38, 44-46, 48, 54-56, and 69.

The one or more substitutions can be at the N-terminus, the C-terminus, within the amino acid sequence, or a combination thereof. The one or more substitutions can be contiguous, non-contiguous, or a combination thereof.

In any of the presently disclosed embodiments, a binding protein can comprise a TCR, a single-chain TCR (scTCR), a scTv, or a chimeric antigen receptor (CAR). Methods for producing engineered TCRs are described in, for example, Bowerman et al., Mol. Immunol., 46(15): 3000 (2009), the techniques of which are herein incorporated by reference. Methods for making CARs are known in the art and are described, for example, in U.S. Pat. Nos. 6,410,319; 7,446,191; U.S. Patent Publication No. 2010/065818; U.S. Pat. No. 8,822,647; PCT Publication No. WO 2014/031687; U.S. Pat. No. 7,514,537; and Brentjens et al., 2007, Clin. Cancer Res. 13:5426, the techniques of which are herein incorporated by reference. In some embodiments, a binding protein comprises a soluble TCR, optionally fused to a binding domain (e.g., a scFv) specific for a CD3 protein. See Elie Dolgin, Nature Biotechnology 40:441-449 (2022).

In any of the presently disclosed embodiments, a polynucleotide encoding a binding protein can further comprise: (i) a polynucleotide encoding a polypeptide that comprises an extracellular portion of a CD8 co-receptor α chain, wherein, optionally, the encoded polypeptide is or comprises a CD8 co-receptor α chain; (ii) a polynucleotide encoding a polypeptide that comprises an extracellular portion of a CD8 co-receptor β chain, wherein, optionally, the encoded polypeptide is or comprises a CD8 co-receptor β chain; or (iii) a polynucleotide of (i) and a polynucleotide of (ii). Without being bound by theory, in certain embodiments, co-expression or concurrent expression of a binding protein and a CD8 co-receptor protein or portion thereof functional to bind to an HLA molecule may improve one or more desired activity of a host cell (e.g., immune cell, such as a T cell, optionally a CD4⁺ T cell) as compared to expression of the binding protein alone. It will be understood that the binding protein-encoding polynucleotide and the CD8 co-receptor polypeptide-encoding polynucleotide may be present on a single nucleic acid molecule (e.g., in a same expression vector), or may be present on separate nucleic acid molecules in a host cell.

In any of the presently disclosed embodiments, a CD8 co-receptor alpha chain can comprise, consist essentially of, or consist of SEQ ID NO.: 87, or SEQ ID NO.: 87 with the signal peptide removed. An example of a polynucleotide encoding SEQ ID NO.: 87 is provided in SEQ ID NO.: 88. In some embodiments, a CD8 co-receptor alpha chain comprises, consists essentially of, or consists of an amino acid sequence with at least 70%, at least 75%, at least 80%, at least 85%, at least 90%, at least 95%, at least 97%, or at least 99% sequence identity to the amino acid sequence of SEQ ID NO.: 87, or SEQ ID NO.: 87 with the signal peptide removed.

In any of the presently disclosed embodiments, a CD8 co-receptor beta chain can comprise, consist essentially of, or consist of SEQ ID NO.: 89, or SEQ ID NO.: 89 with the signal peptide removed. An example of a polynucleotide encoding SEQ ID NO.: 89 is provided in SEQ ID NO.: 90. In some embodiments, a CD8 co-receptor beta chain comprises, consists essentially of, or consists of an amino acid sequence with at least 70%, at least 75%, at least 80%, at least 85%, at least 90%, at least 95%, at least 97%, or at least 99% sequence identity to the amino acid sequence of SEQ ID NO.: 89, or SEQ ID NO.: 89 with the signal peptide removed.

In certain further embodiments, a polynucleotide comprises: (a) the polynucleotide encoding a polypeptide comprising an extracellular portion of a CD8 co-receptor α chain; (b) the polynucleotide encoding a polypeptide comprising an extracellular portion of a CD8 co-receptor β chain; and (c) a polynucleotide encoding a self-cleaving peptide disposed between the polynucleotide of (a) and the polynucleotide of (b). In further embodiments, a polynucleotide comprises a polynucleotide that encodes a self-cleaving peptide and is disposed between: (1) the polynucleotide encoding a binding protein and the polynucleotide encoding a polypeptide comprising an extracellular portion of a CD8 co-receptor α chain; and/or (2) the polynucleotide encoding a binding protein and the polynucleotide encoding a polypeptide comprising an extracellular portion of a CD8 co-receptor β chain.

In still further embodiments, a polynucleotide can comprise, operably linked in-frame: (i) (pnCD8α)-(pnSCP1)-(pnCD8β)-(pnSCP2)-(pnBP); (ii) (pnCD8β)-(pnSCP1)-(pnCD8α)-(pnSCP2)-(pnBP); (iii) (pnBP)-(pnSCP1)-(pnCD8α)-(pnSCP2)-(pnCD8β); (iv) (pnBP)-(pnSCP1)-(pnCD8β)-(pnSCP2)-(pnCD8α); (v) (pnCD8α)-(pnSCP1)-(pnBP)-(pnSCP2)-(pnCD8β); or (vi) (pnCD8β)-(pnSCP1)-(pnBP)-(pnSCP2)-(pnCD8α), wherein pnCD8α is the polynucleotide encoding a polypeptide that comprises an extracellular portion of a CD8 co-receptor α chain, wherein pnCD8β is the polynucleotide encoding a polypeptide that comprises an extracellular portion of a CD8 co-receptor α chain, wherein pnBP is the polynucleotide encoding a binding protein, and wherein pnSCP1 and pnSCP2 are each independently a polynucleotide encoding a self-cleaving peptide, wherein the polynucleotides and/or the encoded self-cleaving peptides are optionally the same or different (e.g., P2A, T2A, F2A, E2A). It will be understood that self-cleaving peptide can comprise a linker N-terminal and/or C-terminal thereto. An example of a linker is GSG. In some embodiments, a T2A peptide is provided that comprises a N-terminal GSG linker. In some embodiments, the GSG-T2A sequence comprises, consists essentially of, or consists of SEQ ID NO.: 82. In some embodiments, a GSG-P2A sequence comprises, consists essentially of, or consists of SEQ ID NO.: 74.

In certain embodiments, the encoded binding protein comprises a TCRα chain and a TCRβ chain, wherein the polynucleotide comprises a polynucleotide encoding a self-cleaving peptide disposed between the polynucleotide encoding a TCRα chain and the polynucleotide encoding a TCRβ chain. In further embodiments, the polynucleotide comprises, operably linked in-frame: (i) (pnCD8α)-(pnSCP1)-(pnCD8β)-(pnSCP2)-(pnTCRβ)-(pnSCP3)-(pnTCRα); (ii) (pnCD8β)-(pnSCP1)-(pnCD8α)-(pnSCP2)-(pnTCRβ)-(pnSCP3)-(pnTCRα); (iii) (pnCD8α)-(pnSCP1)-(pnCD8β)-(pnSCP2)-(pnTCRα)-(pnSCP3)-(pnTCRβ); (iv) (pnCD8β)-(pnSCP1)-(pnCD8α)-(pnSCP2)-(pnTCRα)-(pnSCP3)-(pnTCRβ); (v) (pnTCRβ)-(pnSCP1)-(pnTCRα)-(pnSCP2)-(pnCD8α)-(pnSCP3)-(pnCD8β); (vi) (pnTCRβ)-(pnSCP1)-(pnTCRα)-(pnSCP2)-(pnCD8β)-(pnSCP3)-(pnCD8α); (vii) (pnTCRα)-(pnSCP1)-(pnTCRβ)-(pnSCP2)-(pnCD8α)-(pnSCP3)-(pnCD8β); (viii) (pnTCRα)-(pnSCP1)-(pnTCRβ)-(pnSCP2)-(pnCD8β)-(pnSCP3)-(pnCD8α), wherein pnCD8α is the polynucleotide encoding a polypeptide that comprises an extracellular portion of a CD8 co-receptor α chain, wherein pnCD8β is the polynucleotide encoding a polypeptide that comprises an extracellular portion of a CD8 co-receptor α chain, wherein pnTCRα is the polynucleotide encoding a TCR α chain, wherein pnTCRβ is the polynucleotide encoding a TCR β chain, and wherein pnSCP1, pnSCP2, and pnSCP3 are each independently a polynucleotide encoding a self-cleaving peptide, wherein the polynucleotides and/or the encoded self-cleaving peptides are optionally the same or different. In some embodiments, SCP1 comprises SEQ ID NO.: 82, SCP2 comprises SEQ ID NO.: 74, and SCP3 comprises SEQ ID NO.: 74.

Additionally or alternatively, a polynucleotide encoding a binding protein can encode a furin cleavage site or other protease cleavage site disposed between two other polypeptides (e.g., between a TCRβ chain and a TCRα chain.

In certain embodiments, an encoded polypeptide of the present disclosure comprises one or more junction amino acids. “Junction amino acids” or “junction amino acid residues” refer to one or more (e.g., 2 to about 10) amino acid residues between two adjacent motifs, regions, or domains of a polypeptide, such as between a binding domain and an adjacent constant domain or between a TCR chain and an adjacent self-cleaving peptide. Junction amino acids can result from the design of a construct that encodes a fusion protein (e.g., amino acid residues resulting from the use of a restriction enzyme site during the construction of a nucleic acid molecule encoding a fusion protein), or from cleavage of, for example, a self-cleaving peptide adjacent one or more domains of an encoded binding protein of this disclosure (e.g., a P2A peptide disposed between a TCR α-chain and a TCR β-chain, the self-cleavage of which can leave one or more junction amino acids in the α-chain, the TCR β-chain, or both).

In further embodiments, a binding protein is expressed as part of a transgene construct that encodes, and/or a host cell of the present disclosure can encode: one or more additional accessory protein, such as a safety switch protein; a tag, a selection marker; a CD8 co-receptor β-chain; a CD8 co-receptor α-chain or both; or any combination thereof. Polynucleotides and transgene constructs useful for encoding and expressing binding proteins and accessory components (e.g., one or more of a safety switch protein, a selection marker, CD8 co-receptor β-chain, or a CD8 co-receptor α-chain) are described in PCT application PCT/US2017/053112, the polynucleotides, transgene constructs, and accessory components, including the nucleotide and amino acid sequences, of which are hereby incorporated by reference. It will be understood that any or all of a binding protein of the present disclosure, a safety switch protein, a tag, a selection marker, a CD8 co-receptor β-chain, or a CD8 co-receptor α-chain may be encoded by a single nucleic acid molecule or may be encoded by polynucleotide sequences that are, or are present on, separate nucleic acid molecules.

Exemplary safety switch proteins include, for example, a truncated EGF receptor polypeptide (huEGFRt) that is devoid of extracellular N-terminal ligand binding domains and intracellular receptor tyrosine kinase activity, but that retains its native amino acid sequence, has type I transmembrane cell surface localization, and has a conformationally intact binding epitope for pharmaceutical-grade anti-EGFR monoclonal antibody, cetuximab (Erbitux) tEGF receptor (tEGFr; Wang et al., Blood 118:1255-1263, 2011); a caspase polypeptide (e.g., iCasp9; Straathof et al., Blood 105:4247-4254, 2005; Di Stasi et al., N. Engl. J. Med. 365:1673-1683, 2011; Zhou and Brenner, Exp. Hematol. pii: S0301-472X (16) 30513-6. doi: 10.1016/j.exphem.2016.07.011), RQR8 (Philip et al., Blood 124:1277-1287, 2014); a 10-amino-acid tag derived from the human c-myc protein (Myc) (Kieback et al., Proc. Natl. Acad. Sci. USA 105:623-628, 2008); and a marker/safety switch polypeptide, such as RQR (CD20+CD34; Philip et al., 2014).

Other accessory components useful for modified host cells of the present disclosure comprise a tag or selection marker that allows the cells to be identified, sorted, isolated, enriched, or tracked. For example, marked host cells having desired characteristics (e.g., an antigen-specific TCR and a safety switch protein) can be sorted away from unmarked cells in a sample and more efficiently activated and expanded for inclusion in a product of desired purity.

As used herein, the term “selection marker” comprises a nucleic acid construct (and the encoded gene product) that confers an identifiable change to a cell permitting detection and positive selection of immune cells transduced with a polynucleotide comprising a selection marker. RQR is a selection marker that comprises a major extracellular loop of CD20 and two minimal CD34 binding sites. In some embodiments, an RQR-encoding polynucleotide comprises a polynucleotide that encodes the 16-amino-acid CD34 minimal epitope. In some embodiments, the CD34 minimal epitope is incorporated at the amino terminal position of a CD8 co-receptor stalk domain (Q8). In further embodiments, the CD34 minimal binding site sequence can be combined with a target epitope for CD20 to form a compact marker/suicide gene for T cells (RQR8) (Philip et al., 2014, incorporated by reference herein). This construct allows for the selection of host cells expressing the construct, with for example, CD34 specific antibody bound to magnetic beads (Miltenyi) and that utilizes clinically accepted pharmaceutical antibody, rituximab, that allows for the selective deletion of a transgene expressing engineered T cell (Philip et al., 2014).

Further exemplary selection markers also include several truncated type I transmembrane proteins normally not expressed on T cells: the truncated low-affinity nerve growth factor, truncated CD19, and truncated CD34 (see for example, Di Stasi et al., N. Engl. J. Med. 365:1673-1683, 2011; Mavilio et al., Blood 83:1988-1997, 1994; Fehse et al., Mol. Ther. 1:448-456, 2000; each incorporated herein in their entirety). A useful feature of CD19 and CD34 is the availability of the off-the-shelf Miltenyi CliniMACs™ selection system that can target these markers for clinical-grade sorting. However, CD19 and CD34 are relatively large surface proteins that may tax the vector packaging capacity and transcriptional efficiency of an integrating vector. Surface markers containing the extracellular, non-signaling domains or various proteins (e.g., CD19, CD34, LNGFR) also can be employed. Any selection marker may be employed and should be acceptable for Good Manufacturing Practices. In certain embodiments, selection markers are expressed with a polynucleotide that encodes a gene product of interest (e.g., a binding protein of the present disclosure, such as a TCR or CAR). Further examples of selection markers include, for example, reporters such as GFP, EGFP, β-gal or chloramphenicol acetyltransferase (CAT). In certain embodiments, a selection marker, such as, for example, CD34 is expressed by a cell and the CD34 can be used to select enrich for, or isolate (e.g., by immunomagnetic selection) the transduced cells of interest for use in the methods described herein. As used herein, a CD34 marker is distinguished from an anti-CD34 antibody, or, for example, a scFv, TCR, or another antigen recognition moiety that binds to CD34.

In certain embodiments, a selection marker comprises an RQR polypeptide, a truncated low-affinity nerve growth factor (tNGFR), a truncated CD19 (tCD19), a truncated CD34 (tCD34), or any combination thereof.

Regarding RQR polypeptides, without wishing to be bound by theory, it is believed that distance from the host cell surface is important for RQR polypeptides to function as selection markers/safety switches (Philip et al., 2010 (supra)). In some embodiments, the encoded RQR polypeptide is contained in a β-chain, an α-chain, or both, or a fragment or variant of either or both, of the encoded CD8 co-receptor. In specific embodiments, a modified host cell comprises a heterologous polynucleotide encoding iCasp9 and a heterologous polynucleotide encoding a recombinant CD8 co-receptor protein that comprises a β-chain containing a RQR polypeptide and further comprises a CD8 α-chain.

An encoded CD8 co-receptor includes, in some embodiments, an α-chain or a fragment or variant thereof. An amino acid sequence of the human CD8 co-receptor α-chain precursor is known and is provided at, for example, UniProtKB-P30433 (see also UniProtKB-P31783; -P10732; and -P10731). An encoded CD8 co-receptor includes, in some embodiments, a β-chain or a fragment or variant thereof. An amino acid sequence of the human CD8 co-receptor β-chain precursor is known and is provided at, for example, UniProtKB-P10966 (see also UniProtKB-Q9UQ56; -E9PD41; Q8TD28; and -P30434; and -P05541) .

An isolated polynucleotide of this disclosure may further comprise a polynucleotide encoding a safety switch protein, a selection marker, a CD8 co-receptor beta chain, or a CD8 co-receptor alpha chain as disclosed herein, or may comprise a polynucleotide encoding any combination thereof.

In any of the presently disclosed embodiments, a polynucleotide can be codon optimized for expression in a host cell. In some embodiments, the host cell comprises a human immune system cell, such as a T cell, a NK cell, or a NK-T cell (Scholten et al., Clin. Immunol. 119:135, 2006). Codon optimization can be performed using known techniques and tools, e.g., using the GenScript® OptimumGene™ tool, or GeneArt (Life Technologies). Codon-optimized sequences include sequences that are partially codon-optimized (i.e., one or more of the codons is optimized for expression in the host cell) and those that are fully codon-optimized. It will be appreciated that in embodiments wherein a polynucleotide encodes more than one polypeptide (e.g., a TCR α chain, a TCR β chain, a CD8 co-receptor α chain, a CD8 co-receptor β chain, and one or more self-cleaving peptides), each polypeptide can independently fully codon optimized, partially codon optimized, or not codon optimized.

Amino acid and polynucleotide sequences for exemplary binding proteins “11N4A” and “11N6” are shown in Table 1.

TABLE 1
Certain Polynucleotide and Amino Acid Sequences
related to TCRs 11N4A and 11N6

| | TCR | |
|---|---|---|
| | 11N4A | 11N6 |
| Polynucleotide Sequences | | |
| TCR α-chain with signal peptide, original | 5 | 33 |
| polynucleotide | | |
| TCR α-chain with signal peptide, cys-modified | 86 | |
| TCR β-chain with signal peptide, original | 6 | 34 |
| polynucleotide | | |
| TCR β-chain with signal peptide, cys-modified | 84 | |
| TCRβ-P2A-TCRα, codon-optimization (A) | 7 | 35 |
| TCRβ-P2A-TCRα, codon-optimization (B) | 8 | — |
| CD8α-T2A-CD8β-P2A-TCRβ-P2A-TCRα, codon- | 9 | 36 |
| optimization (A) | | |
| CD8α-T2A-CD8β-P2A-TCRβ-P2A-TCRα, codon- | 10 | — |
| optimization (B) | | |
| Amino acid Sequences | | |
| TCR α-chain with signal peptide, original | 11 | 37 |
| TCR α-chain without signal peptide, original | 12 | 38 |
| TCR α-chain variable domain, without signal peptide | 13 | 39 |
| TCR α-chain variable domain, CDR1α | 14 | 40 |
| TCR α-chain variable domain, CDR2α | 15 | 41 |
| TCR α-chain variable domain, CDR3α-IMGT junction | 16 | 42 |
| TCR α-chain variable domain, CDR3α-IMGT | 17 | 43 |
| TCR α-chain constant domain, original | 18 | 44 |
| TCR α-chain constant domain, cys-modified | 19 | 45 |
| TCR α-chain without signal peptide, cys-modified | 20 | 46 |
| TCR α-chain with signal peptide, cys-modified | 85 | |
| TCR β-chain with signal peptide, original | 21 | 47 |
| TCR β-chain without signal peptide, original | 22 | 48 |
| TCR β-chain variable domain, without signal peptide | 23 | 49 |
| TCR β-chain variable domain, CDR1β | 24 | 50 |
| TCR β-chain variable domain, CDR2β | 25 | 51 |
| TCR β-chain variable domain, CDR3β-IMGT junction | 26 | 52 |
| TCR β-chain variable domain, CDR3β-IMGT | 27 | 53 |
| TCR β-chain constant domain, original | 28 | 54 |
| TCR β-chain constant domain, cys-modified | 29 | 55 |
| TCR β-chain without signal peptide, cys-modified | 30 | 56 |
| TCR β-chain with signal peptide, cys-modified | 83 | |
| TCRβ-P2A-TCRα | 31 | 57 |
| CD8α-T2A-CD8β-P2A-TCRβ-P2A-TCRα | 32 | 58 |

Also provided is a polynucleotide comprising (i) an expression control sequence operably linked to (ii) a sequence encoding the amino acid sequence set forth in any one of SEQ ID NOs.: 17, 27, 16, 26, 53, 43, 52, and 42. The expression control sequence can be heterologous to the sequence of (ii). The sequence of (ii) can be codon-optimized, e.g. for expression in a human T cell.

### Vectors

In another aspect, the present disclosure provides an expression vector, comprising any polynucleotide as provided herein operably linked to an expression control sequence.

Also provided herein are vectors that comprise a polynucleotide or transgene construct of the instant disclosure. Some examples of vectors include plasmids, viral vectors, cosmids, and others. Some vectors may be capable of autonomous replication in a host cell into which they are introduced (e.g., bacterial vectors having a bacterial origin of replication and episomal mammalian vectors), whereas other vectors may be integrated into the genome of a host cell or promote integration of the polynucleotide insert upon introduction into the host cell and thereby replicate along with the host genome (e.g., lentiviral vector, retroviral vector). Additionally, some vectors are capable of directing the expression of genes to which they are operatively linked (these vectors may be referred to as “expression vectors”). According to related embodiments, it is further understood that, if one or more agents (e.g., polynucleotides encoding polypeptides as described herein) are co administered to a subject, that each agent may reside in separate or the same vectors, and multiple vectors (each containing a different agent or the same agent) may be introduced to a cell or cell population or administered to a subject.

In certain embodiments, polynucleotides of the present disclosure may be operatively linked to certain elements of a vector. For example, polynucleotide sequences that are needed to effect the expression and processing of coding sequences to which they are ligated may be operatively linked. Expression control sequences may include appropriate transcription initiation, termination, promoter, and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion. Expression control sequences may be operatively linked if they are contiguous with the gene of interest and expression control sequences that act in trans or at a distance to control the gene of interest.

In certain embodiments, the vector comprises a plasmid vector or a viral vector (e.g., a vector selected from lentiviral vector or a γ-retroviral vector). Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomega-lovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, and spumavirus (Coffin, J. M., Retroviridae: The viruses and their replication, In Fundamental Virology, Third Edition, B. N. Fields et al., Eds., Lippincott-Raven Publishers, Philadelphia, 1996).

“Retroviruses” are viruses having an RNA genome, which is reverse-transcribed into DNA using a reverse transcriptase enzyme, the reverse-transcribed DNA is then incorporated into the host cell genome. “Gammaretrovirus” refers to a genus of the retroviridae family. Examples of gammaretroviruses include mouse stem cell virus, murine leukemia virus, feline leukemia virus, feline sarcoma virus, and avian reticuloendotheliosis viruses. “Lentiviral vector,” as used herein, means HIV-based lentiviral vectors for gene delivery, which can be integrative or non-integrative, have relatively large packaging capacity, and can transduce a range of different cell types. Lentiviral vectors are usually generated following transient transfection of three (packaging, envelope, and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells. In some embodiments, a lentiviral vector is a self-inactivating lentiviral vector. A self-inactivating lentiviral vector can comprise a modification to prevent the transfer of enhancer and promoter elements in the 5′ long terminal repeat (LTR) of the vector to transduced cells, for example, comprising a deletion in the 3′LTR of the viral genome that is transferred into the 5′LTR after one round of reverse transcription, resulting in a provirus that contains no LTR derived enhancer or promoter elements. In some embodiments, a lentiviral vector is a third generation lentiviral vector. A third generation lentiviral vector can utilize a packaging system split into two or more plasmids, e.g., one encoding Rev and one encoding Gag and Pol. A third generation lentiviral vector can utilize a packaging system that lacks Tat or does not require Tat expression, and instead comprises, e.g., a chimeric 5′ LTR fused to a heterologous promoter on the transfer plasmid.

In certain embodiments, the viral vector can be a gammaretrovirus, e.g., Moloney murine leukemia virus (MLV)-derived vectors. In other embodiments, the viral vector can be a more complex retrovirus-derived vector, e.g., a lentivirus-derived vector. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host cells with viral particles containing TCR or CAR transgenes are known in the art and have been previous described, for example, in: U.S. Pat. No. 8,119,772; Walchli et al., PLOS One 6:327930, 2011; Zhao et al., J. Immunol. 174:4415, 2005; Engels et al., Hum. Gene Ther. 14:1155, 2003; Frecha et al., Mol. Ther. 18:1748, 2010; and Verhoeyen et al., Methods Mol. Biol. 506:97, 2009. Retroviral and lentiviral vector constructs and expression systems are also commercially available. Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5:1517, 1998).

Other vectors developed for gene therapy uses can also be used with the compositions and methods of this disclosure. Such vectors include those derived from baculoviruses and α-viruses. (Jolly, D J. 1999. Emerging Viral Vectors. pp 209-40 in Friedmann T. ed. The Development of Human Gene Therapy. New York: Cold Spring Harbor Lab), or plasmid vectors (such as Sleeping Beauty or other transposon vectors).

When a viral vector genome comprises a plurality of polynucleotides to be expressed in a host cell as separate transcripts, the viral vector may also comprise additional sequences between the two (or more) transcripts allowing for bicistronic or multicistronic expression. Examples of such sequences used in viral vectors include internal ribosome entry sites (IRES), furin cleavage sites, viral 2A peptide, or any combination thereof.

In certain embodiments, a vector is capable of delivering the polynucleotide or transgene construct to a host cell (e.g., a hematopoietic progenitor cell or a human immune system cell). In specific embodiments, a vector is capable of delivering a polynucleotide or transgene construct to human immune system cell, such as, for example, a CD4⁺ T cell, a CD8⁺ T cell, a CD4⁻ CD8⁻ double negative T cell, a stem cell memory T cell, a γδ T cell, a natural killer cell, a dendritic cell, or any combination thereof. In further embodiments, a vector is capable of delivering a transgene construct to a naïve T cell, a central memory T cell, an effector memory T cell, or any combination thereof. In some embodiments, a vector that encodes a polynucleotide or transgene construct of the present disclosure may further comprise a polynucleotide that encodes a nuclease that can be used to perform a chromosomal knockout in a host cell (e.g., a CRISPR-Cas endonuclease or another endonuclease as disclosed herein) or that can be used to deliver a therapeutic polynucleotide or transgene or portion thereof to a host cell in a gene therapy replacement or gene repair therapy. Alternatively, a nuclease used for a chromosomal knockout or a gene replacement or gene repair therapy can be delivered to a host cell independent of a vector that encodes a polynucleotide or transgene construct of this disclosure.

In certain embodiments, the vector is capable of delivering the polynucleotide to a host cell. In further embodiments, the host cell is a hematopoietic progenitor cell or a human immune system cell. In still further embodiments, the human immune system cell is a CD4+ T cell, a CD8+ T cell, a CD4−CD8− double negative T cell, a γδ T cell, a natural killer cell, a natural killer T cell, a macrophage, a monocyte, a dendritic cell, or any combination thereof. In yet further embodiments, the T cell is a naïve T cell, a central memory T cell, an effector memory T cell, or any combination thereof.

In any of the presently disclosed embodiments, the vector is a viral vector. In certain embodiments, the viral vector is a lentiviral vector or a γ-retroviral vector.

Examples of transposon-based systems that can be used include, but are not limited to, sleeping beauty (e.g., derived from the genome of salmonid fish); piggyback (e.g., derived from lepidopteran cells and/or the Myotis lucifugus); mariner (e.g., derived from Drosophila); frog prince (e.g., derived from Rana pipiens); Tol2 (e.g., derived from medaka fish); and spinON.

### Host Cells

Also provided herein are host cells that encode and/or express a binding protein (and, optionally, one or more accessory protein, such as a transduction marker, a CD8 co-receptor polypeptide, or the like, as provided herein). In certain embodiments, a host cell is provided that is modified to comprise a polynucleotide and/or an expression vector of the present disclosure, and/or to express a binding protein of the present disclosure.

Any suitable host cell may be modified to include a heterologous polynucleotide encoding a binding protein of this disclosure, including, for example, an immune cell, such as T cell, a NK cell, or a NK-T cell modified to include the heterologous polynucleotide. In some embodiments, a modified immune cell comprises a CD4⁺ T cell, a CD8⁺ T cell, or both. Methods for transfecting/transducing T cells with desired nucleic acids have been described (e.g., U.S. Patent Application Pub. No. US 2004/0087025) as have adoptive transfer procedures using T cells of desired target-specificity (e.g., Schmitt et al., Hum. Gen. 20:1240, 2009; Dossett et al., Mol. Ther. 17:742, 2009; Till et al., Blood 112:2261, 2008; Wang et al., Hum. Gene Ther. 18:712, 2007; Kuball et al., Blood 109:2331, 2007; US 2011/0243972; US 2011/0189141; Leen et al., Ann. Rev. Immunol. 25:243, 2007), such that adaptation of these methodologies to the presently disclosed embodiments is contemplated, based on the teachings herein.

Any appropriate method can be used to transfect or transduce the cells, for example, the T cells, or to administer the polynucleotides or compositions of the present methods. Known methods for delivering polynucleotides to host cells include, for example, use of cationic polymers, lipid-like molecules, and certain commercial products such as, for example, IN-VIVO-JET PEI. Other methods include ex vivo transduction, injection, electroporation, DEAE-dextran, sonication loading, liposome-mediated transfection, receptor-mediated transduction, microprojectile bombardment, transposon-mediated transfer, and the like. Still further methods of transfecting or transducing host cells employ vectors, described in further detail herein.

In certain embodiments, the host cell or modified cell comprises a hematopoietic progenitor cell, stem cell (e.g., iPSC), and/or or human immune cell. In some embodiments, the immune cell comprises a T cell, a NK cell, a NK-T cell, a dendritic cell, a macrophage, a monocyte, or any combination thereof. In further embodiments, the immune cell comprises a CD4+ T cell, a CD8+ T cell, a CD4-CD8-double negative T cell, a γδ T cell, or any combination thereof. In certain further embodiments, the immune cell comprises a CD4+ T cell and a CD8+ T cell. In certain still further embodiments, the CD4+ T cell, the CD8+ T cell, or both comprise (i) a polynucleotide encoding a polypeptide that comprises an extracellular portion of a CD8 co-receptor α chain, wherein, optionally, the encoded polypeptide is or comprises a CD8 co-receptor α chain; (ii) a polynucleotide encoding a polypeptide that comprises an extracellular portion of a CD8 co-receptor β chain, wherein, optionally, the encoded polypeptide is or comprises a CD8 co-receptor β chain; or (iii) a polynucleotide of (i) and a polynucleotide of (ii).

A host cell can be a peripheral blood mononuclear cell (PBMC). A host cell can be a lymphoid cell. A host cell can be a lymphocyte. A host cell can be a T cell. A host cell can be an alpha beta T cell (whether expressing or not expressing an endogenous alpha-beta TCR). A host cell can be a gamma delta T cell (whether expressing or not expressing an endogenous gamma-delta TCR). A host cell can be a B cell. A host cell can be a natural killer (NK) cell. A host cell can be a Natural Killer T (NKT) cell. A host cell can be a mammalian cell. A host cell can be a human cell.

A host cell can be a primary cell. A host cell can be an immortalized cell. A host cell can be of a cell line. A host cell can be differentiated from a stem cell, for example, an induced pluripotent stem cell (iPSC), embryonic stem cell, hematopoietic stem cell (HSC), or the like.

In any of the foregoing embodiments, a host cell (e.g., an immune cell) may modified to reduce or eliminate expression of one or more endogenous genes that encode a polypeptide involved in immune signaling or other related activities. Exemplary gene knockouts include those that encode PD-1, LAG-3, CTLA4, TIM3, TIGIT, FasL, an HLA molecule, a TCR molecule, or the like. Without wishing to be bound by theory, certain endogenously expressed immune cell proteins may be recognized as foreign by an allogeneic host receiving the modified immune cells, which may result in elimination of the modified immune cells (e.g., an HLA allele), or may downregulate the immune activity of the modified immune cells (e.g., PD-1, LAG-3, CTLA4, FasL, TIGIT, TIM3), or may interfere with the binding activity of a heterologously expressed binding protein of the present disclosure (e.g., an endogenous TCR of a modified T cell that binds a non-Ras antigen and thereby interferes with the modified immune cell binding a cell that expresses a Ras antigen).

Accordingly, decreasing or eliminating expression or activity of such endogenous genes or proteins can improve the activity, tolerance, or persistence of the modified cells in an autologous or allogeneic host setting, and may allow for universal administration of the cells (e.g., to any recipient regardless of HLA type). In certain embodiments, a modified cell is a donor cell (e.g., allogeneic) or an autologous cell. In certain embodiments, a modified cell of this disclosure comprises a chromosomal gene knockout of one or more of a gene that encodes PD-1, LAG-3, CTLA4, TIM3, TIGIT, FasL, an HLA component (e.g., a gene that encodes an α1 macroglobulin, an α2 macroglobulin, an α3 macroglobulin, a β1 microglobulin, or a β2 microglobulin), or a TCR component (e.g., a gene that encodes a TCR variable region or a TCR constant region) (see, e.g., Torikai et al., Nature Sci. Rep. 6:21757 (2016); Torikai et al., Blood 119(24): 5697 (2012); and Torikai et al., Blood 122(8): 1341 (2013), the gene-editing techniques, compositions, and adoptive cell therapies of which are herein incorporated by reference in their entirety).

As used herein, the term “chromosomal gene knockout” refers to a genetic alteration or introduced inhibitory agent in a host cell that prevents (e.g., reduces, delays, suppresses, or abrogates) production, by the host cell, of a functionally active endogenous polypeptide product. Alterations resulting in a chromosomal gene knockout can include, for example, introduced nonsense mutations (including the formation of premature stop codons), missense mutations, gene deletion, and strand breaks, as well as the heterologous expression of inhibitory nucleic acid molecules that inhibit endogenous gene expression in the host cell.

In certain embodiments, a chromosomal gene knock-out or gene knock-in is made by chromosomal editing of a host cell. Chromosomal editing can be performed using, for example, endonucleases. As used herein “endonuclease” refers to an enzyme capable of catalyzing cleavage of a phosphodiester bond within a polynucleotide chain. In certain embodiments, an endonuclease is capable of cleaving a targeted gene thereby inactivating or “knocking out” the targeted gene. An endonuclease may be a naturally occurring, recombinant, genetically modified, or fusion endonuclease. The nucleic acid strand breaks caused by the endonuclease are commonly repaired through the distinct mechanisms of homologous recombination or non-homologous end joining (NHEJ). During homologous recombination, a donor nucleic acid molecule may be used for a donor gene “knock-in”, for target gene “knock-out”, and optionally to inactivate a target gene through a donor gene knock in or target gene knock out event. NHEJ is an error-prone repair process that often results in changes to the DNA sequence at the site of the cleavage, e.g., a substitution, deletion, or addition of at least one nucleotide. NHEJ may be used to “knock-out” a target gene. Examples of endonucleases include zinc finger nucleases, TALE-nucleases, CRISPR-Cas nucleases, meganucleases, and megaTALs.

As used herein, a “zinc finger nuclease” (ZFN) refers to a fusion protein comprising a zinc finger DNA-binding domain fused to a non-specific DNA cleavage domain, such as a Fok1 endonuclease. Each zinc finger motif of about 30 amino acids binds to about 3 base pairs of DNA, and amino acids at certain residues can be changed to alter triplet sequence specificity (see, e.g., Desjarlais et al., Proc. Natl. Acad. Sci. 90:2256-2260, 1993; Wolfe et al., J. Mol. Biol. 285:1917-1934, 1999). Multiple zinc finger motifs can be linked in tandem to create binding specificity to desired DNA sequences, such as regions having a length ranging from about 9 to about 18 base pairs. By way of background, ZFNs mediate genome editing by catalyzing the formation of a site-specific DNA double strand break (DSB) in the genome, and targeted integration of a transgene comprising flanking sequences homologous to the genome at the site of DSB is facilitated by homology directed repair. Alternatively, a DSB generated by a ZFN can result in knock out of target gene via repair by non-homologous end joining (NHEJ), which is an error-prone cellular repair pathway that results in the insertion or deletion of nucleotides at the cleavage site. In certain embodiments, a gene knockout comprises an insertion, a deletion, a mutation or a combination thereof, made using a ZFN molecule.

As used herein, a “transcription activator-like effector nuclease” (TALEN) refers to a fusion protein comprising a TALE DNA-binding domain and a DNA cleavage domain, such as a FokI endonuclease. A “TALE DNA binding domain” or “TALE” is composed of one or more TALE repeat domains/units, each generally having a highly conserved 33-35 amino acid sequence with divergent 12th and 13th amino acids. The TALE repeat domains are involved in binding of the TALE to a target DNA sequence. The divergent amino acid residues, referred to as the Repeat Variable Diresidue (RVD), correlate with specific nucleotide recognition. The natural (canonical) code for DNA recognition of these TALEs has been determined such that an HD (histine-aspartic acid) sequence at positions 12 and 13 of the TALE leads to the TALE binding to cytosine (C), NG (asparagine-glycine) binds to a T nucleotide, NI (asparagine-isoleucine) to A, NN (asparagine-asparagine) binds to a G or A nucleotide, and NG (asparagine-glycine) binds to a T nucleotide. Non-canonical (atypical) RVDs are also known (see, e.g., U.S. Patent Publication No. US 2011/0301073, which atypical RVDs are incorporated by reference herein in their entirety). TALENs can be used to direct site-specific double-strand breaks (DSB) in the genome of T cells. Non-homologous end joining (NHEJ) ligates DNA from both sides of a double-strand break in which there is little or no sequence overlap for annealing, thereby introducing errors that knock out gene expression. Alternatively, homology directed repair can introduce a transgene at the site of DSB providing homologous flanking sequences are present in the transgene. In certain embodiments, a gene knockout comprises an insertion, a deletion, a mutation or a combination thereof, and made using a TALEN molecule.

As used herein, a “clustered regularly interspaced short palindromic repeats/Cas” (CRISPR/Cas) nuclease system refers to a system that employs a CRISPR RNA (crRNA)-guided Cas nuclease to recognize target sites within a genome (known as protospacers) via base-pairing complementarity and then to cleave the DNA if a short, conserved protospacer associated motif (PAM) immediately follows 3′ of the complementary target sequence. CRISPR/Cas systems are classified into three types (i.e., type I, type II, and type III) based on the sequence and structure of the Cas nucleases. The crRNA-guided surveillance complexes in types I and III need multiple Cas subunits. Type II system, the most studied, comprises at least three components: an RNA-guided Cas9 nuclease, a crRNA, and a trans-acting crRNA (tracrRNA). The tracrRNA comprises a duplex forming region. A crRNA and a tracrRNA form a duplex that is capable of interacting with a Cas9 nuclease and guiding the Cas9/crRNA: tracrRNA complex to a specific site on the target DNA via Watson-Crick base-pairing between the spacer on the crRNA and the protospacer on the target DNA upstream from a PAM. Cas9 nuclease cleaves a double-stranded break within a region defined by the crRNA spacer. Repair by NHEJ results in insertions and/or deletions which disrupt expression of the targeted locus. Alternatively, a transgene with homologous flanking sequences can be introduced at the site of DSB via homology directed repair. The crRNA and tracrRNA can be engineered into a single guide RNA (sgRNA or gRNA) (see, e.g., Jinek et al., Science 337:816-21, 2012). Further, the region of the guide RNA complementary to the target site can be altered or programed to target a desired sequence (Xie et al., PLOS One 9: e100448, 2014; U.S. Pat. Appl. Pub. No. US 2014/0068797, U.S. Pat. Appl. Pub. No. US 2014/0186843; U.S. Pat. No. 8,697,359, and PCT Publication No. WO 2015/071474; each of which is incorporated by reference). In certain embodiments, a gene knockout comprises an insertion, a deletion, a mutation or a combination thereof, and made using a CRISPR/Cas nuclease system.

Exemplary gRNA sequences and methods of using the same to knock out endogenous genes that encode immune cell proteins include those described in Ren et al., Clin. Cancer Res. 23(9): 2255-2266 (2017), the gRNAs, CAS9 DNAs, vectors, and gene knockout techniques of which are hereby incorporated by reference in their entirety.

As used herein, a “meganuclease,” also referred to as a “homing endonuclease,” refers to an endodeoxyribonuclease characterized by a large recognition site (double stranded DNA sequences of about 12 to about 40 base pairs). Meganucleases can be divided into five families based on sequence and structure motifs: LAGLIDADG, GIY-YIG, HNH, His-Cys box and PD-(D/E) XK. Exemplary meganucleases include I-Scel, I-Ceul, PI-PspI, PI-Sce, I-SceIV, I-CsmI, I-PanI, I-SceII, I-PpoI, I-SceIII, I-CreI, I-TevI, I-TevII and I-TevIII, whose recognition sequences are known (see, e.g., U.S. Pat. Nos. 5,420,032 and 6,833,252; Belfort et al., Nucleic Acids Res. 25:3379-3388, 1997; Dujon et al., Gene 82:115-118, 1989; Perler et al., Nucleic Acids Res. 22:1125-1127, 1994; Jasin, Trends Genet. 12:224-228, 1996; Gimble et al., J. Mol. Biol. 263:163-180, 1996; Argast et al., J. Mol. Biol. 280:345-353, 1998).

In certain embodiments, naturally occurring meganucleases may be used to promote site-specific genome modification of a target selected from PD-1, LAG3, TIM3, CTLA4, TIGIT, FasL, an HLA-encoding gene, or a TCR component-encoding gene. In other embodiments, an engineered meganuclease having a novel binding specificity for a target gene is used for site-specific genome modification (see, e.g., Porteus et al., Nat. Biotechnol. 23:967-73, 2005; Sussman et al., J. Mol. Biol. 342:31-41, 2004; Epinat et al., Nucleic Acids Res. 31:2952-62, 2003; Chevalier et al., Molec. Cell 10:895-905, 2002; Ashworth et al., Nature 441:656-659, 2006; Paques et al., Curr. Gene Ther. 7:49-66, 2007; U.S. Patent Publication Nos. US 2007/0117128; US 2006/0206949; US 2006/0153826; US 2006/0078552; and US 2004/0002092). In further embodiments, a chromosomal gene knockout is generated using a homing endonuclease that has been modified with modular DNA binding domains of TALENs to make a fusion protein known as a megaTAL. MegaTALs can be utilized to not only knock-out one or more target genes, but to also introduce (knock in) heterologous or exogenous polynucleotides when used in combination with an exogenous donor template encoding a polypeptide of interest.

In certain embodiments, a chromosomal gene knockout comprises an inhibitory nucleic acid molecule that is introduced into a host cell (e.g., an immune cell) comprising a heterologous polynucleotide encoding an antigen-specific receptor that specifically binds to a tumor associated antigen, wherein the inhibitory nucleic acid molecule encodes a target-specific inhibitor and wherein the encoded target-specific inhibitor inhibits endogenous gene expression (e.g., of PD-1, TIM3, LAG3, CTLA4, TIGIT, FasL, an HLA component, or a TCR component, or any combination thereof) in the host cell.

In certain embodiments, a gene knockout comprises an insertion, a deletion, a mutation or a combination thereof, and made using a CRISPR/Cas nuclease system or base editing system (Komor, A. C.; Kim, Y. B.; Packer, M. S.; Zuris, J. A.; Liu, D. R. Nature 533, 420-424 (2016). Briefly, base editing is a genome-editing approach that uses components from CRISPR systems together with other enzymes to directly introduce point mutations into cellular DNA or RNA without making double-stranded DNA breaks. Certain DNA base editors comprise a catalytically disabled nuclease fused to a nucleobase deaminase enzyme and, in some cases, a DNA glycosylase inhibitor. RNA base editors function similarly, using components that target RNA. Base editors directly convert one base or base pair into another, enabling the efficient installation of point mutations in non-dividing cells without generating excess undesired editing by-products. See e.g., Rees H et al. Nature Reviews Genetics (2018).

A chromosomal gene knockout can be confirmed directly by DNA sequencing of the host immune cell following use of the knockout procedure or agent. Chromosomal gene knockouts can also be inferred from the absence of gene expression (e.g., the absence of an mRNA or polypeptide product encoded by the gene) following the knockout.

In certain embodiments, a chromosomal gene knockout comprises a knockout of an HLA component gene selected from an α1 macroglobulin gene, an α2 macroglobulin gene, an α3 macroglobulin gene, a β1 microglobulin gene, or a β2 microglobulin gene.

In certain embodiments, a chromosomal gene knockout comprises a knockout of a TCR component gene selected from a TCR α variable region gene, a TCR β variable region gene, a TCR constant region gene, or a combination thereof.

In some embodiments, a population of host cells comprising a binding protein disclosed herein exhibits at least 5%, at least 10%, at least 20%, at least 30%, at least 40%, at least 50%, at least 60%, at least 70%, at least 80%, at least 90%, at least 2-fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 20 fold, at least 30 fold, at least 40 fold, at least 50 fold, at least 60 fold, at least 70 fold, at least 80 fold, at least 90 fold, at least 100 fold, at least 150 fold, at least 200 fold, at least 250 fold, at least 300 fold, at least 350 fold, at least 400 fold, at least 500 fold, at least 600 fold, at least 700 fold, at least 800 fold, at least 900 fold, at least 1000 fold, or at least 5000 fold increased functional avidity for a target antigen of the binding protein as compared to a population of control cells (for example, cells expressing a control binding protein specific for the same target antigen). The host cells can comprise a binding protein (e.g., a TCR comprising Vα and Vβ regions and/or CDRs disclosed herein) that binds a target antigen (for example, a KRAS G12 mutant peptide, such as KRAS G12V mutant peptide, e.g., present in a peptide: HLA complex). The increase in avidity can be, for example, as determined by an assay for determining expression an activation marker (e.g., CD137, CD69, Granzyme B, CD107a, IFN-gamma, TNF-a, IL-12, a cytokine, an interleukin, an interferon) upon exposure to target cells that express or present the target antigen, or and/or an assay to determine EC50 (e.g., peptide dose at which a half-maximal activation of a T cell population is reached). In some embodiments, the host cells and the control cells are both T cells, and the host cell and control cell populations can comprise the same, about the same, or substantially the same composition or amount(s) of T cell type(s) (e.g., CD4+, CD8+, or both).

In some embodiments, a population of host cells comprising a binding protein disclosed herein exhibits at least 5%, at least 10%, at least 20%, at least 30%, at least 40%, at least 50%, at least 60%, at least 70%, at least 80%, at least 90%, at least 2-fold, at least 3 fold, at least 4 fold, at least 5 fold, at least 6 fold, at least 7 fold, at least 8 fold, at least 9 fold, at least 10 fold, at least 15 fold, at least 20 fold, at least 30 fold, at least 40 fold, at least 50 fold, at least 100 fold, at least 250 fold, or at least 1000 fold increased killing of target cells as compared to a population of control cells (for example, cells expressing a control binding protein specific for the same target antigen). The killing of target cells can be, for example, as determined by an in vitro cytotoxicity assay, for example, at an effector to target ratio of about 0.5:1, 1:1, 2:1, 3:1, 4:1, 5:1, 6:1, 7:1, 8:1, 9:1, 10:1, 20:1, 25:1, 50:1, or 100:1. In some embodiments, the host cells and the control cells are both T cells, and the host cell and control cell populations can comprise the same, about the same, or substantially the same composition or amount(s) of T cell type(s) (e.g., CD4+, CD8+, or both).

In some embodiments, a population of host cells comprising a binding protein disclosed herein exhibits at least 5%, at least 10%, at least 20%, at least 30%, at least 40%, at least 50%, at least 60%, at least 70%, at least 80%, at least 90%, at least 2-fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 20 fold, at least 30 fold, at least 40 fold, at least 50 fold, at least 60 fold, at least 70 fold, at least 80 fold, at least 90 fold, at least 100 fold, at least 150 fold, at least 200 fold, at least 250 fold, at least 300 fold, at least 350 fold, at least 400 fold, at least 500 fold, at least 600 fold, at least 700 fold, at least 800 fold, at least 900 fold, at least 1000 fold, or at least 5000 fold increased activation as compared to a population of control cells (for example, cells expressing a control binding protein specific for the same target antigen). The activation can be, for example, as determined by an assay for determining expression an activation marker (e.g., CD137, CD69, Granzyme B, CD107a, IFN-gamma, TNF-α, IL-12, a cytokine, an interleukin, an interferon) upon exposure to target cells that express or present the target antigen. In some embodiments, the host cells and the control cells are both T cells, and the host cell and control cell populations can comprise the same, about the same, or substantially the same composition or amount(s) of T cell type(s) (e.g., CD4+, CD8+, or both).

In some embodiments, a population of host cells comprising a binding protein disclosed herein is resistant to exhaustion, for example, exhibits effective tumor cell killing upon multiple rechallenges in vitro (e.g., for at least 50 hours, at least 100 hours, at least 150 hours, at least 200 hours, or at least 250 hours, optionally with one or more rechallenges), or exhibits persistent control of tumor growth in vivo.

In some embodiments, a population of host cells comprising a binding protein disclosed herein is resistant to exhaustion compared to a population of control cells, for example, exhibits superior tumor cell killing upon multiple rechallenges in vitro (e.g., for at least 50 hours, at least 100 hours, at least 150 hours, at least 200 hours, or at least 250 hours, optionally with one or more rechallenges), or exhibits superior control of tumor growth in vivo. In some embodiments, the host cells and the control cells are both T cells, and the host cell and control cell populations can comprise the same, about the same, or substantially the same composition or amount(s) of T cell type(s) (e.g., CD4+, CD8+, or both).

### Host Cell Compositions and Unit Doses

In another aspect, compositions and unit doses are provided herein that comprise a modified host cell of the present disclosure and a pharmaceutically acceptable carrier, diluent, or excipient.

In certain embodiments, a host cell composition or unit dose comprises (i) a composition comprising at least about 30%, at least about 40%, at least about 50%, at least about 60%, at least about 70%, at least about 80%, at least about 85%, at least about 90%, or at least about 95% modified CD4⁺ T cells, combined with (ii) a composition comprising at least about 30%, at least about 40%, at least about 50%, at least about 60%, at least about 70%, at least about 80%, at least about 85%, at least about 90%, or at least about 95% modified CD8⁺ T cells, in about a 1:1 ratio, wherein the unit dose contains a reduced amount or substantially no naïve T cells (i.e., has less than about 50%, less than about 40%, less than about 30%, less than about 20%, less than about 10%, less than about 5%, or less then about 1% the population of naïve T cells present in a unit dose as compared to a patient sample having a comparable number of PBMCs).

In some embodiments, a host cell composition or unit dose comprises (i) a composition comprising at least about 50% modified CD4⁺ T cells, combined with (ii) a composition comprising at least about 50% modified CD8⁺ T cells, in about a 1:1 ratio, wherein the host cell composition or unit dose contains a reduced amount or substantially no naïve T cells. In further embodiments, a host cell composition or unit dose comprises (i) a composition comprising at least about 60% modified CD4⁺ T cells, combined with (ii) a composition comprising at least about 60% modified CD8⁺ T cells, in about a 1:1 ratio, wherein the unit dose contains a reduced amount or substantially no naïve T cells. In still further embodiments, a host cell composition or unit dose comprises (i) a composition comprising at least about 70% engineered CD4⁺ T cells, combined with (ii) a composition comprising at least about 70% engineered CD8⁺ T cells, in about a 1:1 ratio, wherein the unit dose contains a reduced amount or substantially no naïve T cells. In some embodiments, a host cell composition or unit dose comprises (i) a composition comprising at least about 80% modified CD4⁺ T cells, combined with (ii) a composition comprising at least about 80% modified CD8⁺ T cells, in about a 1:1 ratio, wherein the host cell composition or unit dose contains a reduced amount or substantially no naïve T cells. In some embodiments, a host cell composition or unit dose comprises (i) a composition comprising at least about 85% modified CD4⁺ T cells, combined with (ii) a composition comprising at least about 85% modified CD8⁺ T cells, in about a 1:1 ratio, wherein the host cell composition or unit dose contains a reduced amount or substantially no naïve T cells. In some embodiments, a host cell composition or unit dose comprises (i) a composition comprising at least about 90% modified CD4⁺ T cells, combined with (ii) a composition comprising at least about 90% modified CD8⁺ T cells, in about a 1:1 ratio, wherein the host cell composition or unit dose contains a reduced amount or substantially no naïve T cells.

In some embodiments, the composition comprises a CD4+ cell population comprising (i) at least about 30%, at least about 40%, at least about 50%, at least about 60%, at least about 70%, at least about 80%, at least about 85%, at least about 90%, or at least about 95% modified CD4+ T cells. In some embodiments, the composition further comprises a CD8+ cell population comprising (ii) at least about 30%, at least about 40%, at least about 50%, at least about 60%, at least about 70%, at least about 80%, at least about 85%, at least about 90%, or at least about 95% modified CD8+ T cells.

In some embodiments, a host cell composition or unit dose comprises about a 1:1 ratio, about a 1:2 ratio, about a 1:3 ratio, about a 1:4 ratio, about a 1:5 ratio, about a 1:6 ratio, about a 1:7 ratio, about a 1:8 ratio, about a 1:9 ratio, about a 1:10 ratio, about a 2:1 ratio, about a 3:1 ratio, about a 4:1 ratio, about a 5:1 ratio, about a 6:1 ratio, about a 7:1 ratio, about an 8:1 ratio, about a 9:1 ratio, about a 10:1 ratio, about a 3:2 ratio, or about a 2:3 ratio of CD4+ to CD8+ T cells (for example, of CD4+ T cells modified to comprise or express a binding protein disclosed herein to CD8+ T cells modified to comprise or express a binding protein disclosed herein).

In some embodiments, a host cell composition or unit dose comprises a ratio of CD4+ to CD8+ T cells that is at least 1:1, at least 1:2, at least 1:3, at least 1:4, at least 1:5, at least 1:6, at least 1:7, at least 1:8, at least 1:9, at least 1:10, at least 2:1, at least 3:1, at least 4:1, at least 5:1, at least 6:1, at least 7:1, at least 8:1, at least 9:1, at least 10:1, at least 3:2, or at least 2:3.

In some embodiments, a host cell composition or unit dose comprises a ratio of CD4+ to CD8+ T cells that is at most 1:1, at most 1:2, at most 1:3, at most 1:4, at most 1:5, at most 1:6, at most 1:7, at most 1:8, at most 1:9, at most 1:10, at most 2:1, at most 3:1, at most 4:1, at most 5:1, at most 6:1, at most 7:1, at most 8:1, at most 9:1, at most 10:1, at most 3:2, or at most 2:3.

In some embodiments, a host cell composition or unit dose comprises a ratio of CD4+ to CD8+ T cells that is between about 1:10 and 10:1, 1:10 and 8:1, 1:10 and 7:1, 1:10 and 6:1, 1:10 and 5:1, 1:10 and 4:1, 1:10 and 3:1, 1:10 and 2:1, 1:10 and 1:1, 1:10 and 1:2, 1:10 and 1:3, 1:10 and 1:4, 1:10 and 1:5, 1:10 and 1:7, 1:5 and 10:1, 1:5 and 8:1, 1:5 and 7:1, 1:5 and 6:1, 1:5 and 5:1, 1:5 and 4:1, 1:5 and 3:1, 1:5 and 2:1, 1:5 and 1:1, 1:5 and 1:2, 1:5 and 1:3, 1:5 and 1:4, 1:3 and 10:1, 1:3 and 8:1, 1:3 and 7:1, 1:3 and 6:1, 1:3 and 5:1, 1:3 and 4:1, 1:3 and 3:1, 1:3 and 2:1, 1:3 and 1:1, 1:3 and 1:2, 1:2 and 10:1, 1:2 and 8:1, 1:2 and 7:1, 1:2 and 6:1, 1:2 and 5:1, 1:2 and 4:1, 1:2 and 3:1, 1:2 and 2:1, 1:2 and 1:1, 1:1 and 10:1, 1:1 and 8:1, 1:1 and 7:1, 1:1 and 6:1, 1:1 and 5:1, 1:1 and 4:1, 1:1 and 3:1, 1:1 and 2:1, 2:1 and 10:1, 2:1 and 8:1, 2:1 and 7:1, 2:1 and 6:1, 2:1 and 5:1, 2:1 and 4:1, 2:1 and 3:1, 3:1 and 10:1, 3:1 and 8:1, 3:1 and 7:1, 3:1 and 6:1, 3:1 and 5:1, 3:1 and 4:1, 5:1 and 10:1, 5:1 and 8:1, 5:1 and 7:1, or 5:1 and 6:1.

CD4+ T cells in a composition, host cell composition, or unit dose can be CD4+ T cells that are modified or engineered to express a CD8 co-receptor disclosed herein, for example, using a vector or polynucleotide disclosed herein.

It will be appreciated that a host cell composition or unit dose of the present disclosure may comprise any host cell as described herein, or any combination of host cells. In certain embodiments, for example, a host cell composition or unit dose comprises modified CD8+ T cells, modified CD4+ T cells, or both, wherein these T cells are modified to encode a binding protein specific for a Ras peptide: HLA-A*11:01 complex. In addition or alternatively, a host cell composition or unit dose of the present disclosure can comprise any host cell or combination of host cells as described herein, and can further comprise a modified cell (e.g., immune cell, such as a T cell) expressing a binding protein specific for a different antigen (e.g., a different Ras antigen, or an antigen from a different protein or target, such as, for example, BCMA, CD3, CEACAM6, c-Met, EGFR, EGFRvIII, ErbB2, ErbB3, ErbB4, EphA2, IGF1R, GD2, O-acetyl GD2, O-acetyl GD3, GHRHR, GHR, FLT1, KDR, FLT4, CD44v6, CD151, CA125, CEA, CTLA-4, GITR, BTLA, TGFBR2, TGFBR1, IL6R, gp130, Lewis A, Lewis Y, TNFR1, TNFR2, PD1, PD-L1, PD-L2, HVEM, MAGE-A (e.g., including MAGE-A1, MAGE-A3, and MAGE-A4), mesothelin, NY-ESO-1, PSMA, RANK, ROR1, TNFRSF4, CD40, CD137, TWEAK-R, HLA, tumor- or pathogen-associated peptide bound to HLA, hTERT peptide bound to HLA, tyrosinase peptide bound to HLA, WT-1 peptide bound to HLA, LTβR, LIFRβ, LRP5, MUC1, OSMRβ, TCRα, TCRβ, CD19, CD20, CD22, CD25, CD28, CD30, CD33, CD52, CD56, CD79a, CD79b, CD80, CD81, CD86, CD123, CD171, CD276, B7H4, TLR7, TLR9, PTCHI, WT-1, HA¹-H, Robol, α-fetoprotein (AFP), Frizzled, OX40, PRAME, and SSX-2. or the like). For example, a unit dose can comprise modified CD8⁺ T cells expressing a binding protein that specifically binds to a Ras-HLA complex and modified CD4⁺ T cells (and/or modified CD8 T cells) expressing a binding protein (e.g., a CAR) that specifically binds to a PSMA antigen. It will also be appreciated that any of the host cells disclosed herein may be administered in a combination therapy.

In any of the embodiments described herein, a host cell composition or unit dose comprises equal, or approximately equal numbers of engineered CD45RA⁻ CD3⁺ CD8⁺ and modified CD45RA⁻ CD3⁺ CD4⁺ T_{M} cells.

In any of the embodiments described herein, a host cell composition or unit dose comprises one or more populations of cells (e.g., CD4+ or CD8+ cells) that have undergone CD62L positive selection, for example, to improve in vivo persistence.

Host cells can be genetically engineered to comprise or express a binding protein ex vivo, in vitro, or in vivo. In some embodiments, a host cell is genetically engineered ex vivo to express a binding protein. In some embodiments, a host cell is genetically engineered in vitro to express a binding protein. In some embodiments, a host cell is genetically engineered in vivo to express a binding protein.

### Uses

In additional aspects, the present disclosure provides methods for treating or for preventing a relapse of a disease or disorder associated with a KRAS G12V or a NRAS G12V mutation or a HRAS G12V mutation in a subject. Such diseases or disorders include, for example, cancers, such as solid cancers and hematological malignancies. In certain exemplary embodiments, the disease or disorder comprises a pancreas cancer or carcinoma, optionally a pancreatic ductal adenocarcinoma (PDAC); a colorectal cancer or carcinoma; a colon cancer; a colorectal adenocarcinoma; a lung cancer, optionally a non-small-cell lung carcinoma; a biliary cancer; an endometrial cancer or carcinoma; a cervical cancer; an ovarian cancer; a bladder cancer; a liver cancer; a myeloid leukemia, optionally myeloid leukemia such as acute myeloid leukemia; a myelodysplastic syndrome; a lymphoma such as Non-Hodgkin lymphoma; Chronic Melyomonocytic Leukemia; Acute Lymphoblastic Leukemia (ALL); a cancer of the urinary tract; a cancer of the small intestine; a breast cancer or carcinoma; a melanoma (optionally a cutaneous melanoma, an anal melanoma, or a mucosal melanoma); a glioma; a poorly differentiated thyroid gland carcinoma; a neuroblastoma; a histiocytic and dendritic cell neoplasm; neurofibromatisis Type 1; rhabdomyosarcoma; a soft tissue sarcoma; a bladder carcinoma; a sarcoma; a glioblastoma; a squamous cell lung carcinoma; an anaplastic astrocytoma; chronic myeloid leukemia; diffuse large B-cell lymphoma; double-hit lymphoma; head and neck carcinoma; head and neck squamous cell carcinoma; hepatocellular carcinoma; malignant peripheral nerve sheath tumor; mantle cell lymphoma; myelodyspastic/myeloproliferative neoplasm, unclassifiable; peripheral T cell lymphoma; prostate carcinoma; refractory anemia with excess blasts-2; renal cell carcinoma; rhabdoid tumor; schwannoma; secondary AML; small cell lung carcinoma; therapy-related AML; thymic carcinoma; thyroid gland follyicular carcinoma; malignant thyroid gland neoplasm; thyroid gland carcinoma; thyroid gland adenocarcinoma; urothelial carcinoma; or thyroid gland papillary carcinoma. A disease or disorder that can be treated by a composition or method disclosed herein includes an advanced or metastatic form of a cancer disclosed herein.

“Treat” or “treatment” or “ameliorate” refers to medical management of a disease, disorder, or condition of a subject (e.g., a human or non-human mammal, such as a primate, horse, cat, dog, goat, mouse, or rat). In general, an appropriate dose or treatment regimen comprising a composition (e.g., comprising a binding protein, polynucleotide, vector, host cell, host cell composition, unit dose, and/or immunogenic polypeptide) of the present disclosure is administered in an amount sufficient to elicit a therapeutic or prophylactic benefit. Therapeutic or prophylactic/preventive benefit includes improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; prolonged survival; or any combination thereof.

A “therapeutically effective amount” or “effective amount”, as used herein, refers to an amount of a composition sufficient to result in a therapeutic effect, including improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; or prolonged survival in a statistically significant manner. When referring to an individual active ingredient or a cell expressing a single active ingredient, administered alone, a therapeutically effective amount refers to the effects of that ingredient or cell expressing that ingredient alone. When referring to a combination, a therapeutically effective amount refers to the combined amounts of active ingredients or combined adjunctive active ingredient with a cell expressing an active ingredient that results in a therapeutic effect, whether administered serially or simultaneously. A combination may also be a cell expressing more than one active ingredient.

The term “pharmaceutically acceptable excipient or carrier” or “physiologically acceptable excipient or carrier” refer to biologically compatible vehicles, e.g., physiological saline, which are described in greater detail herein, that are suitable for administration to a human or other non-human mammalian subject and generally recognized as safe or not causing a serious adverse event.

As used herein, “statistically significant” refers to a p value of 0.050 or less when calculated using the Students t-test or other appropriate statistical test and indicates that it is unlikely that a particular event or result being measured has arisen by chance.

Subjects that can be treated by the present invention are, in general, human and other primate subjects, such as monkeys and apes for veterinary medicine purposes. In any of the aforementioned embodiments, the subject may be a human subject. The subjects can be male or female and can be any suitable age, including infant, juvenile, adolescent, adult, and geriatric subjects. The subject can be a mammal. Compositions according to the present disclosure may be administered in a manner appropriate to the disease, condition, or disorder to be treated as determined by persons skilled in the medical art. In any of the above embodiments, a modified host cell, host cell composition, or unit dose as described herein is administered intravenously, intraperitoneally, intratumorally, into the bone marrow, into a lymph node, or into the cerebrospinal fluid so as to encounter target cells (e.g., leukemia cells). An appropriate dose, suitable duration, and frequency of administration of the compositions will be determined by such factors as a condition of the patient; size, type, and severity of the disease, condition, or disorder; the particular form of the active ingredient; and the method of administration.

As used herein, the term “adoptive immune therapy” or “adoptive immunotherapy” refers to administration of naturally occurring or genetically engineered, disease- or antigen-specific immune cells (e.g., T cells). Adoptive cellular immunotherapy may be autologous (immune cells are from the recipient), allogeneic (immune cells are from a donor of the same species that is not the recipient) or syngeneic (immune cells are from a donor genetically identical or substantially genetically identical to the recipient, for example, monozygotic twins).

In some embodiments, the subject (e.g., at least one cell in the subject) expresses a Ras antigen comprising or consisting of the amino acid sequence set forth in any one of SEQ ID NOs: 2-3.

In some embodiments, the subject is HLA-A*11⁺ (e.g., HLA-A*11:01⁺).

In certain embodiments, a method comprises determining the HLA type or types of a subject and/or identifying the presence of a Ras antigen, prior to administering therapy according to the present disclosure. In some embodiments, the HLA type or types of the subject and/or presence of a Ras antigen (e.g., G12V mutation) has been determined prior to administering the therapy, e.g., and the therapy is administered based at least in part on the HLA type(s) and/or presence of the Ras antigen. In some embodiments, the method further comprises genotyping a tumor of the subject for a KRAS G12 allele prior to the administering. In some cases, the subject is determined to carry a KRAS G12V allele prior to the administering.

Expression of an HLA allele can be determined by, for example, genetic sequencing (e.g., high throughput Next Generation Sequencing (NGS)). This genetic determination of the HLA expression is referred to herein as “HLA typing” and can determined though molecular approaches in a clinical laboratory licensed for HLA typing. In some embodiments, HLA typing is performed using PCR amplification followed by high throughput NGS and subsequent HLA determination. Herein, the HLA haplotype can be determined at the major HLA loci (e.g., HLA-A, HLA-B, HLA-C, etc.).

HLA typing can be performed using any known method, including, for example, protein or nucleic acid testing. Examples of nucleic acid testing include sequence-based typing (SBT) and use of sequence-specific oligonucleotide probes (SSOP) or sequence-specific primers (SSP). In certain embodiments, HLA typing is performed using PCR amplification followed by high throughput Next Generation Sequencing (NGS) and subsequent HLA determination. In some embodiments, sequence typing is performed using a system available through Scisco Genetics (sciscogenetics.com/pages/technology.html, the contents of which is incorporated herein by reference in its entirety). Other methods for HLA typing include, e.g., those disclosed in Mayor et al. PLOS One 10(5): e0127153 (2015), which methods and reagents are incorporated herein by reference.

In particular embodiments, a method comprises administering a composition comprising modified CD8+ and/or modified CD4+ T cells that comprise a heterologous polynucleotide encoding a second binding protein as provided herein, when the subject expresses HLA-A*11:01.

In the case of host cell compositions or unit doses, the amount of cells therein is at least one cell (for example, one modified CD8⁺ T cell subpopulation (e.g., optionally comprising memory and/or naïve CD8⁺ T cells); one modified CD4⁺ T cell subpopulation (e.g., optionally comprising memory and/or naïve CD4⁺ T cells)) or is more typically greater than 10² cells, for example, up to 10⁴, up to 10⁵, up to 10⁶, up to 10⁷, up to 10⁸, up to 10⁹, or more than 10¹⁰ cells. In certain embodiments, the cells are administered in a range from about 10⁴ to about 10¹⁰ cells/m², preferably in a range of about 10⁵ to about 10⁹ cells/m². In some embodiments, an administered dose comprises up to about 3.3×10⁵ cells/kg. In some embodiments, an administered dose comprises up to about 1×10⁶ cells/kg. In some embodiments, an administered dose comprises up to about 3.3×10⁶ cells/kg. In some embodiments, an administered dose comprises up to about 1×10⁷ cells/kg. In certain embodiments, a modified immune cell is administered to a subject at a dose comprising up to about 5×10⁴ cells/kg, 5×10⁵ cells/kg, 5×10⁶ cells/kg, or up to about 5×10⁷ cells/kg. In certain embodiments, a modified immune cell is administered to a subject at a dose comprising at least about 5×10⁴ cells/kg, 5×10⁵ cells/kg, 5×10⁶ cells/kg, or up to about 5×10⁷ cells/kg. The number of cells will depend upon the ultimate use for which the composition is intended as well as the type of cells included therein. For example, cells modified to contain a binding protein can comprise a cell population containing at least 30%, at least 35%, at least 40%, at least 45%, at least 50%, at least 55%, at least 60%, at least 65%, at least 70%, at least 75%, at least 80%, at least 85%, at least 90%, at least 95%, or more of such cells. For uses provided herein, cells are generally in a volume of a liter or less, 500 mls or less, 250 mls or less, or 100 mls or less. In embodiments, the density of the desired cells is typically greater than 10⁴ cells/ml and generally is greater than 10⁷ cells/ml, generally 10⁸ cells/ml or greater. The cells may be administered as a single infusion or in multiple infusions over a range of time. A clinically relevant number of immune cells can be apportioned into multiple infusions that cumulatively equal or exceed 10⁶, 10⁷, 10⁸, 10⁹, 10¹⁰, or 10¹¹ cells. In certain embodiments, a unit dose of the modified immune cells can be co-administered with (e.g., simultaneously or contemporaneously with) hematopoietic stem cells from an allogeneic donor. In some embodiments, one or more of the modified immune cells comprised in the unit dose is autologous to the subject.

In some embodiments, a unit dose comprises, consists essentially of, or consists of, or a plurality of unit doses comprises, consists essentially of, or consists of, at least 5×10\hat{ }7, at least 1×10\hat{ }8, at least 5×10\hat{ }8, at least 1×10\hat{ }9, at least 2.5×10\hat{ }9, at least 5×10\hat{ }9, at least 1×10\hat{ }10, at least 1.5×10\hat{ }10, at least 2×10\hat{ }10, at least 3×10\hat{ }10, at least 5×10\hat{ }10, or at least 1×10\hat{ }11 viable host cells, e.g., that encode, comprise, or express a binding protein as disclosed herein.

In some embodiments, a unit dose comprises, consists essentially of, or consists of, or a plurality of unit doses comprises, consists essentially of, or consists of, at most 1×10\hat{ }8, at most 5×10\hat{ }9, at most 1×10\hat{ }10, at most 1.5×10\hat{ }10, at most 2×10\hat{ }10, at most 2.5×10\hat{ }10, at most 3×10\hat{ }10, at most 4×10\hat{ }10, at most 5×10\hat{ }10, at most 1×10\hat{ }11, at most 5×10\hat{ }11, or at most 2×10\hat{ }12 viable host cells, e.g., that encode, comprise, or express a binding protein as disclosed herein.

In some embodiments, a unit dose comprises, consists essentially of, or consists of, or a plurality of unit doses comprises, consists essentially of, or consists of, about 1×10\hat{ }8, about 5×10\hat{ }8, about 1×10\hat{ }9, about 2×10\hat{ }9, about 3×10\hat{ }9, about 4×10\hat{ }9, about 5×10\hat{ }9, about 6×10\hat{ }9, about 7×10\hat{ }9, about 8×10\hat{ }9, about 9×10\hat{ }9, about 1×10\hat{ }10, about 1.1×10\hat{ }10, about 1.2×10\hat{ }10, about 1.3×10\hat{ }10, about 1.4×10\hat{ }10, about 1.5×10\hat{ }10, about 1.6×10\hat{ }10, about 1.7×10\hat{ }10, about 1.8×10\hat{ }10, about 1.9×10\hat{ }10, about 2×10\hat{ }10, about 3×10\hat{ }10, about 4×10\hat{ }10, about 5×10\hat{ }10, about 7.5×10\hat{ }10, about 10×10\hat{ }10, or about 1×10\hat{ }11 viable host cells, e.g., that encode, comprise, or express a binding protein as disclosed herein.

In some embodiments, a unit dose comprises, consists essentially of, or consists of, or a plurality of unit doses comprises, consists essentially of, or consists of, about 1×10\hat{ }8 to about 1×10\hat{ }11, about 1×10\hat{ }8 to about 5×10\hat{ }10, about 1×10\hat{ }8 to about 2×10\hat{ }10, about 1×10\hat{ }8 to about 1.5×10\hat{ }10, about 1×10\hat{ }8 to about 1×10\hat{ }10, about 1×10\hat{ }8 to about 5×10\hat{ }9, about 1×10\hat{ }9 to about 1×10\hat{ }11, about 1×10\hat{ }9 to about 5×10\hat{ }10, about 1×10\hat{ }9 to about 2×10\hat{ }10, about 1×10\hat{ }9 to about 1.5×10\hat{ }10, about 1×10\hat{ }9 to about 1×10\hat{ }10, about 1×10\hat{ }9 to about 5×10\hat{ }9, about 5×10\hat{ }9 to about 1×10\hat{ }11, about 5×10\hat{ }9 to about 5×10\hat{ }10, about 5×10\hat{ }9 to about 2×10\hat{ }10, about 5×10\hat{ }9 to about 1.5×10\hat{ }10, about 5×10\hat{ }9 to about 1×10\hat{ }10, about 1×10\hat{ }10 to about 1×10\hat{ }11, about 1×10\hat{ }10 to about 5×10\hat{ }10, about 1×10\hat{ }10 to about 2×10\hat{ }10, about 1×10\hat{ }10 to about 1.5×10\hat{ }10, about 1.5×10\hat{ }10 to about 1×10\hat{ }11, about 1.5×10\hat{ }10 to about 5×10\hat{ }10, or about 1.5×10\hat{ }10 to about 2×10\hat{ }10 viable host cells, e.g., that encode, comprise, or express a binding protein as disclosed herein.

In some embodiments, the subject receiving the modified immune cell has previously received lymphodepleting chemotherapy. In further embodiments, the lymphodepleting chemotherapy comprises cyclophosphamide, fludarabine, anti-thymocyte globulin, or a combination thereof.

In some embodiments, the method further comprises administering an inhibitor of an immune checkpoint molecule, as disclosed herein, to the subject.

Also contemplated are pharmaceutical compositions (i.e., compositions) that comprise a composition (binding protein, polynucleotide, vector, host cell, host cell composition, unit dose, and/or immunogenic polypeptide) as disclosed herein and a pharmaceutically acceptable carrier, diluents, or excipient. Suitable excipients include water, saline, dextrose, glycerol, or the like and combinations thereof. In embodiments, compositions comprising fusion proteins or host cells as disclosed herein further comprise a suitable infusion media. Suitable infusion media can be any isotonic medium formulation, typically normal saline, Normosol R (Abbott) or Plasma-Lyte A (Baxter), 5% dextrose in water, Ringer's lactate can be utilized. An infusion medium can be supplemented with human serum albumin or other human serum components.

Pharmaceutical compositions may be administered in a manner appropriate to the disease or condition to be treated (or prevented) as determined by persons skilled in the medical art. An appropriate dose and a suitable duration and frequency of administration of the compositions will be determined by such factors as the health condition of the patient, size of the patient (i.e., weight, mass, or body area), the type and severity of the patient's condition, the particular form of the active ingredient, and the method of administration. In general, an appropriate dose and treatment regimen provide the composition(s) in an amount sufficient to provide therapeutic and/or prophylactic benefit (such as described herein, including an improved clinical outcome, such as more frequent complete or partial remissions, or longer disease-free and/or overall survival, or a lessening of symptom severity).

An effective amount of a pharmaceutical composition refers to an amount sufficient, at dosages and for periods of time needed, to achieve the desired clinical results or beneficial treatment, as described herein. An effective amount may be delivered in one or more administrations. If the administration is to a subject already known or confirmed to have a disease or disease-state, the term “therapeutic amount” may be used in reference to treatment, whereas “prophylactically effective amount” may be used to describe administrating an effective amount to a subject that is susceptible or at risk of developing a disease or disease-state (e.g., recurrence) as a preventative course.

The pharmaceutical compositions described herein may be presented in unit-dose or multi-dose containers, such as sealed ampoules or vials. Such containers may be frozen to preserve the stability of the formulation until infusion into the patient. Doses will vary, but a preferred dose for administration of a modified immune cell as described herein is about 10⁴ cells/m², about 5×10⁴ cells/m², about 10⁵ cells/m², about 5×10⁵ cells/m², about 10⁶ cells/m², about 5×10⁶ cells/m², about 10⁷ cells/m², about 5×10⁷ cells/m², about 10⁸ cells/m², about 5×10⁸ cells/m², about 10⁹ cells/m², about 5×10⁹ cells/m², about 10¹⁰ cells/m², about 5×10¹⁰ cells/m², or about 10¹¹ cells/m². In certain embodiments, a unit dose comprises a modified immune cell as described herein at a dose of about 10⁴ cells/m² to about 10¹¹ cells/m². The development of suitable dosing and treatment regimens for using the particular compositions described herein in a variety of treatment regimens, including e.g., parenteral or intravenous administration or formulation.

If the subject composition is administered parenterally, the composition may also include sterile aqueous or oleaginous solution or suspension. Suitable non-toxic parenterally acceptable diluents or solvents include water, Ringer's solution, isotonic salt solution, 1,3-butanediol, ethanol, propylene glycol or polyethylene glycols in mixtures with water. Aqueous solutions or suspensions may further comprise one or more buffering agents, such as sodium acetate, sodium citrate, sodium borate or sodium tartrate. Of course, any material used in preparing any dosage unit formulation should be pharmaceutically pure and substantially non-toxic in the amounts employed. In addition, the active compounds may be incorporated into sustained-release preparation and formulations. Dosage unit form, as used herein, refers to physically discrete units suited as unitary dosages for the subject to be treated; each unit may contain a predetermined quantity of engineered immune cells or active compound calculated to produce the desired effect in association with an appropriate pharmaceutical carrier.

In general, an appropriate dosage and treatment regimen provides the active molecules or cells in an amount sufficient to provide a benefit. Such a response can be monitored by establishing an improved clinical outcome (e.g., more frequent remissions, complete or partial, or longer disease-free survival) in treated subjects as compared to non-treated subjects. Increases in preexisting immune responses to a tumor protein generally correlate with an improved clinical outcome. Such immune responses may generally be evaluated using standard proliferation, cytotoxicity or cytokine assays, which are routine.

For prophylactic use, a dose should be sufficient to prevent, delay the onset of, or diminish the severity of a disease associated with disease or disorder. Prophylactic benefit of the immunogenic compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by a person skilled in the art.

As used herein, administration of a composition refers to delivering the same to a subject, regardless of the route or mode of delivery. Administration may be effected continuously or intermittently, and parenterally. A composition can be administered locally (e.g., intratumoral) or systemically (e.g., intravenously). Administration may be for treating a subject already confirmed as having a recognized condition, disease or disease state, or for treating a subject susceptible to or at risk of developing such a condition, disease or disease state. Co-administration with an adjunctive therapy may include simultaneous and/or sequential delivery of multiple agents in any order and on any dosing schedule (e.g., modified immune cells with one or more cytokines; immunosuppressive therapy such as calcineurin inhibitors, corticosteroids, microtubule inhibitors, low dose of a mycophenolic acid prodrug, or any combination thereof).

In certain embodiments, a plurality of doses of a composition described herein is administered to the subject, which may be administered at intervals between administrations of about two to about four weeks.

Treatment or prevention methods of this disclosure may be administered to a subject as part of a treatment course or regimen, which may comprise additional treatments prior to, or after, administration of the instantly disclosed unit doses, cells, or compositions. For example, in certain embodiments, a subject receiving a unit dose of the modified immune cell is receiving or had previously received a hematopoietic cell transplant (HCT; including myeloablative and non-myeloablative HCT). Techniques and regimens for performing HCT are known in the art and can comprise transplantation of any suitable donor cell, such as a cell derived from umbilical cord blood, bone marrow, or peripheral blood, a hematopoietic stem cell, a mobilized stem cell, or a cell from amniotic fluid. Accordingly, in certain embodiments, a modified immune cell of the present disclosure can be administered with or shortly after hematopoietic stem cells in a modified HCT therapy. In some embodiments, the HCT comprises a donor hematopoietic cell comprising a chromosomal knockout of a gene that encodes an HLA component, a chromosomal knockout of a gene that encodes a TCR component, or both.

In further embodiments, the subject had previously received lymphodepleting chemotherapy prior to receiving the composition or HCT. In certain embodiments, a lymphodepleting chemotherapy comprises a conditioning regimen comprising cyclophosphamide, fludarabine, anti-thymocyte globulin, or a combination thereof.

Methods according to this disclosure may further include administering one or more additional agents to treat the disease or disorder in a combination therapy. For example, in certain embodiments, a combination therapy comprises administering a composition of the present disclosure with (concurrently, simultaneously, or sequentially) an immune checkpoint inhibitor. In some embodiments, a combination therapy comprises administering a composition of the present disclosure with an agonist of a stimulatory immune checkpoint agent. In further embodiments, a combination therapy comprises administering a composition of the present disclosure with a secondary therapy, such as chemotherapeutic agent, a radiation therapy, a surgery, an antibody, or any combination thereof.

As used herein, the term “immune suppression agent” or “immunosuppression agent” refers to one or more cells, proteins, molecules, compounds or complexes providing inhibitory signals to assist in controlling or suppressing an immune response. For example, immune suppression agents include those molecules that partially or totally block immune stimulation; decrease, prevent or delay immune activation; or increase, activate, or up regulate immune suppression. Exemplary immunosuppression agents to target (e.g., with an immune checkpoint inhibitor) include PD-1, PD-L1, PD-L2, LAG3, CTLA4, B7-H3, B7-H4, CD244/2B4, HVEM, BTLA, CD160, TIM3, GAL9, KIR, PVRIG (CD112R), PVRL2, adenosine, A2aR, immunosuppressive cytokines (e.g., IL-10, IL-4, IL-IRA, IL-35), IDO, arginase, VISTA, TIGIT, LAIR1, CEACAM-1, CEACAM-3, CEACAM-5, Treg cells, or any combination thereof.

An immune suppression agent inhibitor (also referred to as an immune checkpoint inhibitor) may be a compound, an antibody, an antibody fragment or fusion polypeptide (e.g., Fc fusion, such as CTLA4-Fc or LAG3-Fc), an antisense molecule, a ribozyme or RNAi molecule, or a low molecular weight organic molecule. In any of the embodiments disclosed herein, a method may comprise a composition of the present disclosure with one or more inhibitor of any one of the following immune suppression components, singly or in any combination.

In certain embodiments, a composition of the present disclosure is used in combination with a PD-1 inhibitor, for example a PD-1-specific antibody or binding fragment thereof, such as pidilizumab, nivolumab, pembrolizumab, MEDI0680 (formerly AMP-514), AMP-224, BMS-936558 or any combination thereof. In further embodiments, a composition of the present disclosure is used in combination with a PD-L1 specific antibody or binding fragment thereof, such as BMS-936559, durvalumab (MEDI4736), atezolizumab (RG7446), avelumab (MSB0010718C), MPDL3280A, or any combination thereof. Also contemplated are cemiplimab; IBI-308; nivolumab+relatlimab; BCD-100; camrelizumab; JS-001; spartalizumab; tislelizumab; AGEN-2034; BGBA-333+tislelizumab; CBT-501; dostarlimab; durvalumab+MEDI-0680; JNJ-3283; pazopanib hydrochloride+pembrolizumab; pidilizumab; REGN-1979+cemiplimab; ABBV-181; ADUS-100+spartalizumab; AK-104; AK-105; AMP-224; BAT-1306; BI-754091; CC-90006; cemiplimab+REGN-3767; CS-1003; GLS-010; LZM-009; MEDI-5752; MGD-013; PF-06801591; Sym-021; tislelizumab+pamiparib; XmAb-20717; AK-112; ALPN-202; AM-0001; an antibody to antagonize PD-1 for Alzheimer's disease; BH-2922; BH-2941; BH-2950; BH-2954; a biologic to antagonize CTLA-4 and PD-1 for solid tumor; a bispecific monoclonal antibody to target PD-1 and LAG-3 for oncology; BLSM-101; CB-201; CB-213; CBT-103; CBT-107; a cellular immunotherapy+PD-1 inhibitor; CX-188; HAB-21; HEISCOIII-003; IKT-202; JTX-4014; MCLA-134; MD-402; mDX-400; MGD-019; a monoclonal antibody to antagonize PDCD1 for oncology; a monoclonal antibody to antagonize PD-1 for oncology; an oncolytic virus to inhibit PD-1 for oncology; OT-2; PD-1 antagonist+ropeginterferon alfa-2b; PEGMP-7; PRS-332; RXI-762; STIA-1110; TSR-075; a vaccine to target HER2 and PD-1 for oncology; a vaccine to target PD-1 for oncology and autoimmune disorders; XmAb-23104; an antisense oligonucleotide to inhibit PD-1 for oncology; AT-16201; a bispecific monoclonal antibody to inhibit PD-1 for oncology; IMM-1802; monoclonal antibodies to antagonize PD-1 and CTLA-4 for solid tumor and hematological tumor; nivolumab biosimilar; a recombinant protein to agonize CD278 and CD28 and antagonize PD-1 for oncology; a recombinant protein to agonize PD-1 for autoimmune disorders and inflammatory disorders; SNA-01; SSI-361; YBL-006; AK-103; JY-034; AUR-012; BGB-108; drug to inhibit PD-1, Gal-9, and TIM-3 for solid tumor; ENUM-244C8; ENUM-388D4; MEDI-0680; monoclonal antibodies to antagonize PD-1 for metastatic melanoma and metastatic lung cancer; a monoclonal antibody to inhibit PD-1 for oncology; monoclonal antibodies to target CTLA-4 and PD-1 for oncology; a monoclonal antibody to antagonize PD-1 for NSCLC; monoclonal antibodies to inhibit PD-1 and TIM-3 for oncology; a monoclonal antibody to inhibit PD-1 for oncology; a recombinant protein to inhibit PD-1 and VEGF-A for hematological malignancies and solid tumor; a small molecule to antagonize PD-1 for oncology; Sym-016; inebilizumab+MEDI-0680; a vaccine to target PDL-1 and IDO for metastatic melanoma; an anti-PD-1 monoclonal antibody plus a cellular immunotherapy for glioblastoma; an antibody to antagonize PD-1 for oncology; monoclonal antibodies to inhibit PD-1/PD-L1 for hematological malignancies and bacterial infections; a monoclonal antibody to inhibit PD-1 for HIV; or a small molecule to inhibit PD-1 for solid tumor.

In certain embodiments, a composition of the present disclosure of the present disclosure is used in combination with a LAG3 inhibitor, such as LAG525, IMP321, IMP701, 9H12, BMS-986016, or any combination thereof.

In certain embodiments, a composition of the present disclosure is used in combination with an inhibitor of CTLA4. In particular embodiments, a composition of the present disclosure is used in combination with a CTLA4 specific antibody or binding fragment thereof, such as ipilimumab, tremelimumab, CTLA4-Ig fusion proteins (e.g., abatacept, belatacept), or any combination thereof.

In certain embodiments, a composition of the present disclosure is used in combination with a B7-H3 specific antibody or binding fragment thereof, such as enoblituzumab (MGA271), 376.96, or both. A B7-H4 antibody binding fragment may be a scFv or fusion protein thereof, as described in, for example, Dangaj et al., Cancer Res. 73:4820, 2013, as well as those described in U.S. Pat. No. 9,574,000 and PCT Patent Publication Nos. WO/201640724A1 and WO 2013/025779A1.

In certain embodiments, a composition of the present disclosure is used in combination with an inhibitor of CD244.

In certain embodiments, a composition of the present disclosure is used in combination with an inhibitor of BLTA, HVEM, CD160, or any combination thereof. Anti CD-160 antibodies are described in, for example, PCT Publication No. WO 2010/084158.

In certain embodiments, a composition of the present disclosure cell is used in combination with an inhibitor of TIM3.

In certain embodiments, a composition of the present disclosure is used in combination with an inhibitor of Gal9.

In certain embodiments, a composition of the present disclosure is used in combination with an inhibitor of adenosine signaling, such as a decoy adenosine receptor.

In certain embodiments, a composition of the present disclosure is used in combination with an inhibitor of A2aR.

In certain embodiments, a composition of the present disclosure is used in combination with an inhibitor of KIR, such as lirilumab (BMS-986015).

In certain embodiments, a composition of the present disclosure is used in combination with an inhibitor of an inhibitory cytokine (typically, a cytokine other than TGFβ) or Treg development or activity.

In certain embodiments, a composition of the present disclosure is used in combination with an IDO inhibitor, such as levo-1-methyl tryptophan, epacadostat (INCB024360; Liu et al., Blood 115:3520-30, 2010), ebselen (Terentis et al., Biochem. 49:591-600, 2010), indoximod, NLG919 (Mautino et al., American Association for Cancer Research 104th Annual Meeting 2013; Apr. 6-10, 2013), 1-methyl-tryptophan (1-MT)-tira-pazamine, or any combination thereof.

In certain embodiments, a composition of the present disclosure is used in combination with an arginase inhibitor, such as N (omega)-Nitro-L-arginine methyl ester (L-NAME), N-omega-hydroxy-nor-1-arginine (nor-NOHA), L-NOHA, 2 (S)-amino-6-boronohexanoic acid (ABH), S-(2-boronoethyl)-L-cysteine (BEC), or any combination thereof.

In certain embodiments, a composition of the present disclosure is used in combination with an inhibitor of VISTA, such as CA-170 (Curis, Lexington, Mass.).

In certain embodiments, a composition of the present disclosure is used in combination with an inhibitor of TIGIT such as, for example, COM902 (Compugen, Toronto, Ontario Canada), an inhibitor of CD155, such as, for example, COM701 (Compugen), or both.

In certain embodiments, a composition of the present disclosure is used in combination with an inhibitor of PVRIG, PVRL2, or both. Anti-PVRIG antibodies are described in, for example, PCT Publication No. WO 2016/134333. Anti-PVRL2 antibodies are described in, for example, PCT Publication No. WO 2017/021526.

In certain embodiments, a composition of the present disclosure is used in combination with a LAIR1 inhibitor.

In certain embodiments, a composition of the present disclosure is used in combination with an inhibitor of CEACAM-1, CEACAM-3, CEACAM-5, or any combination thereof.

In certain embodiments, a composition of the present disclosure is used in combination with an agent that increases the activity (i.e., is an agonist) of a stimulatory immune checkpoint molecule. For example a composition of the present disclosure can be used in combination with a CD137 (4-1BB) agonist (such as, for example, urelumab), a CD134 (OX-40) agonist (such as, for example, MEDI6469, MEDI6383, or MEDI0562), lenalidomide, pomalidomide, a CD27 agonist (such as, for example, CDX-1127), a CD28 agonist (such as, for example, TGN1412, CD80, or CD86), a CD40 agonist (such as, for example, CP-870,893, rhuCD40L, or SGN-40), a CD 122 agonist (such as, for example, IL-2) an agonist of GITR (such as, for example, humanized monoclonal antibodies described in PCT Patent Publication No. WO 2016/054638), an agonist of ICOS (CD278) (such as, for example, GSK3359609, mAb 88.2, JTX-2011, Icos 145-1, Icos 314-8, or any combination thereof). In any of the embodiments disclosed herein, a method may comprise administering a composition of the present disclosure with one or more agonist of a stimulatory immune checkpoint molecule, including any of the foregoing, singly or in any combination.

In certain embodiments, a combination therapy comprises a composition of the present disclosure and a secondary therapy comprising one or more of: an antibody or antigen binding-fragment thereof that is specific for a cancer antigen expressed by the non-inflamed solid tumor, a radiation treatment, a surgery, a chemotherapeutic agent, a cytokine, RNAi, or any combination thereof.

In certain embodiments, a combination therapy method comprises administering a composition of the present disclosure and further administering a radiation treatment or a surgery. Radiation therapy is well-known in the art and includes X-ray therapies, such as gamma-irradiation, and radiopharmaceutical therapies. Surgeries and surgical techniques appropriate to treating a given cancer in a subject are well-known to those of ordinary skill in the art.

In certain embodiments, a combination therapy method comprises administering a composition of the present disclosure and further administering a chemotherapeutic agent. A chemotherapeutic agent includes, but is not limited to, an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), and a DNA repair inhibitor. Illustrative chemotherapeutic agents include, without limitation, the following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, Cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, temozolamide, teniposide, triethylenethiophosphoramide and etoposide (VP 16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab, rituximab); chimeric antigen receptors; cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers, toxins such as Cholera toxin, ricin, Pseudomonas exotoxin, Bordetella pertussis adenylate cyclase toxin, or diphtheria toxin, and caspase activators; and chromatin disruptors.

Cytokines may be used to manipulate host immune response towards anticancer activity. See, e.g., Floros & Tarhini, Semin. Oncol. 42(4): 539-548, 2015. Cytokines useful for promoting immune anticancer or antitumor response include, for example, IFN-α, IL-2, IL-3, IL-4, IL-10, IL-12, IL-13, IL-15, IL-16, IL-17, IL-18, IL-21, IL-24, and GM-CSF, singly or in any combination with a composition of the present disclosure.

Also provided herein are methods for modulating an adoptive immunotherapy, wherein the methods comprise administering, to a subject who has previously received a modified host cell of the present disclosure that comprises a heterologous polynucleotide encoding a safety switch protein, a cognate compound of the safety switch protein in an amount effective to ablate in the subject the previously administered modified host cell.

In certain embodiments, the safety switch protein comprises tEGFR and the cognate compound is cetuximab, or the safety switch protein comprises iCasp9 and the cognate compound is AP1903 (e.g., dimerized AP1903), or the safety switch protein comprises a RQR polypeptide and the cognate compound is rituximab, or the safety switch protein comprises a myc binding domain and the cognate compound is an antibody specific for the myc binding domain.

In still further aspects, methods are provided for manufacturing a composition, or a unit dose of the present disclosure. In certain embodiments, the methods comprise combining (i) an aliquot of a host cell transduced with a vector of the present disclosure with (ii) a pharmaceutically acceptable carrier. In certain embodiments, vectors of the present disclosure are used to transfect/transduce a host cell (e.g., a T cell) for use in adoptive transfer therapy (e.g., targeting a cancer antigen).

In some embodiments, the methods further comprise, prior to the aliquotting, culturing the transduced host cell and selecting the transduced cell as having incorporated (i.e., expressing) the vector. In further embodiments, the methods comprise, following the culturing and selection and prior to the aliquotting, expanding the transduced host cell. In any of the embodiments of the instant methods, the manufactured composition or unit dose may be frozen or cryopreserved for later use. Any appropriate host cell can be used for manufacturing a composition or unit dose according to the instant methods, including, for example, a hematopoietic stem cell, a T cell, a primary T cell, a T cell line, a NK cell, or a NK-T cell. In specific embodiments, the methods comprise a host cell which is a CD8⁺ T cell, a CD4⁺ T cell, or both.

Also provided are any of the binding proteins, polynucleotides, expression vectors, host cells, host cell compositions, unit doses, and immunogenic polypeptides, taken singly or in any combination, for use in treating a disease or disorder associated with a KRAS G12D mutation or a KRAS G12V or a NRAS G12D mutation or a NRAS G12V mutation or a HRAS G12V mutation or a HRAS G12D mutation in a subject.

Also provided are any of the binding proteins, polynucleotides, expression vectors, host cells, host cell compositions, unit doses, and immunogenic polypeptides, taken singly or in any combination, for use the manufacture of a medicament for treating a disease or disorder associated with a KRAS G12D mutation or a KRAS G12V or a NRAS G12D mutation or a NRAS G12V mutation or a HRAS G12V mutation or a HRAS G12D mutation in a subject.

In certain embodiments, the disease or disorder comprises a cancer. In some embodiments, the cancer is a solid cancer or a hematological malignancy. In certain embodiments, the disease or disorder is selected from a pancreas cancer or carcinoma, optionally a pancreatic ductal adenocarcinoma (PDAC); a colorectal cancer or carcinoma; a lung cancer, optionally a non-small-cell lung carcinoma; a biliary cancer; an endometrial cancer or carcinoma; a cervical cancer; an ovarian cancer; a bladder cancer; a liver cancer; a myeloid leukemia, optionally myeloid leukemia such as acute myeloid leukemia; a myelodysplastic syndrome; a lymphoma such as Non-Hodgkin lymphoma; Chronic Melyomonocytic Leukemia; Acute Lymphoblastic Leukemia (ALL); a cancer of the urinary tract; a cancer of the small intestine; a breast cancer or carcinoma; a melanoma (optionally a cutaneous melanoma, an anal melanoma, or a mucosal melanoma); a glioma; a poorly differentiated thyroid gland carcinoma; a neuroblastoma; a histiocytic and dendritic cell neoplasm; neurofibromatisis Type 1; rhabdomyosarcoma; a soft tissue sarcoma; a bladder carcinoma; a sarcoma; a glioblastoma; a squamous cell lung carcinoma; an anaplastic astrocytoma; chronic myeloid leukemia; diffuse large B-cell lymphoma; double-hit lymphoma; head and neck carcinoma; head and neck squamous cell carcinoma; hepatocellular carcinoma; malignant peripheral nerve sheath tumor; mantle cell lymphoma; myelodysplastic/myeloproliferative neoplasm, unclassifiable; peripheral T cell lymphoma; prostate carcinoma; refractory anemia with excess blasts-2; renal cell carcinoma; rhabdoid tumor; schwannoma; secondary AML; small cell lung carcinoma; therapy-related AML; thymic carcinoma; thyroid gland follicular carcinoma; malignant thyroid gland neoplasm; thyroid gland carcinoma; thyroid gland adenocarcinoma; urothelial carcinoma; or thyroid gland papillary carcinoma. In some embodiments, the method comprises parenteral or intravenous administration of the subject composition. In some embodiments, the method comprises administering a plurality of doses of the binding protein, polynucleotide, expression vector, host cell, host cell composition, unit dose, and/or immunogenic polypeptide the subject.

In certain embodiments, the plurality of doses is administered at intervals between administrations of about two to about four weeks.

In certain embodiments, the composition comprises the modified host cell. In some embodiments, the method comprises administering the modified host cell to the subject at a dose of about 10⁴ cells/kg to about 10¹¹ cells/kg.

In certain embodiments, wherein the method further comprises administering a cytokine to the subject. In some embodiments, the cytokine comprises IL-2, IL-15, or IL-21.

In certain embodiments, the subject has received or is receiving an immune checkpoint inhibitor and/or an agonist of a stimulatory immune checkpoint agent.

Also provided are methods that comprise introducing, into a host (e.g., T) cell, a polynucleotide encoding a binding protein of the present disclosure.

The present disclosure also provides the following, non-limiting, enumerated Embodiments.

Embodiment 1. A binding protein comprising:
(a) a T cell receptor (TCR) α chain variable (Vα) domain comprising the complementarity determining region 3 (CDR3α) amino acid sequence set forth in any one of SEQ ID NOs.: 16, 17, 42, and 43, or a variant thereof having one, two, or three, optionally conservative, amino acid substitutions; and/or
(b) a TCR β chain variable (Vβ) domain comprising the CDR3β amino acid sequence set forth in any one of SEQ ID NOs.: 26, 27, 52, and 53, or a variant thereof having one, two, or three, optionally conservative, amino acid substitutions, wherein the binding protein is capable of binding to a peptide: HLA complex, wherein the peptide comprises, consists essentially of, or consists of the amino acid sequence VVVGAVGVGK (SEQ ID NO.: 2) or VVGAVGVGK (SEQ ID NO.: 3) and wherein the HLA comprises an HLA-A*11.

Embodiment 2. The binding protein of Embodiment 1, wherein the HLA comprises HLA-A*11:01.

Embodiment 3. The binding protein of Embodiment 1 or 2, wherein the Vα domain and/or the Vβ domain is human, humanized, or chimeric, and is preferably human.

Embodiment 4. The binding protein of any one of Embodiments 1-3, comprising the CDR3α and CDR3β amino acid sequences set forth in SEQ ID NOs.: (i) 17 and 27, respectively, or variants thereof having one, two, or three, optionally conservative, amino acid substitutions; (ii) 16 and 26, respectively, or variants thereof having one, two, or three, optionally conservative, amino acid substitutions; (iii) 53 and 43, respectively, or variants thereof having one, two, or three, optionally conservative, amino acid substitutions; or (iv) 52 and 42, respectively, or variants thereof having one, two, or three, optionally conservative, amino acid substitutions.

Embodiment 5. The binding protein of any one of Embodiments 1-4, comprising: (i) in the Vα domain, the CDR1α amino acid sequence set forth in SEQ ID NO.: 14 or 40, or a variant thereof having one or two, optionally conservative, amino acid substitutions; (ii) in the Vα domain, the CDR2α amino acid sequence set forth in SEQ ID NO.: 15 or 41, or a variant thereof having one or two, optionally conservative, amino acid substitutions; (iii) in the Vβ domain, the CDR1β acid sequence set forth in SEQ ID NO.: 24 or 50, or a variant thereof having one or two, optionally conservative, amino acid substitutions; (iv) in the Vβ domain, the CDR2β acid sequence set forth in SEQ ID NO.: 25 or 51, or a variant thereof having one or two, optionally conservative, amino acid substitutions; or (v) any combination of (i)-(iv).

Embodiment 6. The binding protein of any one of Embodiments 1-5, comprising the CDR1α, CDR2α, CDR3α, CDR1β, CDR2β, and CDR3β amino acid sequences set forth in SEQ ID NOs.: 14, 15, 16 or 17, 24, 25, and 26 or 27, respectively.

Embodiment 7. The binding protein of any one of Embodiments 1-5, comprising the CDR1α, CDR2α, CDR3α, CDR1β, CDR2β, and CDR3β amino acid sequences set forth in SEQ ID NOs.: 40, 41, 42 or 43, 50, 51, and 52 or 53, respectively.

Embodiment 8. The binding protein of any one of Embodiments 1-7, wherein:
(i) the Vα domain comprises, consists essentially of, or consists of an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the amino acid sequence set forth in SEQ ID NO.: 13 or 39; and/or
(ii) the Vβ domain comprises, consists essentially of, or consists of an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the amino acid sequence set forth in SEQ ID NO.: 23 or 49.

Embodiment 9. The binding protein of any one of Embodiments 1-8, wherein the Vα domain comprises, consists essentially of, or consists of an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the amino acid sequence set forth in SEQ ID NO.: 13, and wherein the Vβ domain comprises, consists essentially of, or consists of an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the amino acid sequence set forth in SEQ ID NO.: 23, wherein, optionally, the binding protein comprises the amino acid sequence set forth in SEQ ID NO.: 154.

Embodiment 10. The binding protein of any one of Embodiments 1-8, wherein the Vα domain comprises, consists essentially of, or consists of an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the amino acid sequence set forth in SEQ ID NO.: 39, and wherein the Vβ domain comprises, consists essentially of, or consists of an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the amino acid sequence set forth in SEQ ID NO.: 49.

Embodiment 11. The binding protein of any one of Embodiments 1-10, wherein the Vα domain comprises, consists essentially of, or consists of the amino acid sequence set forth in SEQ ID NO.: 13 and the Vβ domain comprises, consists essentially of, or consists of amino acid sequence set forth in SEQ ID NO.: 23, wherein, optionally, the binding protein comprises the amino acid sequence set forth in SEQ ID NO.: 154.

Embodiment 12. The binding protein of any one of Embodiments 1-10, wherein the Vα domain comprises, consists essentially of, or consists of the amino acid sequence set forth in SEQ ID NO.: 39 and the Vβ domain comprises, consists essentially of, or consists of amino acid sequence set forth in SEQ ID NO.: 49.

Embodiment 13. The binding protein of any one of Embodiments 1-12, further comprising a TCR α chain constant domain (Cα) and/or a TCR β chain constant domain (Cβ).

Embodiment 14. The binding protein of Embodiment 13, wherein the Cα comprises, consists essentially of, or consists of an amino acid sequence having at least 90%, at least 91%, at least 92%, at least 93%, at least 94%, at least 95%, at least 96%, at least 97%, at least 98%, or at least 99% identity to, or comprising or consisting of, the amino acid sequence set forth in any one of SEQ ID NOs.: 18, 19, 44, 45, and 69.

Embodiment 15. The binding protein of Embodiment 13 or 14, wherein the Cβ comprises, consists essentially of, or consists of an amino acid sequence having at least 90%, at least 91%, at least 92%, at least 93%, at least 94%, at least 95%, at least 96%, at least 97%, at least 98%, or at least 99% identity to, or comprising or consisting of, the amino acid sequence set forth in any one of SEQ ID NOs.: 28, 29, 54, 55, and 70-73.

Embodiment 16. The binding protein of any one of Embodiments 13-15, wherein the Cα and the Cβ comprise or consist of amino acid sequences having at least 90%, at least 91%, at least 92%, at least 93%, at least 94%, at least 95%, at least 96%, at least 97%, at least 98%, or at least 99% identity to, or comprising or consisting of, the amino acid sequences set forth in SEQ ID NOs.:
(i) 18 and 28, respectively;
(ii) 19 and 29, respectively;
(iii) 44 and 54, respectively; or
(iv) 45 and 55, respectively.

Embodiment 17. The binding protein of any one of Embodiments 1-16, comprising a TCR α chain and a TCR β chain, wherein the TCR α chain and the TCR β chain comprise or consist of amino acid sequences having at least 90%, at least 91%, at least 92%, at least 93%, at least 94%, at least 95%, at least 96%, at least 97%, at least 98%, or at least 99% identity to, or comprising or consisting of, the amino acid sequences set forth in:
(i) SEQ ID NOs.: 12 and 22, respectively;
(ii) SEQ ID NOs.: 20 and 30 or 155, respectively;
(iii) SEQ ID NOS.: 12 and 30 or 155, respectively;
(iv) SEQ ID NOs.: 20 and 22, respectively;
(v) SEQ ID NOs.: 38 and 48, respectively;
(vi) SEQ ID NOs.: 46 and 56, respectively;
(vii) SEQ ID NOs.: 38 and 56, respectively; or
(viii) SEQ ID NOs.: 46 and 48, respectively.

Embodiment 18. The binding protein of any one of Embodiments 1-17, wherein the binding protein comprises a TCR, a single-chain TCR (scTCR), a single-chain T cell receptor variable fragment (scTv), or a chimeric antigen receptor (CAR).

Embodiment 19. The binding protein of Embodiment 18, wherein the binding protein comprises a TCR.

Embodiment 20. An isolated polynucleotide encoding the binding protein of any one of Embodiments 1-19.

Embodiment 21. The polynucleotide of Embodiment 20, comprising a polynucleotide having at least 75%, at least 80%, at least 85%, at least 90%, at least 91%, at least 92%, at least 93%, at least 94%, at least 95%, at least 96%, at least 97%, at least 98%, or at least 99% identity to, or comprising or consisting of, the polynucleotide sequence set forth in any one of SEQ ID NOs.: 5-10 and 33-36, or any combination thereof.

Embodiment 22. The polynucleotide of Embodiment 20 or 21, further comprising:
(i) a polynucleotide encoding a polypeptide that comprises an extracellular portion of a CD8 co-receptor α chain, wherein, optionally, the encoded polypeptide is or comprises a CD8 co-receptor α chain;
(ii) a polynucleotide encoding a polypeptide that comprises an extracellular portion of a CD8 co-receptor β chain, wherein, optionally, the encoded polypeptide is or comprises a CD8 co-receptor β chain; or
(iii) a polynucleotide of (i) and a polynucleotide of (ii).

Embodiment 23. The polynucleotide of Embodiment 22, comprising:
(a) the polynucleotide encoding a polypeptide comprising an extracellular portion of a CD8 co-receptor α chain;
(b) the polynucleotide encoding a polypeptide comprising an extracellular portion of a CD8 co-receptor β chain; and
(c) a polynucleotide encoding a self-cleaving peptide disposed between the polynucleotide of (a) and the polynucleotide of (b).

Embodiment 24. The polynucleotide of Embodiment 22 or 23, further comprising a polynucleotide that encodes a self-cleaving peptide and is disposed between:
(1) the polynucleotide encoding a binding protein and the polynucleotide encoding a polypeptide comprising an extracellular portion of a CD8 co-receptor α chain; and/or (2) the polynucleotide encoding a binding protein and the polynucleotide encoding a polypeptide comprising an extracellular portion of a CD8 co-receptor β chain.

Embodiment 25. The polynucleotide of any one of Embodiments 22-24, comprising, operably linked in-frame:
(i) (pnCD8α)-(pnSCP₁)-(pnCD8β)-(pnSCP₂)-(pnBP);
(ii) (pnCD8β)-(pnSCP₁)-(pnCD8α)-(pnSCP₂)-(pnBP);
(iii) (pnBP)-(pnSCP₁)-(pnCD8α)-(pnSCP₂)-(pnCD8β);
(iv) (pnBP)-(pnSCP₁)-(pnCD8β)-(pnSCP₂)-(pnCD8α);
(v) (pnCD8α)-(pnSCP₁)-(pnBP)-(pnSCP₂)-(pnCD8β); or
(vi) (pnCD8β)-(pnSCP₁)-(pnBP)-(pnSCP₂)-(pnCD8α),
wherein pnCD8α is the polynucleotide encoding a polypeptide that comprises an extracellular portion of a CD8 co-receptor α chain,
wherein pnCD8β is the polynucleotide encoding a polypeptide that comprises an extracellular portion of a CD8 co-receptor α chain,
wherein pnBP is the polynucleotide encoding a binding protein,
and wherein pnSCP₁ and pnSCP₂ are each independently a polynucleotide encoding a self-cleaving peptide, wherein the polynucleotides and/or the encoded self-cleaving peptides are optionally the same or different.

Embodiment 26. The polynucleotide of any one of Embodiments 22-25, wherein the encoded binding protein comprises a TCRα chain and a TCRβ chain, wherein the polynucleotide comprises a polynucleotide encoding a self-cleaving peptide disposed between the polynucleotide encoding a TCRα chain and the polynucleotide encoding a TCRβ chain.

Embodiment 27. The polynucleotide of Embodiment 26, comprising, operably linked in-frame:
(i) (pnCD8α)-(pnSCP₁)-(pnCD8β)-(pnSCP₂)-(pnTCRβ)-(pnSCP₃)-(pnTCRα);
(ii) (pnCD8β)-(pnSCP₁)-(pnCD8α)-(pnSCP₂)-(pnTCRβ)-(pnSCP₃)-(pnTCRα);
(iii) (pnCD8α)-(pnSCP₁)-(pnCD8β)-(pnSCP₂)-(pnTCRα)-(pnSCP₃)-(pnTCRβ);
(iv) (pnCD8β)-(pnSCP₁)-(pnCD8α)-(pnSCP₂)-(pnTCRα)-(pnSCP₃)-(pnTCRβ);
(v) (pnTCRβ)-(pnSCP₁)-(pnTCRα)-(pnSCP₂)-(pnCD8α)-(pnSCP₃)-(pnCD8β);
(vi) (pnTCRβ)-(pnSCP₁)-(pnTCRα)-(pnSCP₂)-(pnCD8β)-(pnSCP₃)-(pnCD8α);
(vii) (pnTCRα)-(pnSCP₁)-(pnTCRβ)-(pnSCP₂)-(pnCD8α)-(pnSCP₃)-(pnCD8β);
(viii) (pnTCRα)-(pnSCP₁)-(pnTCRβ)-(pnSCP₂)-(pnCD8β)-(pnSCP₃)-(pnCD8α),
wherein pnCD8α is the polynucleotide encoding a polypeptide that comprises an extracellular portion of a CD8 co-receptor α chain,
wherein pnCD8β is the polynucleotide encoding a polypeptide that comprises an extracellular portion of a CD8 co-receptor α chain,
wherein pnTCRα is the polynucleotide encoding a TCR α chain,
wherein pnTCRβ is the polynucleotide encoding a TCR β chain,
and wherein pnSCP₁, pnSCP₂, and pnSCP₃ are each independently a polynucleotide encoding a self-cleaving peptide, wherein the polynucleotides and/or the encoded self-cleaving peptides are optionally the same or different.

Embodiment 28. The polynucleotide of any one of Embodiments 20-27, encoding an amino acid sequence having at least 90%, at least 91%, at least 92%, at least 93%, at least 94%, at least 95%, at least 96%, at least 97%, at least 98%, or at least 99% identity to, or comprising or consisting of the amino acid sequence set forth in any one of SEQ ID NOs.: 11, 21, 37, 47, 31, 32, 57, and 58.

Embodiment 29. The polynucleotide of Embodiment 28, encoding (i) an amino acid sequence having at least 90%, at least 91%, at least 92%, at least 93%, at least 94%, at least 95%, at least 96%, at least 97%, at least 98%, or at least 99% identity to, or comprising or consisting of the amino acid sequence set forth in SEQ ID NO.: 11, and (ii) an amino acid sequence having at least 90%, at least 91%, at least 92%, at least 93%, at least 94%, at least 95%, at least 96%, at least 97%, at least 98%, or at least 99% identity to, or comprising or consisting of the amino acid sequence set forth in SEQ ID NO.: 21.

Embodiment 30. The polynucleotide of Embodiment 29, encoding (i) an amino acid sequence having at least 90%, at least 91%, at least 92%, at least 93%, at least 94%, at least 95%, at least 96%, at least 97%, at least 98%, or at least 99% identity to, or comprising or consisting of the amino acid sequence set forth in SEQ ID NO.: 37, and (ii) an amino acid sequence having at least 90%, at least 91%, at least 92%, at least 93%, at least 94%, at least 95%, at least 96%, at least 97%, at least 98%, or at least 99% identity to, or comprising or consisting of the amino acid sequence set forth in SEQ ID NO.: 47.

Embodiment 31. The polynucleotide of any one of Embodiments 20-30, which is or comprises a polynucleotide sequence that is codon optimized for expression in a host cell, wherein, optionally, the host cell is a human immune system cell, and wherein, further optionally, is a T cell.

Embodiment 32. An expression vector, comprising a polynucleotide of any one of Embodiments 20-31 operably linked to an expression control sequence.

Embodiment 33. The expression vector of Embodiment 32, wherein the vector is capable of delivering the polynucleotide to a host cell.

Embodiment 34. The expression vector of Embodiment 33, wherein the host cell is a hematopoietic progenitor cell or a human immune system cell.

Embodiment 35. The expression vector of Embodiment 34, wherein the human immune system cell is a CD4⁺ T cell, a CD8⁺ T cell, a CD4⁻CD8⁻ double negative T cell, a γδ T cell, a natural killer cell, a natural killer T cell, a macrophage, a monocyte, a dendritic cell, or any combination thereof.

Embodiment 36. The expression vector of Embodiment 35, wherein the T cell is a naïve T cell, a central memory T cell, an effector memory T cell, or any combination thereof. Embodiment 37. The expression vector of any one of Embodiments 32-36, wherein the vector is a viral vector.

Embodiment 38. The expression vector of Embodiment 37, wherein the viral vector is a lentiviral vector or a γ-retroviral vector.

Embodiment 39. A host cell modified to comprise the polynucleotide of any one of Embodiments 20-31 and/or the expression vector of any one of Embodiments 32-38 and/or to express the binding protein of any one of Embodiments 1-19.

Embodiment 40. The host cell of Embodiment 39, wherein the modified cell comprises a hematopoietic progenitor cell and/or or human immune cell.

Embodiment 41. The host cell of Embodiment 40, wherein the immune cell comprises a T cell, a NK cell, a NK-T cell, a dendritic cell, a macrophage, a monocyte, or any combination thereof.

Embodiment 42. The host cell of Embodiment 41, wherein the immune cell comprises a CD4⁺ T cell, a CD8⁺ T cell, a CD4⁻CD8⁻ double negative T cell, a γδ T cell, a naïve T cell, a central memory T cell, a stem cell memory T cell, an effector memory T cell, or any combination thereof,
wherein, optionally, the immune cell comprises a CD4⁺ T cell and a CD8⁺ T cell, wherein, further optionally, the CD4⁺ T cell, the CD8⁺ T cell, or both comprise (i) a polynucleotide encoding a polypeptide that comprises an extracellular portion of a CD8 co-receptor α chain, wherein, optionally, the encoded polypeptide is or comprises a CD8 co-receptor α chain; (ii) a polynucleotide encoding a polypeptide that comprises an extracellular portion of a CD8 co-receptor β chain, wherein, optionally, the encoded polypeptide is or comprises a CD8 co-receptor β chain; or (iii) a polynucleotide of (i) and a polynucleotide of (ii).

Embodiment 43. The host cell of any one of Embodiments 39-42, wherein the modified cell comprises a chromosomal gene knockout of a PD-1 gene; a LAG3 gene; a TIM3 gene; a CTLA4 gene; an HLA component gene; a TIGIT gene; a TCR component gene, a FasL gene, or any combination thereof.

Embodiment 44. The host cell of Embodiment 43, wherein the chromosomal gene knockout comprises a knockout of an HLA component gene selected from an α1 macroglobulin gene, an α2 macroglobulin gene, an α3 macroglobulin gene, a β1 microglobulin gene, or a β2 microglobulin gene.

Embodiment 45. The host cell of Embodiment 33 or 34, wherein the chromosomal gene knockout comprises a knockout of a TCR component gene selected from a TCR α variable region gene, a TCR β variable region gene, a TCR constant region gene, or a combination thereof.

Embodiment 46. A composition comprising the host cell of any one of Embodiments 39-45 and a pharmaceutically acceptable carrier, diluent, or excipient.

Embodiment 47. The composition of Embodiment 46, comprising at least about 30% modified CD4⁺ T cells, combined with (ii) a composition comprising at least about 30% modified CD8⁺ T cells, in about a 1:1 ratio.

Embodiment 48. The composition of Embodiment 46 or 47, wherein the composition contains substantially no naïve T cells.

Embodiment 49. A composition comprising:
(i) the binding protein of any one of Embodiments 1-19;
(ii) the polynucleotide of any one of Embodiments 20-31;
(iii) the expression vector of any one of Embodiments 32-38; and/or
(iv) the host cell of any one of Embodiments 39-45,
and a pharmaceutically acceptable carrier, excipient, or diluent.

Embodiment 50. A method for treating a disease or disorder associated with a KRAS G12V mutation or a NRAS G12V mutation or a HRAS G12V mutation in a subject, the method comprising administering to the subject an effective amount of:
(i) the binding protein of any one of Embodiments 1-19;
(ii) the polynucleotide of any one of Embodiments 20-31;
(iii) the expression vector of any one of Embodiments 32-38;
(iv) the host cell of any one of Embodiments 39-45, wherein, optionally, the host cell comprises a CD8+ T cell, a CD4+ T cell, or both, and wherein, optionally, the host cell is autologous, allogeneic, or syngeneic to the subject; and/or
(v) the composition of any one of Embodiments 46-49.

Embodiment 51. The method of Embodiment 50, wherein the disease or disorder comprises a cancer, wherein the cancer is optionally a solid cancer or a hematological malignancy.

Embodiment 52. The method of Embodiment 50 or 51, wherein the disease or disorder is selected from a pancreas cancer or carcinoma, optionally a pancreatic ductal adenocarcinoma (PDAC); a colorectal cancer or carcinoma; a lung cancer, optionally a non-small-cell lung carcinoma; a biliary cancer; an endometrial cancer or carcinoma; a cervical cancer; an ovarian cancer; a bladder cancer; a liver cancer; a myeloid leukemia, optionally myeloid leukemia such as acute myeloid leukemia; a myelodysplastic syndrome; a lymphoma such as Non-Hodgkin lymphoma; Chronic Melyomonocytic Leukemia; Acute Lymphoblastic Leukemia (ALL); a cancer of the urinary tract; a cancer of the small intestine; a breast cancer or carcinoma; a melanoma (optionally a cutaneous melanoma, an anal melanoma, or a mucosal melanoma); a glioma; a poorly differentiated thyroid gland carcinoma; a neuroblastoma; a histiocytic and dendritic cell neoplasm; neurofibromatisis Type 1; rhabdomyosarcoma; a soft tissue sarcoma; a bladder carcinoma; a sarcoma; a glioblastoma; a squamous cell lung carcinoma; an anaplastic astrocytoma; chronic myeloid leukemia; diffuse large B-cell lymphoma; double-hit lymphoma; head and neck carcinoma; head and neck squamous cell carcinoma; hepatocellular carcinoma; malignant peripheral nerve sheath tumor; mantle cell lymphoma; myelodysplastic/myeloproliferative neoplasm, unclassifiable; peripheral T cell lymphoma; prostate carcinoma; refractory anemia with excess blasts-2; renal cell carcinoma; rhabdoid tumor; schwannoma; secondary AML; small cell lung carcinoma; therapy-related AML; thymic carcinoma; thyroid gland follicular carcinoma; malignant thyroid gland neoplasm; thyroid gland carcinoma; thyroid gland adenocarcinoma; urothelial carcinoma; or thyroid gland papillary carcinoma.

Embodiment 53. The method of any one of Embodiments 50-52, wherein the binding protein, polynucleotide, vector, host cell, or composition is administered to the subject parenterally or intravenously.

Embodiment 54. The method of any one of Embodiments 50-53, wherein the method comprises administering a plurality of doses of any one or more of (i)-(v) to the subject. Embodiment 55. The method of Embodiment 54, wherein the plurality of doses is administered at intervals between administrations of about two to about four weeks.

Embodiment 56. The method of any one of Embodiments 50-55, wherein the composition comprises the host cell or the composition comprising the host cell, and wherein the method comprises administering the host cell or composition to the subject at a dose of about 10⁴ cells/kg to about 10¹¹ cells/kg.

Embodiment 57. The method of any one of Embodiments 50-56, further comprising determining that the subject expresses HLA-A*11, optionally HLA-A*11:01, prior to administering the binding protein, polynucleotide, vector, host cell, or composition.

Embodiment 58. The method of any one of Embodiments 50-57, wherein the method further comprises administering a cytokine to the subject.

Embodiment 59. The method of Embodiment 58, wherein the cytokine comprises IL-2, IL-15, or IL-21.

Embodiment 60. The method of any one of Embodiments 50-59, wherein the subject has received or is receiving an immune checkpoint inhibitor and/or an agonist of a stimulatory immune checkpoint agent.

Embodiment 61. The binding protein of any one of Embodiments 1-19, the polynucleotide of any one of Embodiments 20-31, the expression vector of any one of Embodiments 32-38, the host cell of any one of Embodiments 39-45, wherein, optionally, the host cell comprises a CD8+ T cell, a CD4+ T cell, or both, and/or the composition of any one of Embodiments 46-49, for use in a method for treating a disease or disorder associated with a KRAS G12V or a NRAS G12V mutation or a HRAS G12V mutation in a subject, wherein, optionally, the disease or disorder comprises a cancer, wherein, further optionally, the cancer is a solid cancer or a hematological malignancy, and wherein, optionally, the disease or disorder is selected from a pancreas cancer or carcinoma, optionally a pancreatic ductal adenocarcinoma (PDAC); a colorectal cancer or carcinoma; a lung cancer, optionally a non-small-cell lung carcinoma; a biliary cancer; an endometrial cancer or carcinoma; a cervical cancer; an ovarian cancer; a bladder cancer; a liver cancer; a myeloid leukemia, optionally myeloid leukemia such as acute myeloid leukemia; a myelodysplastic syndrome; a lymphoma such as Non-Hodgkin lymphoma; Chronic Melyomonocytic Leukemia; Acute Lymphoblastic Leukemia (ALL); a cancer of the urinary tract; a cancer of the small intestine; a breast cancer or carcinoma; a melanoma (optionally a cutaneous melanoma, an anal melanoma, or a mucosal melanoma); a glioma; a poorly differentiated thyroid gland carcinoma; a neuroblastoma; a histiocytic and dendritic cell neoplasm; neurofibromatisis Type 1; rhabdomyosarcoma; a soft tissue sarcoma; a bladder carcinoma; a sarcoma; a glioblastoma; a squamous cell lung carcinoma; an anaplastic astrocytoma; chronic myeloid leukemia; diffuse large B-cell lymphoma; double-hit lymphoma; head and neck carcinoma; head and neck squamous cell carcinoma; hepatocellular carcinoma; malignant peripheral nerve sheath tumor; mantle cell lymphoma; myelodysplastic/myeloproliferative neoplasm, unclassifiable; peripheral T cell lymphoma; prostate carcinoma; refractory anemia with excess blasts-2; renal cell carcinoma; rhabdoid tumor; schwannoma; secondary AML; small cell lung carcinoma; therapy-related AML; thymic carcinoma; thyroid gland follicular carcinoma; malignant thyroid gland neoplasm; thyroid gland carcinoma; thyroid gland adenocarcinoma; urothelial carcinoma; or thyroid gland papillary carcinoma.

Embodiment 62. The binding protein of any one of Embodiments 1-19, the polynucleotide of any one of Embodiments 20-31, the expression vector of any one of Embodiments 32-38, the host cell of any one of Embodiments 39-45, wherein, optionally, the host cell comprises a CD8+ T cell, a CD4+ T cell, or both, and/or the composition of any one of Embodiments 46-49, for use the manufacture of a medicament for treating a disease or disorder associated with a KRAS G12V or a NRAS G12V mutation or a HRAS G12V mutation in a subject, wherein, optionally, the disease or disorder comprises a cancer, wherein, further optionally, the cancer is a solid cancer or a hematological malignancy. and, wherein, optionally, the disease or disorder is selected from a pancreas cancer or carcinoma, optionally a pancreatic ductal adenocarcinoma (PDAC); a colorectal cancer or carcinoma; a lung cancer, optionally a non-small-cell lung carcinoma; a biliary cancer; an endometrial cancer or carcinoma; a cervical cancer; an ovarian cancer; a bladder cancer; a liver cancer; a myeloid leukemia, optionally myeloid leukemia such as acute myeloid leukemia; a myelodysplastic syndrome; a lymphoma such as Non-Hodgkin lymphoma; Chronic Melyomonocytic Leukemia; Acute Lymphoblastic Leukemia (ALL); a cancer of the urinary tract; a cancer of the small intestine; a breast cancer or carcinoma; a melanoma (optionally a cutaneous melanoma, an anal melanoma, or a mucosal melanoma); a glioma; a poorly differentiated thyroid gland carcinoma; a neuroblastoma; a histiocytic and dendritic cell neoplasm; neurofibromatisis Type 1; rhabdomyosarcoma; a soft tissue sarcoma; a bladder carcinoma; a sarcoma; a glioblastoma; a squamous cell lung carcinoma; an anaplastic astrocytoma; chronic myeloid leukemia; diffuse large B-cell lymphoma; double-hit lymphoma; head and neck carcinoma; head and neck squamous cell carcinoma; hepatocellular carcinoma; malignant peripheral nerve sheath tumor; mantle cell lymphoma; myelodysplastic/myeloproliferative neoplasm, unclassifiable; peripheral T cell lymphoma; prostate carcinoma; refractory anemia with excess blasts-2; renal cell carcinoma; rhabdoid tumor; schwannoma; secondary AML; small cell lung carcinoma; therapy-related AML; thymic carcinoma; thyroid gland follicular carcinoma; malignant thyroid gland neoplasm; thyroid gland carcinoma; thyroid gland adenocarcinoma; urothelial carcinoma; or thyroid gland papillary carcinoma.

Embodiment 1a. A binding protein comprising:
(a) a T cell receptor (TCR) α chain variable (Vα) domain comprising the complementarity determining region 3 (CDR3α) amino acid sequence set forth in any one of SEQ ID NOs.: 16, 17, 42, and 43, or a variant thereof having one, two, or three, optionally conservative, amino acid substitutions; and/or
(b) a TCR β chain variable (Vβ) domain comprising the CDR3β amino acid sequence set forth in any one of SEQ ID NOs.: 26, 27, 52, and 53, or a variant thereof having one, two, or three, optionally conservative, amino acid substitutions, wherein the binding protein is capable of binding to a peptide: HLA complex, wherein the peptide comprises, consists essentially of, or consists of the amino acid sequence VVVGAVGVGK (SEQ ID NO.: 2) or VVGAVGVGK (SEQ ID NO.: 3) and wherein the HLA comprises an HLA-A*11.

Embodiment 2a. The binding protein of Embodiment 1a, wherein the HLA comprises HLA-A*11:01.

Embodiment 3a. The binding protein of Embodiment 1a or 2a, wherein the Vα domain and/or the Vβ domain is human, humanized, or chimeric, and is preferably human.

Embodiment 4a. The binding protein of any one of Embodiments 1a-3a, comprising the CDR3α and CDR3β amino acid sequences set forth in SEQ ID NOs.: (i) 17 and 27, respectively, or variants thereof having one, two, or three, optionally conservative, amino acid substitutions; (ii) 16 and 26, respectively, or variants thereof having one, two, or three, optionally conservative, amino acid substitutions; (iii) 53 and 43, respectively, or variants thereof having one, two, or three, optionally conservative, amino acid substitutions; or (iv) 52 and 42, respectively, or variants thereof having one, two, or three, optionally conservative, amino acid substitutions.

Embodiment 5a. The binding protein of any one of Embodiments 1a-4a, comprising: (i) in the Vα domain, the CDR1α amino acid sequence set forth in SEQ ID NO.: 14 or 40, or a variant thereof having one or two, optionally conservative, amino acid substitutions; (ii) in the Vα domain, the CDR2α amino acid sequence set forth in SEQ ID NO.: 15 or 41, or a variant thereof having one or two, optionally conservative, amino acid substitutions; (iii) in the Vβ domain, the CDR1β acid sequence set forth in SEQ ID NO.: 24 or 50, or a variant thereof having one or two, optionally conservative, amino acid substitutions; (iv) in the Vβ domain, the CDR2 acid sequence set forth in SEQ ID NO.: 25 or 51, or a variant thereof having one or two, optionally conservative, amino acid substitutions; or (v) any combination of (i)-(iv).

Embodiment 6a. The binding protein of any one of Embodiments 1a-5a, comprising the CDR1α, CDR2α, CDR3α, CDR1β, CDR2β, and CDR3 amino acid sequences set forth in SEQ ID NOs.: 14, 15, 16 or 17, 24, 25, and 26 or 27, respectively.

Embodiment 7a. The binding protein of any one of Embodiments 1a-5a, comprising the CDR1α, CDR2α, CDR3α, CDR1β, CDR2β, and CDR3β amino acid sequences set forth in SEQ ID NOs.: 40, 41, 42 or 43, 50, 51, and 52 or 53, respectively.

Embodiment 8a. The binding protein of any one of Embodiments 1a-7a, wherein:
(i) the Vα domain comprises, consists essentially of, or consists of an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the amino acid sequence set forth in SEQ ID NO.: 13 or 39; and/or
(ii) the Vβ domain comprises, consists essentially of, or consists of an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the amino acid sequence set forth in SEQ ID NO.: 23 or 49.

Embodiment 9a. The binding protein of any one of Embodiments 1a-8a, wherein the Vα domain comprises, consists essentially of, or consists of an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the amino acid sequence set forth in SEQ ID NO.: 13, and wherein the Vβ domain comprises, consists essentially of, or consists of an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the amino acid sequence set forth in SEQ ID NO.: 23.

Embodiment 10a. The binding protein of any one of Embodiments 1a-8a, wherein the Vα domain comprises, consists essentially of, or consists of an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the amino acid sequence set forth in SEQ ID NO.: 39, and wherein the Vβ domain comprises, consists essentially of, or consists of an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the amino acid sequence set forth in SEQ ID NO.: 49.

Embodiment 11a. The binding protein of any one of Embodiments 1a-10a, wherein the Vα domain comprises, consists essentially of, or consists of the amino acid sequence set forth in SEQ ID NO.: 13 and the Vβ domain comprises, consists essentially of, or consists of amino acid sequence set forth in SEQ ID NO.: 23.

Embodiment 12a. The binding protein of any one of Embodiments 1a-10a, wherein the Vα domain comprises, consists essentially of, or consists of the amino acid sequence set forth in SEQ ID NO.: 39 and the Vβ domain comprises, consists essentially of, or consists of amino acid sequence set forth in SEQ ID NO.: 49.

Embodiment 13a. The binding protein of any one of Embodiments 1a-12a, further comprising a TCR α chain constant domain (Cα) and/or a TCR β chain constant domain (Cβ).

Embodiment 14a. The binding protein of Embodiment 13a, wherein the Ca comprises, consists essentially of, or consists of an amino acid sequence having at least 90%, at least 91%, at least 92%, at least 93%, at least 94%, at least 95%, at least 96%, at least 97%, at least 98%, or at least 99% identity to, or comprising or consisting of, the amino acid sequence set forth in any one of SEQ ID NOs.: 18, 19, 44, 45, and 69.

Embodiment 15a. The binding protein of Embodiment 13a or 14a, wherein the Cβ comprises, consists essentially of, or consists of an amino acid sequence having at least 90%, at least 91%, at least 92%, at least 93%, at least 94%, at least 95%, at least 96%, at least 97%, at least 98%, or at least 99% identity to, or comprising or consisting of, the amino acid sequence set forth in any one of SEQ ID NOs.: 28, 29, 54, 55, and 70-73.

Embodiment 16a. The binding protein of any one of Embodiments 13a-15a, wherein the Cα and the Cβ comprise or consist of amino acid sequences having at least 90%, at least 91%, at least 92%, at least 93%, at least 94%, at least 95%, at least 96%, at least 97%, at least 98%, or at least 99% identity to, or comprising or consisting of, the amino acid sequences set forth in SEQ ID NOs.:
(i) 18 and 28, respectively;
(ii) 19 and 29, respectively;
(iii) 44 and 54, respectively; or
(iv) 45 and 55, respectively.

Embodiment 17a. The binding protein of any one of Embodiments 13a-16a, wherein the Cα, the Cβ, or both comprise modification(s) that promote preferential pairing of the Cα to the Cβ.

Embodiment 18. The binding protein of any one of Embodiments 13a-16a, wherein the Cα and the Cβ each comprises an introduced cysteine residue that promotes preferential pairing of the Cα to the Cβ.

Embodiment 19a. The binding protein of any one of Embodiments 13a-16a, wherein the Cα comprises a T48C substitution and the Cβ comprises a S57C substitution to promote preferential pairing of the Cα to the Cβ.

Embodiment 20a. The binding protein of any one of Embodiments 1a-19a, comprising a TCR α chain and a TCR β chain, wherein the TCR α chain and the TCR β chain comprise or consist of amino acid sequences having at least 90%, at least 91%, at least 92%, at least 93%, at least 94%, at least 95%, at least 96%, at least 97%, at least 98%, or at least 99% identity to, or comprising or consisting of, the amino acid sequences set forth in:
(i) SEQ ID NOs.: 12 and 22, respectively;
(ii) SEQ ID NOs.: 20 and 30, respectively;
(iii) SEQ ID NOS.: 12 and 30, respectively;
(iv) SEQ ID NOs.: 20 and 22, respectively;
(v) SEQ ID NOs.: 38 and 48, respectively;
(vi) SEQ ID NOs.: 46 and 56, respectively;
(vii) SEQ ID NOs.: 38 and 56, respectively;
(viii) SEQ ID NOs.: 46 and 48, respectively; or
(ix) SEQ ID NOs.: 85 and 83, respectively.

Embodiment 21a. The binding protein of any one of Embodiments 1a-20a, wherein the binding protein comprises a TCR, a single-chain TCR (scTCR), a single-chain T cell receptor variable fragment (scTv), or a chimeric antigen receptor (CAR).

Embodiment 22a. The binding protein of Embodiment 21a, wherein the binding protein comprises a TCR.

Embodiment 23a. The binding protein of any one of Embodiments 1-22a, wherein the binding protein comprises an EC50 of at most 100 nM, at most 50 nM, at most 25 nM, at most 10 nM, at most 1 nM, at most 750 pM, at most 500 pM, at most 250 pM, at most 100 pM, at most 75 pM, or at most 60 pM in a CD137 surface expression assay for functional avidity to the peptide.

Embodiment 24a. An isolated polynucleotide encoding the binding protein of any one of Embodiments 1a-23a.

Embodiment 25a. The polynucleotide of Embodiment 24a, comprising a polynucleotide having at least 75%, at least 80%, at least 85%, at least 90%, at least 91%, at least 92%, at least 93%, at least 94%, at least 95%, at least 96%, at least 97%, at least 98%, or at least 99% identity to, or comprising or consisting of, the polynucleotide sequence set forth in any one of SEQ ID NOs.: 5-10 and 33-36, or any combination thereof.

Embodiment 26a. The polynucleotide of Embodiment 24a or 25a, further comprising:
(i) a polynucleotide encoding a polypeptide that comprises an extracellular portion of a CD8 co-receptor α chain, wherein, optionally, the encoded polypeptide is or comprises a CD8 co-receptor α chain;
(ii) a polynucleotide encoding a polypeptide that comprises an extracellular portion of a CD8 co-receptor β chain, wherein, optionally, the encoded polypeptide is or comprises a CD8 co-receptor β chain; or
(iii) a polynucleotide of (i) and a polynucleotide of (ii).

Embodiment 27a. The polynucleotide of Embodiment 26a, comprising:
(a) the polynucleotide encoding a polypeptide comprising an extracellular portion of a CD8 co-receptor α chain;
(b) the polynucleotide encoding a polypeptide comprising an extracellular portion of a CD8 co-receptor β chain; and
(c) a polynucleotide encoding a self-cleaving peptide disposed between the polynucleotide of (a) and the polynucleotide of (b).

Embodiment 28a. The polynucleotide of Embodiment 26a or 27a, further comprising a polynucleotide that encodes a self-cleaving peptide and is disposed between:
(1) the polynucleotide encoding a binding protein and the polynucleotide encoding a polypeptide comprising an extracellular portion of a CD8 co-receptor α chain; and/or
(2) the polynucleotide encoding a binding protein and the polynucleotide encoding a polypeptide comprising an extracellular portion of a CD8 co-receptor β chain.

Embodiment 29a. The polynucleotide of any one of Embodiments 26a-28a, comprising, operably linked in-frame:
(i) (pnCD8α)-(pnSCP₁)-(pnCD8β)-(pnSCP₂)-(pnBP);
(ii) (pnCD8β)-(pnSCP₁)-(pnCD8α)-(pnSCP₂)-(pnBP);
(iii) (pnBP)-(pnSCP₁)-(pnCD8α)-(pnSCP₂)-(pnCD8β);
(iv) (pnBP)-(pnSCP₁)-(pnCD8β)-(pnSCP₂)-(pnCD8α);
(v) (pnCD8α)-(pnSCP₁)-(pnBP)-(pnSCP₂)-(pnCD8β); or
(vi) (pnCD8β)-(pnSCP₁)-(pnBP)-(pnSCP₂)-(pnCD8α),
wherein pnCD8α is the polynucleotide encoding a polypeptide that comprises an extracellular portion of a CD8 co-receptor α chain,
wherein pnCD8β is the polynucleotide encoding a polypeptide that comprises an extracellular portion of a CD8 co-receptor α chain,
wherein pnBP is the polynucleotide encoding a binding protein,
and wherein pnSCP₁ and pnSCP₂ are each independently a polynucleotide encoding a self-cleaving peptide, wherein the polynucleotides and/or the encoded self-cleaving peptides are optionally the same or different.

Embodiment 30a. The polynucleotide of any one of Embodiments 26a-29a, wherein the encoded binding protein comprises a TCRα chain and a TCRβ chain, wherein the polynucleotide comprises a polynucleotide encoding a self-cleaving peptide disposed between the polynucleotide encoding a TCRα chain and the polynucleotide encoding a TCRβ chain.

Embodiment 31a. The polynucleotide of Embodiment 30a, comprising, operably linked in-frame:
(i) (pnCD8α)-(pnSCP₁)-(pnCD8β)-(pnSCP₂)-(pnTCRβ)-(pnSCP₃)-(pnTCRα);
(ii) (pnCD8β)-(pnSCP₁)-(pnCD8α)-(pnSCP₂)-(pnTCRβ)-(pnSCP₃)-(pnTCRα);
(iii) (pnCD8α)-(pnSCP₁)-(pnCD8β)-(pnSCP₂)-(pnTCRα)-(pnSCP₃)-(pnTCRβ);
(iv) (pnCD8β)-(pnSCP₁)-(pnCD8α)-(pnSCP₂)-(pnTCRα)-(pnSCP₃)-(pnTCRβ);
(v) (pnTCRβ)-(pnSCP₁)-(pnTCRα)-(pnSCP₂)-(pnCD8α)-(pnSCP₃)-(pnCD8β);
(vi) (pnTCRβ)-(pnSCP₁)-(pnTCRα)-(pnSCP₂)-(pnCD8β)-(pnSCP₃)-(pnCD8α);
(vii) (pnTCRα)-(pnSCP₁)-(pnTCRβ)-(pnSCP₂)-(pnCD8α)-(pnSCP₃)-(pnCD8β);
(viii) (pnTCRα)-(pnSCP₁)-(pnTCRβ)-(pnSCP₂)-(pnCD8β)-(pnSCP₃)-(pnCD8α),
wherein pnCD8α is the polynucleotide encoding a polypeptide that comprises an extracellular portion of a CD8 co-receptor α chain,
wherein pnCD8β is the polynucleotide encoding a polypeptide that comprises an extracellular portion of a CD8 co-receptor α chain,
wherein pnTCRα is the polynucleotide encoding a TCR α chain,
wherein pnTCRβ is the polynucleotide encoding a TCR β chain,
and wherein pnSCP₁, pnSCP₂, and pnSCP₃ are each independently a polynucleotide encoding a self-cleaving peptide, wherein the polynucleotides and/or the encoded self-cleaving peptides are optionally the same or different.

Embodiment 32a. The polynucleotide of Embodiment 31a, wherein the pnSCP1 encodes a T2A peptide, the pnSCP2 encodes a P2A peptide, and the pnSCP3 encodes a P2A peptide.

Embodiment 33a. The polynucleotide of any one of Embodiments 24a-32a, encoding an amino acid sequence having at least 90%, at least 91%, at least 92%, at least 93%, at least 94%, at least 95%, at least 96%, at least 97%, at least 98%, or at least 99% identity to, or comprising or consisting of the amino acid sequence set forth in any one of SEQ ID NOs.: 11, 21, 37, 47, 31, 32, 57, 58, 84, 86, 88, and 90.

Embodiment 34a. The polynucleotide of Embodiment 33a, encoding (i) an amino acid sequence having at least 90%, at least 91%, at least 92%, at least 93%, at least 94%, at least 95%, at least 96%, at least 97%, at least 98%, or at least 99% identity to, or comprising or consisting of the amino acid sequence set forth in SEQ ID NO.: 11, and (ii) an amino acid sequence having at least 90%, at least 91%, at least 92%, at least 93%, at least 94%, at least 95%, at least 96%, at least 97%, at least 98%, or at least 99% identity to, or comprising or consisting of the amino acid sequence set forth in SEQ ID NO.: 21.

Embodiment 35a. The polynucleotide of Embodiment 33a, encoding (i) an amino acid sequence having at least 90%, at least 91%, at least 92%, at least 93%, at least 94%, at least 95%, at least 96%, at least 97%, at least 98%, or at least 99% identity to, or comprising or consisting of the amino acid sequence set forth in SEQ ID NO.: 37, and (ii) an amino acid sequence having at least 90%, at least 91%, at least 92%, at least 93%, at least 94%, at least 95%, at least 96%, at least 97%, at least 98%, or at least 99% identity to, or comprising or consisting of the amino acid sequence set forth in SEQ ID NO.: 47.

Embodiment 36a. The polynucleotide of any one of Embodiments 24a-35a, which is or comprises a polynucleotide sequence that is codon optimized for expression in a host cell, wherein, optionally, the host cell is a human immune system cell, and wherein, further optionally, is a T cell.

Embodiment 37a. An expression vector, comprising a polynucleotide of any one of Embodiments 24a-36a operably linked to an expression control sequence.

Embodiment 38a. The expression vector of Embodiment 37a, wherein the expression control sequence comprises an MSCV promoter.

Embodiment 39a. The expression vector of Embodiment 37a or Embodiment 38a, wherein the expression control sequence drives expression of a single mRNA encoding the extracellular portion of the CD8 co-receptor α chain, the extracellular portion of the CD8 co-receptor β chain, the TCR α chain, and the TCR β chain.

Embodiment 40a. The expression vector of any one of Embodiments 37a-39a, wherein the vector is capable of delivering the polynucleotide to a host cell.

Embodiment 41a. The expression vector of Embodiment 40a, wherein the host cell is a hematopoietic progenitor cell or a human immune system cell.

Embodiment 42a. The expression vector of Embodiment 41a, wherein the human immune system cell is a CD4⁺ T cell, a CD8⁺ T cell, a CD4⁻CD8⁻ double negative T cell, a γδ T cell, a natural killer cell, a natural killer T cell, a macrophage, a monocyte, a dendritic cell, or any combination thereof.

Embodiment 43a. The expression vector of Embodiment 42a, wherein the T cell is a naïve T cell, a central memory T cell, an effector memory T cell, or any combination thereof. Embodiment 44a. The expression vector of any one of Embodiments 37a-43a, wherein the vector is a viral vector.

Embodiment 45a. The expression vector of Embodiment 44, wherein the viral vector is a lentiviral vector or a y-retroviral vector.

Embodiment 46a. The expression vector of Embodiment 44a, wherein the viral vector is a self-inactivating lentiviral vector.

Embodiment 47a. The expression vector of Embodiment 44a or Embodiment 46a, wherein the viral vector is a third generation lentiviral vector.

Embodiment 48a. A host cell modified to comprise the polynucleotide of any one of Embodiments 24a-36a and/or the expression vector of any one of Embodiments 37a-47a and/or to express the binding protein of any one of Embodiments 1a-23a.

Embodiment 49a. The host cell of Embodiment 48a, wherein the modified cell comprises a hematopoietic progenitor cell and/or a human immune cell.

Embodiment 50a. The host cell of Embodiment 49a, wherein the immune cell comprises a T cell, a NK cell, a NK-T cell, a dendritic cell, a macrophage, a monocyte, or any combination thereof.

Embodiment 51a. The host cell of Embodiment 50a, wherein the immune cell comprises a CD4⁺ T cell, a CD8⁺ T cell, a CD4⁻CD8⁻ double negative T cell, a γδ T cell, a naïve T cell, a central memory T cell, a stem cell memory T cell, an effector memory T cell, or any combination thereof,
wherein, optionally, the immune cell comprises a CD4⁺ T cell and a CD8⁺ T cell, wherein, further optionally, the CD4⁺ T cell, the CD8⁺ T cell, or both comprise (i) a polynucleotide encoding a polypeptide that comprises an extracellular portion of a CD8 co-receptor α chain, wherein, optionally, the encoded polypeptide is or comprises a CD8 co-receptor α chain; (ii) a polynucleotide encoding a polypeptide that comprises an extracellular portion of a CD8 co-receptor β chain, wherein, optionally, the encoded polypeptide is or comprises a CD8 co-receptor β chain; or (iii) a polynucleotide of (i) and a polynucleotide of (ii).

Embodiment 52a. The host cell of any one of Embodiments 48a-51a, wherein the modified cell comprises a chromosomal gene knockout of a PD-1 gene; a LAG3 gene; a TIM3 gene; a CTLA4 gene; an HLA component gene; a TIGIT gene; a TCR component gene, a FasL gene, or any combination thereof.

Embodiment 53a. The host cell of Embodiment 52a, wherein the chromosomal gene knockout comprises a knockout of an HLA component gene selected from an α1 macroglobulin gene, an α2 macroglobulin gene, an α3 macroglobulin gene, a β1 microglobulin gene, or a β2 microglobulin gene.

Embodiment 54a. The host cell of Embodiment 52a or 53a, wherein the chromosomal gene knockout comprises a knockout of a TCR component gene selected from a TCR α variable region gene, a TCR β variable region gene, a TCR constant region gene, or a combination thereof.

Embodiment 55a. A composition comprising the host cell of any one of Embodiments 48a-54a and a pharmaceutically acceptable carrier, diluent, or excipient.

Embodiment 56a. The composition of Embodiment 55a, comprising at least about 30% modified CD4⁺ T cells, combined with (ii) a composition comprising at least about 30% modified CD8⁺ T cells, in about a 1:1 ratio.

Embodiment 57a. The composition of Embodiment 55a or 56a, wherein the composition contains substantially no naïve T cells.

Embodiment 58a. A composition comprising:
(v) the binding protein of any one of Embodiments 1a-23a;
(vi) the polynucleotide of any one of Embodiments 24a-36a;
(vii) the expression vector of any one of Embodiments 37a-47a; and/or
(viii) the host cell of any one of Embodiments 48a-54a,
and a pharmaceutically acceptable carrier, excipient, or diluent.

Embodiment 59a. A method for treating a disease or disorder associated with a KRAS G12V mutation or a NRAS G12V mutation or a HRAS G12V mutation in a subject, the method comprising administering to the subject an effective amount of:
(i) the binding protein of any one of Embodiments 1-23a;
(ii) the polynucleotide of any one of Embodiments 24a-36a;
(iii) the expression vector of any one of Embodiments 37a-47a;
(iv) the host cell of any one of Embodiments 48a-54a, wherein, optionally, the host cell comprises a CD8+ T cell, a CD4+ T cell, or both, and wherein, optionally, the host cell is autologous, allogeneic, or syngeneic to the subject; and/or
(v) the composition of any one of Embodiments 55a-58a.

Embodiment 60a. The method of Embodiment 59a, wherein the disease or disorder comprises a cancer, wherein the cancer is optionally a solid cancer or a hematological malignancy.

Embodiment 61a. The method of Embodiment 59a or 60a, wherein the disease or disorder is selected from a pancreas cancer or carcinoma, optionally a pancreatic ductal adenocarcinoma (PDAC); a colorectal cancer or carcinoma; a lung cancer, optionally a non-small-cell lung carcinoma; a biliary cancer; an endometrial cancer or carcinoma; a cervical cancer; an ovarian cancer; a bladder cancer; a liver cancer; a myeloid leukemia, optionally myeloid leukemia such as acute myeloid leukemia; a myelodysplastic syndrome; a lymphoma such as Non-Hodgkin lymphoma; Chronic Melyomonocytic Leukemia; Acute Lymphoblastic Leukemia (ALL); a cancer of the urinary tract; a cancer of the small intestine; a breast cancer or carcinoma; a melanoma (optionally a cutaneous melanoma, an anal melanoma, or a mucosal melanoma); a glioma; a poorly differentiated thyroid gland carcinoma; a neuroblastoma; a histiocytic and dendritic cell neoplasm; neurofibromatisis Type 1; rhabdomyosarcoma; a soft tissue sarcoma; a bladder carcinoma; a sarcoma; a glioblastoma; a squamous cell lung carcinoma; an anaplastic astrocytoma; chronic myeloid leukemia; diffuse large B-cell lymphoma; double-hit lymphoma; head and neck carcinoma; head and neck squamous cell carcinoma; hepatocellular carcinoma; malignant peripheral nerve sheath tumor; mantle cell lymphoma; myelodysplastic/myeloproliferative neoplasm, unclassifiable; peripheral T cell lymphoma; prostate carcinoma; refractory anemia with excess blasts-2; renal cell carcinoma; rhabdoid tumor; schwannoma; secondary AML; small cell lung carcinoma; therapy-related AML; thymic carcinoma; thyroid gland follicular carcinoma; malignant thyroid gland neoplasm; thyroid gland carcinoma; thyroid gland adenocarcinoma; urothelial carcinoma; colon cancer; colorectal adenocarcinoma; thyroid gland papillary carcinoma; or an advanced or metastatic version thereof.

Embodiment 62a. The method of any one of Embodiments 59a-61a, wherein the binding protein, polynucleotide, vector, host cell, or composition is administered to the subject parenterally or intravenously.

Embodiment 63a. The method of any one of Embodiments 59a-62a, wherein the method comprises administering a plurality of doses of any one or more of (i)-(v) to the subject. Embodiment 64a. The method of Embodiment 63a, wherein the plurality of doses are administered at intervals between administrations of about two to about four weeks.

Embodiment 65a. The method of any one of Embodiments 59a-64a, wherein the composition comprises the host cell or the composition comprising the host cell, and wherein the method comprises administering the host cell or composition to the subject at a dose of about 10⁴ cells/kg to about 10¹¹ cells/kg.

Embodiment 66a. The method of any one of Embodiments 59a-65a, wherein the method comprises administering to the subject at least 5×10\hat{ }8, at least 1×10\hat{ }9, at least 5×10\hat{ }9, at least 1×10\hat{ }10, at least 1.5×10\hat{ }10, at least 2×10\hat{ }10, or at least 5×10\hat{ }10 viable host cells that comprise the binding protein, optionally in a single dose.

Embodiment 67a. The method of any one of Embodiments 59a-65a, wherein the method comprises administering to the subject at most 5×10\hat{ }9, at most 1×10\hat{ }10, at most 1.5×10\hat{ }10, at most 2×10\hat{ }10, at most 5×10\hat{ }10, at most 1×10\hat{ }11, or at most 5×10\hat{ }11 viable host cells that comprise the binding protein, optionally in a single dose.

Embodiment 68a. The method of any one of Embodiments 59a-65a, wherein the method comprises administering to the subject about 5×10\hat{ }9, about 6×10\hat{ }9, about 7×10\hat{ }9, about 8×10\hat{ }9, about 9×10\hat{ }9, about 1×10\hat{ }10, about 1.1×10\hat{ }10, about 1.2×10\hat{ }10, about 1.3×10\hat{ }10, about 1.4×10\hat{ }10, about 1.5×10\hat{ }10, about 1.6×10\hat{ }10, about 1.7×10\hat{ }10, about 1.8×10\hat{ }10, about 1.9×10\hat{ }10, or about 2×10\hat{ }10 viable host cells that comprise the binding protein, optionally in a single dose.

Embodiment 69a. The method of any one of Embodiments 59a-65a, wherein the method comprises administering to the subject about 5×10\hat{ }9 to about 1×10\hat{ }11, about 5×10\hat{ }9 to about 5×10\hat{ }10, about 5×10\hat{ }9 to about 2×10\hat{ }10, about 5×10\hat{ }9 to about 1.5×10\hat{ }10, about 5×10\hat{ }9 to about 1×10\hat{ }10, about 1×10\hat{ }10 to about 1×10\hat{ }11, about 1×10\hat{ }10 to about 5×10\hat{ }10, about 1×10\hat{ }10 to about 2×10\hat{ }10, or about 1×10\hat{ }10 to about 1.5×10\hat{ }10 viable host cells that comprise the binding protein, optionally in a single dose.

Embodiment 70a. The method of any one of Embodiments 59a-69a, further comprising determining that the subject expresses HLA-A*11, optionally HLA-A*11:01, prior to administering the binding protein, polynucleotide, vector, host cell, or composition.

Embodiment 71a. The method of any one of Embodiments 59a-70a, wherein the method further comprises administering a cytokine to the subject.

Embodiment 72a. The method of Embodiment 71a, wherein the cytokine comprises IL-2, IL-15, or IL-21.

Embodiment 73a. The method of any one of Embodiments 59a-72a, wherein the subject has received or is receiving an immune checkpoint inhibitor and/or an agonist of a stimulatory immune checkpoint agent.

Embodiment 74a. The binding protein of any one of Embodiments 1a-23a, the polynucleotide of any one of Embodiments 24a-36a, the expression vector of any one of Embodiments 37a-47a, the host cell of any one of Embodiments 48a-54a, wherein, optionally, the host cell comprises a CD8+ T cell, a CD4+ T cell, or both, and/or the composition of any one of Embodiments 55a-58a, for use in a method for treating a disease or disorder associated with a KRAS G12V or a NRAS G12V mutation or a HRAS G12V mutation in a subject, wherein, optionally, the disease or disorder comprises a cancer, wherein, further optionally, the cancer is a solid cancer or a hematological malignancy, and wherein, optionally, the disease or disorder is selected from a pancreas cancer or carcinoma, optionally a pancreatic ductal adenocarcinoma (PDAC); a colorectal cancer or carcinoma; a lung cancer, optionally a non-small-cell lung carcinoma; a biliary cancer; an endometrial cancer or carcinoma; a cervical cancer; an ovarian cancer; a bladder cancer; a liver cancer; a myeloid leukemia, optionally myeloid leukemia such as acute myeloid leukemia; a myelodysplastic syndrome; a lymphoma such as Non-Hodgkin lymphoma; Chronic Melyomonocytic Leukemia; Acute Lymphoblastic Leukemia (ALL); a cancer of the urinary tract; a cancer of the small intestine; a breast cancer or carcinoma; a melanoma (optionally a cutaneous melanoma, an anal melanoma, or a mucosal melanoma); a glioma; a poorly differentiated thyroid gland carcinoma; a neuroblastoma; a histiocytic and dendritic cell neoplasm; neurofibromatisis Type 1; rhabdomyosarcoma; a soft tissue sarcoma; a bladder carcinoma; a sarcoma; a glioblastoma; a squamous cell lung carcinoma; an anaplastic astrocytoma; chronic myeloid leukemia; diffuse large B-cell lymphoma; double-hit lymphoma; head and neck carcinoma; head and neck squamous cell carcinoma; hepatocellular carcinoma; malignant peripheral nerve sheath tumor; mantle cell lymphoma; myelodysplastic/myeloproliferative neoplasm, unclassifiable; peripheral T cell lymphoma; prostate carcinoma; refractory anemia with excess blasts-2; renal cell carcinoma; rhabdoid tumor; schwannoma; secondary AML; small cell lung carcinoma; therapy-related AML; thymic carcinoma; thyroid gland follicular carcinoma; malignant thyroid gland neoplasm; thyroid gland carcinoma; thyroid gland adenocarcinoma; urothelial carcinoma; colon cancer; colorectal adenocarcinoma; thyroid gland papillary carcinoma; or an advanced or metastatic version thereof.

Embodiment 75a. The binding protein of any one of Embodiments 1a-23a, the polynucleotide of any one of Embodiments 24a-36a, the expression vector of any one of Embodiments 37a-47a, the host cell of any one of Embodiments 48a-54a, wherein, optionally, the host cell comprises a CD8+ T cell, a CD4+ T cell, or both, and/or the composition of any one of Embodiments 55a-58a, for use the manufacture of a medicament for treating a disease or disorder associated with a KRAS G12V or a NRAS G12V mutation or a HRAS G12V mutation in a subject, wherein, optionally, the disease or disorder comprises a cancer, wherein, further optionally, the cancer is a solid cancer or a hematological malignancy. and, wherein, optionally, the disease or disorder is selected from a pancreas cancer or carcinoma, optionally a pancreatic ductal adenocarcinoma (PDAC); a colorectal cancer or carcinoma; a lung cancer, optionally a non-small-cell lung carcinoma; a biliary cancer; an endometrial cancer or carcinoma; a cervical cancer; an ovarian cancer; a bladder cancer; a liver cancer; a myeloid leukemia, optionally myeloid leukemia such as acute myeloid leukemia; a myelodysplastic syndrome; a lymphoma such as Non-Hodgkin lymphoma; Chronic Melyomonocytic Leukemia; Acute Lymphoblastic Leukemia (ALL); a cancer of the urinary tract; a cancer of the small intestine; a breast cancer or carcinoma; a melanoma (optionally a cutaneous melanoma, an anal melanoma, or a mucosal melanoma); a glioma; a poorly differentiated thyroid gland carcinoma; a neuroblastoma; a histiocytic and dendritic cell neoplasm; neurofibromatisis Type 1; rhabdomyosarcoma; a soft tissue sarcoma; a bladder carcinoma; a sarcoma; a glioblastoma; a squamous cell lung carcinoma; an anaplastic astrocytoma; chronic myeloid leukemia; diffuse large B-cell lymphoma; double-hit lymphoma; head and neck carcinoma; head and neck squamous cell carcinoma; hepatocellular carcinoma; malignant peripheral nerve sheath tumor; mantle cell lymphoma; myelodysplastic/myeloproliferative neoplasm, unclassifiable; peripheral T cell lymphoma; prostate carcinoma; refractory anemia with excess blasts-2; renal cell carcinoma; rhabdoid tumor; schwannoma; secondary AML; small cell lung carcinoma; therapy-related AML; thymic carcinoma; thyroid gland follicular carcinoma; malignant thyroid gland neoplasm; thyroid gland carcinoma; thyroid gland adenocarcinoma; urothelial carcinoma; colon cancer; colorectal adenocarcinoma; thyroid gland papillary carcinoma; or an advanced or metastatic version thereof.

SEQUENCES
wt KRAS full (UniProt: P01116)
SEQ ID NO: 1
MTEYKLVVVG AGGVGKSALT IQLIQNHFVD EYDPTIEDSY RKQVVIDGET
CLLDILDTAG QEEYSAMRDQ YMRTGEGFLC VFAINNTKSF EDIHHYREQI
KRVKDSEDVP MVLVGNKCDL PSRTVDTKQA QDLARSYGIP FIETSAKTRQ
RVEDAFYTLV REIRQYRLKK ISKEEKTPGC VKIKKCIIM
KRAS 7-16 G12V
SEQ ID NO: 2
VVVGAVGVGK
KRAS 8-16 G12V
SEQ ID NO: 3
VVGAVGVGK
KRAS 8-16 G12V binding motif for TCR 11N4A
SEQ ID NO: 4
x-V-G-A-x-G-x-x-K
TCR 11N4A alpha chain with signal peptide-original (WT) nucleotide
sequence
SEQ ID NO: 5
atggccatgctcctgggggcatcagtgctgattctgtggcttcagccagactggg
taaacagtcaacagaagaatgatgaccagcaagttaagcaaaattcaccatccct
gagcgtccaggaaggaagaatttctattctgaactgtgactatactaacagcatg
tttgattatttcctatggtacaaaaaataccctgctgaaggtcctacattcctga
tatctataagttccattaaggataaaaatgaagatggaagattcactgtcttctt
aaacaaaagtgccaagcacctctctctgcacattgtgccctcccagcctggagac
tctgcagtgtacttctgtgcagcaagtggggtttcaggaaacacacctcttgtct
ttggaaagggcacaagactttctgtgattgcaaatatccagaaccctgaccctgc
cgtgtaccagctgagagactctaaatccagtgacaagtctgtctgcctattcacc
gattttgattctcaaacaaatgtgtcacaaagtaaggattctgatgtgtatatca
cagacaaaactgtgctagacatgaggtctatggacttcaagagcaacagtgctgt
ggcctggagcaacaaatctgactttgcatgtgcaaacgccttcaacaacagcatt
attccagaagacaccttcttccccagcccagaaagttcctgtgatgtcaagctgg
tcgagaaaagctttgaaacagatacgaacctaaactttcaaaacctgtcagtgat
tgggttccgaatcctcctcctgaaagtggccgggtttaatctgctcatgacgctg
cggctgtggtccagctga
TCR 11N4A beta chain with signal peptide-original (WT) nucleotide
sequence
SEQ ID NO: 6
atgggctccaggctgctctgttgggtgctgctttgtctcctgggagcaggcccag
taaaggctggagtcactcaaactccaagatatctgatcaaaacgagaggacagca
agtgacactgagctgctcccctatctctgggcataggagtgtatcctggtaccaa
cagaccccaggacagggccttcagttcctctttgaatacttcagtgagacacaga
gaaacaaaggaaacttccctggtcgattctcagggcgccagttctctaactctcg
ctctgagatgaatgtgagcaccttggagctgggggactcggccctttatctttgc
gccagcagcgtcgggactgtggagcagtacttcgggccgggcaccaggctcacgg
tcacagaggacctgaaaaacgtgttcccacccgaggtcgctgtgtttgagccatc
agaagcagagatctcccacacccaaaaggccacactggtgtgcctggccacaggc
ttctaccccgaccacgtggagctgagctggtgggtgaatgggaaggaggtgcaca
gtggggtcagcacagacccgcagcccctcaaggagcagcccgccctcaatgactc
cagatactgcctgagcagccgcctgagggtctcggccaccttctggcagaacccc
cgcaaccacttccgctgtcaagtccagttctacgggctctcggagaatgacgagt
ggacccaggatagggccaaacctgtcacccagatcgtcagcgccgaggcctgggg
tagagcagactgtggcttcacctccgagtcttaccagcaaggggtcctgtctgcc
accatcctctatgagatcttgctagggaaggccaccttgtatgccgtgctggtca
gtgccctcgtgctgatggccatggtcaagagaaaggattccagaggctag
TCR 11N4A TCRbeta-P2A-TCRalpha polynucleotide-Codon-
optimization A
SEQ ID NO: 7
ATGGGCTCTAGACTGTTGTGTTGGGTTCTGCTGTGTCTGCTTGGAGCTGGACCTG
TGAAAGCTGGAGTTACCCAGACACCCAGATATCTGATCAAGACCAGAGGACAGCA
GGTGACACTGAGCTGTAGCCCTATTTCTGGCCACAGGAGCGTTAGCTGGTATCAG
CAAACACCCGGGCAGGGACTACAATTTCTATTCGAGTACTTCAGCGAGACCCAGC
GGAATAAGGGCAATTTTCCTGGCAGATTTAGCGGCAGGCAGTTCAGCAACAGCAG
AAGCGAGATGAACGTGAGCACCCTGGAATTAGGCGATTCTGCTCTGTACCTGTGT
GCCTCTTCTGTGGGAACAGTGGAGCAGTACTTTGGCCCCGGCACGAGACTGACAG
TGACAGAGGACCTGAAGAACGTGTTCCCCCCAGAGGTGGCCGTGTTCGAGCCTAG
CGAGGCCGAGATCAGCCACACCCAGAAAGCCACCCTCGTGTGCCTGGCCACCGGC
TTTTACCCCGACCACGTGGAACTGTCTTGGTGGGTCAACGGCAAAGAGGTGCACA
GCGGCGTCTGCACCGACCCCCAGCCCCTGAAAGAGCAGCCCGCCCTGAACGACAG
CCGGTACTGTCTGAGCAGCAGACTGAGAGTGTCCGCCACCTTCTGGCAGAACCCC
CGGAACCACTTCAGATGCCAGGTGCAGTTCTACGGCCTGAGCGAGAACGACGAGT
GGACCCAGGACCGGGCCAAGCCCGTGACCCAGATCGTGTCTGCTGAGGCCTGGGG
CAGAGCCGATTGCGGCTTCACCAGCGAGAGCTACCAGCAGGGCGTGCTGAGCGCC
ACCATCCTGTACGAGATCCTGCTGGGCAAGGCCACCCTGTACGCCGTGCTGGTGT
CCGCCCTGGTGCTGATGGCCATGGTCAAGCGGAAGGACAGCCGGGGCGGTTCCGG
AGCCACGAACTTCTCTCTGTTAAAGCAAGCAGGAGACGTGGAAGAAAACCCCGGT
CCCATGGCCATGTTACTAGGAGCGAGCGTGCTGATTCTGTGGTTACAGCCTGATT
GGGTGAACTCTCAGCAGAAGAACGACGATCAGCAGGTGAAGCAGAATAGCCCCTC
TCTGTCTGTGCAGGAGGGCAGAATCTCTATCCTGAATTGCGACTACACCAACAGC
ATGTTCGACTATTTTCTGTGGTACAAAAAATACCCCGCCGAGGGCCCTACATTCC
TGATCAGCATCAGCTCTATCAAGGACAAGAACGAGGATGGCAGATTTACCGTGTT
CCTGAACAAGAGCGCCAAGCACCTGAGCCTGCACATTGTGCCTTCTCAACCTGGC
GATTCTGCTGTGTACTTTTGTGCTGCCTCTGGAGTGAGCGGCAATACACCTCTAG
TGTTCGGGAAGGGCACAAGACTGTCTGTTATTGCAAACATTCAAAACCCCGACCC
TGCTGTGTACCAGCTGCGGGACAGCAAGAGCAGCGACAAGAGCGTGTGCCTGTTC
ACCGACTTCGACAGCCAGACCAACGTGTCCCAGAGCAAGGACAGCGACGTGTACA
TCACCGATAAGTGCGTGCTGGACATGCGGAGCATGGACTTCAAGAGCAACAGCGC
CGTGGCCTGGTCCAACAAGAGCGACTTCGCCTGCGCCAACGCCTTCAACAACAGC
ATTATCCCCGAGGACACATTCTTCCCAAGCCCCGAGAGCAGCTGCGACGTGAAGC
TGGTGGAAAAGAGCTTCGAGACAGACACCAACCTGAACTTCCAGAACCTCAGCGT
GATCGGCTTCCGGATCCTGCTGCTGAAGGTGGCCGGCTTCAACCTGCTGATGACC
CTGCGGCTGTGGTCCAGCTGA
11N4A TCRbeta-P2A-TCRalpha polynucleotide Codon-optimization B
SEQ ID NO: 8
ATGGGATCTAGATTGCTTTGTTGGGTGCTGCTGTGCCTGCTCGGAGCCGGACCTG
TGAAAGCTGGCGTTACCCAGACACCTAGATACCTGATCAAGACCAGAGGCCAGCA
AGTGACCCTGAGCTGCTCTCCTATCAGCGGCCACAGAAGCGTGTCCTGGTATCAG
CAGACACCTGGACAGGGCCTGCAGTTCCTGTTCGAGTACTTCAGCGAGACACAGC
GGAACAAGGGCAACTTCCCCGGCAGATTTTCCGGCAGACAGTTCAGCAACAGCCG
CAGCGAGATGAACGTGTCCACACTGGAACTGGGCGACAGCGCCCTGTATCTGTGT
GCCTCTTCTGTGGGCACCGTGGAACAGTACTTTGGCCCTGGCACCAGACTGACCG
TGACCGAGGATCTGAAGAACGTGTTCCCACCTGAGGTGGCCGTGTTCGAGCCTTC
TGAGGCCGAGATCAGCCACACACAGAAAGCCACACTCGTGTGTCTGGCCACCGGC
TTCTATCCCGATCACGTGGAACTGTCTTGGTGGGTCAACGGCAAAGAGGTGCACA
GCGGCGTCTGTACCGATCCTCAGCCTCTGAAAGAGCAGCCCGCTCTGAACGACAG
CAGATACTGCCTGAGCAGCAGACTGAGAGTGTCCGCCACCTTCTGGCAGAACCCC
AGAAACCACTTCAGATGCCAGGTGCAGTTCTACGGCCTGAGCGAGAACGATGAGT
GGACCCAGGATAGAGCCAAGCCTGTGACACAGATCGTGTCTGCCGAAGCCTGGGG
CAGAGCCGATTGTGGCTTTACCAGCGAGAGCTACCAGCAGGGCGTGCTGTCTGCC
ACAATCCTGTACGAGATCCTGCTGGGCAAAGCCACTCTGTACGCCGTGCTGGTTT
CTGCCCTGGTGCTGATGGCCATGGTCAAGCGGAAGGATTCTAGAGGCGGATCCGG
AGCCACCAACTTCAGCCTGCTTAAACAGGCCGGCGACGTGGAAGAGAACCCTGGA
CCTATGGCTATGCTGCTGGGAGCCTCTGTGCTGATCCTGTGGCTGCAACCCGATT
GGGTCAACAGCCAGCAGAAGAACGACGACCAGCAAGTCAAGCAGAACAGCCCCAG
CCTGAGCGTGCAAGAGGGCAGAATCAGCATCCTGAACTGCGACTACACCAACTCT
ATGTTCGACTACTTTCTGTGGTACAAGAAGTACCCCGCCGAGGGACCCACCTTCC
TGATCAGCATCAGCAGCATCAAGGACAAGAACGAGGACGGCCGGTTCACCGTGTT
TCTGAACAAGAGCGCCAAGCACCTGAGCCTGCACATCGTGCCTTCTCAGCCTGGC
GATAGCGCCGTGTACTTTTGTGCTGCCAGCGGCGTGTCAGGCAACACCCCTCTGG
TTTTTGGCAAGGGCACACGCCTGTCCGTGATCGCCAACATTCAGAACCCTGATCC
TGCCGTGTACCAGCTGAGAGACAGCAAGAGCAGCGACAAGAGCGTGTGCCTGTTC
ACCGACTTCGACAGCCAGACCAACGTGTCCCAGAGCAAGGACAGCGACGTGTACA
TCACCGATAAGTGCGTGCTGGACATGCGGAGCATGGACTTCAAGAGCAACAGCGC
CGTGGCCTGGTCCAACAAGTCCGATTTCGCCTGCGCCAACGCCTTCAACAACAGC
ATTATCCCCGAGGACACATTCTTCCCAAGTCCTGAGTCCAGCTGCGACGTGAAGC
TGGTGGAAAAGAGCTTCGAGACAGACACCAACCTGAACTTCCAGAATCTGAGCGT
GATCGGCTTCAGAATCCTGCTGCTGAAGGTGGCCGGATTCAACCTGCTGATGACC
CTCAGACTGTGGTCCAGCTGA
CD8alpha-T2A-CD8beta-P2A-11N4A TCRbeta-P2A-TCRalpha
polynucleotide Codon-optimization A
SEQ ID NO: 9
ATGGCTCTGCCTGTGACAGCTCTGCTGCTGCCTCTGGCTCTGCTTCTGCATGCCG
CTAGACCCAGCCAGTTCAGAGTGTCCCCTCTGGACAGAACCTGGAACCTGGGCGA
GACAGTGGAACTGAAGTGCCAGGTGCTGCTGAGCAATCCTACCAGCGGCTGCAGC
TGGCTGTTTCAGCCTAGAGGTGCTGCCGCCTCTCCTACCTTTCTGCTGTACCTGA
GCCAGAACAAGCCCAAGGCCGCCGAAGGACTGGACACCCAGAGATTCAGCGGCAA
GAGACTGGGCGACACCTTCGTGCTGACCCTGAGCGACTTCAGAAGAGAGAACGAG
GGCTACTACTTCTGCAGCGCCCTGAGCAACAGCATCATGTACTTCAGCCACTTCG
TGCCCGTGTTTCTGCCCGCCAAGCCTACAACAACCCCTGCTCCTAGACCTCCTAC
ACCAGCTCCTACAATCGCCAGCCAGCCTCTGTCTCTGAGGCCAGAAGCTTGTAGA
CCTGCTGCTGGCGGAGCCGTGCATACAAGAGGACTGGATTTCGCCTGCGACATCT
ACATCTGGGCCCCTCTGGCTGGAACATGTGGCGTGCTGCTGCTGTCCCTGGTCAT
CACCCTGTACTGCAACCACCGGAACAGGCGGAGAGTGTGCAAGTGCCCTAGACCT
GTGGTCAAGAGCGGCGACAAGCCTAGCCTGAGCGCCAGATATGTTGGCAGCGGAG
AAGGCAGAGGCTCCCTGCTTACATGCGGCGACGTGGAAGAGAACCCCGGACCTAT
GAGGCCTAGACTGTGGCTGCTTCTGGCTGCCCAGCTGACAGTGCTGCACGGCAAT
TCTGTCCTGCAGCAGACCCCTGCCTACATCAAGGTGCAGACCAACAAGATGGTCA
TGCTGAGCTGCGAGGCCAAGATCAGCCTGTCCAACATGCGGATCTACTGGCTGCG
GCAGAGACAGGCCCCTAGCTCTGATAGCCACCACGAGTTTCTGGCCCTGTGGGAT
TCTGCCAAGGGCACCATTCACGGCGAGGAAGTGGAACAAGAGAAGATCGCCGTGT
TCCGGGACGCCAGCAGATTCATCCTGAACCTGACCAGCGTGAAGCCCGAGGACAG
CGGCATCTATTTCTGCATGATCGTGGGCAGCCCCGAGCTGACATTTGGCAAGGGA
ACACAGCTGAGCGTGGTGGACTTCCTGCCTACTACAGCCCAGCCTACCAAGAAGT
CTACCCTGAAGAAACGCGTGTGCAGACTGCCCAGGCCTGAGACACAAAAGGGCCC
TCTGTGCAGCCCTATCACACTGGGATTGCTGGTGGCTGGCGTTCTGGTCCTGCTG
GTGTCTCTGGGAGTTGCCATCCACCTGTGCTGTAGAAGAAGGCGGGCCAGACTGC
GGTTCATGAAGCAGTTCTACAAAGGCAGCGGCGCCACCAACTTCAGCCTGCTGAA
ACAAGCCGGCGACGTCGAGGAAAATCCTGGACCTATGGGCTCTAGACTGTTGTGT
TGGGTTCTGCTGTGTCTGCTTGGAGCTGGACCTGTGAAAGCTGGAGTTACCCAGA
CACCCAGATATCTGATCAAGACCAGAGGACAGCAGGTGACACTGAGCTGTAGCCC
TATTTCTGGCCACAGGAGCGTTAGCTGGTATCAGCAAACACCCGGGCAGGGACTA
CAATTTCTATTCGAGTACTTCAGCGAGACCCAGCGGAATAAGGGCAATTTTCCTG
GCAGATTTAGCGGCAGGCAGTTCAGCAACAGCAGAAGCGAGATGAACGTGAGCAC
CCTGGAATTAGGCGATTCTGCTCTGTACCTGTGTGCCTCTTCTGTGGGAACAGTG
GAGCAGTACTTTGGCCCCGGCACGAGACTGACAGTGACAGAGGACCTGAAGAACG
TGTTCCCCCCAGAGGTGGCCGTGTTCGAGCCTAGCGAGGCCGAGATCAGCCACAC
CCAGAAAGCCACCCTCGTGTGCCTGGCCACCGGCTTTTACCCCGACCACGTGGAA
CTGTCTTGGTGGGTCAACGGCAAAGAGGTGCACAGCGGCGTCTGCACCGACCCCC
AGCCCCTGAAAGAGCAGCCCGCCCTGAACGACAGCCGGTACTGTCTGAGCAGCAG
ACTGAGAGTGTCCGCCACCTTCTGGCAGAACCCCCGGAACCACTTCAGATGCCAG
GTGCAGTTCTACGGCCTGAGCGAGAACGACGAGTGGACCCAGGACCGGGCCAAGC
CCGTGACCCAGATCGTGTCTGCTGAGGCCTGGGGCAGAGCCGATTGCGGCTTCAC
CAGCGAGAGCTACCAGCAGGGCGTGCTGAGCGCCACCATCCTGTACGAGATCCTG
CTGGGCAAGGCCACCCTGTACGCCGTGCTGGTGTCCGCCCTGGTGCTGATGGCCA
TGGTCAAGCGGAAGGACAGCCGGGGCGGTTCCGGAGCCACGAACTTCTCTCTGTT
AAAGCAAGCAGGAGACGTGGAAGAAAACCCCGGTCCCATGGCCATGTTACTAGGA
GCGAGCGTGCTGATTCTGTGGTTACAGCCTGATTGGGTGAACTCTCAGCAGAAGA
ACGACGATCAGCAGGTGAAGCAGAATAGCCCCTCTCTGTCTGTGCAGGAGGGCAG
AATCTCTATCCTGAATTGCGACTACACCAACAGCATGTTCGACTATTTTCTGTGG
TACAAAAAATACCCCGCCGAGGGCCCTACATTCCTGATCAGCATCAGCTCTATCA
AGGACAAGAACGAGGATGGCAGATTTACCGTGTTCCTGAACAAGAGCGCCAAGCA
CCTGAGCCTGCACATTGTGCCTTCTCAACCTGGCGATTCTGCTGTGTACTTTTGT
GCTGCCTCTGGAGTGAGCGGCAATACACCTCTAGTGTTCGGGAAGGGCACAAGAC
TGTCTGTTATTGCAAACATTCAAAACCCCGACCCTGCTGTGTACCAGCTGCGGGA
CAGCAAGAGCAGCGACAAGAGCGTGTGCCTGTTCACCGACTTCGACAGCCAGACC
AACGTGTCCCAGAGCAAGGACAGCGACGTGTACATCACCGATAAGTGCGTGCTGG
ACATGCGGAGCATGGACTTCAAGAGCAACAGCGCCGTGGCCTGGTCCAACAAGAG
CGACTTCGCCTGCGCCAACGCCTTCAACAACAGCATTATCCCCGAGGACACATTC
TTCCCAAGCCCCGAGAGCAGCTGCGACGTGAAGCTGGTGGAAAAGAGCTTCGAGA
CAGACACCAACCTGAACTTCCAGAACCTCAGCGTGATCGGCTTCCGGATCCTGCT
GCTGAAGGTGGCCGGCTTCAACCTGCTGATGACCCTGCGGCTGTGGTCCAGCTGA
CD8alpha-T2A-CD8beta-P2A-11N4A TCRbeta-P2A-TCRalpha
polynucleotide Codon-optimization B
SEQ ID NO: 10
ATGGCATTGCCTGTTACAGCTCTGCTGCTGCCCCTGGCTCTGCTTCTGCATGCTG
CTAGACCCAGCCAGTTCAGAGTGTCCCCTCTGGACAGAACCTGGAACCTGGGCGA
GACAGTGGAACTGAAGTGCCAGGTGCTGCTGAGCAATCCTACCAGCGGCTGCAGC
TGGCTGTTTCAGCCTAGAGGTGCTGCCGCCTCTCCTACCTTTCTGCTGTACCTGA
GCCAGAACAAGCCCAAGGCCGCCGAAGGACTGGACACCCAGAGATTCAGCGGCAA
GAGACTGGGCGACACCTTCGTGCTGACCCTGAGCGACTTCAGAAGAGAGAACGAG
GGCTACTACTTCTGCAGCGCCCTGAGCAACAGCATCATGTACTTCAGCCACTTCG
TGCCCGTGTTTCTGCCCGCCAAGCCTACAACAACCCCTGCTCCTAGACCTCCTAC
ACCAGCTCCTACAATCGCCAGCCAGCCTCTGTCTCTGAGGCCAGAAGCTTGTAGA
CCTGCTGCTGGCGGAGCCGTGCATACAAGAGGACTGGATTTCGCCTGCGACATCT
ACATCTGGGCCCCTCTGGCTGGAACATGTGGCGTGCTGCTGCTGTCTCTGGTCAT
CACCCTGTACTGCAACCACCGGAACAGGCGGAGAGTGTGCAAGTGCCCTAGACCT
GTGGTCAAGAGCGGCGACAAGCCTAGCCTGAGCGCCAGATATGTTGGCAGCGGAG
AAGGCAGAGGCAGCCTGCTTACATGCGGCGACGTGGAAGAGAACCCCGGACCTAT
GAGGCCTAGACTGTGGCTGCTTCTGGCTGCCCAGCTGACAGTGCTGCACGGCAAT
TCTGTCCTGCAGCAGACCCCTGCCTACATCAAGGTGCAGACCAACAAGATGGTCA
TGCTGAGCTGCGAGGCCAAGATCAGCCTGTCCAACATGCGGATCTACTGGCTGCG
GCAGAGACAGGCCCCTAGCAGCGATTCTCACCACGAGTTTCTGGCCCTGTGGGAT
AGCGCCAAGGGAACCATTCACGGCGAGGAAGTGGAACAAGAGAAGATCGCCGTGT
TCCGGGACGCCAGCAGATTCATCCTGAACCTGACCAGCGTGAAGCCCGAGGACAG
CGGCATCTATTTCTGCATGATCGTGGGCAGCCCCGAGCTGACATTTGGCAAGGGA
ACACAGCTGAGCGTGGTGGACTTCCTGCCTACTACAGCCCAGCCTACCAAGAAGT
CTACCCTGAAGAAACGCGTGTGCAGACTGCCCAGGCCTGAGACACAAAAGGGCCC
TCTGTGCAGCCCTATCACACTGGGATTGCTGGTGGCTGGCGTTCTGGTCCTGCTG
GTTTCTCTGGGAGTTGCCATCCACCTGTGCTGCAGACGCAGAAGGGCCAGACTGC
GGTTCATGAAGCAGTTCTACAAAGGCAGCGGCGCCACCAACTTCAGCCTGCTGAA
ACAAGCCGGCGACGTCGAAGAAAATCCTGGACCAATGGGCAGCAGACTGCTGTGC
TGGGTTCTGCTGTGTCTGCTTGGAGCCGGACCTGTGAAAGCTGGCGTGACCCAGA
CACCTAGATACCTGATCAAGACCAGAGGCCAGCAAGTGACACTGAGCTGTAGCCC
CATCAGCGGCCACAGAAGCGTGTCCTGGTATCAGCAGACTCCTGGACAGGGCCTG
CAGTTCCTGTTCGAGTACTTCTCCGAGACACAGAGGAACAAGGGCAACTTCCCCG
GCAGATTCTCCGGCAGACAGTTCAGCAACTCCCGCAGCGAGATGAACGTGTCCAC
ACTGGAACTGGGAGATAGCGCCCTGTACCTGTGTGCCTCTTCTGTGGGAACCGTG
GAACAGTACTTCGGCCCTGGCACAAGACTGACCGTGACCGAGGACCTGAAGAACG
TGTTCCCACCTGAGGTGGCCGTGTTCGAGCCTTCTGAGGCCGAGATCTCTCACAC
CCAGAAAGCCACACTCGTGTGTCTGGCCACCGGCTTCTATCCCGATCACGTGGAA
CTGTCTTGGTGGGTCAACGGCAAAGAGGTGCACAGCGGCGTCTGTACCGATCCTC
AGCCACTGAAAGAGCAGCCCGCTCTGAACGACAGCAGATACTGCCTGTCCTCCAG
ACTGAGAGTGTCCGCCACCTTCTGGCAGAACCCCAGAAACCACTTCAGGTGTCAG
GTGCAGTTTTACGGCCTGAGCGAGAACGACGAGTGGACCCAGGATAGAGCCAAGC
CTGTGACACAGATCGTGTCTGCCGAAGCCTGGGGCAGAGCCGATTGTGGCTTTAC
CAGCGAGAGCTACCAGCAGGGCGTTCTGTCTGCCACCATCCTGTACGAGATCCTG
CTGGGCAAAGCCACTCTGTACGCCGTGTTGGTGTCTGCCCTGGTGCTGATGGCCA
TGGTCAAGCGGAAGGATTCTAGAGGCGGATCCGGAGCCACAAATTTCTCACTGCT
GAAGCAGGCCGGGGATGTTGAGGAAAACCCAGGACCTATGGCTATGCTGCTGGGA
GCCTCTGTGCTGATCCTGTGGCTGCAACCCGATTGGGTCAACAGCCAGCAGAAGA
ACGACGACCAGCAAGTCAAGCAGAACAGCCCCAGCCTGAGCGTGCAAGAGGGCAG
AATCAGCATCCTGAACTGCGACTACACCAACTCTATGTTCGACTACTTTCTGTGG
TACAAGAAGTACCCCGCCGAGGGACCCACCTTCCTGATCAGCATCAGCAGCATCA
AGGACAAGAACGAGGACGGCCGGTTCACCGTGTTTCTGAACAAGAGCGCCAAGCA
CCTGAGCCTGCACATCGTGCCTTCTCAGCCTGGCGATAGCGCCGTGTACTTTTGT
GCTGCCAGCGGCGTGTCAGGCAACACCCCTCTGGTTTTTGGCAAGGGCACACGCC
TGTCCGTGATCGCCAACATTCAGAACCCTGATCCTGCCGTGTACCAGCTGAGAGA
CAGCAAGAGCAGCGACAAGAGCGTGTGCCTGTTCACCGACTTCGACAGCCAGACC
AACGTGTCCCAGAGCAAGGACAGCGACGTGTACATCACCGATAAGTGCGTGCTGG
ACATGCGGAGCATGGACTTCAAGAGCAACAGCGCCGTGGCCTGGTCCAACAAGTC
CGATTTCGCCTGCGCCAACGCCTTCAACAACAGCATTATCCCCGAGGACACATTC
TTCCCAAGTCCTGAGTCCAGCTGCGACGTGAAGCTGGTGGAAAAGAGCTTCGAGA
CAGACACCAACCTGAACTTCCAGAATCTGAGCGTGATCGGCTTCAGAATCCTGCT
GCTGAAGGTGGCCGGATTCAACCTGCTGATGACCCTCAGACTGTGGTCCAGCTGA
11N4A TCR alpha chain-original protein, with signal peptide underlined
SEQ ID NO: 11
MAMLLGASVLILWLQPDWVNSQQKNDDQQVKQNSPSLSVQEGRISILNCDYTNSMFDY
FLWYKKYPAEGPTFLISISSIKDKNEDGRFTVFLNKSAKHLSLHIVPSQPGDSAVYFCAAS
GVSGNTPLVFGKGTRLSVIANIQNPDPAVYQLRDSKSSDKSVCLFTDFDSQTNVSQSKDS
DVYITDKTVLDMRSMDFKSNSAVAWSNKSDFACANAFNNSIIPEDTFFPSPESSCDVKLV
EKSFETDTNLNFQNLSVIGFRILLLKVAGFNLLMTLRLWSS*
11N4A TCR alpha chain-original protein, without signal peptide
SEQ ID NO: 12
QQKNDDQQVKQNSPSLSVQEGRISILNCDYTNSMFDYFLWYKKYPAEGPTFLISISSIKD
KNEDGRFTVFLNKSAKHLSLHIVPSQPGDSAVYFCAASGVSGNTPLVFGKGTRLSVIANI
QNPDPAVYQLRDSKSSDKSVCLFTDFDSQTNVSQSKDSDVYITDKTVLDMRSMDFKSNS
AVAWSNKSDFACANAFNNSIIPEDTFFPSPESSCDVKLVEKSFETDTNLNFQNLSVIGFRI
LLLKVAGFNLLMTLRLWSS*
11N4A TCR alpha chain variable domain, without signal peptide
SEQ ID NO: 13
QQKNDDQQVKQNSPSLSVQEGRISILNCDYTNSMFDYFLWYKKYPAEGPTFLISISSIKD
KNEDGRFTVFLNKSAKHLSLHIVPSQPGDSAVYFCAASGVSGNTPLVFGKGTRLSVIA
11N4A TCR alpha chain variable domain CDRla
SEQ ID NO: 14
NSMFDY
11N4A TCR alpha chain variable domain CDR2a
SEQ ID NO: 15
ISSIKDK
11N4A TCR alpha chain variable domain CDR3a-IMGT junction
SEQ ID NO: 16
CAASGVSGNTPLVF
11N4A TCR alpha chain variable domain CDR3a-IMGT
SEQ ID NO: 17
AASGVSGNTPLV
11N4A TCR alpha chain constant domain (original protein)
SEQ ID NO: 18
NIQNPDPAVYQLRDSKSSDKSVCLFTDFDSQTNVSQSKDSDVYITDKTVLDMRSMDFKS
NSAVAWSNKSDFACANAFNNSIIPEDTFFPSPESSCDVKLVEKSFETDTNLNFQNLSVIGF
RILLLKVAGFNLLMTLRLWSS
11N4A TCR alpha chain constant domain (cys-modified protein)
SEQ ID NO: 19
NIQNPDPAVYQLRDSKSSDKSVCLFTDFDSQTNVSQSKDSDVYITDKCVLDMRSMDFKS
NSAVAWSNKSDFACANAFNNSIIPEDTFFPSPESSCDVKLVEKSFETDTNLNFQNLSVIGF
RILLLKVAGFNLLMTLRLWSS
11N4A TCR alpha chain, without signal peptide, cys-modified
SEQ ID NO: 20
QQKNDDQQVKQNSPSLSVQEGRISILNCDYTNSMFDYFLWYKKYPAEGPTFLISISSIKD
KNEDGRFTVFLNKSAKHLSLHIVPSQPGDSAVYFCAASGVSGNTPLVFGKGTRLSVIANI
QNPDPAVYQLRDSKSSDKSVCLFTDFDSQTNVSQSKDSDVYITDKCVLDMRSMDFKSNSA
VAWSNKSDFACANAFNNSIIPEDTFFPSPESSCDVKLVEKSFETDTNLNFQNLSVIGFR
ILLLKVAGFNLLMTLRLWSS
11N4A TCR beta chain-original protein, with signal peptide underlined
SEQ ID NO: 21
MGSRLLCWVLLCLLGAGPVKAGVTQTPRYLIKTRGQQVTLSCSPISGHRSVSWYQQTPG
QGLQFLFEYFSETQRNKGNFPGRFSGRQFSNSRSEMNVSTLELGDSALYLCASSVGTVE
QYFGPGTRLTVTEDLKNVFPPEVAVFEPSEAEISHTQKATLVCLATGFYPDHVELSWWV
NGKEVHSGVSTDPQPLKEQPALNDSRYCLSSRLRVSATFWQNPRNHFRCQVQFYGLSE
NDEWTQDRAKPVTQIVSAEAWGRADCGFTSESYQQGVLSATILYEILLGKATLYAVLVS
ALVLMAMVKRKDSRG
11N4A TCR beta chain-original protein, without signal peptide
SEQ ID NO: 22
GVTQTPRYLIKTRGQQVTLSCSPISGHRSVSWYQQTPGQGLQFLFEYFSETQRNKGNFPG
RFSGRQFSNSRSEMNVSTLELGDSALYLCASSVGTVEQYFGPGTRLTVTEDLKNVFPPEV
AVFEPSEAEISHTQKATLVCLATGFYPDHVELSWWVNGKEVHSGVSTDPQPLKEQPALN
DSRYCLSSRLRVSATFWQNPRNHFRCQVQFYGLSENDEWTQDRAKPVTQIVSAEAWGR
ADCGFTSESYQQGVLSATILYEILLGKATLYAVLVSALVLMAMVKRKDSRG
11N4A TCR beta chain variable domain, without signal peptide
SEQ ID NO: 23
GVTQTPRYLIKTRGQQVTLSCSPISGHRSVSWYQQTPGQGLQFLFEYFSETQRNKGNFPG
RFSGRQFSNSRSEMNVSTLELGDSALYLCASSVGTVEQYFGPGTRLTVT
11N4A TCR beta chain variable domain CDR1B
SEQ ID NO: 24
SGHRS
11N4A TCR beta chain variable domain CDR2B
SEQ ID NO: 25
YFSETQ
11N4A TCR beta chain variable domain CDR3B-IMGT junction
SEQ ID NO: 26
CASSVGTVEQYF
11N4A TCR beta chain variable domain CDR3B-IMGT
SEQ ID NO: 27
ASSVGTVEQY
11N4A TCR beta chain constant domain (original protein)
SEQ ID NO: 28
EDLKNVFPPEVAVFEPSEAEISHTQKATLVCLATGFYPDHVELSWWVNGKEVHSGVSTD
PQPLKEQPALNDSRYCLSSRLRVSATFWQNPRNHFRCQVQFYGLSENDEWTQDRAKPV
TQIVSAEAWGRADCGFTSESYQQGVLSATILYEILLGKATLYAVLVSALVLMAMVKRK
DSRG*
11N4A TCR beta chain constant domain (cys-modified protein)
SEQ ID NO: 29
EDLKNVFPPEVAVFEPSEAEISHTQKATLVCLATGFYPDHVELSWWVNGKEVHSGVCT
DPQPLKEQPALNDSRYCLSSRLRVSATFWQNPRNHFRCQVQFYGLSENDEWTQDRAKP
VTQIVSAEAWGRADCGFTSESYQQGVLSATILYEILLGKATLYAVLVSALVLMAMVKR
KDSRG
11N4A TCR beta chain, without signal peptide (cys-modified protein)
SEQ ID NO: 30
GVTQTPRYLIKTRGQQVTLSCSPISGHRSVSWYQQTPGQGLQFLFEYFSETQRNKGNFPG
RFSGRQFSNSRSEMNVSTLELGDSALYLCASSVGTVEQYFGPGTRLTVTEDLKNVFPPEV
AVFEPSEAEISHTQKATLVCLATGFYPDHVELSWWVNGKEVHSGVCTDPQPLKEQPAL
NDSRYCLSSRLRVSATFWQNPRNHFRCQVQFYGLSENDEWTQDRAKPVTQIVSAEAWG
RADCGFTSESYQQGVLSATILYEILLGKATLYAVLVSALVLMAMVKRKDSRG
11N4A TCRbeta-P2A-TCRalpha-Protein, with signal peptides
underlined
SEQ ID NO: 31
MGSRLLCWVLLCLLGAGPVKAGVTQTPRYLIKTRGQQVTLSCSPISGHRSVSWYQQTPG
QGLQFLFEYFSETQRNKGNFPGRFSGRQFSNSRSEMNVSTLELGDSALYLCASSVGTVE
QYFGPGTRLTVTEDLKNVFPPEVAVFEPSEAEISHTQKATLVCLATGFYPDHVELSWWV
NGKEVHSGVCTDPQPLKEQPALNDSRYCLSSRLRVSATFWQNPRNHFRCQVQFYGLSE
NDEWTQDRAKPVTQIVSAEAWGRADCGFTSESYQQGVLSATILYEILLGKATLYAVLVS
ALVLMAMVKRKDSRGGSGATNFSLLKQAGDVEENPGPMAMLLGASVLILWLQPDWV
NSQQKNDDQQVKQNSPSLSVQEGRISILNCDYTNSMFDYFLWYKKYPAEGPTFLISISSI
KDKNEDGRFTVFLNKSAKHLSLHIVPSQPGDSAVYFCAASGVSGNTPLVFGKGTRLSVI
ANIQNPDPAVYQLRDSKSSDKSVCLFTDFDSQTNVSQSKDSDVYITDKCVLDMRSMDF
KSNSAVAWSNKSDFACANAFNNSIIPEDTFFPSPESSCDVKLVEKSFETDTNLNFQNLSVI
GFRILLLKVAGFNLLMTLRLWSS*
CD8alpha-T2A-CD8beta-P2A-11N4A TCRbeta-P2A-alpha-Protein, with
signal peptides underlined
SEQ ID NO: 32
MALPVTALLLPLALLLHAARPSQFRVSPLDRTWNLGETVELKCQVLLSNPTSGCSWLFQ
PRGAAASPTFLLYLSQNKPKAAEGLDTQRFSGKRLGDTFVLTLSDFRRENEGYYFCSAL
SNSIMYFSHFVPVFLPAKPTTTPAPRPPTPAPTIASQPLSLRPEACRPAAGGAVHTRGLDF
ACDIYIWAPLAGTCGVLLLSL VITLYCNHRNRRR VCKCPRPVVKSGDKPSLSARYVGSG
EGRGSLLTCGDVEENPGPMRPRLWLLLAAQLTVLHGNSVLQQTPAYIKVQTNKMVMLS
CEAKISLSNMRIYWLRQRQAPSSDSHHEFLALWDSAKGTIHGEEVEQEKIAVFRDASRFI
LNLTSVKPEDSGIYFCMIVGSPELTFGKGTQLSVVDFLPTTAQPTKKSTLKKRVCRLPRPE
TQKGPLCSPITLGLLVAGVLVLLVSLGVAIHLCCRRRRARLRFMKQFYKGSGATNFSLL
KQAGDVEENPGPMGSRLLCWVLLCLLGAGPVKAGVTQTPRYLIKTRGQQVTLSCSPISG
HRSVSWYQQTPGQGLQFLFEYFSETQRNKGNFPGRFSGRQFSNSRSEMNVSTLELGDSA
LYLCASSVGTVEQYFGPGTRLTVTEDLKNVFPPEVAVFEPSEAEISHTQKATLVCLATGF
YPDHVELSWWVNGKEVHSGVCTDPQPLKEQPALNDSRYCLSSRLRVSATFWQNPRNH
FRCQVQFYGLSENDEWTQDRAKPVTQIVSAEAWGRADCGFTSESYQQGVLSATILYEIL
LGKATLYAVLVSALVLMAMVKRKDSRGGSGATNFSLLKQAGDVEENPGPMAMLLGAS
VLILWLQPDWVNSQQKNDDQQVKQNSPSLSVQEGRISILNCDYTNSMFDYFLWYKKYP
AEGPTFLISISSIKDKNEDGRFTVFLNKSAKHLSLHIVPSQPGDSAVYFCAASGVSGNTPL
VFGKGTRLSVIANIQNPDPAVYQLRDSKSSDKSVCLFTDFDSQTNVSQSKDSDVYITDKC
VLDMRSMDFKSNSAVAWSNKSDFACANAFNNSIIPEDTFFPSPESSCDVKLVEKSFETDT
NLNFQNLSVIGFRILLLKVAGFNLLMTLRLWSS*
11N6 TCR alpha-original nucleotide sequence
SEQ ID NO: 33
Atggaaactctcctgggagtgtctttggtgattctatggcttcaactggctaggg
tgaacagtcaacagggagaagaggatcctcaggccttgagcatccaggagggtga
aaatgccaccatgaactgcagttacaaaactagtataaacaatttacagtggtat
agacaaaattcaggtagaggccttgtccacctaattttaatacgttcaaatgaaa
gagagaaacacagtggaagattaagagtcacgcttgacacttccaagaaaagcag
ttccttgttgatcacggcttcccgggcagcagacactgcttcttacttctgtgct
acggaccctatgaacaccaatgcaggcaaatcaacctttggggatgggactacgc
tcactgtgaagccaaatatccagaaccctgaccctgccgtgtaccagctgagaga
ctctaaatccagtgacaagtctgtctgcctattcaccgattttgattctcaaaca
aatgtgtcacaaagtaaggattctgatgtgtatatcacagacaaaactgtgctag
acatgaggtctatggacttcaagagcaacagtgctgtggcctggagcaacaaatc
tgactttgcatgtgcaaacgccttcaacaacagcattattccagaagacaccttc
ttccccagcccagaaagttcctgtgatgtcaagctggtcgagaaaagctttgaaa
cagatacgaacctaaactttcaaaacctgtcagtgattgggttccgaatcctcct
cctgaaagtggccgggtttaatctgctcatgacgctgcggctgtggtccagctga
11N6 TCR beta-original nucleotide sequence
SEQ ID NO: 34
atgggcaccaggctcctctgctgggcggccctctgtctcctgggagcagaactca
cagaagctggagttgcccagtctcccagatataagattatagagaaaaggcagag
tgtggctttttggtgcaatcctatatctggccatgctaccctttactggtaccag
cagatcctgggacagggcccaaagcttctgattcagtttcagaataacggtgtag
tggatgattcacagttgcctaaggatcgattttctgcagagaggctcaaaggagt
agactccactctcaagatccaacctgcaaagcttgaggactcggccgtgtatctc
tgtgccagcagcccctacggggggagcgtctcctacgagcagtacttcgggccgg
gcaccaggctcacggtcacagaggacctgaaaaacgtgttcccacccgaggtcgc
tgtgtttgagccatcagaagcagagatctcccacacccaaaaggccacactggtg
tgcctggccacaggcttctaccccgaccacgtggagctgagctggtgggtgaatg
ggaaggaggtgcacagtggggtcagcacagacccgcagcccctcaaggagcagcc
cgccctcaatgactccagatactgcctgagcagccgcctgagggtctcggccacc
ttctggcagaacccccgcaaccacttccgctgtcaagtccagttctacgggctct
cggagaatgacgagtggacccaggatagggccaaacctgtcacccagatcgtcag
cgccgaggcctggggtagagcagactgtggcttcacctccgagtcttaccagcaa
ggggtcctgtctgccaccatcctctatgagatcttgctagggaaggccaccttgt
atgccgtgctggtcagtgccctcgtgctgatggccatggtcaagagaaaggattc
cagaggctag
11N6 TCRbeta-P2A-alpha Codon-optimized
SEQ ID NO: 35
ATGGGCACAAGACTTCTCTGTTGGGCTGCACTGTGCTTGCTTGGAGCTGAGCTGAC
AGAAGCTGGAGTTGCCCAATCTCCTAGGTACAAGATCATCGAGAAGCGGCAGTCT
GTGGCCTTTTGGTGCAATCCCATTAGCGGACATGCCACCCTGTACTGGTATCAGC
AAATTCTGGGACAGGGCCCTAAACTGCTGATCCAGTTCCAGAATAACGGCGTGGT
GGACGATTCTCAACTGCCTAAGGACCGGTTTTCTGCCGAGAGACTGAAAGGCGTT
GATAGCACCCTGAAGATCCAACCTGCCAAACTGGAGGATTCTGCCGTGTACCTGT
GTGCTAGCAGCCCTTATGGAGGATCTGTGTCTTATGAGCAGTACTTCGGACCTGG
CACCAGACTGACCGTGACTGAAGACCTGAAGAACGTGTTCCCCCCAGAGGTGGCC
GTGTTCGAGCCTAGCGAGGCCGAGATCAGCCACACCCAGAAAGCCACCCTCGTGT
GCCTGGCCACCGGCTTTTACCCCGACCACGTGGAACTGTCTTGGTGGGTCAACGG
CAAAGAGGTGCACAGCGGCGTCTGCACCGACCCCCAGCCCCTGAAAGAGCAGCCC
GCCCTGAACGACAGCCGGTACTGTCTGAGCAGCAGACTGAGAGTGTCCGCCACCT
TCTGGCAGAACCCCCGGAACCACTTCAGATGCCAGGTGCAGTTCTACGGCCTGAG
CGAGAACGACGAGTGGACCCAGGACCGGGCCAAGCCCGTGACCCAGATCGTGTCT
GCTGAGGCCTGGGGCAGAGCCGATTGCGGCTTCACCAGCGAGAGCTACCAGCAGG
GCGTGCTGAGCGCCACCATCCTGTACGAGATCCTGCTGGGCAAGGCCACCCTGTA
CGCCGTGCTGGTGTCCGCCCTGGTGCTGATGGCCATGGTCAAGCGGAAGGACAGC
CGGGGCGGTTCCGGAGCCACGAACTTCTCTCTGTTAAAGCAAGCAGGAGACGTGG
AAGAAAACCCCGGTCCCATGGAGACACTGCTTGGCGTATCACTGGTGATTCTGTG
GCTGCAACTGGCTAGAGTGAACTCTCAGCAGGGAGAAGAGGATCCTCAAGCTCTG
AGCATTCAGGAAGGCGAAAACGCAACCATGAATTGCTCATACAAGACCAGCATCA
ACAACCTGCAGTGGTACCGGCAGAATAGCGGAAGAGGACTGGTTCACCTGATTTT
AATCAGGTCTAATGAAAGGGAGAAGCACAGCGGCAGACTGAGAGTTACCCTGGAC
ACATCCAAGAAATCTTCTTCTCTGCTGATTACAGCCTCTAGAGCCGCCGATACAG
CCAGCTACTTTTGTGCCACAGATCCCATGAACACCAATGCCGGAAAGAGCACATT
CGGCGATGGCACAACCCTGACAGTTAAGCCCAATATCCAGAATCCCGATCCTGCC
GTGTACCAGCTGCGGGACAGCAAGAGCAGCGACAAGAGCGTGTGCCTGTTCACCG
ACTTCGACAGCCAGACCAACGTGTCCCAGAGCAAGGACAGCGACGTGTACATCAC
CGATAAGTGCGTGCTGGACATGCGGAGCATGGACTTCAAGAGCAACAGCGCCGTG
GCCTGGTCCAACAAGAGCGACTTCGCCTGCGCCAACGCCTTCAACAACAGCATTA
TCCCCGAGGACACATTCTTCCCAAGCCCCGAGAGCAGCTGCGACGTGAAGCTGGT
GGAAAAGAGCTTCGAGACAGACACCAACCTGAACTTCCAGAACCTCAGCGTGATC
GGCTTCCGGATCCTGCTGCTGAAGGTGGCCGGCTTCAACCTGCTGATGACCCTGC
GGCTGTGGTCCAGCTGA
CD8alpha-T2A-CD8beta-P2A-11N6 TCRbeta-P2A-alpha Codon-
optimized
SEQ ID NO: 36
ATGGCTCTGCCTGTGACAGCTCTGCTGCTGCCTCTGGCTCTGCTTCTGCATGCCG
CTAGACCCAGCCAGTTCAGAGTGTCCCCTCTGGACAGAACCTGGAACCTGGGCGA
GACAGTGGAACTGAAGTGCCAGGTGCTGCTGAGCAATCCTACCAGCGGCTGCAGC
TGGCTGTTTCAGCCTAGAGGTGCTGCCGCCTCTCCTACCTTTCTGCTGTACCTGA
GCCAGAACAAGCCCAAGGCCGCCGAAGGACTGGACACCCAGAGATTCAGCGGCAA
GAGACTGGGCGACACCTTCGTGCTGACCCTGAGCGACTTCAGAAGAGAGAACGAG
GGCTACTACTTCTGCAGCGCCCTGAGCAACAGCATCATGTACTTCAGCCACTTCG
TGCCCGTGTTTCTGCCCGCCAAGCCTACAACAACCCCTGCTCCTAGACCTCCTAC
ACCAGCTCCTACAATCGCCAGCCAGCCTCTGTCTCTGAGGCCAGAAGCTTGTAGA
CCTGCTGCTGGCGGAGCCGTGCATACAAGAGGACTGGATTTCGCCTGCGACATCT
ACATCTGGGCCCCTCTGGCTGGAACATGTGGCGTGCTGCTGCTGTCCCTGGTCAT
CACCCTGTACTGCAACCACCGGAACAGGCGGAGAGTGTGCAAGTGCCCTAGACCT
GTGGTCAAGAGCGGCGACAAGCCTAGCCTGAGCGCCAGATATGTTGGCAGCGGAG
AAGGCAGAGGCTCCCTGCTTACATGCGGCGACGTGGAAGAGAACCCCGGACCTAT
GAGGCCTAGACTGTGGCTGCTTCTGGCTGCCCAGCTGACAGTGCTGCACGGCAAT
TCTGTCCTGCAGCAGACCCCTGCCTACATCAAGGTGCAGACCAACAAGATGGTCA
TGCTGAGCTGCGAGGCCAAGATCAGCCTGTCCAACATGCGGATCTACTGGCTGCG
GCAGAGACAGGCCCCTAGCTCTGATAGCCACCACGAGTTTCTGGCCCTGTGGGAT
TCTGCCAAGGGCACCATTCACGGCGAGGAAGTGGAACAAGAGAAGATCGCCGTGT
TCCGGGACGCCAGCAGATTCATCCTGAACCTGACCAGCGTGAAGCCCGAGGACAG
CGGCATCTATTTCTGCATGATCGTGGGCAGCCCCGAGCTGACATTTGGCAAGGGA
ACACAGCTGAGCGTGGTGGACTTCCTGCCTACTACAGCCCAGCCTACCAAGAAGT
CTACCCTGAAGAAACGCGTGTGCAGACTGCCCAGGCCTGAGACACAAAAGGGCCC
TCTGTGCAGCCCTATCACACTGGGATTGCTGGTGGCTGGCGTTCTGGTCCTGCTG
GTGTCTCTGGGAGTTGCCATCCACCTGTGCTGTAGAAGAAGGCGGGCCAGACTGC
GGTTCATGAAGCAGTTCTACAAAGGCAGCGGCGCCACCAACTTCAGCCTGCTGAA
ACAAGCCGGCGACGTCGAGGAAAATCCTGGACCTATGGGCACAAGACTTCTCTGT
TGGGCTGCACTGTGCTTGCTTGGAGCTGAGCTGACAGAAGCTGGAGTTGCCCAAT
CTCCTAGGTACAAGATCATCGAGAAGCGGCAGTCTGTGGCCTTTTGGTGCAATCC
CATTAGCGGACATGCCACCCTGTACTGGTATCAGCAAATTCTGGGACAGGGCCCT
AAACTGCTGATCCAGTTCCAGAATAACGGCGTGGTGGACGATTCTCAACTGCCTA
AGGACCGGTTTTCTGCCGAGAGACTGAAAGGCGTTGATAGCACCCTGAAGATCCA
ACCTGCCAAACTGGAGGATTCTGCCGTGTACCTGTGTGCTAGCAGCCCTTATGGA
GGATCTGTGTCTTATGAGCAGTACTTCGGACCTGGCACCAGACTGACCGTGACTG
AAGACCTGAAGAACGTGTTCCCCCCAGAGGTGGCCGTGTTCGAGCCTAGCGAGGC
CGAGATCAGCCACACCCAGAAAGCCACCCTCGTGTGCCTGGCCACCGGCTTTTAC
CCCGACCACGTGGAACTGTCTTGGTGGGTCAACGGCAAAGAGGTGCACAGCGGCG
TCTGCACCGACCCCCAGCCCCTGAAAGAGCAGCCCGCCCTGAACGACAGCCGGTA
CTGTCTGAGCAGCAGACTGAGAGTGTCCGCCACCTTCTGGCAGAACCCCCGGAAC
CACTTCAGATGCCAGGTGCAGTTCTACGGCCTGAGCGAGAACGACGAGTGGACCC
AGGACCGGGCCAAGCCCGTGACCCAGATCGTGTCTGCTGAGGCCTGGGGCAGAGC
CGATTGCGGCTTCACCAGCGAGAGCTACCAGCAGGGCGTGCTGAGCGCCACCATC
CTGTACGAGATCCTGCTGGGCAAGGCCACCCTGTACGCCGTGCTGGTGTCCGCCC
TGGTGCTGATGGCCATGGTCAAGCGGAAGGACAGCCGGGGCGGTTCCGGAGCCAC
CAACTTCAGCCTGCTTAAACAGGCCGGCGACGTGGAAGAGAACCCTGGACCTATG
GAGACACTGCTTGGCGTATCACTGGTGATTCTGTGGCTGCAACTGGCTAGAGTGA
ACTCTCAGCAGGGAGAAGAGGATCCTCAAGCTCTGAGCATTCAGGAAGGCGAAAA
CGCAACCATGAATTGCTCATACAAGACCAGCATCAACAACCTGCAGTGGTACCGG
CAGAATAGCGGAAGAGGACTGGTTCACCTGATTTTAATCAGGTCTAATGAAAGGG
AGAAGCACAGCGGCAGACTGAGAGTTACCCTGGACACATCCAAGAAATCTTCTTC
TCTGCTGATTACAGCCTCTAGAGCCGCCGATACAGCCAGCTACTTTTGTGCCACA
GATCCCATGAACACCAATGCCGGAAAGAGCACATTCGGCGATGGCACAACCCTGA
CAGTTAAGCCCAATATCCAGAATCCCGATCCTGCCGTGTACCAGCTGCGGGACAG
CAAGAGCAGCGACAAGAGCGTGTGCCTGTTCACCGACTTCGACAGCCAGACCAAC
GTGTCCCAGAGCAAGGACAGCGACGTGTACATCACCGATAAGTGCGTGCTGGACA
TGCGGAGCATGGACTTCAAGAGCAACAGCGCCGTGGCCTGGTCCAACAAGAGCGA
CTTCGCCTGCGCCAACGCCTTCAACAACAGCATTATCCCCGAGGACACATTCTTC
CCAAGCCCCGAGAGCAGCTGCGACGTGAAGCTGGTGGAAAAGAGCTTCGAGACAG
ACACCAACCTGAACTTCCAGAACCTCAGCGTGATCGGCTTCCGGATCCTGCTGCT
GAAGGTGGCCGGCTTCAACCTGCTGATGACCCTGCGGCTGTGGTCCAGCTGA
11N6 TCR alpha chain-original protein, with signal peptide underlined
SEQ ID NO: 37
METLLGVSLVILWLQLARVNSQQGEEDPQALSIQEGENATMNCSYKTSINNLQWYRQN
SGRGL VHLILIRSNEREKHSGRLRVTLDTSKKSSSLLITASRAADTASYFCATDPMNTNA
GKSTFGDGTTLTVKPNIQNPDPAVYQLRDSKSSDKSVCLFTDFDSQTNVSQSKDSDVYIT
DKTVLDMRSMDFKSNSAVAWSNKSDFACANAFNNSIIPEDTFFPSPESSCDVKL VEKSFE
TDTNLNFQNLSVIGFRILLLKVAGFNLLMTLRLWSS*
11N6 TCR alpha chain-original protein, without signal peptide
SEQ ID NO: 38
QQGEEDPQALSIQEGENATMNCSYKTSINNLQWYRQNSGRGLVHLILIRSNEREKHSGR
LRVTLDTSKKSSSLLITASRAADTASYFCATDPMNTNAGKSTFGDGTTLTVKPNIQNPDP
AVYQLRDSKSSDKSVCLFTDFDSQTNVSQSKDSDVYITDKTVLDMRSMDFKSNSAVAW
SNKSDFACANAFNNSIIPEDTFFPSPESSCDVKLVEKSFETDTNLNFQNLSVIGFRILLLKV
AGFNLLMTLRLWSS*
11N6 TCR alpha chain variable domain, without signal peptide
SEQ ID NO: 39
QQGEEDPQALSIQEGENATMNCSYKTSINNLQWYRQNSGRGLVHLILIRSNEREKHSGR
LRVILDTSKKSSSLLITASRAADTASYFCATDPMNTNAGKSTFGDGTTLTVKP
11N6 TCR alpha chain variable domain CDR1a
SEQ ID NO: 40
TSINN
11N6 TCR alpha chain variable domain CDR2a
SEQ ID NO: 41
IRSNERE
11N6 TCR alpha chain variable domain CDR3a-IMGT junction
SEQ ID NO: 42
CATDPMNTNAGKSTF
11N6 TCR alpha chain variable domain CDR3a-IMGT
SEQ ID NO: 43
ATDPMNTNAGKST
11N6 TCR alpha chain constant domain (original protein)
SEQ ID NO: 44
NIQNPDPAVYQLRDSKSSDKSVCLFTDFDSQTNVSQSKDSDVYITDKTVLDMRSMDFKS
NSAVAWSNKSDFACANAFNNSIIPEDTFFPSPESSCDVKLVEKSFETDTNLNFQNLSVIGF
RILLLKVAGFNLLMTLRLWSS
11N6 TCR alpha chain constant domain (cys-modified protein)
SEQ ID NO: 45
NIQNPDPAVYQLRDSKSSDKSVCLFTDFDSQTNVSQSKDSDVYITDKCVLDMRSMDFKS
NSAVAWSNKSDFACANAFNNSIIPEDTFFPSPESSCDVKLVEKSFETDTNLNFQNLSVIGF
RILLLKVAGFNLLMTLRLWSS
11N6 TCR alpha chain, without signal peptide, cys-modified
SEQ ID NO: 46
QQGEEDPQALSIQEGENATMNCSYKTSINNLQWYRQNSGRGLVHLILIRSNEREKHSGR
LRVTLDTSKKSSSLLITASRAADTASYFCATDPMNTNAGKSTFGDGTTLTVKPNIQNPDP
AVYQLRDSKSSDKSVCLFTDFDSQTNVSQSKDSDVYITDKCVLDMRSMDFKSNSAVAW
SNKSDFACANAFNNSIIPEDTFFPSPESSCDVKL VEKSFETDTNLNFQNLSVIGFRILLLKV
AGFNLLMTLRLWSS
11N6 TCR beta chain original protein, with signal peptide underlined
SEQ ID NO: 47
MGTRLLCWAALCLLGAELTEAGVAQSPRYKIIEKRQSVAFWCNPISGHATLYWYQQILG
QGPKLLIQFQNNGVVDDSQLPKDRFSAERLKGVDSTLKIQPAKLEDSAVYLCASSPYGG
SVSYEQYFGPGTRLTVTEDLKNVFPPEVAVFEPSEAEISHTQKATLVCLATGFYPDHVEL
SWWVNGKEVHSGVSTDPQPLKEQPALNDSRYCLSSRLRVSATFWQNPRNHFRCQVQFY
GLSENDEWTQDRAKPVTQIVSAEAWGRADCGFTSESYQQGVLSATILYEILLGKATLYA
VLVSALVLMAMVKRKDSRG
11N6 TCR beta chain original protein, without signal peptide
SEQ ID NO: 48
GVAQSPRYKIIEKRQSVAFWCNPISGHATLYWYQQILGQGPKLLIQFQNNGVVDDSQLP
KDRFSAERLKGVDSTLKIQPAKLEDSAVYLCASSPYGGSVSYEQYFGPGTRLTVTEDLK
NVFPPEVAVFEPSEAEISHTQKATLVCLATGFYPDHVELSWWVNGKEVHSGVSTDPQPL
KEQPALNDSRYCLSSRLRVSATFWQNPRNHFRCQVQFYGLSENDEWTQDRAKPVTQIV
SAEAWGRADCGFTSESYQQGVLSATILYEILLGKATLYAVLVSALVLMAMVKRKDSRG
11N6 TCR beta chain variable domain, without signal peptide
SEQ ID NO: 49
GVAQSPRYKIIEKRQSVAFWCNPISGHATLYWYQQILGQGPKLLIQFQNNGVVDDSQLP
KDRFSAERLKGVDSTLKIQPAKLEDSAVYLCASSPYGGSVSYEQYFGPGTRLTVT
11N6 TCR beta chain variable domain CDR1B
SEQ ID NO: 50
SGHAT
11N6 TCR beta chain variable domain CDR2B
SEQ ID NO: 51
FQNNGV
11N6 TCR beta chain variable domain CDR3B-IMGT junction
SEQ ID NO: 52
CASSPYGGSVSYEQYF
11N6 TCR beta chain variable domain CDR3B-IMGT
SEQ ID NO: 53
ASSPYGGSVSYEQY
11N6 TCR beta chain constant domain (original protein)
SEQ ID NO: 54
EDLKNVFPPEVAVFEPSEAEISHTQKATLVCLATGFYPDHVELSWWVNGKEVHSGVSTD
PQPLKEQPALNDSRYCLSSRLRVSATFWQNPRNHFRCQVQFYGLSENDEWTQDRAKPV
TQIVSAEAWGRADCGFTSESYQQGVLSATILYEILLGKATLYAVLVSALVLMAMVKRK
DSRG
11N6 TCR beta chain constant domain (cys-modified protein)
SEQ ID NO: 55
EDLKNVFPPEVAVFEPSEAEISHTQKATLVCLATGFYPDHVELSWWVNGKEVHSGVCT
DPQPLKEQPALNDSRYCLSSRLRVSATFWQNPRNHFRCQVQFYGLSENDEWTQDRAKP
VTQIVSAEAWGRADCGFTSESYQQGVLSATILYEILLGKATLYAVLVSALVLMAMVKR
KDSRG
11N6 TCR beta chain (cys-modified protein)
SEQ ID NO: 56
GVAQSPRYKIIEKRQSVAFWCNPISGHATLYWYQQILGQGPKLLIQFQNNGVVDDSQLP
KDRFSAERLKGVDSTLKIQPAKLEDSAVYLCASSPYGGSVSYEQYFGPGTRLTVTEDLK
NVFPPEVAVFEPSEAEISHTQKATLVCLATGFYPDHVELSWWVNGKEVHSGVCTDPQPL
KEQPALNDSRYCLSSRLRVSATFWQNPRNHFRCQVQFYGLSENDEWTQDRAKPVTQIV
SAEAWGRADCGFTSESYQQGVLSATILYEILLGKATLYAVLVSALVLMAMVKRKDSRG
11N6 TCRbeta-P2A-alpha-Protein, with signal peptides underlined
SEQ ID NO: 57
MGTRLLCWAALCLLGAELTEAGVAQSPRYKIIEKRQSVAFWCNPISGHATLYWYQQILG
QGPKLLIQFQNNGVVDDSQLPKDRFSAERLKGVDSTLKIQPAKLEDSAVYLCASSPYGG
SVSYEQYFGPGTRLTVTEDLKNVFPPEVAVFEPSEAEISHTQKATLVCLATGFYPDHVEL
SWWVNGKEVHSGVCTDPQPLKEQPALNDSRYCLSSRLRVSATFWQNPRNHFRCQVQF
YGLSENDEWTQDRAKPVTQIVSAEAWGRADCGFTSESYQQGVLSATILYEILLGKATLY
AVLVSALVLMAMVKRKDSRGGSGATNFSLLKQAGDVEENPGPMETLLGVSLVILWLQL
ARVNSQQGEEDPQALSIQEGENATMNCSYKTSINNLQWYRQNSGRGLVHLILIRSNERE
KHSGRLRVTLDTSKKSSSLLITASRAADTASYFCATDPMNTNAGKSTFGDGTTLTVKPNI
QNPDPAVYQLRDSKSSDKSVCLFTDFDSQTNVSQSKDSDVYITDKCVLDMRSMDFKSN
SAVAWSNKSDFACANAFNNSIIPEDTFFPSPESSCDVKLVEKSFETDTNLNFQNLSVIGFR
ILLLKVAGFNLLMTLRLWSS*
CD8alpha-T2A-CD8beta-P2A-11N6 TCRbeta-P2A-alpha-Protein, with
signal peptides underlined
SEQ ID NO: 58
MALPVTALLLPLALLLHAARPSQFRVSPLDRTWNLGETVELKCQVLLSNPTSGCSWLFQ
PRGAAASPTFLLYLSQNKPKAAEGLDTQRFSGKRLGDTFVLTLSDFRRENEGYYFCSAL
SNSIMYFSHFVPVFLPAKPTTTPAPRPPTPAPTIASQPLSLRPEACRPAAGGAVHTRGLDF
ACDIYIWAPLAGTCGVLLLSLVITLYCNHRNRRRVCKCPRPVVKSGDKPSLSARYVGSG
EGRGSLLTCGDVEENPGPMRPRLWLLLAAQLTVLHGNSVLQQTPAYIKVQTNKMVMLS
CEAKISLSNMRIYWLRQRQAPSSDSHHEFLALWDSAKGTIHGEEVEQEKIAVFRDASRFI
LNLTSVKPEDSGIYFCMIVGSPELTFGKGTQLSVVDFLPTTAQPTKKSTLKKRVCRLPRPE
TQKGPLCSPITLGLLVAGVLVLLVSLGVAIHLCCRRRRARLRFMKQFYKGSGATNFSLL
KQAGDVEENPGPMGTRLLCWAALCLLGAELTEAGVAQSPRYKIIEKRQSVAFWCNPISG
HATLYWYQQILGQGPKLLIQFQNNGVVDDSQLPKDRFSAERLKGVDSTLKIQPAKLEDS
AVYLCASSPYGGSVSYEQYFGPGTRLTVTEDLKNVFPPEVAVFEPSEAEISHTQKATLVC
LATGFYPDHVELSWWVNGKEVHSGVCTDPQPLKEQPALNDSRYCLSSRLRVSATFWQ
NPRNHFRCQVQFYGLSENDEWTQDRAKPVTQIVSAEAWGRADCGFTSESYQQGVLSAT
ILYEILLGKATLYAVLVSALVLMAMVKRKDSRGGSGATNFSLLKQAGDVEENPGPMET
LLGVSLVILWLQLARVNSQQGEEDPQALSIQEGENATMNCSYKTSINNLQWYRQNSGR
GLVHLILIRSNEREKHSGRLRVTLDTSKKSSSLLITASRAADTASYFCATDPMNTNAGKS
TFGDGTTLTVKPNIQNPDPAVYQLRDSKSSDKSVCLFTDFDSQTNVSQSKDSDVYITDK
CVLDMRSMDFKSNSAVAWSNKSDFACANAFNNSIIPEDTFFPSPESSCDVKLVEKSFETD
TNLNFQNLSVIGFRILLLKVAGFNLLMTLRLWSS*
TCR BNT Vβ, with signal peptide
SEQ ID NO: 59
MGTRLLCWAALCLLGAELTEAGVAQSPRYKIIEKRQSVAFWCNPISGHATLYWYQQILG
QGPKLLIQFQNNGVVDDSQLPKDRFSAERLKGVDSTLKIQPAKLEDSAVYLCASSLADIY
EQYFGPGTRLTVT
TCR BNT Vα, with signal peptide
SEQ ID NO: 60
METLLGVSLVILWLQLARVNSQQGEEDPQALSIQEGENATMNCSYKTSINNLQWYRQN
SGRGL VHLILIRSNEREKHSGRLRVTLDTSKKSSSLLITASRAADTASYFCATDRQSSGDK
LTFGTGTRLAVRP
(TCR 220_21 Vα)
SEQ ID NO: 61
GEDVEQSLFLSVREGDSSVINCTYTDSSSTYLYWYKQEPGAGLQLLTYIFSNMDMKQDQ
RLTVLLNKKDKHLSLRIADTQTGDSAIYFCAEPIIGGNTPLVFGKGTRLSVIAN
(TCR 220_21 Vβ)
SEQ ID NO: 62
GAGVSQSPRYKVAKRGQDVALRCDPISGHVSLFWYQQALGQGPEFLTYFQNEAQLDKS
GLPSDRFFAERPEGSVSTLKIQRTQQEDSAVYLCASSSEGLAGGPTAGELFFGEGSRLTV
L
(TCR 129_5 Vα)
SEQ ID NO: 63
AQSVTQPDIHITVSEGASLELRCNYSYGATPYLFWYVQSPGQGLQLLLKYFSGDTLVQGI
KGFEAEFKRSQSSFNLRKPSVHWSDAAEYFCAVGASGTYKYIFGTGTRLKVLAN
(TCR 129_5 Vβ)
SEQ ID NO: 64
DAGVIQSPRHEVTEMGQEVTLRCKPISGHNSLFWYRQTMMRGLELLIYFNNNVPIDDSG
MPEDRFSAKMPNASFSTLKIQPSEPRDSAVYFCASSLALSYEQYFGPGTRLTVT
SEQ ID NO: 65
[reserved]
SEQ ID NO: 66
[reserved]
SEQ ID NO: 67
[reserved]
SEQ ID NO: 68
[reserved]
TCR Cα amino acid sequence engineered to include threonine-to-cysteine
and LVL mutations
SEQ ID NO: 69
NIQNPDPAVYQLRDSKSSDKSVCLFTDFDSQTNVSQSKDSDVYITDKCVLDMRSMDFKS
NSAVAWSNKSDFACANAFNNSIIPEDTFFPSPESSCDVKLVEKSFETDTNLNFQNLLVIVL
RILLLKVAGFNLLMTLRLWSS
TRBC1 amino acid sequence (UniProt KB P01850)
SEQ ID NO: 70
EDLNKVFPPEV AVFEPSEAEI SHTQKATLVC LATGFFPDHV ELSWWVNGKE
VHSGVSTDPQ PLKEQPALND SRYCLSSRLR VSATFWQNPR NHFRCQVQFY
GLSENDEWTQ DRAKPVTQIV SAEAWGRADC GFTSVSYQQG VLSATIL YEI
LLGKATLYAV LVSALVLMAM VKRKDF
TRBC1 amino acid sequence (cys-modified)
SEQ ID NO: 71
EDLNKVFPPEVAVFEPSEAEISHTQKATLVCLATGFFPDHVELSWWVNGKEVHSGVCTD
PQPLKEQPALNDSRYCLSSRLRVSATFWQNPRNHFRCQVQFYGLSENDEWTQDRAKPV
TQIVSAEAWGRADCGFTSVSYQQGVLSATILYEILLGKATLYAVLVSALVLMAMVKRK
DF
TRBC2 amino acid sequence (UniProt KB A0A5B9)
SEQ ID NO: 72
EDLKNVFPPKV AVFEPSEAEI SHTQKATLVC LATGFYPDHV ELSWWVNGKE
VHSGVSTDPQ PLKEQPALND SRYCLSSRLR VSATFWQNPR NHFRCQVQFY
GLSENDEWTQ DRAKPVTQIV SAEAWGRADC GFTSESYQQG VLSATILYEI
LLGKATLYAV LVSALVLMAM VKRKDSRG
TRBC2 amino acid sequence (cys-modified)
SEQ ID NO: 73
EDLKNVFPPKV AVFEPSEAEI SHTQKATLVC LATGFYPDHV ELSWWVNGKE
VHSGVCTDPQ PLKEQPALND SRYCLSSRLR VSATFWQNPR NHFRCQVQFY
GLSENDEWTQ DRAKPVTQIV SAEAWGRADC GFTSESYQQG VLSATILYEI
LLGKATLYAV LVSALVLMAM VKRKDSRG
Porcine teschovirus-1 2A (P2A) self-cleaving peptide with N-terminal
GSG linker
(SEQ ID NO.: 74)
GSGATNFSLLKQAGDVEENPGP
Thoseaasigna virus 2A (T2A) self-cleaving peptide
(SEQ ID NO.: 75)
LEGGGEGRGSLLTCGDVEENPGPR
Equine rhinitis A virus (ERAV) 2A (E2A) self-cleaving peptide
(SEQ ID NO.: 76)
QCTNYALLKLAGDVESNPGP
Foot-and-Mouth disease virus 2A (F2A) self-cleaving peptide with N-
terminal G-S-G linker
(SEQ ID NO.: 77)
GSGVKQTLNFDLLKLAGDVESNPGP
NRAS (Uniprot KB P01111)
(SEQ ID NO.: 78)
MTEYKLVVVG AGGVGKSALT IQLIQNHFVD EYDPTIEDSY RKQVVIDGET
CLLDILDTAG QEEYSAMRDQ YMRTGEGFLC VFAINNSKSF ADINLYREQI
KRVKDSDDVP MVLVGNKCDL PTRTVDTKQA HELAKSYGIP FIETSAKTRQ
GVEDAFYTLV REIRQYRMKK LNSSDDGTQG CMGLPCVVM
HRAS (Uniprot KB P01112)
(SEQ ID NO.: 79)
MTEYKLVVVG AGGVGKSALT IQLIQNHFVD EYDPTIEDSY RKQVVIDGET
CLLDILDTAG QEEYSAMRDQ YMRTGEGFLC VFAINNTKSF EDIHQYREQI
KRVKDSDDVP MVLVGNKCDL AARTVESRQA QDLARSYGIP YIETSAKTRQ
GVEDAFYTLV REIRQHKLRK LNPPDESGPG CMSCKCVLS
KRAS 8-16 wild-type (G12)
(SEQ ID NO.: 80)
VVGAGGVGK
(SEQ ID NO.: 81)
KRAS 7-16 wild-type (G12)
VVVGAGGVGK
T2A self-cleaving peptide with N-terminal GSG linker
(SEQ ID NO: 82)
GSGEGRGSLLTCGDVEENPGP
11N4A TCR beta chain, with signal peptide (cys-modified protein), with
signal peptide underlined
(SEQ ID NO: 83)
MGSRLLCWVLLCLLGAGPVKAGVTQTPRYLIKTRGQQVTLSCSPISGHRSVSWYQQTPG
QGLQFLFEYFSETQRNKGNFPGRFSGRQFSNSRSEMNVSTLELGDSALYLCASSVGTVE
QYFGPGTRLTVTEDLKNVFPPEVAVFEPSEAEISHTQKATLVCLATGFYPDHVELSWWV
NGKEVHSGVCTDPQPLKEQPALNDSRYCLSSRLRVSATFWQNPRNHFRCQVQFYGLSE
NDEWTQDRAKPVTQIVSAEAWGRADCGFTSESYQQGVLSATILYEILLGKATLYAVLVS
ALVLMAMVKRKDSRG
Polynucleotide encoding 11N4A TCR beta chain, with signal peptide
(cys-modified protein)
(SEQ ID NO: 84)
ATGGGCAGCAGACTGCTGTGCTGGGTTCTGCTGTGTCTGCTTGGAGCCGGACCTG
TGAAAGCTGGCGTGACCCAGACACCTAGATACCTGATCAAGACCAGAGGCCAGCA
AGTGACACTGAGCTGTAGCCCCATCAGCGGCCACAGAAGCGTGTCCTGGTATCAG
CAGACTCCTGGACAGGGCCTGCAGTTCCTGTTCGAGTACTTCTCCGAGACACAGA
GGAACAAGGGCAACTTCCCCGGCAGATTCTCCGGCAGACAGTTCAGCAACTCCCG
CAGCGAGATGAACGTGTCCACACTGGAACTGGGAGATAGCGCCCTGTACCTGTGT
GCCTCTTCTGTGGGAACCGTGGAACAGTACTTCGGCCCTGGCACAAGACTGACCG
TGACCGAGGACCTGAAGAACGTGTTCCCACCTGAGGTGGCCGTGTTCGAGCCTTC
TGAGGCCGAGATCTCTCACACCCAGAAAGCCACACTCGTGTGTCTGGCCACCGGC
TTCTATCCCGATCACGTGGAACTGTCTTGGTGGGTCAACGGCAAAGAGGTGCACA
GCGGCGTCTGTACCGATCCTCAGCCACTGAAAGAGCAGCCCGCTCTGAACGACAG
CAGATACTGCCTGTCCTCCAGACTGAGAGTGTCCGCCACCTTCTGGCAGAACCCC
AGAAACCACTTCAGGTGTCAGGTGCAGTTTTACGGCCTGAGCGAGAACGACGAGT
GGACCCAGGATAGAGCCAAGCCTGTGACACAGATCGTGTCTGCCGAAGCCTGGGG
CAGAGCCGATTGTGGCTTTACCAGCGAGAGCTACCAGCAGGGCGTTCTGTCTGCC
ACCATCCTGTACGAGATCCTGCTGGGCAAAGCCACTCTGTACGCCGTGTTGGTGT
CTGCCCTGGTGCTGATGGCCATGGTCAAGCGGAAGGATTCTAGAGGCG
11N4A TCR alpha chain (cys-modified protein), with signal peptide
underlined
(SEQ ID NO: 85)
MAMLLGASVLILWLQPDWVNSQQKNDDQQVKQNSPSLSVQEGRISILNCDYTNSMFDY
FLWYKKYPAEGPTFLISISSIKDKNEDGRFTVFLNKSAKHLSLHIVPSQPGDSAVYFCAAS
GVSGNTPLVFGKGTRLSVIANIQNPDPAVYQLRDSKSSDKSVCLFTDFDSQTNVSQSKDS
DVYITDKCVLDMRSMDFKSNSAVAWSNKSDFACANAFNNSIIPEDTFFPSPESSCDVKL
VEKSFETDTNLNFQNLSVIGFRILLLKVAGFNLLMTLRLWSS
Polynucleotide encoding 11N4A TCR alpha chain, with signal peptide
(cys-modified)
(SEQ ID NO: 86)
ATGGCTATGCTGCTGGGAGCCTCTGTGCTGATCCTGTGGCTGCAACCCGATTGGG
TCAACAGCCAGCAGAAGAACGACGACCAGCAAGTCAAGCAGAACAGCCCCAGCCT
GAGCGTGCAAGAGGGCAGAATCAGCATCCTGAACTGCGACTACACCAACTCTATG
TTCGACTACTTTCTGTGGTACAAGAAGTACCCCGCCGAGGGACCCACCTTCCTGA
TCAGCATCAGCAGCATCAAGGACAAGAACGAGGACGGCCGGTTCACCGTGTTTCT
GAACAAGAGCGCCAAGCACCTGAGCCTGCACATCGTGCCTTCTCAGCCTGGCGAT
AGCGCCGTGTACTTTTGTGCTGCCAGCGGCGTGTCAGGCAACACCCCTCTGGTTT
TTGGCAAGGGCACACGCCTGTCCGTGATCGCCAACATTCAGAACCCTGATCCTGC
CGTGTACCAGCTGAGAGACAGCAAGAGCAGCGACAAGAGCGTGTGCCTGTTCACC
GACTTCGACAGCCAGACCAACGTGTCCCAGAGCAAGGACAGCGACGTGTACATCA
CCGATAAGTGCGTGCTGGACATGCGGAGCATGGACTTCAAGAGCAACAGCGCCGT
GGCCTGGTCCAACAAGTCCGATTTCGCCTGCGCCAACGCCTTCAACAACAGCATT
ATCCCCGAGGACACATTCTTCCCAAGTCCTGAGTCCAGCTGCGACGTGAAGCTGG
TGGAAAAGAGCTTCGAGACAGACACCAACCTGAACTTCCAGAATCTGAGCGTGAT
CGGCTTCAGAATCCTGCTGCTGAAGGTGGCCGGATTCAACCTGCTGATGACCCTC
AGACTGTGGTCCAGCTGA
CD8 alpha with signal peptide underlined (amino acid)
(SEQ ID NO: 87)
MALPVTALLLPLALLLHAARPSQFRVSPLDRTWNLGETVELKCQVLLSNPTSGCSWLFQ
PRGAAASPTFLLYLSQNKPKAAEGLDTQRFSGKRLGDTFVLTLSDFRRENEGYYFCSAL
SNSIMYFSHFVPVFLPAKPTTTPAPRPPTPAPTIASQPLSLRPEACRPAAGGAVHTRGLDF
ACDIYIWAPLAGTCGVLLLSL VITLYCNHRNRRRVCKCPRPVVKSGDKPSLSARYV
Polynucleotide encoding CD8 alpha (with signal peptide)
(SEQ ID NO: 88)
ATGGCATTGCCTGTTACAGCTCTGCTGCTGCCCCTGGCTCTGCTTCTGCATGCTG
CTAGACCCAGCCAGTTCAGAGTGTCCCCTCTGGACAGAACCTGGAACCTGGGCGA
GACAGTGGAACTGAAGTGCCAGGTGCTGCTGAGCAATCCTACCAGCGGCTGCAGC
TGGCTGTTTCAGCCTAGAGGTGCTGCCGCCTCTCCTACCTTTCTGCTGTACCTGA
GCCAGAACAAGCCCAAGGCCGCCGAAGGACTGGACACCCAGAGATTCAGCGGCAA
GAGACTGGGCGACACCTTCGTGCTGACCCTGAGCGACTTCAGAAGAGAGAACGAG
GGCTACTACTTCTGCAGCGCCCTGAGCAACAGCATCATGTACTTCAGCCACTTCG
TGCCCGTGTTTCTGCCCGCCAAGCCTACAACAACCCCTGCTCCTAGACCTCCTAC
ACCAGCTCCTACAATCGCCAGCCAGCCTCTGTCTCTGAGGCCAGAAGCTTGTAGA
CCTGCTGCTGGCGGAGCCGTGCATACAAGAGGACTGGATTTCGCCTGCGACATCT
ACATCTGGGCCCCTCTGGCTGGAACATGTGGCGTGCTGCTGCTGTCTCTGGTCAT
CACCCTGTACTGCAACCACCGGAACAGGCGGAGAGTGTGCAAGTGCCCTAGACCT
GTGGTCAAGAGCGGCGACAAGCCTAGCCTGAGCGCCAGATATGTT
CD8 beta with signal peptide underlined
(SEQ ID NO: 89)
MRPRLWLLLAAQLTVLHGNSVLQQTPAYIKVQTNKMVMLSCEAKISLSNMRIYWLRQR
QAPSSDSHHEFLALWDSAKGTIHGEEVEQEKIAVFRDASRFILNLTSVKPEDSGIYFCMIV
GSPELTFGKGTQLSVVDFLPTTAQPTKKSTLKKRVCRLPRPETQKGPLCSPITLGLLVAG
VLVLLVSLGVAIHLCCRRRRARLRFMKQFYK
Polynucleotide encoding CD8 beta, with signal peptide
(SEQ ID NO: 90)
ATGAGGCCTAGACTGTGGCTGCTTCTGGCTGCCCAGCTGACAGTGCTGCACGGCA
ATTCTGTCCTGCAGCAGACCCCTGCCTACATCAAGGTGCAGACCAACAAGATGGT
CATGCTGAGCTGCGAGGCCAAGATCAGCCTGTCCAACATGCGGATCTACTGGCTG
CGGCAGAGACAGGCCCCTAGCAGCGATTCTCACCACGAGTTTCTGGCCCTGTGGG
ATAGCGCCAAGGGAACCATTCACGGCGAGGAAGTGGAACAAGAGAAGATCGCCGT
GTTCCGGGACGCCAGCAGATTCATCCTGAACCTGACCAGCGTGAAGCCCGAGGAC
AGCGGCATCTATTTCTGCATGATCGTGGGCAGCCCCGAGCTGACATTTGGCAAGG
GAACACAGCTGAGCGTGGTGGACTTCCTGCCTACTACAGCCCAGCCTACCAAGAA
GTCTACCCTGAAGAAACGCGTGTGCAGACTGCCCAGGCCTGAGACACAAAAGGGC
CCTCTGTGCAGCCCTATCACACTGGGATTGCTGGTGGCTGGCGTTCTGGTCCTGC
TGGTTTCTCTGGGAGTTGCCATCCACCTGTGCTGCAGACGCAGAAGGGCCAGACT
GCGGTTCATGAAGCAGTTCTACAAA
11N4A alpha FR1 (IMGT)
SEQ ID NO: 91
DQQVKQNSPSLSVQEGRISILNCDYT
11N4A alpha FR2 (IMGT)
SEQ ID NO: 92
FLWYKKYPAEGPTFLIS
11N4A alpha FR3 (IMGT)
SEQ ID NO: 93
NEDGRFTVFLNKSAKHLSLHIVPSQPGDSAVYFC
11N4A alpha FR4 (IMGT)
SEQ ID NO: 94
FGKGTRLSVIA
11N4A alpha FR3 (IMGT junction)
SEQ ID NO: 95
NEDGRFTVFLNKSAKHLSLHIVPSQPGDSAVYF
11N4A alpha FR4 (IMGT junction)
SEQ ID NO: 96
GKGTRLSVIA
sequence from 11N4A beta FR1 (IMGT)
SEQ ID NO: 97
GVTQTPRYLIKTRGQQVTLSCSPI
11N4A beta FR2 (IMGT)
SEQ ID NO: 98
VSWYQQTPGQGLQFLFE
11N4A beta FR3 (IMGT)
SEQ ID NO: 99
RNKGNFPGRFSGRQFSNSRSEMNVSTLELGDSALYLC
sequence from 11N4A beta FR4 (IMGT)
SEQ ID NO: 100
FGPGTRLTV
11N4A beta FR3 (IMGT junction)
SEQ ID NO: 101
RNKGNFPGRFSGRQFSNSRSEMNVSTLELGDSALYL
sequence from 11N4A beta FR4 (IMGT junction)
SEQ ID NO: 102
GPGTRLTV
11N6 alpha FR1 (IMGT)
SEQ ID NO: 103
QQGEEDPQALSIQEGENATMNCSYK
11N6 alpha FR2 (IMGT)
SEQ ID NO: 104
LQWYRQNSGRGLVHLIL
11N6 alpha FR3 (IMGT)
SEQ ID NO: 105
KHSGRLRVTLDTSKKSSSLLITASRAADTASYFC
11N6 alpha FR4 (IMGT)
SEQ ID NO: 106
FGDGTTLTVKP
11N6 alpha FR3 (IMGT junction)
SEQ ID NO: 107
KHSGRLRVTLDTSKKSSSLLITASRAADTASYF
11N6 alpha FR4 (IMGT junction)
SEQ ID NO: 108
GDGTTLTVKP
11N6 beta FR1 (IMGT)
SEQ ID NO: 109
GVAQSPRYKIIEKRQSVAFWCNPI
11N6 beta FR2 (IMGT)
SEQ ID NO: 110
LYWYQQILGQGPKLLIQ
11N6 beta FR3 (IMGT)
SEQ ID NO: 111
VDDSQLPKDRFSAERLKGVDSTLKIQPAKLEDSAVYLC
11N6 beta FR4 (IMGT)
SEQ ID NO: 112
FGPGTRLTVT
11N6 beta FR3 (IMGT junction)
SEQ ID NO: 113
VDDSQLPKDRFSAERLKGVDSTLKIQPAKLEDSAVYL
11N6 beta FR4 (IMGT junction)
SEQ ID NO: 114
GPGTRLTVT
sequence comprising 11N4A alpha FR1 (IMGT)
SEQ ID NO: 115
QQKNDDQQVKQNSPSLSVQEGRISILNCDYT
11N4A beta FR4 (IMGT)
SEQ ID NO: 116
FGPGTRLTVT
11N4A beta FR4 (IMGT junction)
SEQ ID NO: 117
GPGTRLTVT
CRNS1 peptide
SEQ ID NO: 118
VVGAGGVSK
RASE peptide
SEQ ID NO: 119
VVGASGVGK
RAB7B peptide
SEQ ID NO: 120
IVGAIGVGK
SEQ ID NO: 121
[reserved]
ITA8 peptide
SEQ ID NO: 122
IVGAFGTGK
MIRO2 peptide
SEQ ID NO: 123
VVGARGVGK
ARRD4 peptide
SEQ ID NO: 124
AVGAEGR VK
SMC5 peptide
SEQ ID NO: 125
IVGANGTGK
MOGS peptide
SEQ ID NO: 126
EVGAKGQLK
GKN2 peptide
SEQ ID NO: 127
NVGAGGCAK
AFDDT peptide
SEQ ID NO: 128
QMGAAGSGR
ANKR9 peptide
SEQ ID NO: 129
PVGAAGSAR
CFTR peptide
SEQ ID NO: 130
VAGSTGAGK
DYH2 peptide
SEQ ID NO: 131
IVGCTGSGK
EP300 peptide
SEQ ID NO: 132
MNGSIGAGR
FOXA2 peptide
SEQ ID NO: 133
AAGAAGSGK
HTR5B peptide
SEQ ID NO: 134
ASGAVGSAK
LARP1 peptide
SEQ ID NO: 135
AAGAAGAGR
MED1 peptide
SEQ ID NO: 136
NVGSTGVAK
MOD5 peptide
SEQ ID NO: 137
ILGATGTGK
MRP5 peptide
SEQ ID NO: 138
ICGSVGSGK
NAL12 peptide
SEQ ID NO: 139
MQGAAGIGK
PCGF6 peptide
SEQ ID NO: 140
TAGSVGAAK
RAB4B peptide
VIGSAGTGK
SEQ ID NO: 141
SCGR4 peptide
SEQ ID NO: 142
TCGSCGCGY
3HIDH peptide
SEQ ID NO: 143
VSGGVGAAR
AFDDT-2 peptide
SEQ ID NO: 144
PMGGTGSGR
BOLA3 peptide
SEQ ID NO: 145
ISGGCGAMY
DYH9 peptide
SEQ ID NO: 146
VVGGAGTGK
KRA53 peptide
SEQ ID NO: 147
SCGGCGSGY
SHRM1 peptide
SEQ ID NO: 148
VNGSVGISR
Alanine scan peptide search string, 9-mer
SEQ ID NO: 149
V-X-G-A-X-G-X-X-K
Alanine scan peptide search string, 10-mer
SEQ ID NO: 150
X-X-V-G-A-V-G-X-X-K
Alanine scan peptide search string, 9-mer
SEQ ID NO: 151
X-X-G-A-X-G-X-X-K
Xscan peptide search string
SEQ ID NO: 152
[ANCQIMPSTV]-[ANCQILMSTV]-G-[CSA]-[ACQHIMTV]-G-[ACISTV]-[AEMG]-[RYK]
11N4A beta FR1 (IMGT)
SEQ ID NO: 153
KAGVTQTPRYLIKTRGQQVTLSCSPI
KRAS G12V-specific binding protein polypeptide sequence
SEQ ID NO: 154
KAGVTQTPRYLIKTRGQQVTLSCSPISGHRSVSWYQQTPGQGLQFLFEYFSETQRN
KGNFPGRFSGRQFSNSRSEMNVSTLELGDSALYLCASSVGTVEQYFGPGTRLTVT
KRAS G12V-specific binding protein polypeptide sequence
SEQ ID NO: 155
KAGVTQTPRYLIKTRGQQVTLSCSPISGHRSVSWYQQTPGQGLQFLFEYFSETQRNKGN
FPGRFSGRQFSNSRSEMNVSTLELGDSALYLCASSVGTVEQYFGPGTRLTVTEDLKNVFP
PEVAVFEPSEAEISHTQKATLVCLATGFYPDHVELSWWVNGKEVHSGVCTDPQPLKEQP
ALNDSRYCLSSRLRVSATFWQNPRNHFRCQVQFYGLSENDEWTQDRAKPVTQIVSAEA
WGRADCGFTSESYQQGVLSATILYEILLGKATLYAVLVSALVLMAMVKRKDSRG

### EXAMPLES

### Example 1

### Identification of KRAS G12V-Specific TCRs from the T Cell Repertoire of Healthy Donors

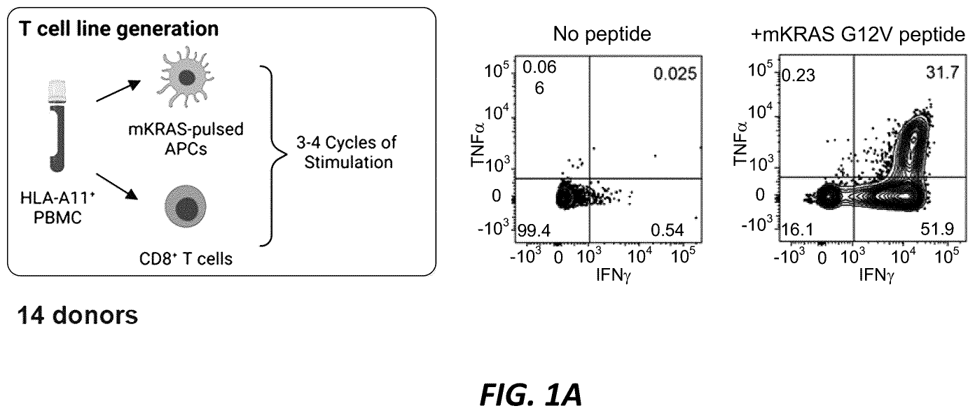

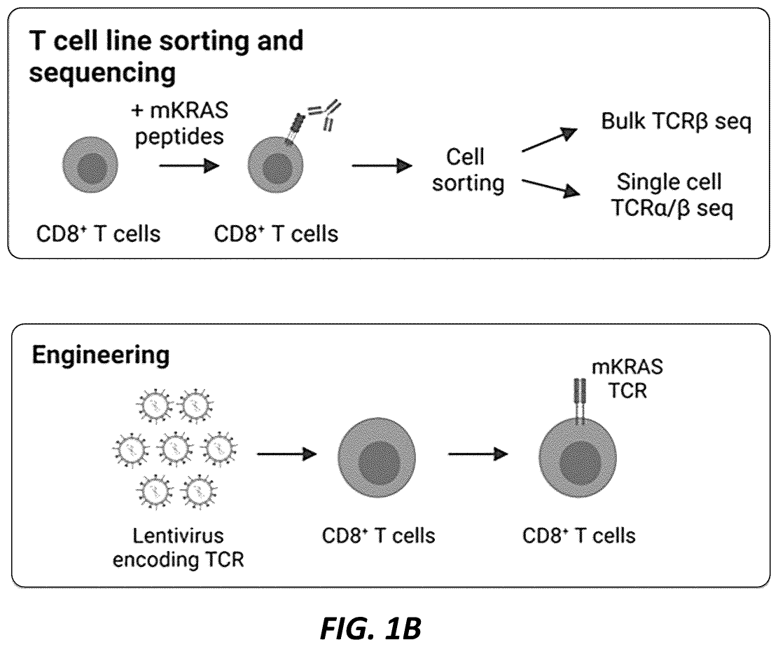

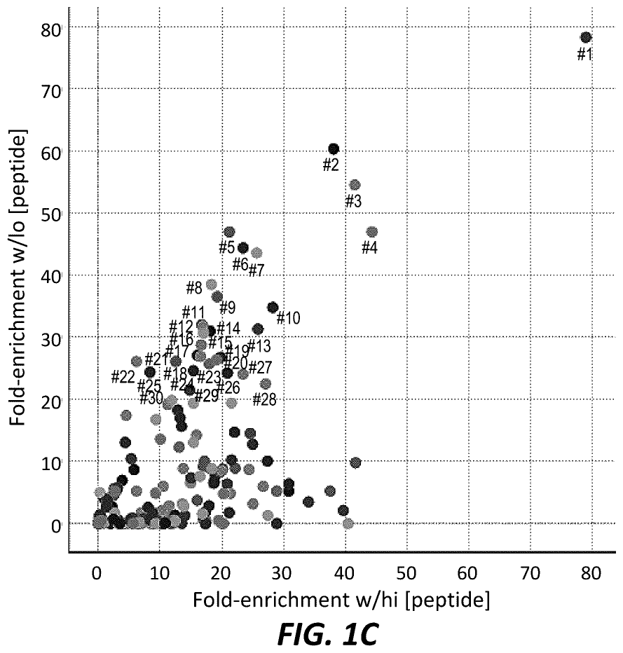

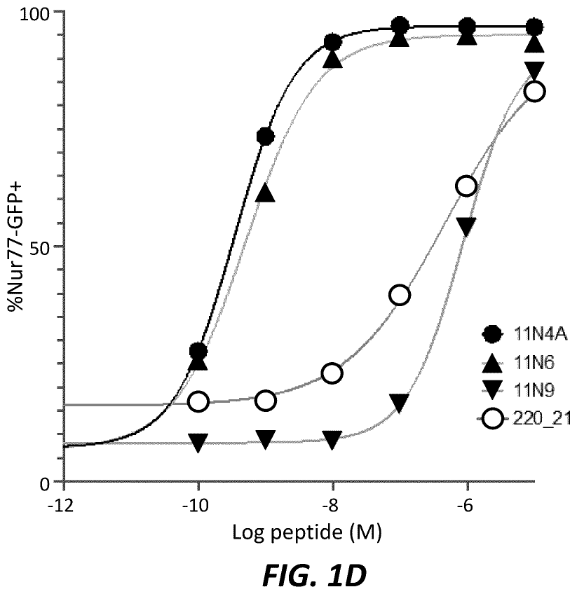

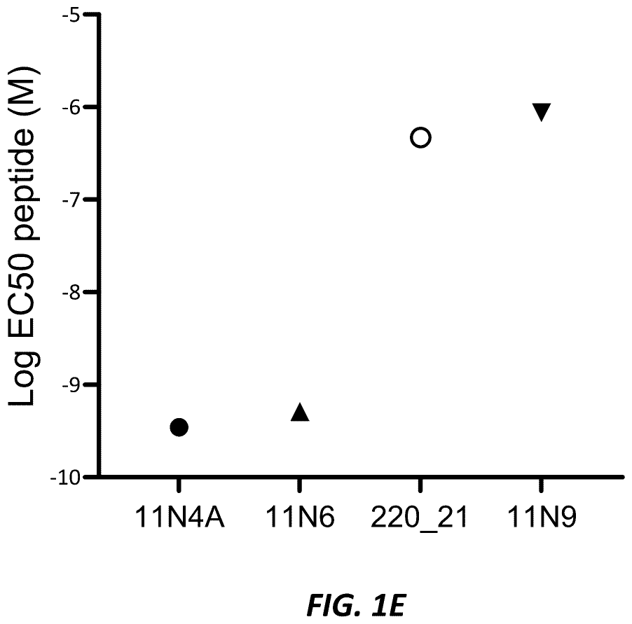

Dendritic cells derived from HLA-A11-positive healthy donor PBMCs were generated, irradiated, and pulsed with KRAS-G12V7-16 and KRAS-G12V₈₋₁₆ peptides. These were incubated for 8-10 days with autologous CD8⁺ T cells to induce activation/expansion of antigen-specific CD8+ T cells. These polyclonal T cell lines were then restimulated and expanded for 8-10 days two times with peptide-pulsed irradiated autologous PBMCs to further expand antigen specific clones. This process was conducted across ten lines of CD8+ T cells from each of 15 HLA-matched donors. (Ho W Y, Nguyen H N, Wolfl M, Kuball J, Greenberg P D. In vitro methods for generating {CD8}+{T}-cell clones for immunotherapy from the naïve repertoire. J Immunol Methods. 2006; 310(1): 40-52. doi:10.1016/j.jim.2005.11.023). FIG. 1A.

To identify TCRs with strong binding, T cells were stimulated overnight with titrated concentrations of cognate KRAS G12V peptides and CD137 upregulation was assessed by flow cytometry. Cells expressing CD137 were isolated by flow cytometric cell sorting and TCR beta repertoire analysis was performed (Adaptive Biotechnologies). TCR clonotypes that were highly enriched in CD137+ populations that responded to low concentrations of peptide were identified, and TCR alpha/beta pairing was determined by 10× single cell RNAseq analysis on similarly sorted populations (10× genomics). (FIG. 1B) Representative analysis of clonotype enrichment in CD137+ sorted populations compared to total unsorted cells treated with low and high peptide concentrations. (FIG. 1C) Paired TCRalpha/beta sequences from identified clonotypes were assembled and synthesized as P2A-linked expression cassettes and lentivirally transduced into reporter Jurkat cells that express GFP under the control of the Nur77 locus (Nur77-GFP-Jurkats). Peptide dose-dependent responses for each TCR were assessed by analyzing GFP expression following overnight culture with A11 target cells pulsed with decreasing concentrations of peptide as indicated. (FIG. 1D) Dose-response curves were fitted by non-linear regression, and EC50 values were calculated using Graphpad Prism.

### Example 2

### Functional Avidity of KRAS-G12V-Specific TCRs Expressed in Primary CD8+ T Cells

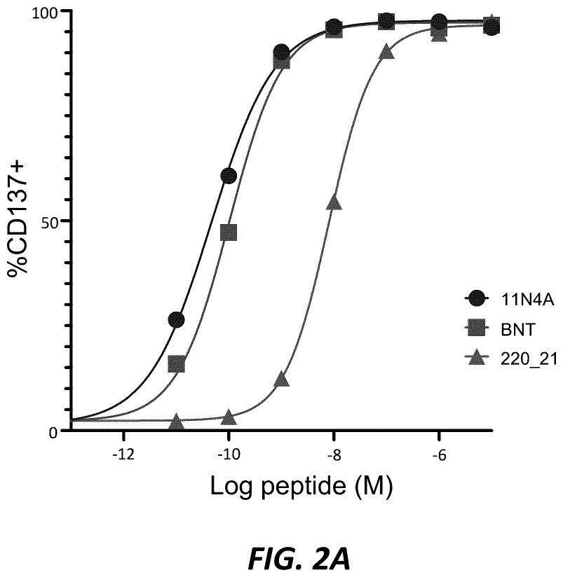

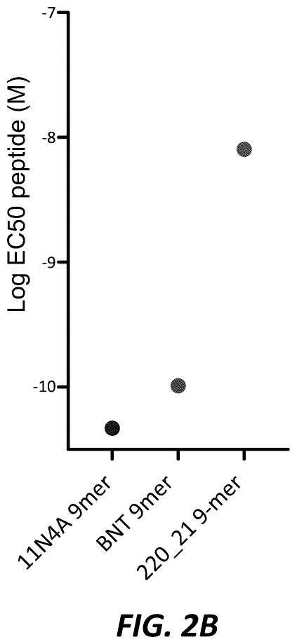

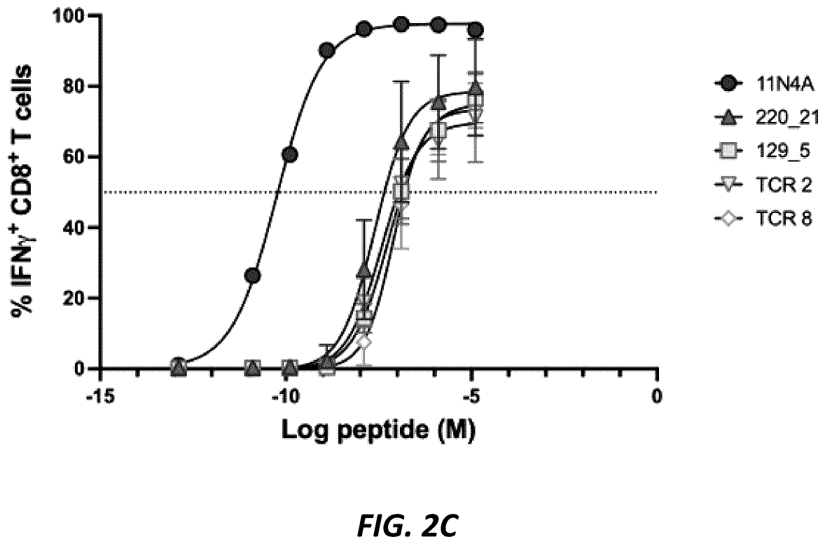

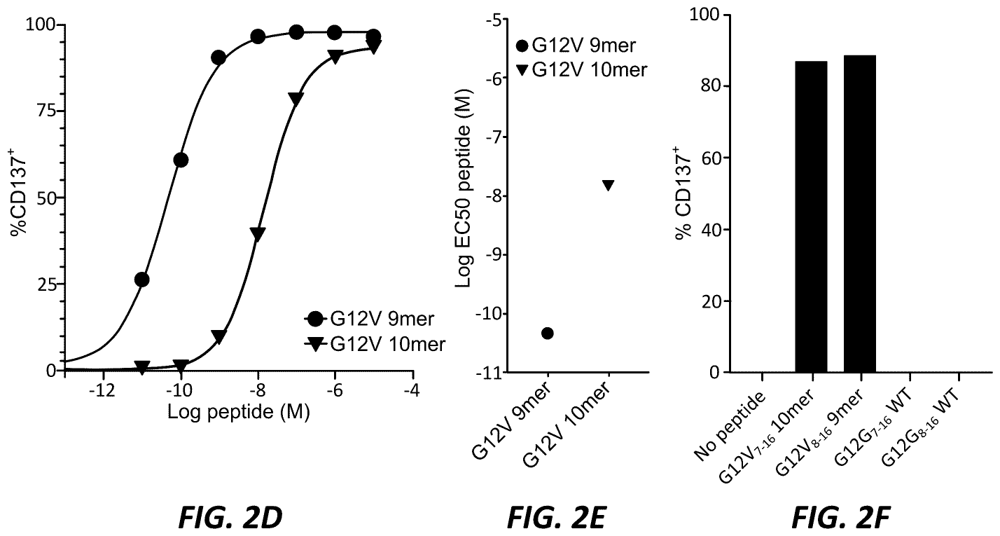

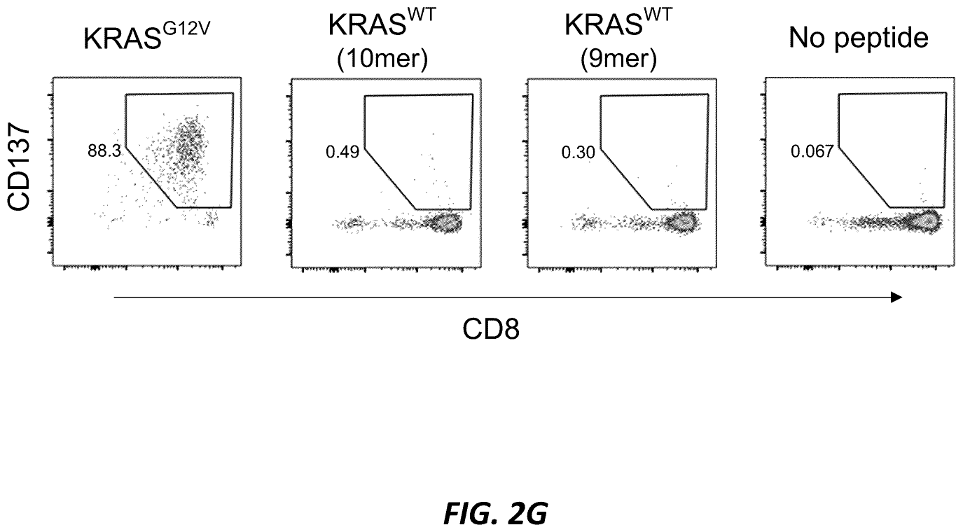

Primary CD8+ T cells were transduced with KRAS-G12V-specific TCRs, sort purified, and expanded. Sort-purified T cells were then cultured overnight with decreasing concentrations of KRAS-G12V₈₋₁₆ peptide and CD137 expression was assessed by flow cytometry. Dose-response curves were fitted by non-linear regression, and EC50 values were calculated using Graphpad Prism. FIGS. 2A, 2B. In this experiment, TCR 11N4A was compared to a KRAS G12V-specific TCR “220_21” (see SEQ ID NOs.: 61 and 62 herein), and to TCR “BNT”, having variable domains encoded by SEQ ID NOs.: 54 (Vα) and 57 (Vβ) of US Pre-Grant Publication No. US 2021/0340215A1 (see also SEQ ID NOs.: 59 and 60 herein). All TCRs were encoded by lentivirus in TCRβ-P2A-TCRα expression cassettes.

TCR 11N4A was compared to 220_21 and other TCRs using a similar assay, measuring peptide antigen dose-response for IFN-γ expression. FIG. 2C. FIGS. 2D-2F show additional functional avidity data for TCR 11N4A.

TCR 11N4A was expressed in primary human CD8+ T cells and further tested in functional avidity studies by a CD137 surface expression assay. TCR 11N4A demonstrated potent functional avidity to the KRAS G12V₈₋₁₆ 9mer (VVGAVGVGK; SEQ ID NO.: 3) and to a lesser extent the KRAS G12V₇₋₁₆ 10mer (VVVGAVGVGK; SEQ ID NO.: 2) peptide, both identified in peptide elution databases (Choi et al., 2021), with an EC50 of 59.5 pM and 18.75 nM, respectively. In contrast, TCR 11N4A did not react to wild-type KRAS 9mer (VVGAGGVGK; SEQ ID NO: 80) or 10mer (VVVGAGGVGK; SEQ ID NO: 81) peptides demonstrating specificity for the tumor-restricted KRASG12V antigen (FIG. 2G).

### Example 3

### KRAS-G12V-Specific TCR-Transduced T Cell Recognition of KRAS-G12V Expressing Tumor Cell Lines

Primary CD8+ T cells were transduced with KRAS-G12V-specific TCRs, sort purified, and expanded. Sort-purified T cells were cultured overnight with tumor cell lines that express mutant KRAS-G12V. T cells cultured with 1 mg/ml of KRAS-G12V₈₋₁₆ peptide were included as a positive control. Tumor lines were first transduced to express HLA-A11 as-needed and sort-purified for HLA-A11 expression. T cell responses were assessed by measuring CD137 expression in response to TCR signaling (FIGS. 3A and 3B). FIG. 3C shows activation of untransduced T cells or 11N4A TCR and CD8αβ co-receptor engineered T cells by endogenous KRAS G12V presentation across diverse tumor cell lines. Activation was assessed by measuring CD137 expression in response to TCR signaling. 11N4A TCR and CD8αβ co-receptor engineered T cells show enhanced activation compared to untransduced T cells.

11N4A-TCR cell products used in the following efficacy and safety studies (except where specified) were generated using methods to be employed for patients. Briefly, healthy donor PBMCs first underwent CD4 positive selection with magnetic beads. The negative fraction flow-through from CD4 selection, containing a majority of CD8+ T cells, was then purified via CD62L positive selection. CD62L selection ensures downstream transduction of CD8+ T cell subsets that have improved in vivo persistence, including naïve, central memory and stem cell memory (Berger et al., 2008; Gattinoni et al., 2011; Wherry & Ahmed, 2004). Pooled CD4+ and CD4−CD62L+ cells were formulated at a 1:1 ratio and stimulated with IL-2, IL-21 and TransAct (anti-CD3/CD28) two days prior to transduction with the 11N4A-TCR lentivirus. Cells were further expanded with exogenous addition of IL-2 and harvested 10 days later to constitute 11N4A-TCR “simulated” products, defined as cells generated similar to the clinical manufacture process but not used for infusion into humans.

To confirm that the 11N4A-TCR simulated product can respond to endogenously expressed and presented KRAS G12V peptide, a panel of tumor cell lines expressing HLA-A*11:01 and KRAS G12V antigen with derivation across the indications intended for the Phase 1 study was tested (FIG. 3D, left). 11N4A-TCR simulated product derived from two different healthy donors was specifically activated by co-culture with all KRAS G12V-expressing tumor cell lines tested, whereas untransduced T cells from the same donors exhibited minimal activation (FIG. 3D, right). Importantly, two cancer cell lines, PANC1 and HUCCT1, which do not express the KRAS G12V mutation were included as negative controls for 11N4A-TCR T cell activation with only background activity observed (FIG. 3D, right). In addition, 11N4A-TCR effector cytokine production was assessed after coculture with the described tumor cell lines (FIG. 3E). All tumor cell lines induced varying but significant secretion of IFNγ, TNFα, and IL-2 after culture with KRAS G12V-expressing cell lines, while negative control tumor cells did not induce cytokine secretion by 11N4A-TCR T cells. In addition to antigen-specific T cell activation and effector cytokine production, 11N4A-TCR T cells specifically proliferated in response to KRAS G12V-expressing tumor cells (FIG. 3F). All KRAS G12V-expressing tumor cell lines tested led to enhanced proliferation of the 11N4A-TCR product while negative control tumor cells only induced background proliferation similar to untransduced T cell controls.

The panel of tumor cell lines tested exhibit a wide range of KRAS G12V expression as determined by western blot analysis (FIG. 3G, left). Analysis of KRAS G12V expression compared to housekeeping protein GAPDH in each cell line revealed a diversity of KRAS G12V expression across the tumor cell line panel (FIG. 3G, right) that could approximate the diversity of KRAS G12V expression expected across the patient population in a clinical setting. As shown above (FIGS. 3D-3F), 11N4A-TCR was able to respond to all KRAS G12V-expressing tumor cell lines, even NCI-H441 that exhibited the lowest KRAS G12V expression of the cell lines tested. These data suggest that 11N4A-TCR has the potential to activate and mediate antitumor activity in response to variable KRAS G12V expression expected in patients.

### Example 4

### Specific Killing of KRAS-G12V-Expressing Tumor Cell Lines by CD8+ T Cells Expressing KRAS-G12V-Specific TCRs

Red fluorescent SW480 cells, a KRAS-G12V expressing tumor cell line transduced to express HLA-A11, were cocultured with TCR-transduced T cells as indicated and enumerated over time by live cell imaging using the IncuCyte S3 microscope and software package. CD8 T cell cytotoxicity is indicated by a decrease in the total red target cell area per well as compared to no treatment wells. Additional tumor cells were added at 72 hours to assess TCR-mediated tumor cell lysis by transduced T cells in the presence of persistent antigen. Three increasingly stringent effector: target cell ratios were used to measure relative TCR-mediated tumor lysis in conditions when T cells are limiting. Data are shown in FIG. 4C. Data from another experiment are shown in FIG. 4B.

Initial studies assessed the efficacy of 11N4A-TCR engineered primary CD4+ and CD8+ T cells (without CD8 α/β coreceptor) against the SW527, SW620, and CFPAC-1 tumor cell lines. 11N4A-TCR engineered primary CD8+ T cells significantly abrogated the in vitro growth and survival of all tumor cell lines in a live tumor-visualization assay, even after repeated tumor challenges within the same assay (FIG. 4D). Subsequent studies using 11N4A-TCR simulated products across two donors, expressing CD8 α/β coreceptor, were tested in in vitro cytotoxicity assays across a large panel of KRAS G12V-tumor cell lines representative of the indications expected in a clinical setting. Against all tumor cell lines, 11N4A-TCR exhibited robust cytotoxic activity (FIGS. 4E-4G). Notably, 11N4A-TCR exhibited potent cytotoxic activity against NCI-H441 that had the lowest expression of KRAS G12V in all tumor cell lines tested by western blot analysis (FIG. 3G). Consistent with the described activation and proliferation studies (FIGS. 3D-3F), 11N4A-TCR did not demonstrate cytotoxic activity against two negative control cancer cell lines, PANC1 and HUCCT1, which do not express the KRAS G12V mutation.

### Example 5

### Mutational Scan to Characterize the Peptide Binding Motif of TCR 11N4A

To assess the potential for cross-reactivity of TCR 11N4A, a mutational scan was performed to identify peptide residues critical for TCR binding. (FIG. 5A) Peptides were synthesized in which each residue of the cognate KRAS-G12V peptide was changed to an alanine. Position 4 of the cognate 9mer peptide (position 5 of the 10mer peptide) already contains an alanine, so peptides were generated that contain a glycine or a threonine at this position. TCR 11N4A-transduced Nur77-GFP-Jurkats were cultured overnight with HLA-A11⁺ B-LCL cells pulsed with 1 mg/ml of each peptide followed by flow cytometric analysis of GFP expression. Peptides that contained a substitution at position 1, 5, 7 or 8 of the 9mer and the corresponding positions of the 10mer could still elicit a response from cells expressing TCR 11N4A, indicating that TCR 11N4A can recognize peptides with other amino acids at these positions (FIGS. 5A and 5B). (FIG. 5C) A search of the human proteome for similar motifs was performed using ScanProsite (prosite.expasy.org/scanprosite/) using the search string: x-V-G-A-x-G-x-x-K (SEQ ID NO.: 4). The resulting potentially cross-reactive peptides are shown in FIG. 5D with predicted HLA-A11 binding data from IEDB (NetPanMHC4.1) shown as percentile rank (lower is better) and score (higher is better). These data include two peptides that each appear in multiple proteins (RASE and RSLBB; wildtype RAS proteins RASH, RASK and RASN).

### Example 6

### Analysis of 11N4A Reactivity to Potentially Cross-Reactive Peptides

(FIGS. 6A, 6B) TCR 11N4A-transduced donor-derived CD8⁺ T cells were cultured overnight with each of the identified potential cross-reactive peptides, or cognate KRAS-G12V peptides (1 mg/ml), and activation-induced CD137 expression was assessed by flow cytometry. No response was detected from any peptides, except for a low-level response (<20%) from a RAB7B-derived peptide. (FIG. 6C) To further assess functional avidity of TCR 11N4A against the RAB7B peptide, sort-purified TCR 11N4A-transduced T cells were cultured overnight with decreasing concentrations of KRAS-G12V₈₋₁₆ peptide or RAB7B peptide and CD137 expression was assessed by flow cytometry. Dose-response curves were fitted by non-linear regression, and EC50 values were calculated using Graphpad Prism (FIG. 6D).

The calculated EC50 for RAB7B peptide was ˜35 mg/ml, a very high concentration of peptide that would result in a density of peptide-loaded MHC on the target cell surface that is several orders of magnitude greater than the density of any particular peptide/HLA-A2 complex presented on the surface of a typical cell. Cells normally present a diverse array of processed cellular proteins, at a density that has been reported to be in the range of 10-150 peptide/MHC complexes per cell for several well-presented self-peptides (Bossi G, Gerry A B, Paston S J, Sutton D H, Hassan N J, Jakobsen B K. Examining the presentation of tumor-associated antigens on peptide-pulsed T2 cells. Oncoimmunology. 2013; 2(11): e26840; Liddy N, Bossi G, Adams K J, Lissina A, Mahon T M, Hassan N J, et al. Monoclonal TCR-redirected tumor cell killing. Nat Med. 2012; 18(6): 980-7; Purbhoo M A, Sutton D H, Brewer J E, Mullings R E, Hill M E, Mahon™, et al. Quantifying and imaging NY-ESO-1/LAGE-1-derived epitopes on tumor cells using high affinity T cell receptors. J Immunol. 2006; 176(12): 7308-16.) To specifically characterize the relationship between peptide concentration and epitope presentation by T2 cells, Jakobson and colleagues used soluble, high-affinity TCRs coupled with single-molecule fluorescence microscopy to quantify several well-characterized self-peptides on peptide-pulsed T2 cells. (Bossi G, Gerry A B, Paston S J, Sutton D H, Hassan N J, Jakobsen B K. Examining the presentation of tumor-associated antigens on peptide-pulsed T2 cells. Oncoimmunology. 2013; 2(11): e26840). The results of this analysis suggest that peptide concentrations in the low nanomolar range (1-10 nM) are required to approximate physiological levels of presented antigen.

In contrast, even at the high dose of 10 mg/ml (˜10 mM), only a low-level response by TCR 11N4A-transduced T cells was observed (˜25% of T cells responding, compared to >80% of T cells responding to the cognate KRAS-G12V peptide). Importantly, no response by TCR 11N4A-transduced T cells was observed with peptide concentrations of 100 nM or lower. These data support that TCR 11N4A-transduced T cells do not have sufficient affinity for the RAB7B peptide to recognize the naturally processed and presented epitope.

To further assess the potential for TCR cross-reactivity, CD8+ T cells expressing TCR 11N4A were cultured overnight with a comprehensive panel of positional scanning peptides containing a substitution of every possible amino acid at each position of the cognate KRAS G12V peptide (172 peptides). The percentage of T cells expressing CD137 in response to each peptide is shown (FIG. 6E), organized by peptide position.

From these data, a potentially cross-reactive peptide motif was determined, and peptides that match that motif were identified by searching the human proteome using ScanProsite (prosite.expasy.org/scanprosite/). Peptides that elicited a response of greater than 15% were considered positive in this assay. The potentially cross-reactive peptides identified from the ScanProsite search are shown in the table (FIG. 6F, left). RAB7B, the only peptide identified as cross-reactive in the mutational scan analysis, was the only peptide that was also identified in the xscan analysis, validating the utility of this type of analysis. The additional peptides identified were synthesized, and added at 100 ng/ml to sort-purified primary CD8⁺ T cells transduced to express TCR 11N4A or TCR 11N4A+CD8αβ. After overnight culture, activation-induced CD137 expression was assessed by flow cytometry. No reactivity was detectable for any of the additional identified peptides (FIG. 6F, right).

Further studies were performed, and the results supported that TCR 11N4A does not recognize candidate human self-peptides. Using the alanine and Xscan data described, search strings were used to identify all potentially cross-reactive self-peptides in the human proteome. Search strings from alanine scan (X-X-G-A-X-G-X-X-K (9mer) (SEQ ID NO.: 151) or X-X-V-G-A-V-G-X-X-K (10mer) (SEQ ID NO.: 150)) and Xscan ([ANCQIMPSTV]-[ANCQILMSTV]-G-[CSA]-[ACQHIMTV]-G-[ACISTV]-[AEMG]-[RYK]) (SEQ ID NO.: 152) were input into the ScanProsite database (prosite.expasy.org/scanprosite/) and self-peptides were identified that are predicted to potentially bind to HLA-A*11:01 using NetMHCpan 4.1 (Table 2; (Andreatta & Nielsen, 2016)).

Table 2 shows results from analyzing potentially cross-reactive peptides identified by in silico analyses. Search strings from alanine scan (V-X-G-A-X-G-X-X-K (9mer) (SEQ ID NO.: 149) or X-X-V-G-A-V-G-X-X-K (10mer) (SEQ ID NO.: 150)) and Xscan ([ANCQIMPSTV]-[ANCQILMSTV]-G-[CSA]-[ACQHIMTV]-G-[ACISTV]-[AEMG]-[RYK]) (SEQ ID NO.: 152) were input into the ScanProsite database (prosite.expasy.org/scanprosite/) against the human proteome to identify potentially self-reactive peptides expressed in normal tissues. No peptides were identified that match the 10mer alanine scan search string. IEDB scores calculated from NetMHCpan 4.1 estimate potential for the identified peptide to bind to HLA-A*11:01 allele with a higher score indicating a greater likelihood of binding.

TABLE 2

| | | | |
|---|---|---|---|
| Gene Name | Sequence | HLA Search | IEDB Score |

Based on Alanine Scan search

| | | | |
|---|---|---|---|
| CRNS1 | VVGAGGVSK | HLA-A*11:01 | 0.74512 |
| | (SEQ ID | | |
| | NO.: 118) | | |
| RASE | VVGASGVGK | HLA-A*11:01 | 0.690829 |
| | (SEQ ID | | |
| | NO.: 119) | | |
| RAB7B | IVGAIGVGK | HLA-A*11:01 | 0.688497 |
| | (SEQ ID | | |
| | NO.: 120) | | |
| RASH | VVGAGGVGK | HLA-A*11:01 | 0.638055 |
| (same as KRAS | (SEQ ID | | |
| peptide of | NO.: 80) | | |
| SEQ ID | | | |
| NO.: 80) | | | |
| ITA8 | IVGAFGTGK | HLA-A*11:01 | 0.561758 |
| | (SEQ ID | | |
| | NO.: 122) | | |
| MIRO2 | VVGARGVGK | HLA-A*11:01 | 0.480429 |
| | (SEQ ID | | |
| | NO.: 123) | | |
| ARRD4 | AVGAEGRVK | HLA-A*11:01 | 0.328175 |
| | (SEQ ID | | |
| | NO.: 124) | | |
| SMC5 | IVGANGTGK | HLA-A*11:01 | 0.308533 |
| | (SEQ ID | | |
| | NO.: 125) | | |
| MOGS | EVGAKGQLK | HLA-A*11:01 | 0.113675 |
| | (SEQ ID | | |
| | NO.: 126) | | |
| GKN2 | NVGAGGCAK | HLA-A*11:01 | 0.06972 |
| | (SEQ ID | | |
| | NO.: 127) | | |

Based on XScan search

| | | | |
|---|---|---|---|
| AFDDT | QMGAAGSGR | HLA-A*11:01 | 0.00474 |
| | (SEQ ID | | |
| | NO.: 128) | | |
| ANKR9 | PVGAAGSAR | HLA-A*11:01 | 0.002816 |
| | (SEQ ID | | |
| | NO.: 129) | | |
| CFTR | VAGSTGAGK | HLA-A*11:01 | 0.150817 |
| | (SEQ ID | | |
| | NO.: 130) | | |
| DYH2 | IVGCTGSGK | HLA-A*11:01 | 0.069626 |
| | (SEQ ID | | |
| | NO.: 131) | | |
| EP300 | MNGSIGAGR | HLA-A*11:01 | 0.002772 |
| | (SEQ ID | | |
| | NO.: 132) | | |
| FOXA2 | AAGAAGSGK | HLA-A*11:01 | 0.271597 |
| | (SEQ ID | | |
| | NO.: 133) | | |
| HTR5B | ASGAVGSAK | HLA-A*11:01 | 0.701693 |
| | (SEQ ID | | |
| | NO.: 134) | | |
| LARP1 | AAGAAGAGR | HLA-A*11:01 | 0.036351 |
| | (SEQ ID | | |
| | NO.: 135) | | |
| MED1 | NVGSTGVAK | HLA-A*11:01 | 0.425281 |
| | (SEQ ID | | |
| | NO.: 136) | | |
| MOD5 | ILGATGTGK | HLA-A*11:01 | 0.116715 |
| | (SEQ ID | | |
| | NO.: 137) | | |
| MRP5 | ICGSVGSGK | HLA-A*11:01 | 0.0073 |
| | (SEQ ID | | |
| | NO.: 138) | | |
| NAL12 | MQGAAGIGK | HLA-A*11:01 | 0.248652 |
| | (SEQ ID | | |
| | NO.: 139) | | |
| PCGF6 | TAGSVGAAK | HLA-A*11:01 | 0.271228 |
| | (SEQ ID | | |
| | NO.: 140) | | |
| RAB4B | VIGSAGTGK | HLA-A*11:01 | 0.401558 |
| | (SEQ ID | | |
| | NO.: 141) | | |
| RAB7B | IVGAIGVGK | HLA-A*11:01 | 0.688497 |
| | (SEQ ID | | |
| | NO.: 120) | | |
| SCGR4 | TCGSCGCGY | HLA-A*11:01 | 0.000111 |
| | (SEQ ID | | |
| | NO.: 142) | | |
| 3HIDH | VSGGVGAAR | HLA-A*11:01 | 0.07473 |
| | (SEQ ID | | |
| | NO.: 143) | | |
| AFDDT-2 | PMGGTGSGR | HLA-A*11:01 | 0.000336 |
| | (SEQ ID | | |
| | NO.: 144) | | |
| BOLA3 | ISGGCGAMY | HLA-A*11:01 | 0.005069 |
| | (SEQ ID | | |
| | NO.: 145) | | |
| DYH9 | VVGGAGTGK | HLA-A*11:01 | 0.496638 |
| | (SEQ ID | | |
| | NO.: 146) | | |
| KRA53 | SCGGCGSGY | HLA-A*11:01 | 0.000368 |
| | (SEQ ID | | |
| | NO.: 147) | | |
| SHRM1 | VNGSVGISR | HLA-A*11:01 | 0.010494 |
| | (SEQ ID | | |
| | NO.: 148) | | |
| KRAS G12V | VVGAVGVGK | HLA-A*11:01 | 0.755168 |
| 8-16 | (SEQ ID | | |
| (Reference) | NO.: 3) | | |

Additional studies were performed using 11N4A-TCR simulated T cell products described in Example 13 using processes resembling clinical manufacturing of drug product. All 10 self-peptides identified by alanine scan motif were re-tested using the 11N4A-TCR simulated T cell products across 2 donors. Significant T cell activation of 11N4A-TCR was not detected for any peptide, even RAB7B (FIG. 6G, left). In addition, all 12 self-peptides identified based on XScan motif (including RAB7B) were tested, with no significant T cell activation of 11N4A-TCR simulated T cell product detected for any peptide (FIG. 6G, right)

### Example 7

### Alloreactivity Screen for TCR 11N4A with or without CD8ab Shows No Alloreactivity Against B-LCLs Expressing Common HLA Alleles

(FIGS. 7A, 7B) To determine whether TCR 11N4A exhibits alloreactivity towards common non-A11 HLA alleles, sort purified primary CD8+ T cells were transduced with either a construct encoding the 11N4A TCR alone, or with an alternative construct that encodes CD8 alpha and CD8 beta genes in addition to the 11N4A TCR alpha and beta chains, and cultured overnight with a panel of B-LCL cell lines that express a diverse set of HLA alleles that are commonly found in the US population. Activation-induced CD137 expression after overnight culture was assessed by flow cytometry.

A large lymphoblastoid cell line library covering >95% of the most common HLA alleles in the U.S. population was also assessed, with no alloreactive responses detected to T cells engineered to express TCR 11N4A. The 11N4A-expressing T cells did not demonstrate cytokine-independent growth with a rapid decline in product cell numbers which was indistinguishable from untransduced T cell controls. As 11N4A-TCR uses autologous patient cells as starting material for T cell engineering, no alloreactive responses are expected with the endogenous TCR repertoire of patient T cells. In addition, a library of EBV-transformed human B-lymphoblastoid cell lines (B-LCL), which express diverse and common HILA alleles found in the human population (Table 3, below), were used to assess potential MHC class I HLA alloreactivity of primary human T cells expressing 11N4A and CD8a/B coreceptor. As the B-LCLs do not commonly express KRAS G12V, they would not be expected to trigger 11N4A-TCR recognition even in HLA-A*11:01 positive B-LCLs. Briefly, healthy donor-derived T cells were transduced with 11N4A plus CD8α/β coreceptor and individually co-cultured with each B-LCL followed by assessment of T cell activation using CD137 surface expression.

TABLE 3

| LCL line | HLA-A | | HLA-B | | HLA-C | |
|---|---|---|---|---|---|---|
| JWP | A*02:01:01 | A*33:01:01 | B*07:02:01 | B*15:01:01 | C*03:04:01 | C*07:02:01 |
| MS | A*11:01:01 | A*11:01:01 | B*13:01:01 | B*15:25:01 | C*04:03:01 | C*12:02:02 |
| JAS | A*01:01:01 | A*68:01:01 | B*44:02:01 | B*55:01:01 | C*03:03:01 | C*05:01:01 |
| LCL-19 | A*02:06:01 | A*26:01:01 | B*35:01:01 | B*38:01:01 | C*04:01:01 | C*12:03:01 |
| LCL-12 | A*02:01:01 | A*31:01:02 | B*18:01:01 | B*40:01:02 | C*03:04:01 | C*07:01:01 |
| LCL-8 | A*02:01:01 | A*68:01:01 | B*45:01:01 | B*58:02:01 | C*06:02:01 | C*16:01:01 |
| LCL-25 | A*23:01:01 | A*23:01:01 | B*41:01:01 | B*51:01:01 | C*15:02:01 | C*17:01:01 |
| LCL-5 | A*24:02:01 | A*02:06:01 | B*40:02:01 | B*55:02:01 | C*01:02:01 | C*15:02:01 |
| LCL-24 | A*02:02:01 | A*29:02:01 | B*15:16:01 | B*44:03:01 | C*14:02:01 | C*16:01:01 |
| LCL-7 | A*24:02:01 | A*31:01:02 | B*35:01:01 | B*52:01:01 | C*04:01:01 | C*12:02:02 |
| LCL-2 | A*24:02:01 | A*24:02:01 | B*48:01:01 | B*54:01:01 | C*01:02:01 | C*08:03:01 |
| PAJ | A*01:01:01 | A*03:01:01 | B*08:01:01 | B*49:01:01 | C*07:01:01 | C*07:01:01 |
| X028 | A*02:01:01 | A*03:01:01 | B*07:02:01 | B*44:02:01 | C*05:01:01 | C*07:02:01 |
| LCL-18 | A*02:01:01 | A*25:01:01 | B*08:01:01 | B*44:02:01 | C*05:01:01 | C*07:01:01 |
| LCL-4 | A*24:02:01 | A*29:01:01 | B*07:05:01 | B*27:02:01 | C*02:02:02 | C*15:05:02 |
| LCL-10 | A*34:02:01 | A*74:01:01 | B*08:01:01 | B*15:03:01 | C*02:10:01 | C*07:01:01 |
| LCL-23 | A*32:01:01 | A*68:02:01 | B*08:01:01 | B*44:02:01 | C*01:02:01 | C*07:01:01 |
| LCL-3 | A*30:01:01 | A*68:02:01 | B*42:01:01 | B*42:01:01 | C*17:01:01 | C*17:01:01 |
| LCL-16 | A*03:01:01 | A*03:01:01 | B*27:05:02 | B*56:01:01 | C*01:02:01 | C*02:02:02 |
| LCL-20 | A*01:01:01 | A*11:01:01 | B*50:01:01 | B*51:01:01 | C*06:06:01 | C*15:02:01 |
| LCL-13 | A*02:01:01 | A*02:05:01 | B*15:01:01 | B*49:01:01 | C*03:03:01 | C*07:01:01 |
| LCL-6 | A*02:05:01 | A*68:02:01 | B*14:02:01 | B*58:01:01 | C*07:18:01 | C*08:02:01 |
| LCL-17 | A*01:01:01 | A*03:01:01 | B*08:01:01 | B*35:01:01 | C*04:01:01 | C*07:01:01 |
| LCL-9 | A*36:01:01 | A*74:01:01 | B*53:01:01 | B*57:03:01 | C*04:01:01 | C*07:01:02 |
| X009 | A*02:01:01 | A*23:01:01 | B*27:05:02 | B*44:03:01 | C*01:02:01 | C*04:01:01 |
| LCL-11 | A*30:01:01 | A*33:01:01 | B*53:01:01 | B*81:01:01 | C*04:01:01 | C*08:04:01 |
| DUCAF | A*30:02:01:01 | | B*18:01:01:01 | | C*05:01:01:01 | |
| KAS116 | A24:02:01:01 | | B*51:01:01:01 | | C*12:03:01:01 | |
| SAVC | A*03:01:01:01 | | B*07:02:01 | | C*07:02:01:03 | |
| KAS011 | A*01:01:01:01 | | B*37:01:01 | | C*06:02:01:01 | |
| WT8 | A*03:01:01:01 | | B*07:02:01 | | C*07:02:01:03 | |
| VAVY | A*01:01:01:01 | | B*08:01:01:01 | | C*07:01:01:01 | |
| SA | A*24:02:01:01 | | B*07:02:01 | | C*07:02:01:03 | |
| MGAR | A*26:01:01:01 | | B*08:01:01:01 | | C*07:01:01:01 | |
| SCHU | A*03:01:01:01 | | B*07:02:01 | | C*07:02:01:03 | |
| JBUSH | A*32:01:01 | | B*38:01:01 | | C*12:03:01:01 | |
| QBL | A*26:01:01:01 | | B*18:01:01:01 | | C*05:01:01:01 | |
| DEU | A*31:01:02:01 | | B*35:01:01:02 | | C*04:01:01:01 | |

To assess whether the coverage of this B-LCL library is sufficiently representative of the HLA genetic diversity in a clinically relevant patient population for a Phase 1 study, United States HLA allele frequencies across five broad terms describing individual races (African American, Asian and Pacific Islander, Caucasian, Hispanic, Native American) were extracted from the BeTheMatch website (Gragert et al., 2013). Using these terms, demographic count values were obtained from the US 2020 census for both single and multi-race identifying responders. Census responders identifying as multiple races were divided by the number of identified races, and those fractional counts added to respective single race sums. Using this method, the fraction of each reported race in the U.S. was calculated. BeTheMatch HLA allelic frequencies per race were adjusted according to the fraction of each race in the U.S. population. The unique set of HLA alleles were extracted from the B-LCL library per loci (HLA-A, HLA-B, and HLA-C) and the allelic frequencies were summed. The summed allelic frequencies were converted to genotypic frequencies according to the Hardy-Weinberg model to calculate population coverage represented by the library. Shown in Table 4 is the percentage of the U.S. population containing at least one of the alleles that was also present within the B-LCL cell line library for each HLA loci (A, B, and C). For HLA-A, HLA-B and HLA-C alleles, >96.2% of the US population has at least one of these alleles. These values are proposed to be sufficiently representative of the genetic diversity in the U.S. population to have clinical relevance.

TABLE 4
Percentage of the U.S. population containing at least one of the
alleles contained in the B-LCL cell line at each HLA loci

| Loci | U.S. Population Coverage in B-LCL library |
|---|---|
| HLA-A | 99.6% |
| HLA-B | 96.2% |
| HLA-C | 99.7% |

### Example 8

### Specific Killing Activity of CD4+ T Cells Expressing TCR 11N4A and a CD8 Co-Receptor

(FIG. 8) CD4+ and CD8+ T cells were transduced to express TCR 11N4A and a CD8αβ co-receptor. Killing activity of the engineered T cells against mKRAS: HLA-A11+ tumor cells was assessed using an IncuCyte assay.

### Example 9

### Cytotoxicity of Primary 11N4A TCR and CD8ab Coreceptor Engineered T Cells Against Various Tumor Cell Lines

(FIGS. 11A, 11B, 11C, 11D) Growth kinetics of indicated HLA-A11+, KRAS G12V-expressing tumor cell lines in a live tumor-visualization assay alone (Tumor cell only), in the presence of untransduced primary T cells (UTD) or in the presence of primary T cells transduced with TCR 11N4A and CD8αβ coreceptor (11N4A TCR+CD8αβ). Tumor cells labelled with Incucyte Nuclight Rapid Red (SW480, SW527 and SW620 tumor cells) or a expressing a green fluorescent protein (CFPAC1 tumor cells) were cultured alone or with TCR-transduced or untransduced T cells for up to 192 hours at the indicated effector to target ratio, and total red object area (for SW480, SW527 and SW620 tumor cells) or green object area (for CFPAC tumor cells) was measured as a metric of tumor cell growth and viability throughout the study as indicated. The effector: target ratio was stringent in this experiment, for example, the effector: target ratio is 0.5:1 for SW527 tumor cells; and 2:1 for CFPAC, SW480, and SW620 respectively. Additional tumor cells were added at the timepoints indicated to assess the ability of the engineered T cells to continue to respond after multiple tumor challenges. The results indicate that the engineered T cells exhibit robust cancer killing even after multiple tumor challenges and therefore are not prone to T cell exhaustion.

### Example 10

### In Vivo Anti-Tumor Efficacy of 11N4A TCR and CD8ab Co-Receptor Engineered T Cells

(FIGS. 12A, 12B, 12C) Primary CD4+ and CD8+ T cells transduced with TCR 11N4A and CD8αβ coreceptor induced robust in vivo anti-tumor activity in SW527, SW620, and CFPAC tumor challenge models. 6-8-week-old NSG immunocompromised mice were subcutaneously inoculated with tumor cell lines: SW527 (breast cancer-derived), CFPAC (pancreatic cancer-derived), and SW620 (colon cancer-derived) respectively. At Day 07 (SW527), Day 10 (CFPAC), and Day 09 (SW620) post tumor inoculation, mice were randomized and five mice per group were dosed IV with untransduced primary T cell control and primary T cells transduced with TCR 11N4A and CD8αβ coreceptor respectively. Specifically, the mice group inoculated with SW527 tumor cells were administered 3×10⁶ T cells transduced with TCR 11N4A and CD8αβ coreceptor; CFPAC1 tumor cells with 1×10⁷ T cells transduced with TCR 11N4A and CD8αβ coreceptor; and SW620 tumor cells with 3×10⁶ T cells transduced with TCR 11N4A and CD8αβ coreceptor. Tumor kinetics were tracked by caliper measurement. The results indicate that TCR 11N4A and CD8αβ coreceptor engineered T cells induce robust anti-tumor activity across multiple in vivo tumor models derived from different cancer types.

### Example 11

### In Vitro and In Vivo Anti-Tumor Efficacy of 11N4A TCR and CD8ab Co-Receptor Engineered CD4+ and CD8+ T Cells

(FIG. 13A) Growth kinetics of CFPAC-1 tumor cell line expressing HLA-A11+, KRAS G12V were tracked for 188 hours in the presence of 11N4A TCR and CD8αβ co-receptor engineered CD4+ T cells (shown as “CD4 TCR”), 11N4A TCR and CD8αβ co-receptor engineered CD8+ T cells (shown as “CD8 TCR”), or a 1:1 ratio of 11N4A TCR and CD8αβ co-receptor engineered CD4+ T cells and CD8+ T cells (shown as “CD4 TCR+CD8 TCR”) at a 2:1 effector to target ratio. CFPAC1 tumor cells expressed green fluorescent protein enabling tumor growth visualization for 188 hours by Incucyte Zoom. Additional tumor cells were added at timepoints indicated to assess the ability of 11N4A TCR and CD8αβ coreceptor engineered T cells to continue to respond after multiple tumor challenges. The results indicate that the 11N4A TCR and CD8αβ coreceptor engineered group that contains both CD4+ and CD8+ T cell subsets exhibited the highest tumor control. Without being bound by theory, this suggests a coordinated response between CD8+ and CD4+ T cells, possibly with CD4+ T cells expressing both 11N4A and CD8αβ coreceptor responding to the KRAS G12V antigen and triggering helper activity to support CD8+ T cell killing.

(FIG. 13B) 6-8-week-old NSG immunocompromised mice were intraperitoneally inoculated with 1.25×10\hat{ }5 CFPAC1-Luc tumor cells which express luciferase for in vivo detection of tumor growth. Mice were randomized and were either mock treated, or treated intraperitoneally with 11N4A TCR and CD8αβ co-receptor engineered CD4+ T cells (shown as “CD4 TCR”), 11N4A TCR and CD8αβ co-receptor engineered CD8+ T cells (shown as “CD8” TCR″), or a 1:1 ratio of 11N4A TCR and CD8αβ co-receptor engineered CD4+ T cells and CD8+ T cells (shown as “CD4 TCR+CD8 TCR”) on days 10 (7×10\hat{ }6 T cells) and 21 (8×10\hat{ }6 T cells) (N=5). Tumor kinetics were tracked by whole body IVIS imaging of tumor cell luminescence. The results indicate that the group that contains both CD4+ and CD8+ T cell subsets exhibits the highest tumor control and without being bound by theory, suggests that CD4+ T cells expressing both 11N4A and CD8αβ co-receptor are responding to the KRAS G12V antigen and triggering helper activity to support CD8+ T cell killing in vivo.

To test whether co-expression of CD8α/β coreceptor with TCR 11N4A in engineered T cells would result in improved pharmacological activity, CD4+ and CD8+ T cells from healthy donors were individually transduced with lentiviral constructs containing either TCR 11N4A or TCR 11N4A combined with CD8α/β coreceptor. Cytotoxic activity of CD4+ T cell only, CD8+ T cells only, or a 1:1 ratio of CD4+:CD8+ T cells for each of the constructs was tested against a KRAS G12V-positive ovarian cancer cell line, OVCAR5, under conditions of low effector to target ratios and repeated tumor rechallenge. Whereas individual or combined T cell subsets containing TCR 11N4A only exhibited weak cytotoxic activity (FIG. 13C), the killing activity of all groups containing TCR 11N4A combined with CD8a/B coreceptor was significantly improved (FIG. 13D). Notably, the CD4+ T cell only group expressing CD8α/β coreceptor and TCR 11N4A exhibited significant anti-tumor activity, which is uncommon for CD4+ T cells. Improvements to the anti-tumor activity of the CD8+ T cell only group were also observed by adding CD8a/B coreceptor and may be due to the higher expression of CD8a/B coreceptor enhancing the binding affinity for the KRAS G12V target. Importantly, the group with combined CD8+ and CD4+ T cells exhibited the most robust anti-tumor activity and supports the hypothesis that a coordinated CD4+/CD8+ T cell response by the TCR 11N4A+CD8a/B coreceptor T cell (1:1: CD4+/CD8+) product could enable CD4+ T cell helper activity that may sustain an anti-tumor response in the harsh tumor microenvironment (FIG. 13D). Testing the same T cell subsets expressing TCR 11N4A combined with CD8a/B coreceptor against a pancreatic cell line, PANC1, which does not express the KRAS G12V target antigen did not exhibit any killing activity (FIG. 13E), supporting low potential of TCR 11N4A to mediate off-target effects even after addition of CD8a/B coreceptor.

### Example 12

### TCR 11N4A T Cells Did not Exhibit Cytokine-Independent Growth

TCR 11N4A can be delivered using a third-generation, self-inactivating lentiviral vector for T cell engineering, a platform with extensive experience and safety demonstrated in clinical studies (Milone & O'Doherty, 2018). These advanced lentiviral vectors prefer insertion into introns and do not preferentially insert into the promoters of active genes. In addition, cytokine-independent growth studies were performed to assess the risk of T cell transformation resulting from lentiviral insertion of TCR 11N4A that could potentially lead to myeloproliferative disorders (FIG. 17). The results show that the 11N4A-TCR simulated T cell products across two donors decline in cell number over time in the absence of cytokine, similar to the untransduced (UTD) controls. No live cell counts for any of the products tested were detected from D8 to D35. These data suggest a low likelihood of TCR 11N4A transformation events that may lead to abnormal growth of the cell product.

FIG. 14 also supports safety of T cells transduced with TCR 11N4A and CD8αβ co-receptor. Following removal of exogenous cytokine support, similar to the wild-type untransduced T cells, TCR 11N4A+CD8αβ co-receptor transduced T cells did not show cytokine-independent growth in vitro. Primary T cells with TCR 11N4A and CD8αβ co-receptor do not show potential for tumorigenic transformation as assessed by cytokine-independent growth assay, further supporting safety of TCR 11N4A.

### Example 13

### Additional Characterization of a Potentially Cross-Reactive Peptide from RAB7B

To fully assess the cross-reactive potential of 11N4A-TCR for RAB7B, additional studies were performed using simulated T cell products derived from two donors. Additional peptide dose titration studies of KRAS G12V and RAB7B were tested to assess reactivity of 11N4A-TCR simulated T cell products. The simulated T cell products differ from the initial RAB7B dose titration studies above in that final construct designs were used in manufacturing including CD8 α/β coreceptor. Across both donors, only background T cell activation was detected with RAB7B (FIG. 15), consistent with FIG. 11. The lack of 11N4A-TCR simulated T cell product reactivity to RAB7B, even at high concentrations of peptide, may reflect dilution of higher avidity CD8⁺ T cells by lower avidity CD4⁺ T cell inclusion but is representative of a clinical-grade manufacturing process for 11N4A-TCR simulated T cell products. Additional studies were conducted to further assess the potential cross-reactivity of TCR 11N4A against naturally processed and presented RAB7B. HeLa or HEK293 cells were transduced to over-express full length RAB7B and HLA-A*11:01. As it is possible that RAB7B could be processed by differing tissue-dependent proteosomal subunits, HEK293 cells expressing the standard proteosome with or without exogenously expressed immunoproteosomal subunits were tested (Lahman et al., 2022). Individual CD4 or CD8 11N4A-TCR or untransduced T cell product was co-cultured with HeLa, or HEK293 cells expressing differing proteosomal subunits for a period of time and assessed for CD137 T cell activation. No T cell activation was detected against any of the RAB7B expressing cell lines tested or the PANC1 negative control (FIG. 16). The positive control CFPAC-1 KRAS G12V-positive tumor cell line was able to activate CD4 or CD8 11N4A-TCR T cells as previously described.

In summary, cross-reactivity of 11N4A to RAB7B was only detected using high concentrations of RAB7B peptide and in an engineered T cell product not fully representative of 11N4A-TCR product. Additional studies using 11N4A-TCR simulated T cell product did not detect RAB7B cross-reactivity either by peptide dose titration or by overexpressed, endogenously processed and presented RAB7B antigen in HEK293 or Hela cells. This comprehensive assessment of the potential RAB7B off-target antigen indicates a low potential for TCR 11N4A to cross-react with this target under physiological expression.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Patent Application No. 63/342,025, filed May 13, 2022, U.S. Provisional Patent Application No. 63/380,551, filed Oct. 21, 2022, and U.S. Provisional Patent Application No. 63/488,758, filed Mar. 6, 2023, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A binding protein comprising:
(a) a T cell receptor (TCR) α chain variable (Vα) domain comprising the complementarity determining region 3 (CDR3α) amino acid sequence set forth in any one of SEQ ID NOs.: 16, 17, 42, and 43, or a variant thereof having one, two, or three, optionally conservative, amino acid substitutions; and/or
(b) a TCR β chain variable (Vβ) domain comprising the CDR3β amino acid sequence set forth in any one of SEQ ID NOs.: 26, 27, 52, and 53, or a variant thereof having one, two, or three, optionally conservative, amino acid substitutions,
wherein the binding protein is capable of binding to a peptide: HLA complex, wherein the peptide comprises or consists of the amino acid sequence VVVGAVGVGK (SEQ ID NO.: 2) or VVGAVGVGK (SEQ ID NO.: 3) and wherein the HLA comprises an HLA-A*11 or HLA-A*11:01.

2. (canceled)

3. The binding protein of claim 1, wherein the Vα domain and/or the Vβ domain is human, humanized, or chimeric, and is preferably human.

4. (canceled)

5. The binding protein of claim 1 4, comprising: (i) in the Vα domain, the CDR1α amino acid sequence set forth in SEQ ID NO.: 14 or 40, or a variant thereof having one or two, optionally conservative, amino acid substitutions; (ii) in the Vα domain, the CDR2α amino acid sequence set forth in SEQ ID NO.: 15 or 41, or a variant thereof having one or two, optionally conservative, amino acid substitutions; (iii) in the Vβ domain, the CDR1β acid sequence set forth in SEQ ID NO.: 24 or 50, or a variant thereof having one or two, optionally conservative, amino acid substitutions; (iv) in the Vβ domain, the CDR2β acid sequence set forth in SEQ ID NO.: 25 or 51, or a variant thereof having one or two, optionally conservative, amino acid substitutions; or (v) any combination of (i)-(iv).

6. The binding protein of claim 1, comprising the CDR1α, CDR2α, CDR3α, CDR1β, CDR2β, and CDR3β amino acid sequences set forth in SEQ ID NOs.: 14, 15, 16 or 17, 24, 25, and 26 or 27, respectively, or the CDR1α, CDR2α, CDR3α, CDR1β, CDR2β, and CDR3β amino acid sequences set forth in SEQ ID NOs.: 40, 41, 42 or 43, 50, 51, and 52 or 53, respectively.

7. (canceled)

8. The binding protein of claim 1, wherein:
(i) the Vα domain comprises or consists of an amino acid sequence having at least 85%, identity to the amino acid sequence set forth in SEQ ID NO.: 13 or 39; and/or
(ii) the Vβ domain comprises or consists of an amino acid sequence having at least 85%, identity to the amino acid sequence set forth in SEQ ID NO.: 23 or 49.

9.-10. (canceled)

11. The binding protein of claim 1, wherein the Vα domain comprises or consists of the amino acid sequence set forth in SEQ ID NO.: 13 and the Vβ domain comprises or consists of the amino acid sequence set forth in SEQ ID NO.: 23; or wherein the Vα domain comprises or consists of the amino acid sequence set forth in SEQ ID NO.: 39 and the Vβ domain comprises or consists of the amino acid sequence set forth in SEQ ID NO.: 49.

12. (canceled)

13. The binding protein of claim 1, further comprising a TCR α chain constant domain (Cα) and/or a TCR β chain constant domain (Cβ).

14. The binding protein of claim 13, wherein the Cα comprises or consists of an amino acid sequence having at least 90%, identity to, or comprising or consisting of, the amino acid sequence set forth in any one of SEQ ID NOs.: 18, 19, 44, 45, and 69; and/or wherein the Cβ comprises or consists of an amino acid sequence having at least 90% identity to, or comprising or consisting of, the amino acid sequence set forth in any one of SEQ ID NOs.: 28, 29, 54, 55, and 70-73.

15.-16. (canceled)

17. The binding protein of claim 13, wherein the Cα, the Cβ, or both comprise modification(s) that promote preferential pairing of the Cα to the Cβ; or wherein the Cα and the Cβ each comprises an introduced cysteine residue that promotes preferential pairing of the Cα to the Cβ; or wherein the Cα comprises a T48C substitution and the Cβ comprises a S57C substitution to promote preferential pairing of the Cα to the Cβ.

18.-19. (canceled)

20. The binding protein of claim 1, comprising a TCR α chain and a TCR β chain, wherein the TCR α chain and the TCR β chain comprise or consist of amino acid sequences having at least 90%, identity to, or comprising or consisting of, the amino acid sequences set forth in:
(i) SEQ ID NOs.: 12 and 22, respectively;
(ii) SEQ ID NOs.: 20 and 30, respectively;
(iii) SEQ ID NOS.: 12 and 30, respectively;
(iv) SEQ ID NOs.: 20 and 22, respectively;
(v) SEQ ID NOs.: 38 and 48, respectively;
(vi) SEQ ID NOs.: 46 and 56, respectively;
(vii) SEQ ID NOs.: 38 and 56, respectively; or
(viii) SEQ ID NOs.: 46 and 48, respectively.

21. The binding protein of claim 1, wherein the binding protein comprises a TCR, a single-chain TCR (scTCR), a single-chain T cell receptor variable fragment (scTv), or a chimeric antigen receptor (CAR).

22.-23. (canceled)

24. An isolated polynucleotide encoding the binding protein of claim 1.

25. The polynucleotide of claim 24, comprising a polynucleotide having at least 90%, identity to, or comprising or consisting of, the polynucleotide sequence set forth in any one of SEQ ID NOs.: 5-10 and 33-36, or any combination thereof.

26. The polynucleotide of claim 24, further comprising:
(i) a polynucleotide encoding a polypeptide that comprises an extracellular portion of a CD8 co-receptor α chain, wherein, optionally, the encoded polypeptide is or comprises a CD8 co-receptor α chain;
(ii) a polynucleotide encoding a polypeptide that comprises an extracellular portion of a CD8 co-receptor β chain, wherein, optionally, the encoded polypeptide is or comprises a CD8 co-receptor β chain; or
(iii) a polynucleotide of (i) and a polynucleotide of (ii).

27. (canceled)

28. The polynucleotide of claim 26, further comprising a polynucleotide that encodes a self-cleaving peptide and is disposed between:
(1) the polynucleotide encoding a binding protein and the polynucleotide encoding a polypeptide comprising an extracellular portion of a CD8 co-receptor α chain; and/or
(2) the polynucleotide encoding a binding protein and the polynucleotide encoding a polypeptide comprising an extracellular portion of a CD8 co-receptor β chain.

29.-32. (canceled)

33. The polynucleotide of claim 24, encoding an amino acid sequence having at least 90% identity to, or comprising or consisting of the amino acid sequence set forth in any one of SEQ ID NOs.: 11, 21, 37, 47, 31, 32, 57, and 58.

34.-36. (canceled)

37. An expression vector, comprising the isolated polynucleotide of claim 24 operably linked to an expression control sequence.

38. (canceled)

39. The expression vector of claim 37, wherein the encoded binding protein comprises a TCR α chain and a TCR β chain, and wherein the expression control sequence drives expression of a single mRNA encoding a polypeptide comprising an extracellular portion of a CD8 co-receptor α chain, a polypeptide comprising an extracellular portion of a CD8 co-receptor β chain, the TCR α chain, and the TCR β chain.

40.-47. (canceled)

48. A host cell modified to comprise the polynucleotide of claim 24.

49. The host cell of claim 48, wherein the modified cell comprises a hematopoietic progenitor cell and/or a human immune cell.

50. A host cell expressing the binding protein of claim 1.

51. The host cell of claim 49, wherein the immune cell comprises a T cell, a CD4⁺ T cell, a CD8⁺ T cell, a CD4⁻CD8⁻ double negative T cell, a γδ T cell, a naïve T cell, a central memory T cell, a stem cell memory T cell, an effector memory T cell, or any combination thereof,
wherein, optionally, the immune cell comprises a CD4⁺ T cell and a CD8⁺ T cell, wherein, further optionally, the CD4⁺ T cell, the CD8⁺ T cell, or both comprise (i) a polynucleotide encoding a polypeptide that comprises an extracellular portion of a CD8 co-receptor α chain, wherein, optionally, the encoded polypeptide is or comprises a CD8 co-receptor α chain; (ii) a polynucleotide encoding a polypeptide that comprises an extracellular portion of a CD8 co-receptor β chain, wherein, optionally, the encoded polypeptide is or comprises a CD8 co-receptor β chain; or (iii) a polynucleotide of (i) and a polynucleotide of (ii).

52. The host cell of claim 48, wherein the modified cell comprises a chromosomal gene knockout of a PD-1 gene; a LAG3 gene; a TIM3 gene; a CTLA4 gene; an HLA component gene; a TIGIT gene; a TCR component gene, a FasL gene, or any combination thereof.

53.-54. (canceled)

55. A composition comprising the host cell of claim 48 and a pharmaceutically acceptable carrier, diluent, or excipient.

56.-57. (canceled)

58. A composition comprising:
the binding protein of claim 1; or a polynucleotide encoding the binding protein,
and a pharmaceutically acceptable carrier, excipient, or diluent.

59. A method for treating a disease or disorder associated with a KRAS G12V mutation or a NRAS G12V mutation or a HRAS G12V mutation in a subject, the method comprising administering to the subject an effective amount of:
the binding protein of claim 1, a polynucleotide encoding the binding protein of claim 1, an expression vector comprising a polynucleotide encoding the binding protein of claim 1, a host cell comprising or expressing the binding protein of claim 1, or a pharmaceutical composition comprising: the binding protein of claim 1, a polynucleotide encoding the binding protein of claim 1, an expression vector comprising a polynucleotide encoding the binding protein of claim 1, or a host cell comprising or expressing the binding protein of claim 1; and a pharmaceutically acceptable carrier, excipient, or diluent.

60. The method of claim 59, wherein the disease or disorder comprises a cancer, wherein the cancer is optionally a solid cancer or a hematological malignancy.

61.-69. (canceled)

70. The method of claim 59, further comprising determining that the subject expresses HLA-A*11, optionally HLA-A*11:01, prior to administering the binding protein, the encoding polynucleotide, or the pharmaceutically acceptable composition.

71.-75. (canceled)

76. The binding protein of claim 1, comprising:
(i) a TCR α-chain and a TCR β-chain, wherein the TCR α-chain comprises the amino acid sequence set forth in SEQ ID NO.: 13 and the TCR β-chain comprises the amino acid sequence set forth in SEQ ID NO.: 23 or 154; or
(ii) a TCR α-chain and a TCR β-chain, wherein the TCR α-chain comprises the amino acid sequence set forth in SEQ ID NO.: 20 and the TCR β-chain comprises the amino acid sequence set forth in SEQ ID NO.: 155.

77. A binding protein comprising a T cell receptor (TCR) or a single-chain TCR variable fragment (scTv), the binding protein comprising
a TCR α chain variable (Vα) domain comprising the complementarity determining region (CDR)1α, CDR2α, and CDR3α amino acid sequences set forth in SEQ ID NOs.: 14, 15, and 16 or 17, respectively, and
a TCR β chain variable (Vβ) domain comprising the CDR1β, CDR2β, and CDR3β amino acid sequences set forth in SEQ ID NOs.: 24, 25, and 26 or 27, respectively.
